(19) United States
(12) Patent Application Publication
(10) Pub. No.: US 2026/0237462 A1
(43) Pub. Date: Aug. 13, 2026

(54) COLORECTAL ADENOMA AND CARCINOMA BIOMARKER DISCOVERY, FUNCTIONAL ANALYSIS, AND DIAGNOSTICS

(71) Applicant: Prescient Metabiomics JV, LLC, Carlsbad, CA (US)

(72) Inventors: Thomas J. KUEHN, Carlsbad, CA (US); Scott N. PETERSON, Carlsbad, CA (US); Alexey M. EROSHKIN, Carlsbad, CA (US); Piotr Z. KOZBIAL, Carlsbad, CA (US); Ermanno FLORIO, Carlsbad, CA (US); Gregory J. KUEHN, Carlsbad, CA (US)

(21) Appl. No.: 19/471,135

(22) PCT Filed: Mar. 29, 2024
(86) PCT No.: PCT/US2024/022123

§ 371 Date: Sep. 30, 2025

Related U.S. Application Data
(60) Provisional application No. 63/455,698, filed on Mar. 30, 2023.

(51) Int. Cl. G16B 30/10; C12Q 1/48; C12Q 1/6837; C12Q 1/6851; C12Q 1/686; C12Q 1/6869; G16B 40/20; G16B 40/30; G16H 10/40; G16H 10/60; G16H 50/20; G16H 50/70

(52) U.S. Cl. CPC G16B 30/10; C12Q 1/48; C12Q 1/6837; C12Q 1/6851; C12Q 1/686; C12Q 1/6869; C12Y 207/07; G16B 40/20; G16B 40/30; G16H 10/40; G16H 10/60; G16H 50/20; G16H 50/70; C12Q 2600/112

(57) ABSTRACT

The present disclosure in various aspects and embodiments provides methods for evaluating subjects for the presence or absence of colorectal neoplasia, such as colorectal cancer (CRC), colorectal adenoma (CRA), and colorectal advanced adenoma (CRAA), by metagenomic and multiomic analysis of fecal or other biological samples. In other aspects, the present disclosure provides methods for generating machine learning models or “signatures” based on metagenomic and multiomic analysis of fecal or other biological samples, to evaluate subjects for the presence or absence of colon disorders, including but not limited to CRC, CRA, and CRAA.

### PRIORITY

This application claims priority to and the benefit of U.S. provisional application No. 63/455,698 filed Mar. 30, 2023, which is hereby incorporated by reference in its entirety.

### SEQUENCE LISTING

The application contains a sequence listing, which has been submitted in XML format via EFS-Web. The contents of the XML copy named “MBI-003PC 108458-5003_Sequence_Listing”, which was created on Mar. 29, 2024 and is 73,728 bytes in size, the contents of which are incorporated herein by reference in their entirety.

### BACKGROUND

Colorectal cancers are among the most prevalent cancers worldwide with an estimated 1.8 million new colon cancer cases and over 700,000 rectal cancer cases reported in 2018. Bray et al., Global cancer statistics 2018: GLOBOCAN estimates of incidence and mortality worldwide for 36 cancers in 185 countries, CA Cancer J Clin. 2018; 68 (6): 394-424. Further, colon cancer is now the leading cause of death in men under 50. Siegel R L, et al. Cancer Statistics, 2024, CA: A Cancer Journal for Clinicians (2024). Despite the strong evidence demonstrating that screening of individuals with average CRC risk reduces mortality, compliance amongst individuals is limited due to the invasiveness, discomfort and fear associated with colonoscopy. Lauby-Secretan et al., The IARC Perspective on Colorectal Cancer Screening, N Engl J Med. 2018; 378 (18): 1734-1740. This has created a significant gap in the health care system and CRC prevention in particular, emphasizing the need for sensitive, accurate, non-invasive diagnostics to detect colon adenomas and carcinomas. The present disclosure fills this gap by providing biomarkers derived from microbial species present in stool and other samples.

### BRIEF DESCRIPTION OF DRAWINGS

FIG. 1A is a Principal Coordinate Analysis (PCoA) plot of microbiota profiles derived from samples within the studies analyzed. FIG. 1B is a PCoA plot of microbiota profiles derived from samples for each disease class. FIG. 1C is a PCoA plot of microbiota profiles derived from samples within the studies analyzed after supervised normalization.

FIG. 1D is a PCoA plot of microbiota profiles derived from samples for each disease class after supervised normalization.

FIG. 2 is a cross-correlation plot. Samples from the studies listed at the top were used individually to train models for CRC. These models were used to predict samples (test set) from each study.

FIG. 3 shows a workflow that illustrates two distinct feature selection methods, feature importance rank ensembling (FIRE) and Statistical Inference of Associations between Microbial Communities and host phenotypes (SIAMCAT), separately or in combination.

FIG. 4 illustrates the independent feature selection method SIAMCAT. Features are ranked according to significance scores. Shown are box plots displaying the relative abundance of samples to visualize differential representation, fold-change, prevalence shift and feature contribution to area under the curve (AUC).

FIG. 5 shows the performance assessment obtained using average AUCs obtained based on a combination of taxonomic and gene features (the KEGG Ortholog (KO) groups, or taxa associated with colorectal adenoma (CRA), colorectal advanced adenoma (CRAA) or colorectal cancer (CRC)) and the feature selection methods FIRE and SIAMCAT applied separately and together.

FIG. 6 shows Venn Diagrams showing top 800, 500, 200, 100, 50 and 20 features generated from a combination of FIRE and SIAMCAT for CRA, CRAA and CRC. The number and % of total of overlapping features between disease classes is shown. The number of features corresponding to taxonomic (T) and gene features (K) are shown separately. The number of features analyzed are shown in decreasing order from left to right and top to bottom (800, 500, 200, 100, 50 and 20 features).

FIG. 7 shows Venn Diagrams illustrating a direction of change in the features. A Venn Diagram of the number of overlapping features for 800 taxonomic and gene features generated from a combination of FIRE and SIAMCAT for CRA, CRAA and CRC is shown in the center plot. The surrounding diagrams consider the direction of change in a pairwise manner to illustrate similarities and differences between features common to disease classes. (FC)=fold-change.

FIG. 8 shows the differential representation of bacterial taxonomic classes across disease classes. The number of features at the class level were summed as either over-(positive values) or under-represented (negative values) compared to control samples.

FIG. 9 shows the differential representation of bacterial families across disease classes. The number of features at the family level were summed as either over-(positive values) or under-represented (negative values) compared to control samples. The families differentiate CRAA from control and other disease classes.

FIG. 10 shows a cladogram illustrating important taxonomic features that show differential representation in CRA, CRAA, and CRC.

FIG. 11 shows the representation of virulence determinants adherence, biofilm formation, invasins, virulence factor (VF) regulator, LPS, secretion system and virulence effectors that are over-represented in CRA, CRAA and CRC compared to healthy controls.

FIG. 12A and FIG. 12B are Euclidean Distance plots illustrating the distance from centroids calculated for all samples within each study for all taxonomic ranks based on relative abundance (FIG. 12A) and following supervised normalization (FIG. 12B).

FIG. 13A-N are graphs showing quantitative detection of CRC taxonomic features by sequencing and PCR: (A) Fusobacterium nucleatum, (B) Streptococcus salivarius, (C) Parvimonas micra, (D) Roseburia intestinalis, (E) Eubacterium ventriosum, (F) Clostridium symbiosum, (G) Gemella morbillorum, (H) Cloacibacillus evryensis, (I) Bacteroides stercoris, (J) Butyricimonas virosa, (K) Collinsella stercoris, (L) Fecalibacterium prausnitzii, (M) Intestimonas butyriciproducens, and (N) Vaillonella parvula.

FIG. 14A-Q are graphs showing quantitative detection of CRA taxonomic features by sequencing and PCR: (A) Bacteroides salyersiae, (B) Dorea formicigenerans, (C) Ruminococcus bicirculans, (D) Clostridium spiroforme, (E) Alistipes shahii, (F) Dorea longicatena, (G) Gemella sanguinis, (H) Streptococcus thermophilus, (I) Bifidobacterium animalis, (J) Bifidobacterium pseudocatenulatum, (K) Escherichia coli, (L) Gordonibacter pamelaeae, (M) Parabacteroides goldsteinii, (N) Bifidobacterium adolescentis, (O) Bacteroides cellulosilyticus, (P) Bacteroides caccae, and (Q) Bacteroides nordii.

FIG. 15A-E are graphs showing quantitative detection of CRAA taxonomic features by sequencing and PCR: (A) Intestinibacter bartletti, (B) Bacteroides xylanisolvens, (C) Bacteroides thetaiotaomicron, (D) Flavinofactor plautii, and (E) Mogibacterium diversum.

### DETAILED DESCRIPTION

The present disclosure in various aspects and embodiments provides methods for evaluating subjects for the presence or absence of colorectal neoplasia, such as colorectal cancer (CRC), colorectal adenoma (CRA), and colorectal advanced adenoma (CRAA), by metagenomic and multi-omic analysis of biological samples such as fecal, blood, serum, plasma, urine, saliva, biopsy tissues, mucosa tissue sample or swab, intestinal lavage or aspirant, and other biofluids and cell samples (referred to herein as “biological samples”) containing human and microbiome DNA, RNA, Proteins, and other molecules for molecular analysis. In other aspects, the present disclosure provides methods for generating machine learning models or “signatures” (biomarker profiles or patterns) based on metagenomic and multi-omic analysis of biological samples, including fecal samples, to evaluate subjects for the presence or absence of colon disorders, such as but not limited to CRC, CRA, and CRAA.

CRC is a heterogeneous disease, the majority of which are considered sporadic without underlying heritable features. Frank et al., Concordant and discordant familial cancer: Familial risks, proportions and population impact, Int J Cancer 2017; 140 (7): 1510-1516. A wide variety of environmental factors including a western diet, obesity, cigarette smoking, alcohol consumption and lack of exercise are known CRC risk factors. Chief amongst these risk factors is diet where an estimated ~38% of incipient CRC cases were linked. Additional evidence for environmental influence of CRC is based on findings that the incidence of CRC is influenced by emigration, wherein a subject's risk of CRC development is altered based on the diet and lifestyle of the recipient country. Each of the above mentioned CRC risk modifiers is also known to modulate the composition of the gut microbiota. Greathouse et al., Gut microbiome meta-analysis reveals dysbiosis is independent of body mass index in predicting risk of obesity-associated CRC, BMJ Open Gastroenterol 2019; 6 (1): e000247; Lee et al., Association between Cigarette Smoking Status and Composition of Gut Microbiota: Population-Based Cross-Sectional Study, J Clin Med 2018; 7 (9): 282; Rodriguez-Gonzalez et al., Microbiota and Alcohol Use Disorder: Are Psychobiotics a Novel Therapeutic Strategy?, Curr Pharm Des 2020; 26 (20): 2426-2437; Allen et al., Exercise Alters Gut Microbiota Composition and Function in Lean and Obese Humans, Med Sci Sports Exerc 2018; 50 (4): 747-757. This association has drawn substantial attention to the gut microbiota as a potential mediator of CRC initiation and/or progression. The large number of species and genes encoded in the gut microbiome represents a source of potential biomarkers for diagnostics and prognostics of early, premalignant adenomas, advanced adenomas, and CRC.

In various aspects and embodiments, the present disclosure enables detection of colorectal adenoma (CRA), colorectal advanced adenoma (CRAA) and/or colorectal cancer (CRC) (as well as other colon disorders) based on the composition of a subject's microbiome (e.g., as present in fecal or other biological samples such as mucosal tissue samples) and/or other multi-omic molecular analytes. In various embodiments, the present disclosure provides taxonomic and gene features to distinguish healthy subjects from those with early and advanced adenomas and those with carcinomas. In some aspects, the present disclosure provides machine learning (ML) models that avoid a variety of pitfalls associated with metagenomic and multi-omic data (e.g., data heterogeneity, noise, overfitting, etc.), including metagenomic and multi-omic data collected using heterogeneous methods and analysis procedures.

The methods disclosed herein leverage the most informative biomarkers for each disease class. For example, in some embodiments the methods provide features of high importance for distinguishing CRC, CRA, or CRAA from healthy controls. While CRC features were disproportionately reliant on taxonomic features, CRA and CRAA features were more balanced in representation of gene and taxonomic features. As disclosed herein, the optimal features for each disease class display little overlap, indicating that the adenoma to carcinoma progression reflects unique selective environments for microbiota that does not follow a simple linear relationship.

In one aspect, the present disclosure provides a method for evaluating a biological subject for the presence of a colorectal neoplasm. In this aspect, the disclosure provides a method for screening subjects as an alternative to invasive procedures such as colonoscopy, to thereby increase screening compliance, and enable early detection of neoplasms. In various embodiments the method comprises quantifying genetic elements from a biological sample from the subject, such as a fecal sample. Other biological samples (e.g., mucosal tissue samples, blood, saliva) that allow for sampling of the microbiome, including the gut microbiome, can also be used. The genetic elements are associated with colorectal cancer (CRC), colorectal adenoma (CRA), or colorectal advanced adenoma (CRAA), and which can be selected using machine learning models as described herein. The genetic elements comprise elements associated with microbial taxonomic classification and elements associated with one or more microbial gene functions. In this manner, the process prepares an abundance profile of the genetic elements, and the abundance profile is evaluated for a signature indicating the presence or absence of CRC, CRA, and/or CRAA in the subject. The subject can therefore be identified as likely to have (or not have) CRC, CRA, and/or CRAA. The process can provide a binary classification (i.e., presence of absence) or a statistical output indicating the likelihood that the subject has CRC, CRA, or CRAA. In various embodiments, the method provides for improved detection of adenomas (CRA and/or CRAA) over known detection tests.

Among the non-invasive CRC detection tests that have been described is the fecal immunochemical test (FIT) that is associated with limited sensitivity of 79% for detecting CRC and a poor sensitivity (~25%) for the detection advanced adenomas. Lee et al., Accuracy of fecal immunochemical tests for colorectal cancer: systematic review and meta-analysis, Ann Intern Med 2014; 160 (3): 171; Hundt et al., Comparative evaluation of immunochemical fecal occult blood tests for colorectal adenoma detection, Ann Intern Med 2009; 150(3):162-9. A multi-target stool assay quantitatively examines KRAS mutations, aberrant NDRG4 and BMP3 methylation, along with β-actin and hemoglobin immunoassays. This assay performs better than FIT, detecting CRC cases (~92% compared to 74%) with greater sensitivity, whereas advanced premalignant lesions were still poorly detected by both assays (~42% and ~24% respectively). Imperiale et al., Multitarget stool DNA testing for colorectal-cancer screening, N Engl J Med. 2014; 370 (14): 1287-97. These outcomes highlight another important gap in the healthcare system: the relatively poor ability of existing non-invasive methods to detect early and advanced adenomas. The development of a diagnostic that addresses this gap could significantly improve the detection of pre-malignant lesions and reduce the number of colonoscopies required for average risk subjects.

In some embodiments, the subject is at low risk for CRC or colorectal polyps such as CRA or CRAA. In such embodiments, low risk individuals screened according to the present disclosure can avoid or delay more invasive colonoscopy procedures. That is, the method can be performed as a screening process as an alternative to colonoscopy. According to these embodiments, low risk subjects can be screened at lower cost and at higher efficiency to the healthcare system, and subjects thereby identified where colonoscopy or other treatments are more warranted. “Low risk subjects” (as understood in the art) are subjects with no previous incidence of colorectal cancer or polyps (e.g., a colonoscopy was previously performed on the subject without detecting colorectal cancer or polyps, such as CRA or CRAA), and do not have a family history of colorectal cancer or colorectal polyps. In some embodiments, subjects at low risk do not have an inflammatory bowel disease such as Crohn's disease or ulcerative colitis. In various embodiments, the subject at low risk is at least 45 years of age, or at least 50 years of age, or at least 55 years of age, or at least 60 years of age. In some embodiments, the subject at low risk is less than 75 years of age or less than 70 years of age or less than 65 years of age. In still other embodiments, the subject is less than 45 years of age or less than 50 years of age.

In other embodiments, the subject is high or medium risk for CRC or colorectal polyps (as understood in the art). In such embodiments, these subjects can be more frequently monitored for development of colorectal neoplasia, enabling early detection and treatment without frequent colonoscopies. For example, subjects at high or medium risk include those with prior incidence of CRC or colorectal polyps (e.g., CRA or CRAA), and/or family history of CRC or colorectal polyps. In some embodiments, subjects at high or medium risk have an inflammatory bowel disease such as Crohn's disease or ulcerative colitis. In various embodiments, the method is performed at a determined frequency, such as at least about annually or at least about every other year. In some embodiments, the method is performed at least twice per year. In various embodiments, the subject of high or medium risk is at least 45 years of age, or at least 50 years of age, or at least 55 years of age, or at least 60 years of age. In some embodiments, the subject at high or medium risk is at least 65 years of age, or at least 70 years of age, or at least 75 years of age. In still other embodiments, the subject is less than 45 years of age or less than 50 years of age.

In various embodiments, the genetic elements from a biological sample (such as but not limited to a fecal sample) are quantified by nucleic acid sequencing, which can include genomic sequencing and/or RNA sequencing (e.g., cDNA sequencing). In various embodiments, the nucleic acid sequencing comprises shotgun metagenomic sequencing, targeted amplicon sequencing, and/or hybridization capture probe sequencing, among any other sequencing technique.

Several studies have examined gut microbiota using either 16S rDNA, shotgun metagenomic, targeted amplicon-based or hybridization capture probe sequencing. These studies have explored fecal and mucosal-associated populations and different stages along the adenoma, carcinoma progression. A meta-analysis of fecal or other microbiota samples datasets resulted in the identification of seven bacterial species enriched in CRC: Bacteroides fragilis, Fusobacterium nucleatum, Parvimonas micra, Porphyromonas assacharolytica, Prevotella intermedia, Alistipes finegoldii, and Thermoanaeroovibrio acidaminovorans. Dai, et al., Multi-cohort analysis of colorectal cancer metagenome identified altered bacteria across populations and universal bacterial markers, Microbiome 2018; 6 (1): 70. A separate pair of meta-analyses identified an expanded set of twenty-nine species enriched over eight distinct geographical regions. Thomas et al., Metagenomic analysis of colorectal cancer datasets identifies cross-cohort microbial diagnostic signatures and a link with choline degradation, Nat Med 2019; 25 (4): 667-678; Wirbel et al., Meta-analysis of fecal metagenomes reveals global microbial signatures that are specific for colorectal cancer, Nat Med 2019; 25 (4): 679-689. A number of studies have analyzed the human gut microbiota associated with colonic tumors and normal adjacent tissue leading to the identification of dysbiotic signatures associated with CRC. While specific taxa vary from study to study, some common themes include the frequent identification of elevated relative abundance of E. coli, Fusobacterium nucleatum, and enterotoxin-producing Bacteroides fragilis (ETBF) strain (Arthur et al., Intestinal inflammation targets cancer-inducing activity of the microbiota, Science 2012; 338 (6103): 120-3; Kostic et al., Fusobacterium nucleatum potentiates intestinal tumorigenesis and modulates the tumor-immune microenvironment, Cell Host Microbe 2013; 14 (2): 207-15; Wu et al., A human colonic commensal promotes colon tumorigenesis via activation of T helper type 17 T cell responses, Nat Med 2009; 15 (9): 1016-22. Additional taxa associated with CRC have also been identified, but they are less uniformly observed across studies.

An important observation established by these studies is that the magnitude of difference in relative abundance derived from tissue samples is substantially greater compared to stool samples, where differentially abundant taxa are more subtle, often relying on AI methods to decipher. A number of characteristics of fecal (and other biological samples comprising microbiota) present specific challenges in the identification of diagnostic biomarkers for the early detection of adenomas and carcinomas, including high dimensionality, data sparsity, and generalizability. Despite the massive quantity of DNA sequence data generated in shotgun metagenomic sequence analysis of stool samples, for example, the best performing biomarkers obtained in such analyses are detected in only a relatively small number of samples. This defines the problem of data sparsity and dictates that a high-performance diagnostic based on next-generation sequencing (NGS) sequence data requires multiple independent biomarkers to compensate for low prevalence of any single biomarker in the human population.

Thus, in various embodiments, the metagenomic sequencing is deep sequencing of genomic DNA isolated from the fecal sample or other biological sample. In various embodiments, the nucleic acid sequencing involves sequencing at least about 20,000,000 reads (i.e., raw reads per fecal sample). In various embodiments, the nucleic acid sequencing involves sequencing at least about 25,000,000 reads, or at least about 30,000,000 reads, or at least about 40,000,000 reads, or at least about 50,000,000 reads, or at least about 60,000,000 reads, or at least about 75,000,000 reads, or at least about 100,000,000 reads per sample. Well known quality control metrics can be utilized to remove low quality reads, which are generally less than about 15%, or less than about 10% of the raw reads. Generally, the reads will have less than about 5% or less than about 4%, or less than about 3%, or less than about 2% human reads. In various embodiments, human reads are removed from the analysis.

In various embodiments, the nucleic acid sequencing comprises one or more of shotgun metagenomic sequencing, rDNA sequencing, and targeted nucleic acid sequencing (e.g., targeted amplicon sequencing or hybridization capture probe sequencing). In embodiments, the nucleic acid sequencing includes multiple workflows, for example, may comprise rDNA sequencing, and one or more of shotgun sequencing, and targeted nucleic acid sequencing. Sequencing can be conducted using any known library preparation protocol, including by employing sample tags for a multiplex workflow. See for example, U.S. Pat. Nos. 8,603,749 and 9,453,262, which are hereby incorporated by reference in their entireties.

In some embodiments, library preparation from DNA samples for sequencing employs total DNA isolated from fecal or other biological samples (e.g., GI mucosal samples). Numerous kits for making sequencing libraries from DNA are available commercially. In some embodiments, library preparation comprises: fragmentation of the DNA, end-repair, addition of sequencing adapters (e.g., by ligation or amplification), and amplification to enrich for products that have adapters ligated to both ends. For example, DNA can be fragmented such that the mean fragment size is in the range of 100 base pairs to about 5000 base pairs, such as in the range of about 250 bps to about 4000 bps, or the range of about 500 bps to about 3000 bps, or in the range of about 1000 bps to about 3000 bps. In some embodiments, the mean fragment size is less than 1000 bps, such as in the range of 200 to 1000 bps (e.g., 200 to 500 bps). To facilitate multiplexing, different barcoded adapters can be used with different biological samples (e.g., from different subjects). In some embodiments, barcodes can be introduced at the PCR amplification step by using different barcoded PCR primers to amplify different biological samples. The library may be subject to shotgun metagenomic sequencing in some embodiments.

In some embodiments, the nucleic acid sequencing focuses on one or more genomic loci to allow for taxonomic analysis, including rDNA analysis. Analysis of rDNA (genes encoding rRNA) can include 16S rDNA, 18S rDNA, and internal transcribed spacer (ITS) sequencing. 16S and ITS sequence analysis allows for taxonomic analysis of bacteria and archaea, and 18S and ITS sequence analysis allows for taxonomic analysis of eukaryotes (e.g., fungi). The 16S rRNA gene comprises nine variable regions interspersed throughout the highly conserved 16S sequence. In some embodiments, sub-regions of the gene are amplified by targeted PCR for sequencing, ranging from single variable regions, such as V4 or V6, to three variable regions, such as V1 to V3 or V3 to V5. Similarly, 18S rRNA genes comprise variable regions (V1 to V9) which can be used to discriminate at the family, order, genus, and species (and sub-species) levels as is known in the art. In some embodiments, sub-regions of the gene are amplified by targeted PCR for sequencing, ranging from single variable regions to a plurality of variable regions. The ITS lies between the large and small rRNA subunit gene loci, and can be species specific. This polymorphism is due to the presence of tRNA genes. The ITS region can be amplified by targeted PCR for sequencing and taxonomic analysis.

In some embodiments, 16S/18S/ITS sequences are clustered based on similarity to generate operational taxonomic units (OTUs). Representative OTU sequences can be compared with reference databases to determine taxonomy. In some embodiments, sequences of >95% identity are considered to represent the same genus, whereas sequences of >97% identity are considered to represent the same species. Methods of determining OTUs are known in the art. In some embodiments, strain or subspecies are further distinguished based on analysis of polymorphisms. Taxonomic analysis of 16S, 18S, and ITS DNA sequences is well known in the art. See Ze-Gang Wei et al., Comparison of Methods for Picking the Operational Taxonomic Units From Amplicon Sequences, Front. Microbiol., 24 Mar. 2021.

Sequence reads other than rDNA can also be analyzed to infer likely taxonomy by comparison to reference microbial genomes. Helene LCF, et al., New Insights into the Taxonomy of Bacteria in the Genomic Era and a Case Study with Rhizobia, International Journal of Microbiology Vol. 2022.

In various embodiments, sequence reads are analyzed to determine the abundance of gene functions. The sequence reads can be analyzed according to the KEGG Orthology database, or similar database. The KEGG Orthology (KO) database is a database of molecular functions represented in terms of functional orthologs. A functional ortholog is manually defined in the context of KEGG molecular networks, namely, KEGG pathway maps, BRITE hierarchies and KEGG modules. Each node of the network, such as a box in the KEGG pathway map, is given a KO identifier (called K number) as a functional ortholog defined from experimentally characterized genes and proteins in specific organisms, which are then used to assign orthologous genes in other organisms based on sequence similarity. The resulting KO grouping may correspond to a group of highly similar sequences within a limited organism group or it may be a more divergent group. Thus, in various embodiments, sequence reads are assigned a gene function (such as according to the KO database), and the abundance of the gene function determined for the biological sample.

In various embodiments, targeted genomic fragments are captured from a metagenomic library, optionally followed by amplification. For example, nucleic acid capture probes can be used that hybridize to conserved regions of rDNA or conserved regions of functional orthologs. Sequence capture allows targeted enrichment of informative DNA. In concert with NGS, capture provides an efficient strategy for high-throughput screening of regions of interest. In various embodiments, a capture strategy reduces the required sequencing depth to less than about 25,000,000 reads, or less than about 20,000,000 reads, or less than about 15,000,000 reads, or less than about 10,000,000 reads, or less than about 5,000,000 reads, or less than about 2,000,000 reads. An exemplary sequence capture protocol comprises: fragmentation of input DNA (e.g., by shearing or with use of enzymes); addition of sequencing adapters (e.g., by ligation or amplification using fusion primers) to form library molecules; incubating the library with pools of capturable oligonucleotide probes designed to target (and hybridize to) specific regions of interest within the DNA fragment library. An exemplary capturable moiety is biotin, which can be conjugated to probe oligonucleotides. Probe/target hybrids are then captured from the library (e.g., using streptavidin-coated magnetic beads). The result is a sequencing-ready library that is highly enriched for the targeted DNA.

In still other embodiments, genetic elements can be quantified by PCR (qPCR) according to known processes. For example, genus-specific or species-specific sequences can be quantitatively amplified and detected (e.g., from rDNA in the sample) as well as conserved sequences in gene function elements. In this manner, abundance profiles of genetic elements (e.g., informative features) can be constructed without a sequencing workflow.

For the detection of CRA, CRAA, and/or CRC, the number of genetic elements quantified will be sufficient to provide for a high performance test (e.g., by allowing for the analysis of numerous informative features). For example, in various embodiments, the genetic elements can be analyzed (with respect to each model) for the presence of at least about 50 features, or at least about 100 features, at least about 200 features, at least about 500 features, or at least about 800 features, or at least about 1000 features. Exemplary features for detecting CRA, CRAA, and CRC are displayed in Tables 3, 4, and 5, respectively. The number of features for each test need not be the same for each model. For example, in some embodiments the model or “signature” for detecting CRC may include at least about 500 features or at least about 750 features, or at least about 1000 features. In some embodiments, the models or signatures for detecting CRAA and CRA are significantly less, and may include less than about 500 features, such as less than about 250 features (for example, in the range of 50 to 200 features). Models with more or less features can nevertheless be constructed according to the present disclosure.

In various embodiments, the genetic elements analyzed are associated with colorectal adenoma (CRA). For example, the genetic elements can comprise one or more taxonomic or gene function features listed in Table 3. In various embodiments, the genetic elements comprise at least five taxonomic or gene function features listed in Table 3. In some embodiments, the genetic elements comprise at least about 10, at least about 25, at least about 50, or at least about 100 taxonomic or gene function features listed in Table 3. In various embodiments, the genetic elements comprise at least one, at least two, at least five, at least about 10, at least about 20, or at least about 50 taxonomic features listed in Table 3; and at least one, at least two, at least five, at least about 10, at least about 20, or at least about 50 gene function features listed in Table 3. As shown in Table 3, certain genetic elements have differential abundance in samples (e.g., fecal samples or other biological samples) from CRA subjects (as compared to controls), and other genetic elements have differential prevalence in fecal or other biological samples from CRA subjects (as compared to control subjects). In some embodiments, the genetic elements include a plurality of those having differential abundance in CRA, and a plurality of those having differential prevalence in CRA. In some embodiments, the difference in relative abundance between disease and non-disease samples (or vice versa) is at least about 1.1 fold, or at least about 1.2 fold, or at least about 1.3 fold, or at least about 1.4 fold, or at least about 1.5 fold, or at least about 2 fold. In some embodiments, the difference in prevalence between disease and non-disease samples (or vice versa) is at least about 1.1 fold, or at least about 1.2 fold, or at least about 1.3 fold, or at least about 1.4 fold, or at least about 1.5 fold, or at least about 2 fold.

In various embodiments, the genetic elements are associated with colorectal advanced adenoma (CRAA). For example, the genetic elements can comprise one or more taxonomic or gene function features listed in Table 4. In various embodiments, the genetic elements comprise at least five taxonomic or gene function features listed in Table 4. In some embodiments, the genetic elements comprise at least about 10, at least about 25, at least about 50, or at least about 100 taxonomic or gene function features listed in Table 4. In various embodiments, the genetic elements comprise at least one, at least two, at least five, at least about 10, at least about 20, or at least about 50 taxonomic features listed in Table 4; and at least one, at least two, at least five, at least about 10, at least about 20, or at least about 50 gene function features listed in Table 4. As shown in Table 4, certain genetic elements have differential abundance in fecal or other biological samples from CRAA subjects (as compared to controls), and other genetic elements have differential prevalence in fecal or other biological samples from CRAA subjects (as compared to control subjects). In some embodiments, the genetic elements include a plurality of those having differential abundance in CRAA, and a plurality of those having differential prevalence in CRAA. In some embodiments, the difference in relative abundance between disease and non-disease biological samples (or vice versa) is at least about 1.1 fold, or at least about 1.2 fold, or at least about 1.3 fold, or at least about 1.4 fold, or at least about 1.5 fold, or at least about 2 fold. In some embodiments, the difference in prevalence between disease and non-disease biological samples (or vice versa) is at least about 1.1 fold, or at least about 1.2 fold, or at least about 1.3 fold, or at least about 1.4 fold, or at least about 1.5 fold, or at least about 2 fold.

In various embodiments, the genetic elements are associated with colorectal cancer (CRC). For example, the genetic elements can comprise one or more taxonomic or gene function features listed in Table 5. In various embodiments, the genetic elements comprise at least five taxonomic or gene function features listed in Table 5. In some embodiments, the genetic elements comprise at least about 10, at least about 25, at least about 50, or at least about 100 taxonomic or gene function features listed in Table 5. In various embodiments, the genetic elements comprise at least one, at least two, at least five, at least about 10, at least about 20, or at least about 50 taxonomic features listed in Table 5; and at least one, at least two, at least five, at least about 10, at least about 20, or at least about 50 gene function features listed in Table 5. As shown in Table 5, certain genetic elements have differential abundance in fecal or other biological samples from CRC subjects (as compared to controls), and other genetic elements have differential prevalence in fecal or other biological samples from CRC subjects (as compared to control subjects). In some embodiments, the genetic elements include a plurality of those having differential abundance in CRC, and a plurality of those having differential prevalence in CRC. In some embodiments, at least five, or at least ten, or at least 20 genetic elements for detecting CRC correspond to bacterial species that generally reside in the oral cavity. In some embodiments, the difference in relative abundance between disease and non-disease samples (or vice versa) is at least about 1.1 fold, or at least about 1.2 fold, or at least about 1.3 fold, or at least about 1.4 fold, or at least about 1.5 fold, or at least about 2 fold. In some embodiments, the difference in prevalence between disease and non-disease samples (or vice versa) is at least about 1.1 fold, or at least about 1.2 fold, or at least about 1.3 fold, or at least about 1.4 fold, or at least about 1.5 fold, or at least about 2 fold.

In various embodiments, the abundance profile of genetic elements is evaluated for signatures indicating the presence or absence of each of CRC, CRA, and CRAA. As disclosed herein, the microbiome profile for CRC, CRA, and CRAA do not exhibit linear relationship with one another, and therefore each are optimally evaluated using separate models or signatures.

In various embodiments, the signature is generated from a training set using a machine learning (ML) model. For example, the signature indicating the presence or absence of CRA is trained with fecal or other biological samples from a CRA cohort and biological samples from a control cohort. The signature indicating the presence or absence of CRAA is trained with fecal or other biological samples from a CRAA cohort and biological samples from a control cohort. The signature indicating the presence or absence of CRC is trained with fecal or other biological samples from a CRC cohort and biological samples from a control cohort. In each instance, the control cohort is considered a healthy cohort, that is, defined by the absence of CRC, CRA, and CRAA. In some embodiments, samples of the control cohort are not from subjects having significant gastrointestinal ailments, such as Crohn's disease or ulcerative colitis.

According to the various aspects and embodiments of this disclosure, the training set comprises at least about 50 samples, or at least about 100 samples, or at least about 150 samples, or at least about 200 samples, or at least about 500 samples, or at least about 1000 samples that are positive for CRA, CRAA, or CRC. In some embodiments, the training set comprises at least about 25 non-disease or healthy controls, or at least about 50 non-disease or healthy controls, or at least about 100 non-disease or healthy controls, or at least about 500 non-disease or healthy controls, or at least about 1000 non-disease or healthy controls. One of skill in the art will be able to assemble training sets representing disease and control samples in a manner that results in adequate statistical powering. The training set need not be sourced from a single study or geographic area. In some embodiments, biological samples are sourced and/or processed at different geographies (e.g., at least two different countries or continents). In these embodiments, the separate procurement, processing, or sequencing provides added diversity of research protocols, and may also provide subject genetic, ethnic, and/or environmental variation (including variation in diet).

Signatures can be trained using one or a plurality of machine learning algorithms. In some embodiments, at least one of the machine learning algorithms utilized is a supervised machine learning algorithm. In these or other embodiments, the machine learning algorithms comprise one or more of unsupervised or semi-supervised machine learning. Various machine learning algorithms are known and can be used according to the present disclosure, including but not limited to one or more of parametric/non-parametric distance measures, logistic regression, support vector machines, decision trees, random forests, neural networks, probit regression, Fisher's linear discriminant, Naive Bayes classifier, perceptron, quadratic classifiers, kernel estimation, k-nearest neighbor, learning vector quantization, and principal components analysis. In embodiments, the machine learning employs an AI-enabled, massively parallel computational and automated machine learning platform and workflow (computer program) for comparative machine learning modeling, optimization, testing, evaluation, and ranking of models, such as one or more of deep learning, gradient boosted, neural networks, ensemble, or blender modeling algorithms, such as but not limited to Gradient Boosted Trees Classifier, eXtreme Gradient Boosted Trees Classifiers, Light Gradient Boosted Trees Classifiers, Light Gradient Boosting on Elastic Net Predictions, Keras Slim Residual Neural Network Classifiers, Generalized Additive Models, Elastic Net Classifiers, Random Forest Classifiers, Deep Forest Classifiers, Average Blender Classifiers, TensorFlow Multilayer Perceptron Classifiers, TensorFlow Neural Network Classifiers, and Rule-Fit Classifiers.

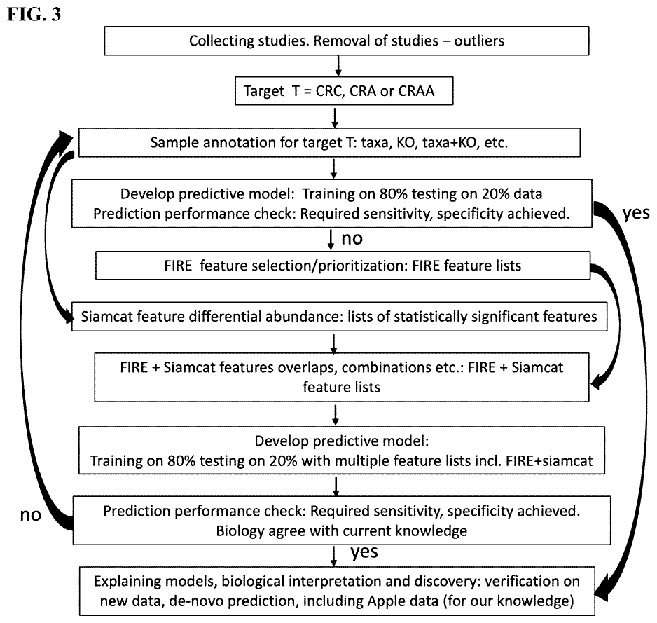

Features can be selected using any known process. In some embodiments, the signature(s) comprise features selected from training cohorts by ensemble ranking of feature importance, which ranks features according to their importance in a predictive model. Alternatively or in addition, features are selected according to their statistical significance (individually) for predicting CRA, CRAA, or CRC. For example, individual features can be selected whose abundance or prevalence is predictive of the presence or absence of CRC, CRA, or CRAA with a p-value less than or equal to 0.05 in the training group, or a p-value less than or equal to 0.01, or a p-value less than or equal to 0.005, or a p-value less than or equal to 0.001 in the training group (or other selected statistical threshold). In some embodiments, the signatures comprise features selected from training cohorts by Feature Importance Rank Ensembling (FIRE) and by statistical inference of associations between microbial communities and phenotypes (SIAMCAT). For example, overlapping features from both processes can be selected. An iterative process for feature selection is shown diagrammatically in FIG. 3.

Accuracy of a model can be assessed using the standard Receiving Operator Characteristics (ROC) curve analysis to calculate true positive, false positive, true negative and false negative rates, overall accuracy and area under the curve. The term “ROC” or “ROC curve,” refers to a Receiver Operator Characteristic curve. A ROC curve can be a graphical representation of the performance of a binary classifier system. For any given method, a ROC curve can be generated by plotting the sensitivity against the specificity at various threshold settings. Furthermore, provided at least one of three parameters (e.g., sensitivity, specificity, and the threshold setting), a ROC curve can determine the value or expected value for any unknown parameter. The unknown parameter can be determined using a curve fitted to a ROC curve. For example, provided the presence/absence or abundance of one or more features, the expected sensitivity and/or specificity of a test can be determined. The term “AUC” or “ROC-AUC” can refer to the area under a receiver operator characteristic curve. This metric can provide a measure of diagnostic utility of a method, considering both the sensitivity and specificity of the method. A ROC-AUC can range from 0.5 to 1.0, where a value closer to 0.5 can indicate a method has limited diagnostic utility (e.g., lower sensitivity and/or specificity) and a value closer to 1.0 indicates that the method has greater diagnostic utility (e.g., higher sensitivity and/or specificity).

In various embodiments, the signature(s) have a sensitivity for classifying samples for the presence or absence of CRA, CRAA, or CRC of at least about 0.70, or at least about 0.75, or at least about 0.80, or at least about 0.90, or at least about 0.95. In embodiments, the signature(s) have a specificity for classifying samples for the presence or absence of CRA, CRAA, or CRC of at least about 0.70, or at least about 0.75, or at least about 0.80, or at least about 0.90, or at least about 0.95. For example, the signatures can provide a sensitivity for classifying each of CRA, CRAA, and CRC with a sensitivity of at least 0.75 and a sensitivity of at least 0.75. For example, the signatures may have an area under the curve (AUC) for classifying samples for the presence or absence of CRA, CRAA, or CRC of at least about 0.70, or at least about 0.75, or at least about 0.80, or at least about 0.90, or at least about 0.95.

In various embodiments, if the subject is not identified according to the process described herein as likely to have CRA, CRAA, or CRC, no further procedure is conducted. That is, the subject is not scheduled for a colonoscopy or other evaluation for colorectal cancer or adenoma. Where the subject is identified as likely having one or more of CRA, CRAA, or CRC according to the processes described herein, a tailored diagnostic or treatment plan is initiated. For example, the subject can undergo a procedure that involves imaging of the colon, such as colonoscopy or CT colonography (or other scan or imaging technique) to confirm the result, which can also involve removal of one or more polyps and/or obtaining a biopsy of growths suspected of involving colorectal cancer. Where colorectal cancer is confirmed, the subject is treated for CRC. For example, the subject can undergo one or more of surgery (e.g., cancer resection, including partial colectomy in some embodiments), chemotherapy, radiation therapy, and immunotherapy for colorectal cancer. Exemplary chemotherapy or immunotherapy for colorectal cancer may include one or more of 5-fluorouracil (5-FU), capecitabine (XELODA) (which is metabolized by the tumor to 5-FU), irinotecan, leucovorin, oxaliplatin, cetuximab, panitumumab, regorafenib, bevacizumab, aflibercept, and ramucirumab. Exemplary combination therapies further comprise FOLFOX (5-FU, leucovorin, and oxaliplatin), FOLFIRI (leucovorin, 5-FU, and irinotecan), CAPEOX (capecitabine and oxaliplatin), FOLFOXIRI (leucovorin, 5-FU, oxaliplatin, and irinotecan), 5-FU with leucovorin or capecitabine alone, and trifluridine and tipiracil combination (LONSURF). In some embodiments, the subject receives an immune checkpoint inhibitor, such as an antibody or other molecule that inhibits PD-1, PD-L1, PD-L2, or cytotoxic T-lymphocyte-associated protein 4 (CTLA-4).

Further, radiation therapy can be used in conjunction with resection, chemotherapy, immunotherapy, or alone. Types of radiation therapy include External-Beam Radiation Therapy (EBRT), Internal Radiation Therapy (brachytherapy), Endocavitary radiation therapy, Interstitial brachytherapy, and Radioembolization.

In other aspects, the present disclosure provides a method for preparing a genetic signature of genetic elements (i.e., informative features) indicative of the presence of a colorectal neoplasm. The method comprises providing a training cohort of fecal or other samples from subjects confirmed to have CRA, CRAA, or CRC (or providing RNA or DNA isolated therefrom), and conducting genomic nucleic acid sequencing of DNA isolated from the fecal or other biological samples as already described. A gene signature is then trained that classifies samples for the presence or absence of CRA, CRAA, or CRC. In this aspect, the gene signature comprises microbial taxonomic classification features and microbial gene function features as described above and as exemplified in Table 3 to 5. In this aspect, the method can employ any sample suitable for evaluating the microbiome of the cohort, including fecal samples as well as other biological samples, including human biofluids (e.g., blood, serum, plasma, urine, saliva), tissues, mucosa, and cell samples.

As described, the nucleic acid sequencing may comprise one or more of shotgun metagenomic sequencing, rDNA sequencing, and targeted nucleic acid sequencing, including targeted amplicon sequencing and hybridization capture probe sequencing. Any sequencing technique can be employed. The genetic elements in the samples are assigned to a reference genome for taxonomic classification (which can include rDNA analysis), and/or genetic elements are assigned to a gene function (as already described). Taxonomic and gene function features can also be analyzed at the protein level using known methods.

For example, microbial taxonomic classification features and microbial gene function features are selected that have a differential abundance or differential prevalence in fecal or other biological samples from CRA subjects, as compared to control subjects. In some embodiments, the features comprise at least five taxonomic and/or gene function features, and which are optionally listed in Table 3. The features may comprise at least about 10, at least about 25, at least about 50, or at least about 100 taxonomic or gene function features, which are optionally listed in Table 3. In some embodiments, the features comprise at least one, at least two, at least five, at least about 10, at least about 20, or at least about 50 taxonomic features that are optionally listed in Table 3; and at least one, at least two, at least five, at least about 10, at least about 20, or at least about 50 gene function features that are optionally listed in Table 3.

In some embodiments, microbial taxonomic classification features and microbial gene function features are selected that have a differential abundance or differential prevalence in fecal or other biological samples from CRAA subjects, as compared to control subjects. In some embodiments, the features comprise at least five taxonomic and/or gene function features, and which are optionally listed in Table 4. The features may comprise at least about 10, at least about 25, at least about 50, or at least about 100 taxonomic or gene function features, which are optionally listed in Table 4. In some embodiments, the features comprise at least one, at least two, at least five, at least about 10, at least about 20, or at least about 50 taxonomic features that are optionally listed in Table 4; and at least one, at least two, at least five, at least about 10, at least about 20, or at least about 50 gene function features that are optionally listed in Table 4.

In some embodiments, microbial taxonomic classification features and microbial gene function features are selected that have a differential abundance or differential prevalence in fecal or other biological samples from CRC subjects, as compared to control subjects. In some embodiments, the features comprise at least five taxonomic and/or gene function features, and which are optionally listed in Table 5. The features may comprise at least about 10, at least about 25, at least about 50, or at least about 100 taxonomic or gene function features, which are optionally listed in Table 5. In some embodiments, the features comprise at least one, at least two, at least five, at least about 10, at least about 20, or at least about 50 taxonomic features that are optionally listed in Table 5; and at least one, at least two, at least five, at least about 10, at least about 20, or at least about 50 gene function features that are optionally listed in Table 5.

In various embodiments, at least three gene signatures are trained that: classify samples for the presence or absence of CRA, classify samples for the presence or absence of CRAA, and classify samples for the presence or absence of CRC. For example, the signature classifying samples for the presence or absence of CRA is trained with fecal or other biological samples from a CRA cohort and samples from a control cohort by machine learning. The signature classifying samples for the presence or absence of CRC is trained with fecal or other biological samples from a CRC cohort and samples from a control cohort by machine learning. The signature classifying samples for the presence or absence of CRAA is trained with fecal or other biological samples from a CRAA cohort and samples from a control cohort by machine learning. The machine learning can be as already described, and can include supervised machine learning, unsupervised machine learning, or semi-supervised machine learning, or a combination thereof.

Features can be selected as already described. For example, the signature(s) may comprise features selected from the training cohorts by ensemble ranking of feature importance. Alternatively, or in addition, features are selected according to their statistical significance (individually) for predicting CRA, CRAA, or CRC. For example, individual features can be selected based on their abundance or prevalence being significantly predictive of the presence or absence of CRC, CRA, or CRAA, demonstrated by a p-value less than or equal to 0.05 in the training group, or a p-value less than or equal to 0.01, or a p-value less than or equal to 0.005, or a p-value less than or equal to 0.001 in the training group (or any selected statistical threshold). In some embodiments, the signatures comprise features selected from training cohorts by Feature Importance Rank Ensembling (FIRE) and by statistical inference of associations between microbial communities and phenotypes (SIAMCAT).

In various embodiments, the signature(s) created have a sensitivity for classifying samples for the presence or absence of CRA, CRAA, or CRC of at least about 0.70, or at least about 0.75, or at least about 0.80, or at least about 0.90, or at least about 0.95. In various embodiments, the signature(s) created have a specificity for classifying samples for the presence or absence of CRA, CRAA, or CRC of at least about 0.70, or at least about 0.75, or at least about 0.80, or at least about 0.90, or at least about 0.95. For example, the signatures can provide a sensitivity for classifying each of CRA, CRAA, and CRC with a sensitivity of at least 0.75. For example, the signatures created may have an area under the curve (AUC) for classifying samples for the presence or absence of CRA, CRAA, or CRC of at least about 0.70, or at least about 0.75, or at least about 0.80, or at least about 0.90, or at least about 0.95.

In other aspects, the present disclosure provides a method for preparing a genetic signature of fecal (or other biological sample) genetic elements indicative of a colon disorder (including but not limited to CRA, CRAA, and CRC). In various embodiments, the method comprises providing a training cohort of fecal or other biological samples from subjects confirmed to have a colon disorder and control subjects (or DNA isolated therefrom) and conducting genomic nucleic acid sequencing of DNA isolated from the samples (as already described). A gene signature is then trained that classifies samples for the presence of the colon disorder, and for the absence of the colon disorder. According to this aspect, the gene signature comprises features selected from training cohorts by ensemble ranking of feature importance and by statistical significance of individual features.

For example, the signature(s) may comprise features selected from the training cohorts by ensemble ranking of feature importance as well as according to their statistical significance (individually) for predicting the colon disorder. For example, individual features can be selected whose abundance or prevalence is predictive of the presence or absence of the colon disorder with a p-value less than or equal to 0.05 in the training group, or a p-value less than or equal to 0.01, or a p-value less than or equal to 0.005, or a p-value less than or equal to 0.001 in the training group (or other selected statistical threshold). In some embodiments, the signatures comprise features selected from training cohorts by Feature Importance Rank Ensembling (FIRE) and by statistical inference of associations between microbial communities and phenotypes (SIAMCAT).

In various embodiments, the colon disorder is selected from Crohn's disease, ulcerative colitis, irritable bowel syndrome (IBS), diverticulitis, colorectal adenoma (CRA), colorectal advanced adenoma (CRAA), and colorectal cancer (CRC).

In various embodiments, the features comprise microbial taxonomic classification features and microbial gene function features as already described. Exemplary taxonomic and gene function features are shown in Tables 3, 4, and 5 for CRA, CRAA, and CRC respectively. For example, microbial taxonomic classification features and microbial gene function features are selected that have a differential abundance or differential prevalence in fecal or other samples from colon disorder subjects, as compared to control subjects. In some embodiments, the features comprise at least five taxonomic and/or gene function features. In some embodiments, the features comprise at least about 10, at least about 25, at least about 50, or at least about 100 taxonomic or gene function features. In embodiments, the features comprise at least one, at least two, at least five, at least about 10, at least about 20, or at least about 50 taxonomic features; and at least one, at least two, at least five, at least about 10, at least about 20, or at least about 50 gene function features.

In various embodiments, the signature(s) are trained using one or more machine learning algorithms as already described including supervised machine learning, unsupervised machine learning, or semi-supervised machine learning, or a combination thereof.

In various embodiments, the signature(s) generated have a sensitivity for classifying samples for the presence or absence of the colon disorder of at least about 0.70, or at least about 0.75, or at least about 0.80, or at least about 0.90, or at least about 0.95. In various embodiments, the signature(s) generated have a specificity for classifying samples for the presence or absence of the colon disorder of at least about 0.70, or at least about 0.75, or at least about 0.80, or at least about 0.90, or at least about 0.95. For example, the signatures created may have an area under the curve (AUC) for classifying samples for the presence or absence of the colon disorder of at least about 0.70, or at least about 0.75, or at least about 0.80, or at least about 0.90, or at least about 0.95.

As used herein, the term “about”, unless the context requires otherwise, means±10% of an associated numerical value.

Other aspects and embodiments of the invention will be apparent from the following non-limiting examples.

### EXAMPLES

### Example 1: Materials and Methods

Data normalization and visualization by PCoA. Discrete taxonomical counts were normalized using weighted trimmed mean of M-values (TMM) using the edgeR package and converted into log-counts per million (log-CPM) using Voom implemented in the ‘limma’ package in R version 4.2.1. Robinson et al., edgeR: a Bioconductor package for differential expression analysis of digital gene expression data, Bioinformatics 2010; 26 (1): 139-40; Law et al., voom: Precision weights unlock linear model analysis tools for RNA-seq read counts, Genome Biol 2014; 15 (2): R29. The data were then log-transformed and further normalized using a supervised normalization method (SNM) to remove significant batch effects between projects while retaining biological differences between disease classes. Poore et al., Microbiome analyses of blood and tissues suggest cancer diagnostic approach, Nature 2020; 579 (7800): 567-574. The Supervised normalization of microarrays (SNM) method was implemented in the ‘snm’ package in R. Mecham et al., Supervised normalization of microarrays, Bioinformatics 2010; 26 (10): 1308-15. The effects of supervised normalization were visualized using Principal Coordinate Analysis (PCoA). PCoA was performed using Euclidean distances on both relative abundance values and SNM transformed count tables including taxa from all ranks (kingdom to species). Differences in the microbial composition between projects and disease class were assessed separately using the adonis function from the vegan package (available on the World Wide Web at it CRAN.R-project.org/package=vegan). Mean distance of samples from the centroids in the PCoA plots was compared between projects using the Kruskal-Wallis test.

Machine learning. Models were created using the automated machine learning platform called DataRobot (DR; available on the World Wide Web at www.datarobot.com). A python script was developed for automated data submission to DR that allows developing models for multiple datasets. The best model of all developed models was selected based on the largest area under the curve (AUC) value for external test dataset prediction. “Blender models” which are obtained using several machine learning algorithms (combining the predictions of two or more models), were not used here. For classification purpose for each target the set of samples was divided randomly into a training set (80% of samples) and a test set (20% of samples). The training set is used to develop the set of high performing predictive models in DR (using more than ten different machine learning algorithms for classification such as eXtreme Gradient Boosted Trees Classifier, Keras Slim Residual Neural Network Classifier using Training Schedule, Elastic-Net Classifier and Light Gradient Boosted Trees Classifier with Early Stopping). The developed models are used to predict the disease state in the remaining 20% of samples (data which were not used in training), and the top model (with highest external test AUC) is defined. The model performance on the test set parameters is finally supplemented with external test sensitivity, specificity, and accuracy.

DataRobot feature lists. Feature lists control the subset of features that DataRobot uses to build models. DataRobot automatically creates several feature lists for each project, including two main lists, Informative Features and DataRobot (DR)-Reduced Features. Informative Features are all features that provide information potentially valuable for modeling (normally all features). DR-Reduced features are a subset of features, selected based on the Feature Impact calculation of the best model. DR Reduced feature list consists of the features that provide 95% of the accumulated impact for the model. Though computational analysis does not require to limit the number of features, practical consideration of further laboratory analysis (by qPCR) points to using models with smaller number of features. Since Informative Features lists usually have almost all features in the dataset (1000-2000 in taxonomy annotation and 5000-10000 in functional annotation) and DR Reduced feature list have no more than 100 features, for comparative purposes we used models built using DR Reduced feature lists.

FIRE feature selection. A feature reduction and selection method “Feature Importance Rank Ensembling” (FIRE, available on the World Wide Web at www.datarobot.com/blog/using-feature-importance-rank-ensembling-fire-for-advanced-feature-selection/) was used. In this method, the features are derived from multiple diverse predictive models which were built by DR. By default, DR sorts the models by selected criterion, for example, external test AUC. The median rank of each feature is calculated by aggregating the ranks for each of the several top models (the number of top models to consider was empirically selected equal to five).

Feature Importance is calculated in DR using an algorithm that measures the information content of the variable—this calculation is done independently for each feature in the dataset. In some embodiments, the FIRE procedure comprises the following steps: (a) calculating the feature importance for the top models (e.g., 3, 4, 5, 6, 7, 8, 9, 10) (determined by the external test AUC), (b) getting the ranking of the features, (c) Computing the median rank of each feature, (d) sorting the aggregated list by the computed median rank, (e) defining the threshold number of features to select, and (f) defining a feature list based on the newly selected features, and (g) removal of redundant features selected by two or more models. By sorting the aggregated list by median rank, we derive a ranked feature importance list.

Since the optimal number of features is not known, we iteratively tested several thresholds with large increments in the first loop (800, 700, 600, 500, 400, 300, 200, 100, 90, 80, 70, 60, 50, 40, 30) and small increments around the first found threshold in the second loop (for example, 85, 84, 83, 82, 81, 79, 78, 77, 76, 75, if the best threshold from the first loop equals 80). Finally, we took the threshold that provided the highest external test AUC. We considered the maximal number of FIRE features as 800 since in some of the analyzed datasets, the total amount of features was between 800 and 900. We have implemented FIRE in a multi-step process with the goal of improving accuracy of disease class predictions and, moreover, exploiting the ensemble nature of FIRE to establish feature selection that has increased generalizability since its basis is not tied to a single model and its associated biases.

SIAMCAT feature selection. Another feature selection method is based on statistical inference of associations between microbial communities and phenotypes and is referred to as Statistical Inference of Associations between Microbial Communities And host phenoTypes (SIAMCAT) version 2.1.0. Wirbel et al., Microbiome meta-analysis and cross-disease comparison enabled by the SIAMCAT machine learning toolbox, Genome Biol 2021; 22 (1): 93. SIAMCAT is a part of the suite of computational microbiome analysis tools developed at EMBL. SIAMCAT provides the sign to the feature abundance (with p-values and adjusted p-values) so it shows the sign of the abundance change between normal and disease states. Additionally, we used an adjusted p-value (adj_pval) of 0.001 in this analysis. The raw data has been preprocessed and taxonomically profiled with the bioBakery 3 pipeline version 3.0.0a7.

Combining FIRE and SIAMCAT. Since the FIRE and SIAMCAT feature lists are selected based on different criteria (ensemble ranking for the first and statistical significance for the second), we decided to also test the lists that combine the FIRE and SIAMCAT lists together to create new, FIRE SIAMCAT feature lists.

### Example 2: Analysis of Publicly Available Data Sets

To evaluate the accuracy and feasibility of developing a non-invasive diagnostic stool test for early and advanced pre-cancerous adenomas and late-stage carcinomas, we imported data generated from 13 studies conducted by laboratories in 8 countries, analyzing stool samples using shotgun metagenomic sequencing. Wirbel et al., Meta-analysis of fecal metagenomes reveals global microbial signatures that are specific for colorectal cancer, Nat Med 2019; 25 (4): 679-689 Thomas et al., of colorectal cancer datasets identifies cross-cohort microbial diagnostic signatures and a link with choline degradation, Nat Med 2019; 25 (4): 667-678; Yu et al., Metagenomic analysis of faecal microbiome as a tool towards targeted non-invasive biomarkers for colorectal cancer, Gut 2017; 66 (1): 70-78; Feng et al., Gut microbiome development along the colorectal adenoma-carcinoma sequence, Nat Commun 2015; 6:6528; Zeller et al., Potential of fecal microbiota for early-stage detection of colorectal cancer, Mol Syst Biol 2014; 10 (11): 766; Yachida et al., Metagenomic and metabolomic analyses reveal distinct stage-specific phenotypes of the gut microbiota in colorectal cancer, Nat Med 2019; 25 (6): 968-976; Gao et al., Alterations, Interactions, and Diagnostic Potential of Gut Bacteria and Viruses in Colorectal Cancer, Front Cell Infect Microbiol 2021; 11:657867; Gupta et al., Association of Flavonifractor plautii, a Flavonoid-Degrading Bacterium, with the Gut Microbiome of Colorectal Cancer Patients in India, mSystems 2019 Nov. 12; 4 (6): e00438-19; Hale et al., Distinct microbes, metabolites, and ecologies define the microbiome in deficient and proficient mismatch repair colorectal cancers, Genome Med 2018; 10 (1): 78; Vogtmann et al., Colorectal Cancer and the Human Gut Microbiome: Reproducibility with Whole-Genome Shotgun Sequencing, PLoS One 2016; 11 (5): e0155362; Spanogiannopoulos et al., Host and gut bacteria share metabolic pathways for anti-cancer drug metabolism, Nat Microbiol 2022; 7 (10): 1605-1620; Hannigan et al., Diagnostic Potential and Interactive Dynamics of the Colorectal Cancer Virome, mBio 2018; 9 (6): e02248-18. In total, our analysis included sequence data generated from 1,705 subjects, including 703 healthy controls, 196 precancerous colorectal adenoma (CRA), 48 advanced precancerous colorectal adenoma (CRAA) and 765 colorectal carcinomas (CRC) cases all confirmed by colonoscopy. The descriptive statistics of the cohorts from each study are shown in Table 1.

To analyze publicly available data sets we first performed data normalization using weighted trimmed mean of M-values (TMM) and log-counts per million (TMM-Voom data) and further normalized using a supervised method (SNM) as described Mecham et al., Supervised normalization of microarrays, Bioinformatics 2010; 26 (10): 1308-15. The effects of supervised normalization were visualized using Principal Coordinate Analysis (PCoA). PCoA was performed using Euclidean distances on both TMM-Voom and TMM-Voom-SNM transformed count tables including taxa from all ranks (kingdom to species). Differences in the microbial composition between projects and disease class were assessed separately.

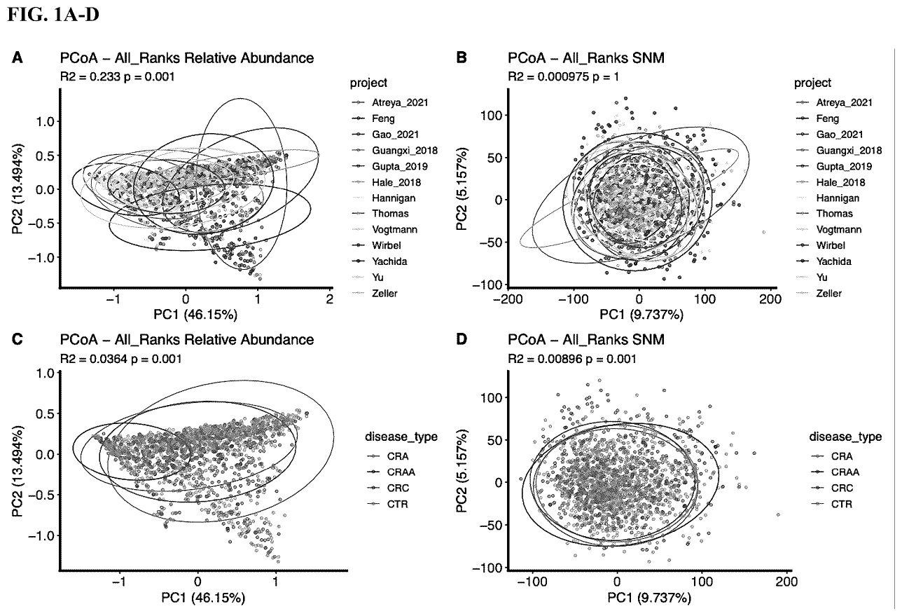

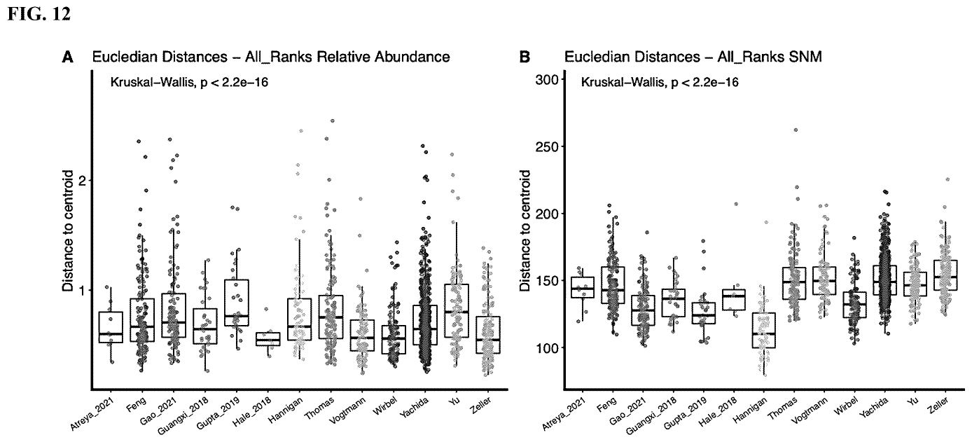

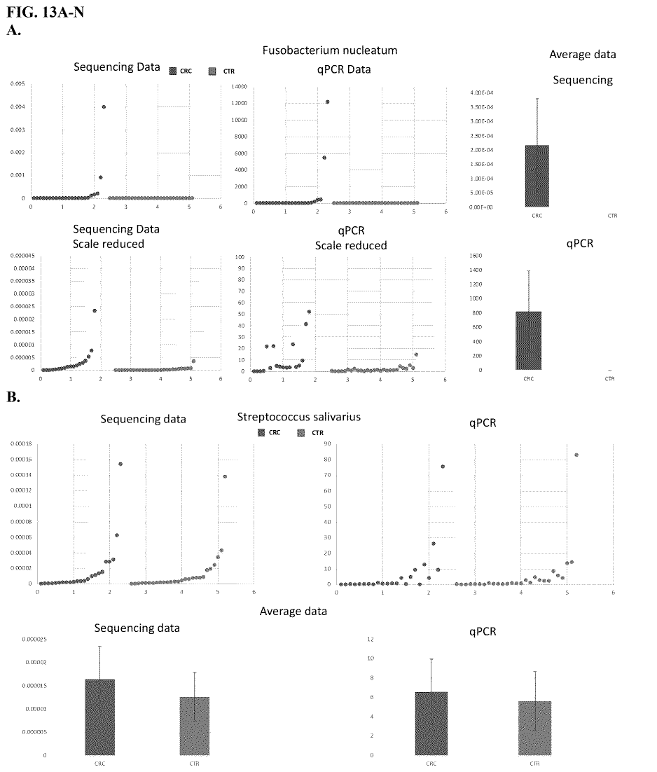

PCoA plots showed that supervised normalization significantly reduced the variation that could be explained by unique projects from an R²=10.3% to 0.0975% (FIG. 1A-B). Only 1.5% of the variation was explained by disease class using the TMM-Voom normalized data (FIG. 1C). This variation decreased to 0.896% following supervised normalization, though the difference between disease types remained significant (FIG. 1D). To identify projects and samples representing potential outliers we determined distance to centroids within each project. These distances were similar across projects for both TMM-Voom and TMM-Voom-SNM data (FIG. 12A, B). Although non-ideal, the project-specific variation was fully expected. We elected to face the challenge of performance optimization of these datasets rather than attempting to remove studies based on ad hoc criteria. Further, it is difficult to distinguish between study and country-specific effects. Therefore, the possibility that biomarkers associated with adenomas and carcinomas are prone to country or regional-specific effects remains unresolved. These results highlight the significant challenges associated with meta-analyses of gut microbiome data. These analyses illustrated, inter alia, that β-dispersion across studies were similar but large sample-to-sample variability existed within each study as expected.

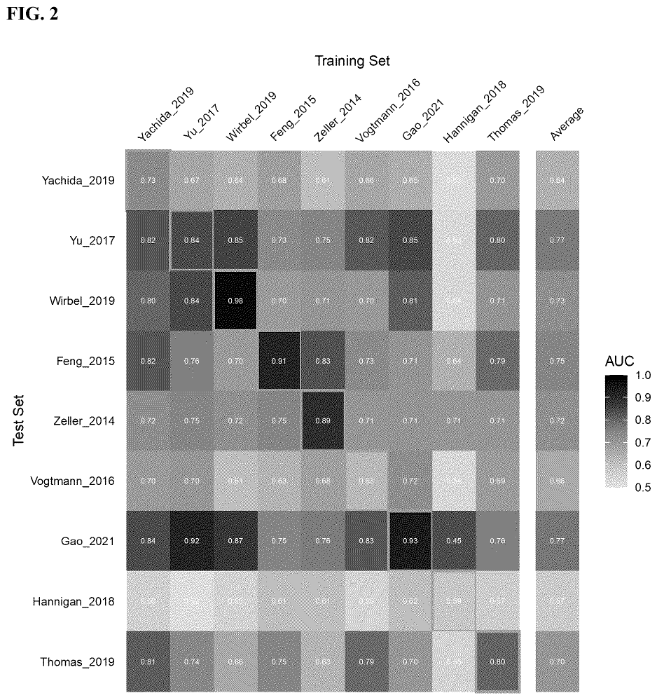

To further examine features associated with meta-analysis, namely study variability and population variability, we used data from individual studies representing different population cohorts to train models and determine how well each model predicted all other studies (FIG. 2). In most instances, models trained with data from a particular study performed well on itself, although not always the best outcome was obtained. Some studies used for training generated relatively higher AUC for test sets across all or most studies. This result may be a way to measure the generalizability of features derived from particular study populations. Other studies used as training sets predicted one or a few studies with high AUC but displayed greater variation overall. Finally, some studies performed relatively poorly across most or all studies. The variability in AUC generated in this analysis illustrate one of the primary challenges associated with meta-analysis of microbiota profiles. The reasons for cross study variability may be numerous and include sampling differences, methodological variability, geographic effects, cohort demographics, and others. This analysis showed, inter alia, that some studies generated high predictive power for CRC samples for their own study and several additional studies. In no case did any study predict CRC well across all studies. It should be noted that even the highest quality study can only perform as well as the weakest study in such an analysis. The factors contributing to study quality are variable and challenging to define.

### Example 3: Feature Generation

In our efforts to develop a stool microbiome diagnostic analysis pipeline, we focused on the evaluation of two related data features. The first, is the relative abundance of taxonomic features enumerated through the bioBakery pipeline. See, for example, McIver et al., bioBakery: a meta'omic analysis environment. Bioinformatics. 2018; 34 (7): 1235-1237. Second, we explored the inclusion of gene features derived from shotgun metagenomic sequence analysis. In this report we evaluated KO gene function annotations. The KEGG Ortholog (KO) groups are a database of molecular functions represented in terms of functional orthologs. Kanehisa et al., KEGG: new perspectives on genomes, pathways, diseases and drugs, Nucleic Acids Res 2017; 45 (D1): D353-D361. Importantly, a gene feature is often of higher relative abundance as each represents the sum of orthologous genes within the entire community. This attribute is reasoned to be potentially beneficial compared to taxonomic features that often suffer from the problem of sparsity. Without wishing to be bound by theory, we hypothesized that gene features may positively contribute to predictive performance and serve as a complementary feature to those derived from taxonomy, and we tested the hypothesis.

### Example 4: Feature Processing and Selection

We implemented strategies to evaluate a large variety of feature reduction methods to compare their overall impact on prediction accuracy. Each of these had specific strengths and weaknesses. Feature selection schemes based on filtering data to remove low prevalence features are dangerous in the context of fecal microbiota since many of the best diagnostic features (species) are of low abundance and often of low prevalence. The implication of this knowledge is the requirement to perform feature selection in such a way to retain those informative features. This fact also dictates that the best performing models are likely to require a larger number of features for optimal accuracy. Here we evaluate a feature reduction method referred to as Feature Importance Rank Ensembling (FIRE). Unique to this method, the highest-ranking features are derived from multiple diverse models. We elected to identify the best 5 models (by an external test AUC) in our ensemble procedures. We have added another feature selection method based on statistics referred to as SIAMCAT (described below) to define a novel workflow (FIG. 3). One important finding based on implementation of FIRE is that the best performing models differ according to disease class, emphasizing that no single model or set of models is optimal to distinguish health and disease (CRA, CRAA, CRC). We therefore perform FIRE using feature selection and training on CRA, CRAA and CRC samples independently to achieve target specific model optimization that in turn achieves optimal disease class-specific diagnostic performance.

As an additional layer of ensembling, we process taxonomic and gene features through the tool known as SIAMCAT, which allows visualization of differential abundance, prevalence, feature AUC and ranks features based on statistical significance. SIAMCAT-based feature selection can set any significance cut-off. In this study we used features with corrected p-values p<0.001. Not surprisingly, the features generated by FIRE and SIAMCAT partially overlap. Our pipeline combines a relatively large number of features generated by FIRE and SIAMCAT, wherein any redundancy is removed. The unique features generated after combining FIRE and SIAMCAT represent a new feature list that is used to classify samples into healthy or disease classes. In practice, any number of features may be selected, but the optimal feature number must be determined empirically (see below). In this example FIRE was run on a mixture of taxonomic and gene features, and the results from the best 5 models are displayed.

### Example 5: FIRE Feature Selection

To establish the optimal number of features, FIRE is performed iteratively starting with a large number of features, e.g. 800-1000 to establish a baseline performance based on external test AUC. We conducted these analyses for each disease target and for taxonomic and gene features separately (Table 2). We did not observe any pattern across disease classes when evaluating taxonomic features and functional gene features separately. For CRC, 800 gene features and 400 taxonomic features provided the best performance. This was strongly contrasted by CRAA and CRA analyses. For CRAA the optimum number gene features was substantially lower (40) as was the number of taxonomic features (100). For CRA we observed 40 gene features and 70 taxonomic features as optimal for performance.

These results surprisingly showed that the optimal number of features for CRC, both for taxonomic and gene features was substantially higher than that determined for CRAA and CRA (Table 2). The reason(s) for this are not clear but may reflect that colorectal tumors have the largest effect on colonic microbiota and their encoded functions, thereby creating a larger spectrum of discriminatory biomarkers. For both CRAA and CRA, a relatively small number of gene features (40) was determined to be optimal. Without wishing to be bound by theory, we speculate that this may reflect a relative paucity of discriminatory gene and taxonomic biomarkers at earlier stages of disease.

Table 3 shows the top 300 Feature List for colorectal adenoma (CRA) showing the taxonomy of the identified genera. Table 3 also shows a fold change in relative abundance compared to CRA negative samples, a prevalence shift and a rank order indicating the weight or importance of the change. The prevalence shift value between the two classes has a positive value when there is a higher prevalence in CRA and a negative value when there is a higher prevalence in the control group. In some cases, the fold change is zero, and thus the Prevalence Shift column indicates prevalence in CRA.

Table 4 shows the top 300 Feature List for colorectal advanced adenoma (CRAA) showing the taxonomy of the identified genera. Table 4 also shows a fold change in relative abundance compared to CRAA negative samples, a prevalence shift and a rank order indicating the weight or importance of the change. The prevalence shift value between the two classes has a positive value when there is a higher prevalence in CRAA and a negative value when there is a higher prevalence in the control group. In some cases, the fold change is zero, and thus the Prevalence Shift column indicates prevalence in CRAA.

Table 5 shows the top 300 Feature List for colorectal cancer (CRC) showing the taxonomy of the identified genera. Table 5 also shows a fold change in relative abundance compared to CRC negative samples, a prevalence shift and a rank order indicating the weight or importance of the change. The prevalence shift value between the two classes has a positive value when there is a higher prevalence in CRC and a negative value when there is a higher prevalence in the control group. In some cases, the fold change is zero, and thus the Prevalence Shift column indicates prevalence in CRC.

CRC taxonomic features are unique as they are highly enriched for those that are over-represented in disease and species normally resident in the oral cavity. The majority of studies examining these microbes have focused on their behavior in the oral cavity rather than the gut, but accumulating evidence suggests that these taxa are pathobionts capable of causing or contributing to disease in various contexts.

Several interesting observations can be made from this output. Perhaps most significant is the fact that the top 24 features reflect bacterial species that generally reside in the oral cavity. While it is evident from the results that these organisms exist in the healthy gut microbiota, each of these taxa display an increase in relative abundance (fold-change) in CRC relative to control and an increase in prevalence (frequency of non-zero measurements). All but 3 taxonomic features are increased in relative abundance in CRC relative to control healthy subjects. Two of these features, Roseburia intestinalis and members of the genus Anaerostipes are part of the normal commensal gut microbiota. The specific reasons for their decreased relative abundance and increased prevalence are unclear. The third case is unexpected involving Streptococcus salivarius, a known oral bacterium. The probable reason for its decreased relative abundance and increased prevalence is unclear.

The over-representation of oral microbes in CRC fecal samples is consistent with the idea that the tumor microenvironment co-selects these oral species through an unknown fitness advantage that is lacking in healthy individuals and/or a defense mechanism that becomes disabled in CRC. While the factors driving this fitness advantage may be complex, one factor that may explain these results is due to the metabolic shift occurring in colonic carcinoma epithelium that accompanies the transition from health and adenomas to carcinoma, namely that the oxygen consumption in the gut resulting from oxidative metabolism of butyrate for energy is replaced by non-oxygen consuming fermentation of lactate. One important result of this metabolic shift is increased oxygen tension in the tumor microenvironment. This increased oxygen content may be sufficient or at least one contributing factor that positively selects for the aerobic oral species observed.

### Example 6: SIAMCAT Feature Selection

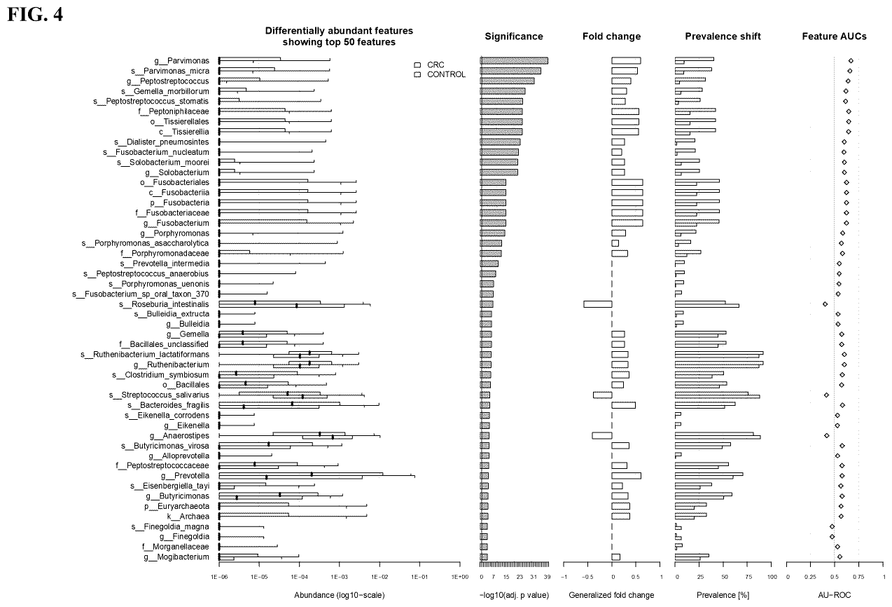

We have explored an independent method for feature selection referred to as SIAMCAT. This method computes and displays the relative abundance of each feature in all samples analyzed, the statistical significance of differentially represented features in datasets, the fold-change observed between healthy control and each disease class, the change in prevalence and the feature AUC (FIG. 4). In this example, the feature importance pertains to CRC using only taxonomic features.

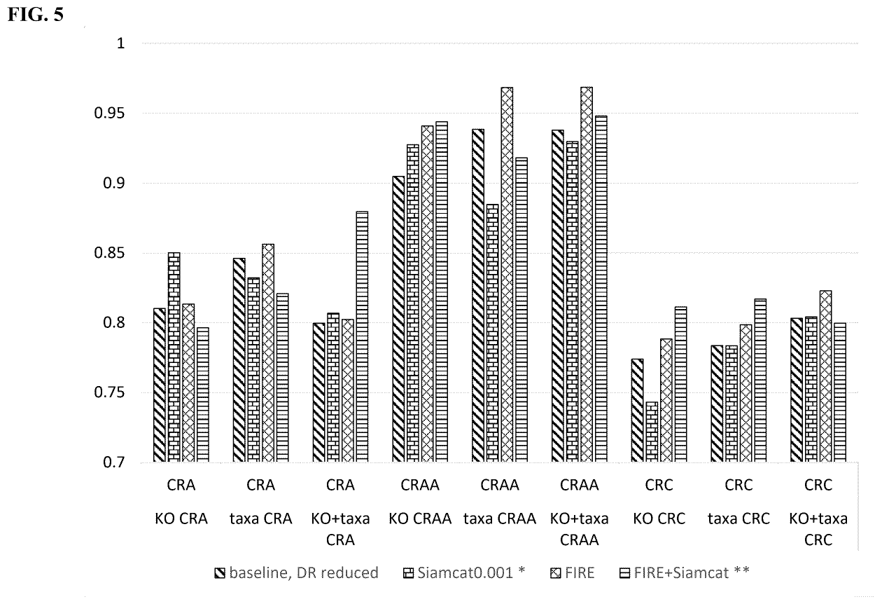

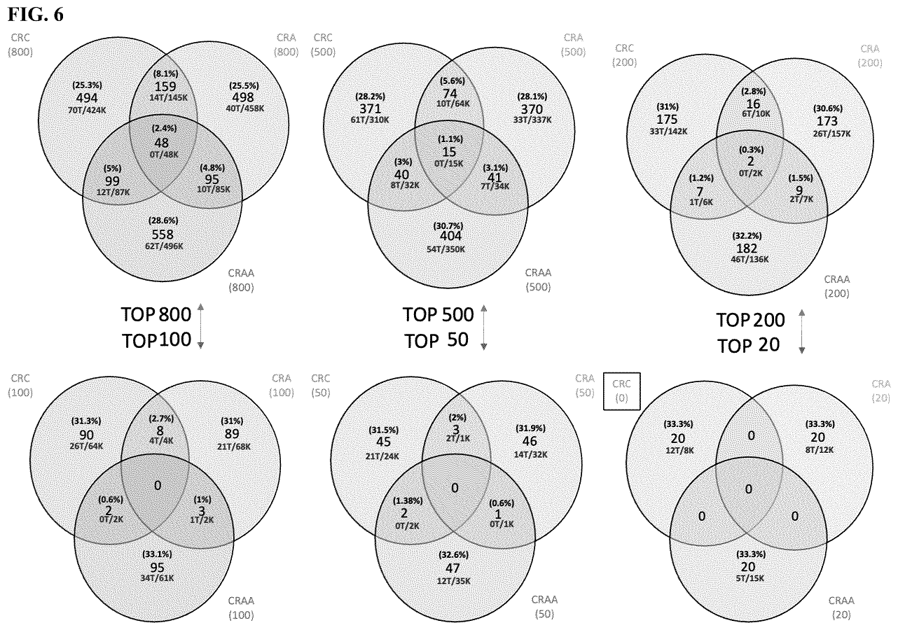

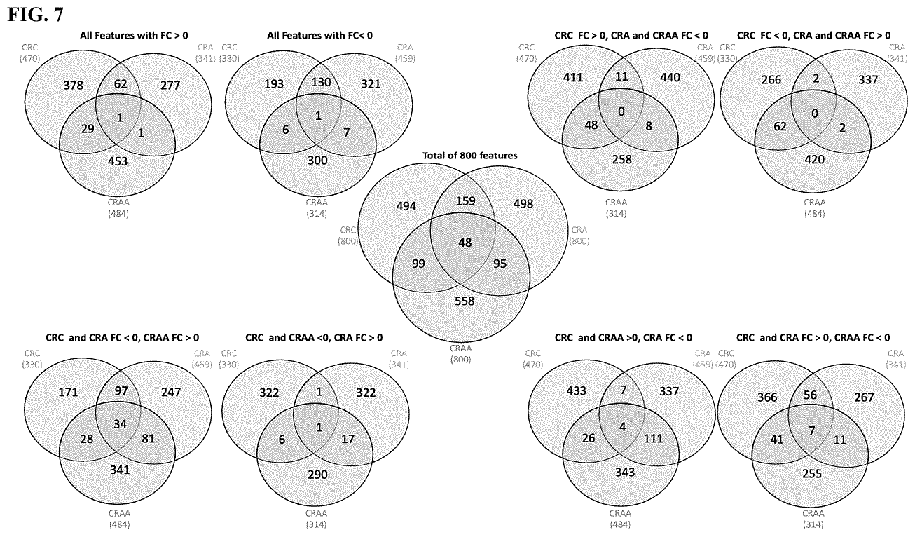

It is evident from the SIAMCAT output that several taxonomic features have negligible fold-change, however these same species display significant shifts in prevalence. Not surprisingly, many of the top features generated by FIRE and SIAMCAT overlap, but several features are unique to one method or the other. This serves as the rational basis for combining the non-redundant features to assess their relative impact on classification performance and to evaluate the relative performance strength of our approach. We evaluated possible incremental improvements of our approach by generating AUCs using the best model from machine learning algorithms to establish a baseline for comparison to FIRE and SIAMCAT alone and in combination (FIG. 5). While FIRE feature selection generally outperformed SIAMCAT, the value of SIAMCAT is evident from cases where the best performance was obtained by combining FIRE and SIAMCAT analytical features and results (FIG. 5). Furthermore, for all analyses involving non-redundant features derived from FIRE and SIAMCAT, SIAMCAT features were always present among the most important features positively contributing to external test AUC. Applying SIAMCAT to control and CRC samples generated a taxonomic feature importance list that is highly consistent with taxa reported by several studies.

We observed that the best performance is dependent on the disease target. The best performance for CRA was achieved when combining taxonomic and KO features selected by combined FIRE-SIAMCAT. This approach yielded a nearly 8% increase in external AUC (baseline AUC-0.80 vs 0.87). Analysis of CRAA performance was somewhat more complex. All feature selection strategies performed best when using FIRE and there was little difference in the performance when using taxonomic features alone or in combination with gene features. The results for CRC followed similar trends as CRAA. We observed that taxonomic and gene features outperformed taxonomic features alone which in turn outperformed gene features alone. FIRE selected features generated a 3% gain in external AUC (baseline AUC=0.94 vs 0.97). The results for CRC showed that the combination of taxonomic and KO features outperformed taxonomic features alone which in turn outperformed KO features alone. The best performance was observed from features selected by FIRE, resulting in a modest 2% increase in external AUC (baseline AUC=0.80 vs 0.82). These results highlight the challenges associated with optimizing model performance. The microbiota and microbiome associated with each disease class demand defining distinct computational workflows, as no single model can perform optimally on all 3 disease classes.

To gain biological insights into the features that contribute most significantly to distinguishing disease class prediction or potential common features shared between disease classes, we evaluated the overlap of features (generated from a combination of FIRE and SIAMCAT) across disease classes (FIG. 6). It is notable that when comparing the top 20 important features (bottom right Venn diagram) for each disease class, there was no overlap in either taxonomic or gene features. Another interesting distinction in this comparison is that 60% of the features for CRC are taxonomic, significantly larger than that observed for CRA (40%) and CRAA (25%). When examining the top 50 features (bottom middle Venn diagram), these differences are maintained but dissipate. Among the top 50 features we begin to observe modest overlap in features across disease classes. Comparison of the top 100 features shows that the proportions of taxonomic features become quite even across disease classes. As more features are compared, the proportion of gene features continue to increase relative to taxonomic features, and we observe increasing overlap across disease classes. Examination of 800 features reveals a potentially interesting biological aspect of microbiota in the context of colonic neoplasia. It is notable that among the overlapping features gene features dominate relative to taxonomic features. Of particular interest we observe 0 taxonomic features overlapping among all 3 disease classes, yet 48 gene features (2.4%) are shared. This imbalance is also evident in all pairwise comparisons of overlapping features such that taxonomic features represent between ~9-14% of overlapping features. Given that shared taxonomic features frequency is similar across disease classes, it is notable that the number of shared gene features is significantly higher between CRC and CRA relative to any other pairwise relationship.

As a next step we refined these comparisons to consider the direction of change of both feature types, i.e., increased or decreased in disease, to determine the true biological similarity of shared taxonomic and gene features (FIG. 7). Considering the top 800 features for each disease class in a binary fashion (top left; all increased >0; or all decreased <0, relative to healthy control) most overlapping features occur between CRA and CRC samples, although some overlap remains between CRAA and CRC. Dissecting these relationships further in different permutations (top right) shows the relationship between CRA and CRAA samples, emphasizing the small number of features in common that display the same direction of change. These diagrams also emphasize that the large number of overlapping features shared between CRC and CRAA display the opposite direction of change. A comparison of CRA and CRC samples is visualized by comparing Venn diagrams in the bottom far left and bottom far right. The number of shared features considering the direction of change remains large, whereas the remaining diagrams (bottom left CRC FC<0, CRA FC>0, CRAA FC<0 and bottom right CRC FC>0, CRA FC<0, CRAA FC >0) illustrate that most shared features between CRA and CRAA represent cases of change in the opposite direction.

Thus, whereas a substantial number of features shared between CRA and CRC were in common and their direction of change was frequently the same (FIG. 7). This result is most surprising and difficult to explain but suggests that the microenvironment and selective pressure of the gut is similar in CRA and CRC but diverges in CRAA. Without being bound by theory, we hypothesize that the higher proportion of shared gene features relative to taxonomic features may reflect the functional redundancy of related and even distantly related taxa that by virtue of shared genes encoded in their respective genomes are essentially inter-changeable within the community. The high proportion of gene features in expanded feature importance lists suggests that detailed analysis of functional attributes over- and under-represented in health and disease is warranted.

### Example 7: Model Validation: Analysis of Taxonomic Features

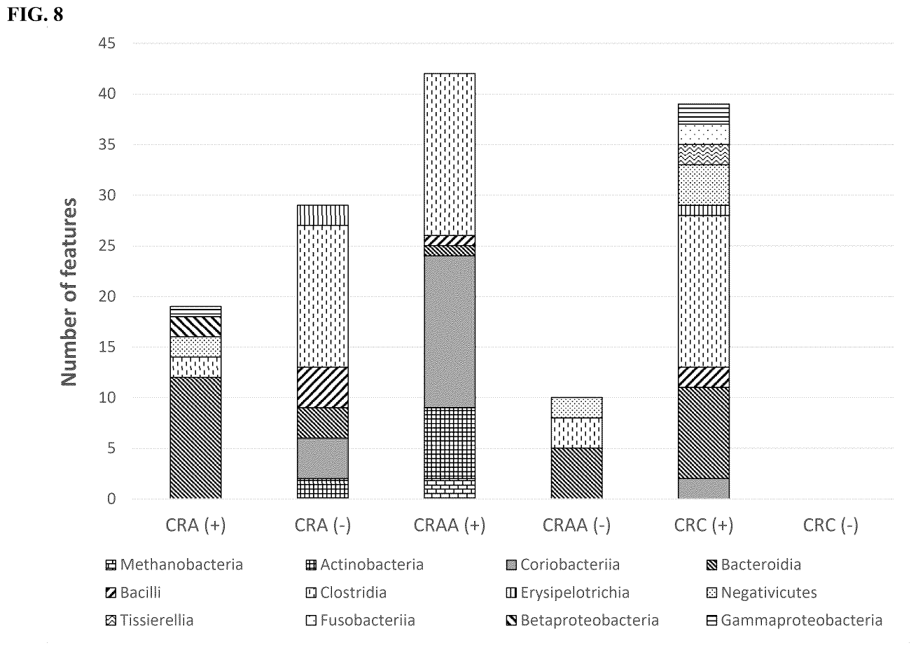

To assess whether taxonomic features among the top 800 for each class behave coherently and/or were biased toward specific phylogenetic groups we analyzed important features at the class and family level. Among the top 800 features, 66 represented taxa over- or under-represented in CRA, 86 taxa for CRAA and 79 taxa for CRC. In total the feature importance list contained taxa from 12 classes (FIG. 8). It should be noted that these features did not necessarily achieve statistical significance in comparisons but were deemed discriminatory based on AI models used. The Clostridia harbored the largest number of under-represented features in CRA. This class was strongly over-represented in CRAA and CRC.

The next most dominant class among the top features is Bacteroidia. Twelve out of 15 taxa in CRA top features displayed positive fold-change, whereas 9 taxa in CRC exhibited positive fold-change. By contrast, fewer taxa from this class were discriminatory for CRAA and predominantly under-represented. Two classes (Tissierellia and Fusobacteriia) were over-represented and exclusive to the CRC high importance lists but not present in CRA or CRAA. The classes most indicative of CRAA are the Methanobacteria, uniquely over-represented in CRAA but not in either CRA or CRC. Two additional classes, Actinobacteria and Coriobacteria, are strongly over-represented in CRAA, whereas they were absent or under-represented in CRA and CRC feature importance lists. The coherence of these results is quite remarkable given the enormous phylogenetic space and evolutionary distance within a bacterial class.

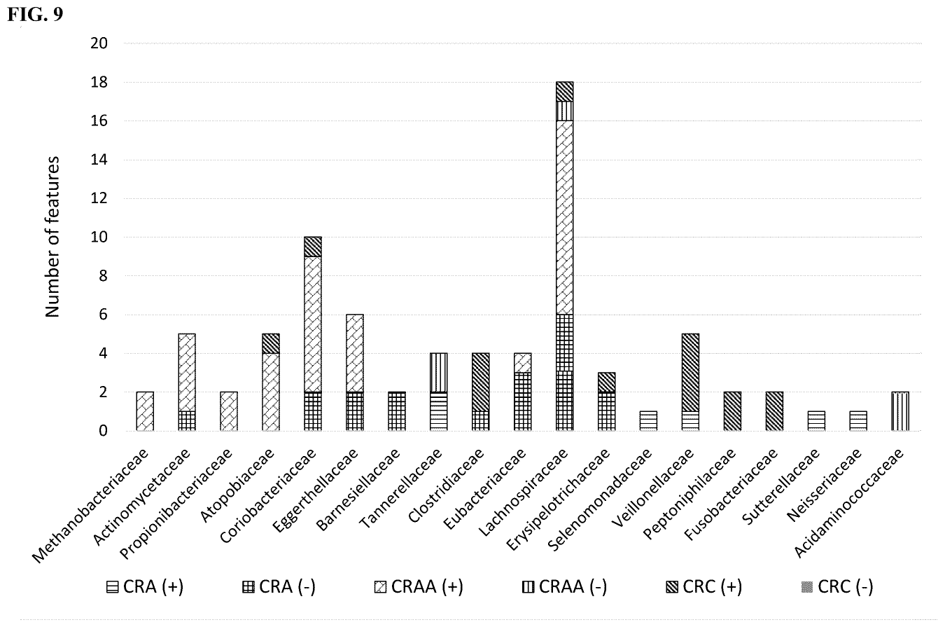

We next examined these results at a higher resolution of Family. The top features for all disease classes resulted in 42 different bacterial families. As we observed when analyzing classes, we see that the distribution of important features for each disease class is biased for particular families. Examination of families belonging to 3 classes, Methanobacteria, Actinobacteria and Coriobacteria, indicate that that the underlying families are providing significant power to discriminate CRAA from other disease classes and healthy subjects (FIG. 9). For CRA, 2 species within Barnesiellaceae were uniquely under-represented, whereas a single species from Selenomonadaceae, Sutterellaceae and Neiseriaceae were all over-represented. In the case of CRAA, two species within Acidaminococcaceae were uniquely under-represented in CRAA. The over-representation of two species within both Methanobactericeae and Propionibacteriaceae were exclusive to the CRAA feature importance list. Two species within Tannerellaceae were uniquely over-represented in CRA and under-represented in CRAA. Species within other families including Actinomycetaceae and Eggerthellaceae were over-represented in CRAA and under-represented in CRA. Taxa belonging to Lachnospiraceae while not exclusively over-represented in CRAA involve 10 underlying taxa that distinguish CRAA from other disease classes. The over-representation of two families (Peptoniphilaceae and Fusobacteriaceae) each harboring 2 species are unique to CRC samples. Two families, including, Clostridiaceae (3 taxa) and Erysipelotrichaceae (2 taxa) discriminate CRC from other disease classes and healthy controls.

Among the most important taxonomic features for CRA, we observed differential representation of 6 Bacteroides spp. Several genera were represented by 2 or more species including Prevotella spp, Parabacteroides spp, and Veillonella spp., all of which were over-represented compared to healthy control samples and Eubacterium spp, and Roseburia spp. both of which were under-represented. All other differentially abundant genera were represented by single species. Important features for CRAA displayed genera over-represented compared to healthy control samples including 4 species belonging to Actinomyces, 3 Collinsiella, 2 Enorma, 2 Lactobacillus, 2 Dorea and 2 Coprococcus. Two species belonging to Bacteroides were under-represented compared to healthy control samples. Two Alistipes spp. were divergent in their representation relative to control samples. Important features for CRC included 2 Actinomyces spp., 3 Porphorymonas spp., 4 Prevotella spp., 2 Peptostreptococcus spp., 3 Fusobacterium spp. 3 Bacteroides spp., 2 Veillonella spp., were over-represented in CRC relative to healthy control samples. Several of these features did not display significant fold-change relative to control but did display significant altered prevalence. All other genera were represented by single species. It is of interest that no taxa on the CRC feature importance list displayed under-representation.

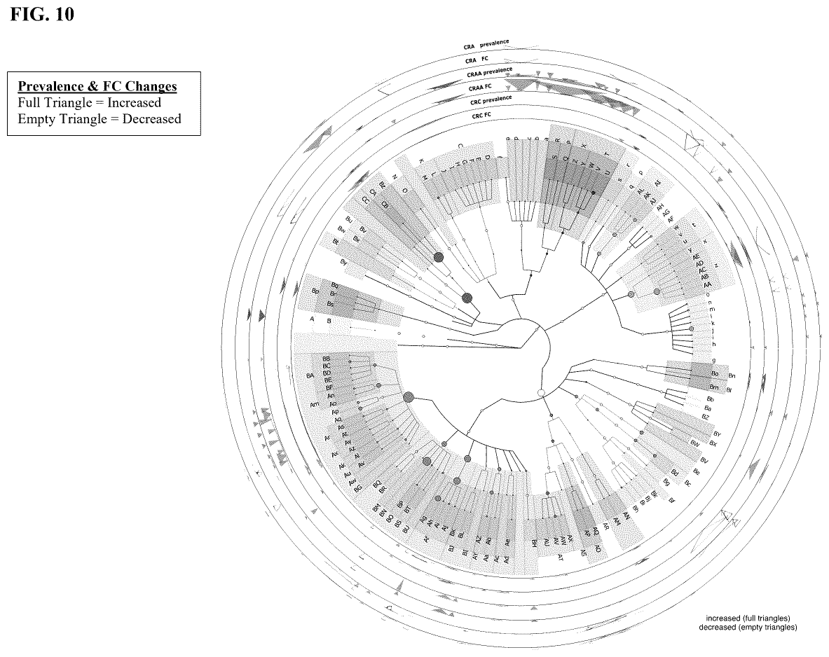

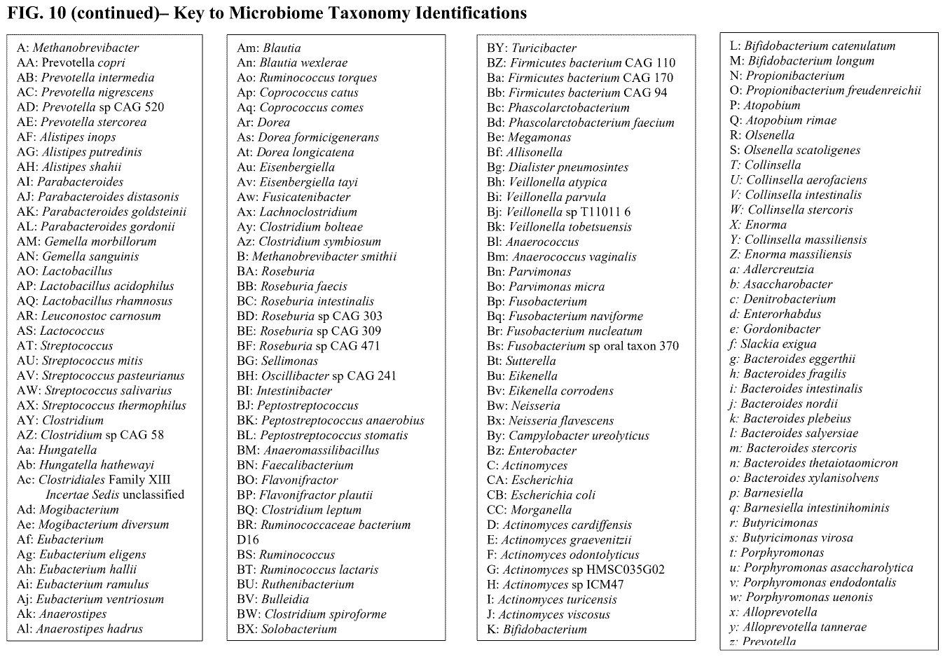

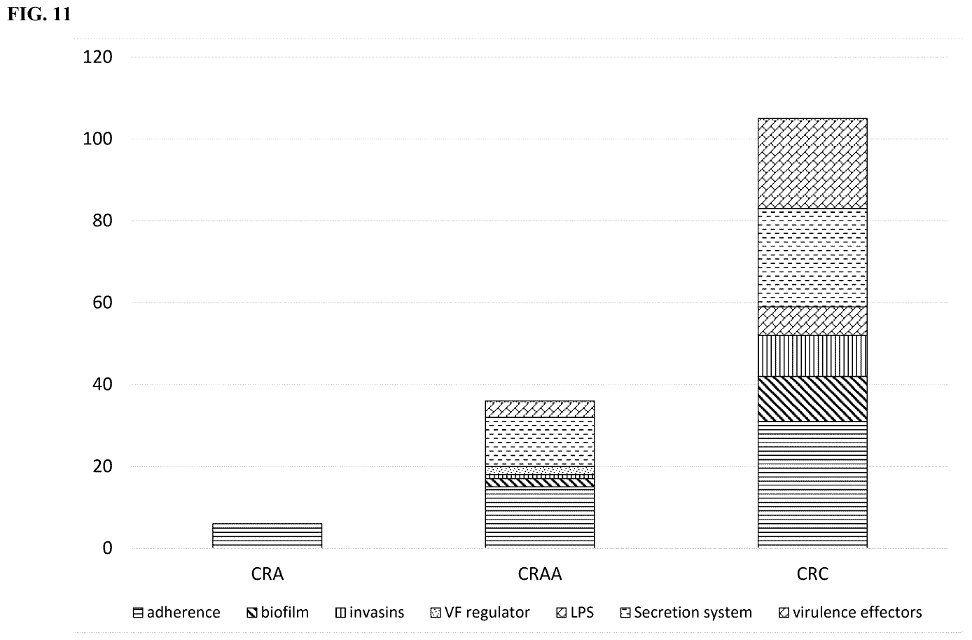

As shown in FIG. 10, the following taxonomic features increased in CRA: Actinomyces odontolyticus, Bifidobacterium adolescentis, Bifidobacterium longum, Enterorhabdus caecimuris, Gordonibacter pamelaeae, Bacteroides eggerthii, Bacteroides intestinalis, Bacteroides nordii, Bacteroides plebeius, Bacteroides salyersiae, Bacteroides stercoris, Barnesiella intestinihomonis, Butyricimonas virosa, Prevotella copri, Prevotella stercorea, Alistipes shahii, Parabacteroides gordonii, Parabacteroides goldsteinii, Gemella sanguinis, Streptococcus thermophilus, Eubacterium ventriosum, Anaerostipes hadrus, Blautia obeum, Blautia wexlerae, Dorea formicigenerans, Dorea longicatena, Fusicatenibacter saccharivorans, Eubacterium rectale, Roseburia sp. CAG 309, Roseburia sp. CAG 431, Oscillibacter sp. CAG 241, Faecalibacterium prausnitzii, Clostridium leptum, Ruminococcaceae bacterium D16, Ruminococcus lactaris, Clostridium spiroforme, Firmicutes bacterium CAG 110, Veillonella atypica, Veillonella tobetsuensis, Neisseria flavescens, Klebsiella pneumoniae, and Klebsiella variicola. Among the most important taxonomic features for CRA, we observed differential representation of 6 Bacteroides spp. Several genera were represented by 2 distinct species including Prevotella spp, Parabacteroides spp, and Veillonella spp., all of which were over-represented compared to healthy control samples and Eubacterium spp, and Roseburia spp. both of which were under-represented. All other genera were represented by single species.

As also shown in FIG. 10, the following taxonomic features increased in CRAA: Methanobrevibacter smithii, Bifidobacterium longum, Propionibacterium freudenreichii, Olsenella scatoligenes, Collinsella aerofaciens, Collinsella intestinalis, Collinsella stercoris, Enorma massiliensis, Adlercreutzia equolifaciens, Asaccharobacter celatus, Gordonibacter pamelaeae, Slackia isoflavoniconvertens, Bacteroides ovatus, Bacteroides thetaiotaomicron, Bacteroides uniformis, Bacteroides vulgatus, Bacteroides xylanisolvens, Alistripes inops, Alistripes putredinis, Parabacteroides distasonis, Streptococcus mitis, Clostridium Sp. CAG 167, Eubacterium hallii, Anaerostipes hadrus, Blautia wexlerae, Ruminococcus torquea, Coprococcus catus, Coprococcus comes, Dorea formicigenerans, Dorea longicatena, Fusicatenibacter saccharivorans, Clostridium bolteae, Roseburia faecis, Oscillibacter sp. CAG 241, Intestinibacter bartlettii, Firmicutes bacterium CAG 170, Firmicutes bacterium CAG 238, Firmicutes bacterium CAG 94, Phascolarctobacterium faecium, and Haemophilus parainfluenzii. Important features for CRAA displayed genera over-represented compared to healthy control samples including 4 species belonging to Actinomyces, 3 species belonging to Collinsiella, 2 species belonging to Enorma, 2 Lactobacillus, 2 Dorea and 2 Coprococcus. Two species belonging to Bacteroides were under-represented compared to healthy control samples. Two Alistipes spp. were divergent in their representation relative to control samples.

As also shown in FIG. 10, the following taxonomic features increased in CRC: Actinomyces turicensis, Bifidobacterium catenulatum, Collinsella aerofaciens, Slackia exigua, Bacteroides fragilis, Bacteroides nordii, Bacteroides plebeius, Butyricimonas virosa, Porphyromonas asaccharolytica, Porphyromonas endodontalis, Porphyromonas uenonis, Alloprevotella tannerae, Prevotella intermedia, Prevotella nigrescens, Prevotella sp CAG 520, Prevotellastercorea, Gemella morbillorum, Streptococcus pasteurianus, Streptococcus salivarius, Clostridium sp CAG 58, Hungatella hathewayi, Mogibacterium diversum, Eubactenum eligens, Eubacterium ramulus, Eubacterium ventriosum, Anaerostipes hadrus, Coprococcus catus, Eisenbergiella tayi, Clostridtum symbiosum, Roseburia intestinalis, Roseburia sp CAG 303, Peptostreptococcus anaerobius, Peptostreptococcus stomatis, Faecalibacterium prausnltzii, Ruminococcaceae bacterium D16, Ruthenibacterium lactatiformans, Solobacterium moorei, Firmicutes bacterium CAG 94, Dialister pneumosintes, Veillonella parvula, Veillonella sp T110116, Parvomonas micra, Fusobacterium naviforme, Fusobacterium nucleatum, Fusobacterium sp oral taxon 370, Eikenella corrodens, Escherichia coli, and Morganella morganii. Important features for CRC included 2 Actinomyces spp., 3 Porphorymonas spp., 4 Prevotella spp., 2 Peptostreptococcus spp., 3 Fusobacterium spp. Several of these features did not display significant fold-change relative to control but did display significant increased prevalence. Three Bacteroides spp., 2 Veillonella spp., were over-represented in CRC relative to healthy control samples. All other genera were represented by single species.

We conducted an in-depth meta-analysis of publicly available microbiome shotgun sequence data from fecal samples of healthy control donors and those diagnosed by colonoscopy as CRA, CRAA and CRC. The characteristics of the 13 studies analyzed varied substantially, including eight different countries, various disease states, cohort size, and number of reads passing quality control metrics (Table 1). Although most studies attempted to balance gender and age within their respective cohorts, male were generally more prevalent than female. Additional factors such as DNA preparation and sequencing methods varied across studies, and importantly, some studies collected fecal or other samples after colonoscopy. These factors are likely to introduce variability into the study outcomes. Despite these confounding factors, the taxonomic features identified in individual studies, while variable, do define a consensus finding, at least for CRC. Given the known inter-personal variability in microbiota and distinct dietary habits of each participating country, the fact that similar taxa are identified strongly suggests that the selective forces operating in CRC are dominant to diet and other known selective pressures.

These findings lead to two surprising conclusions regarding the selective microenvironments generated by colonic lesions and/or the contributions of gut microbiota to disease onset and progression. First, the strong dissimilarity between CRA and CRAA microbiota draw into question whether advanced adenomas are simply larger forms of adenomas. Our results suggest that this assumption requires further investigation and instead indicate that the microbiota adenoma/advanced adenoma interactions represent highly distinct processes. Second, and perhaps even more surprising, is that given the strong opposing behavior of CRA and CRAA microbiota with respect to gene representation, the CRA and CRC microbiota are functionally substantially synonymous. Indeed, the gene features displaying congruent direction of change in CRA and CRC microbiota are not separate from those observed in CRAA. Among the 408 features that were differentially represented in all 3 disease classes compared to healthy controls, 389 (95%) displayed this pattern of agreement in direction between CRA and CRC and disagreement in direction between CRAA and the other disease classes. In this regard, the same gene features that are increased in common between CRA and CRC samples are decreased relative to healthy control samples in CRAA and vice-versa. This result is however considered preliminary since nearly all of the advanced adenoma samples were derived from a single study.

### Example 8: Validation of High-Impact Features Via External Data Validation Testing

Following completion of the formal analysis of FIRE and SIAMCAT, data from a new study became available focused on a Spanish cohort (Front. Microbiol., 2024; 11 (14): 1-17). This data was imported and used for additional external validation. The new dataset consisted of 30 CRC and 30 CONTROL samples. The data included an additional 30 polyp samples that were not analyzed due to a lack of specification to distinguish early (CRA) vs late-stage adenomas (CRAA). We employed BB3 to establish taxonomy assignments. The model developed for taxonomy annotation (eXtreme Gradient Boosted Trees Classifier) was used to score the new external data set using informative features and a threshold value corresponding to that which maximizes the F1 score (0.629). The resulting predictive performance of the model generated the following outcomes: (AUC=0.8089, Sensitivity=0.7333, Specificity=0.8333 and Accuracy-0.7833). Depending on the metric evaluated, these scores were either superior or comparable to that achieved on external HO data analyses, using data from the original metagenomic data. These results indicate that the modelling efforts were largely successful, reflecting a CRC vs CONTROL classification model that exhibits good generalizability and yielded satisfactory performance on new data.

### Example 9: Validation of High-Impact Features Via qPCR

The challenge of designing primers specific to target taxa of interest are multifaceted. The greatest challenge is related to the massive ratio of known sequence space occupied by target taxa compared to unknown sequence space residing on the planet. In this regard, the quality of any primer design must be qualified as acceptable until proven otherwise. The targeting of unique gene sequences present in taxa of interest but absent in near neighbors represents the most straight-forward way to conduct specific qPCR. However, the target gene, while universally present in sequenced isolates may in fact be absent in uncharacterized samples, thereby capable of generating under-estimated abundance in qPCR reactions. Conversely, the mapping of sequence reads from shotgun metagenomic sequencing of stool samples is imperfect and limited in accuracy based on known sequence availability. In this regard the relative abundance measures generated by sequence enumeration may not be perfect and therefore may differ from those measures generated by qPCR. Many of these nuances can be directly evaluated in candidate primer designs by sequencing of PCR products generated from tens or hundreds of reactions to assess the purity of sequences in the products generated. Non-specific priming or amplification of near neighbor sequences can and should be quantified after a comprehensive initial assessment and before deployment for any commercial testing.

Based on a list of 300 taxa generated by applying FIRE feature selection (from each category CRC, CRA, and CRAA) and ensemble modeling, top target species are used for primer design. Multiple primer sets were identified for each target based on gene sequences that were identified as unique for the target. Primer pairs targeting total bacteria using the 16s rRNA gene were used as an internal housekeeping control to normalize results across samples: Total Bacteria_16S Fw GCAGGCCTAACACATGCAAGTC (SEQ ID NO: 1), Total Bacteria_16S Rv CTGCTGCCTCCCGTAGGAGT (SEQ ID NO: 2), product size 120 base pair). A list of all working primers for each category are shown in Table 6.

Each primer was tested on 10-48 samples, with approximately 50% from CRC/CRA/CRAA subjects and ~50% from CTR (control subjects). Theoretical and results-based evaluation of primer designs took several metrics into account: Tm, Melting Curve, Presence or absence of primer dimers, Presence or absence of harpin, Ct amplification number, Number of bases, Product size, Specificity of primers couple (based on Primer 3 blast alignment), Reproducibility of sequencing data.

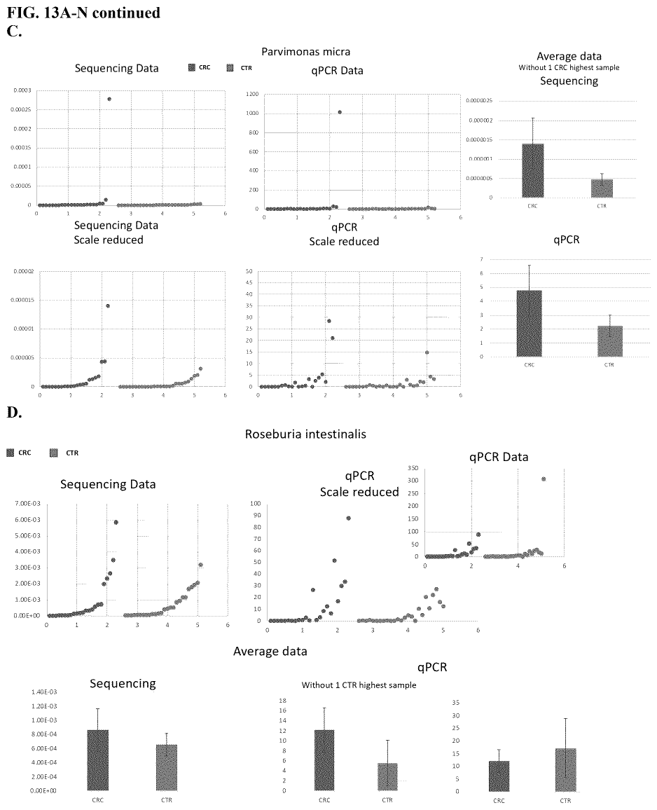

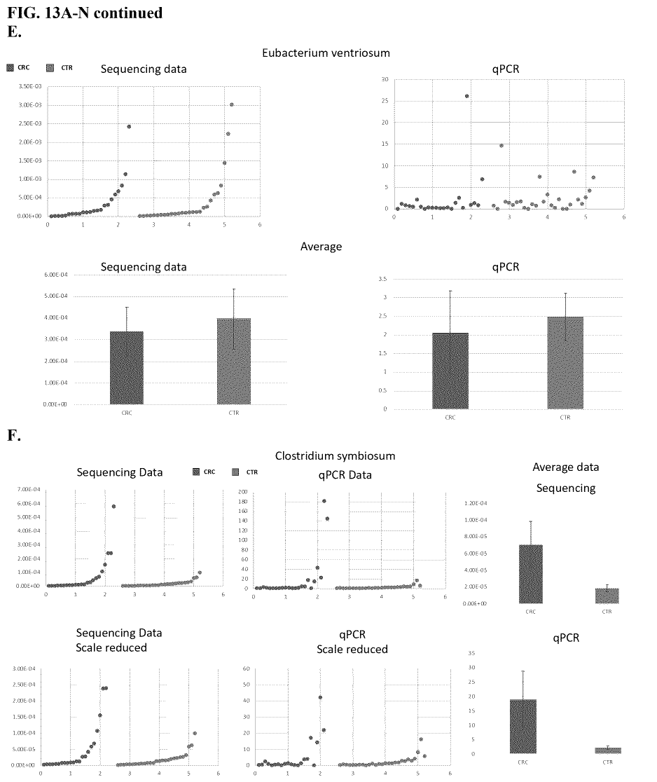

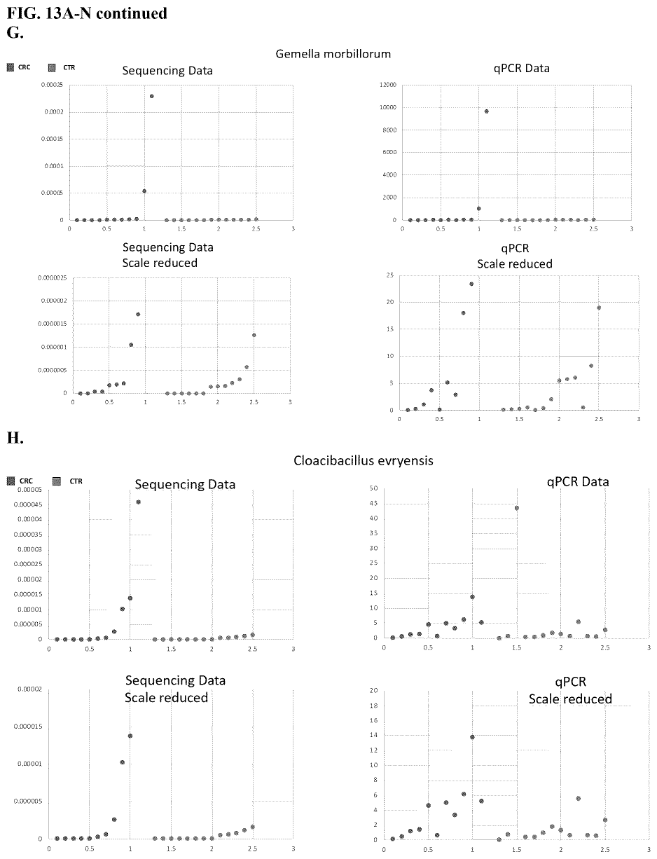

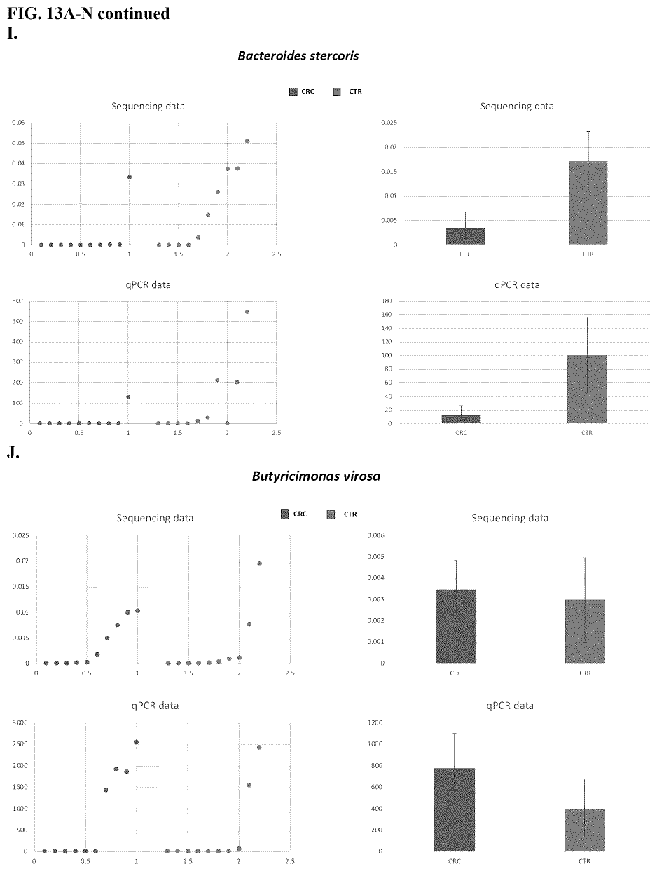

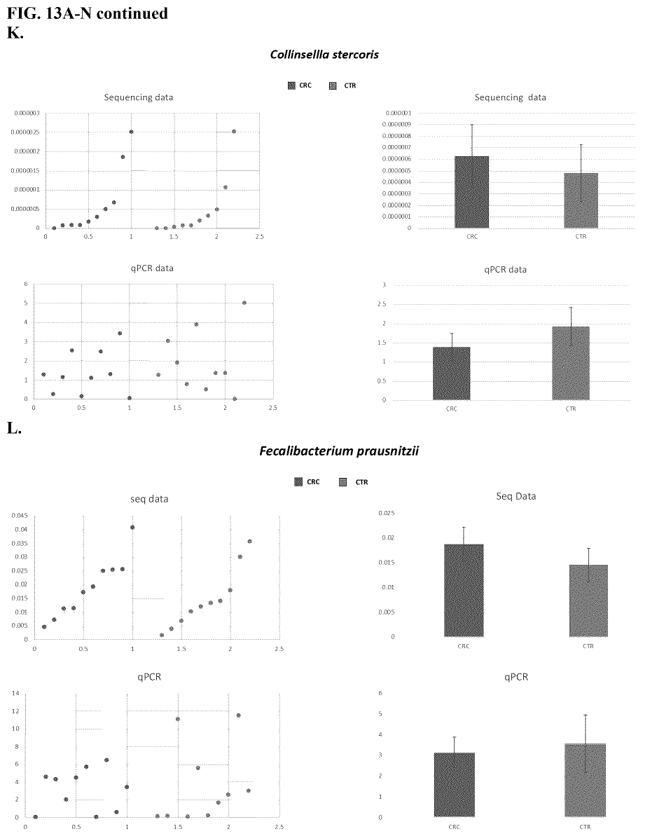

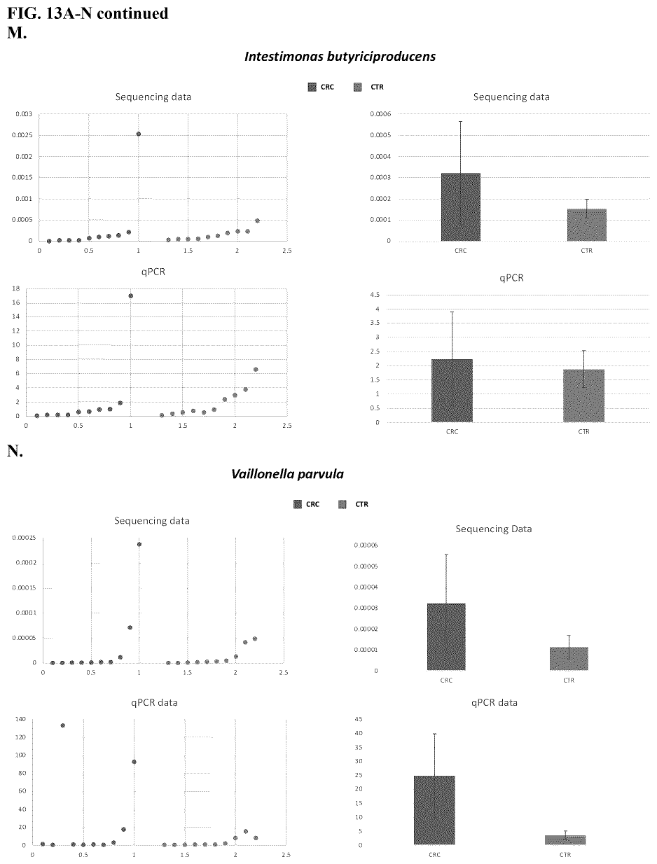

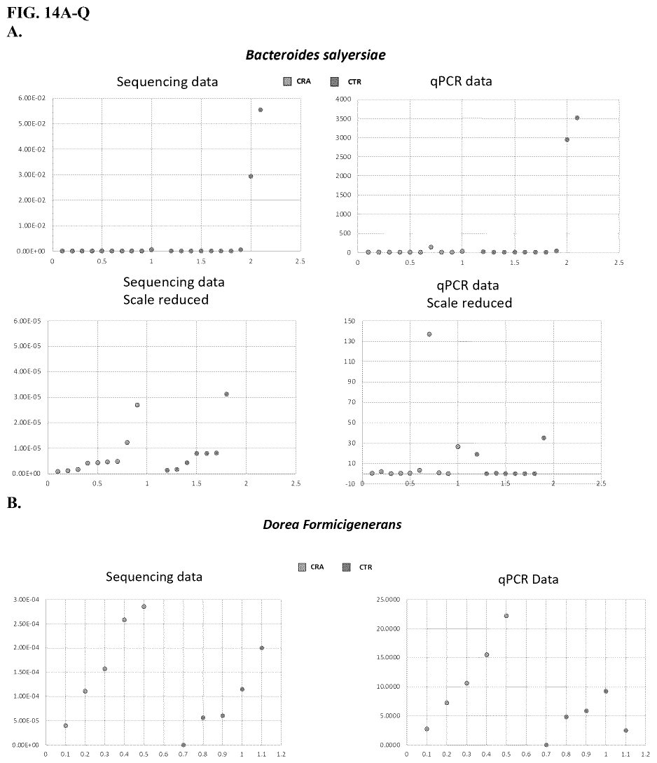

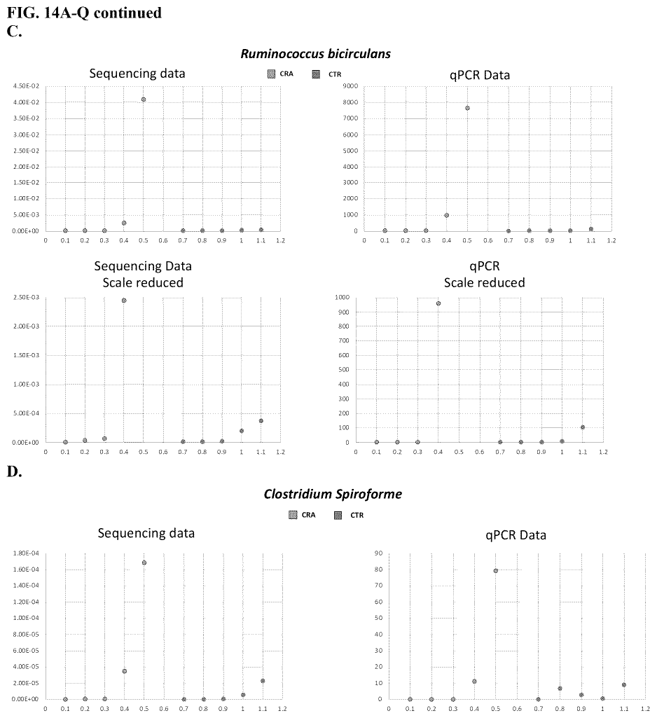

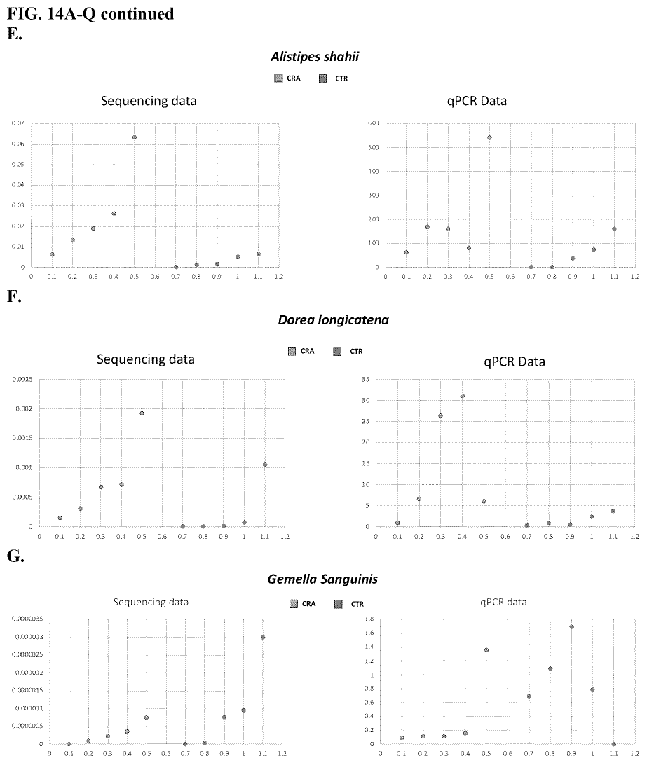

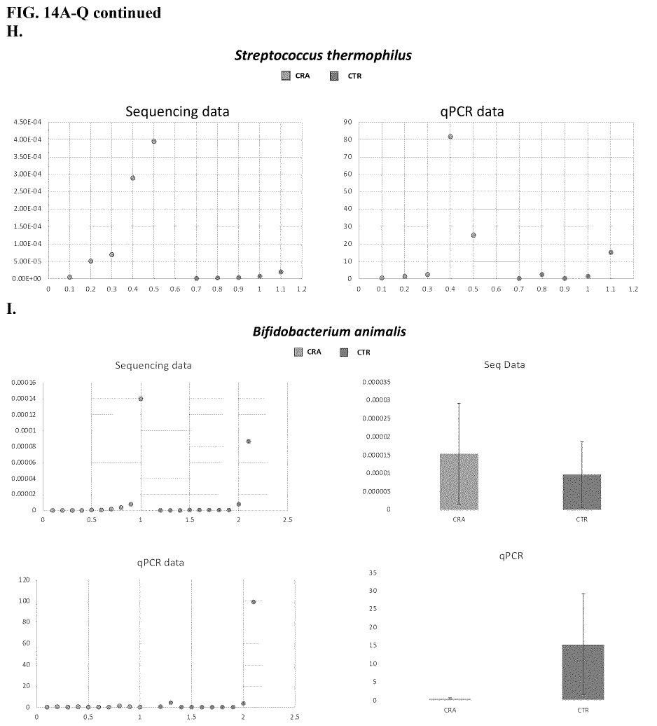

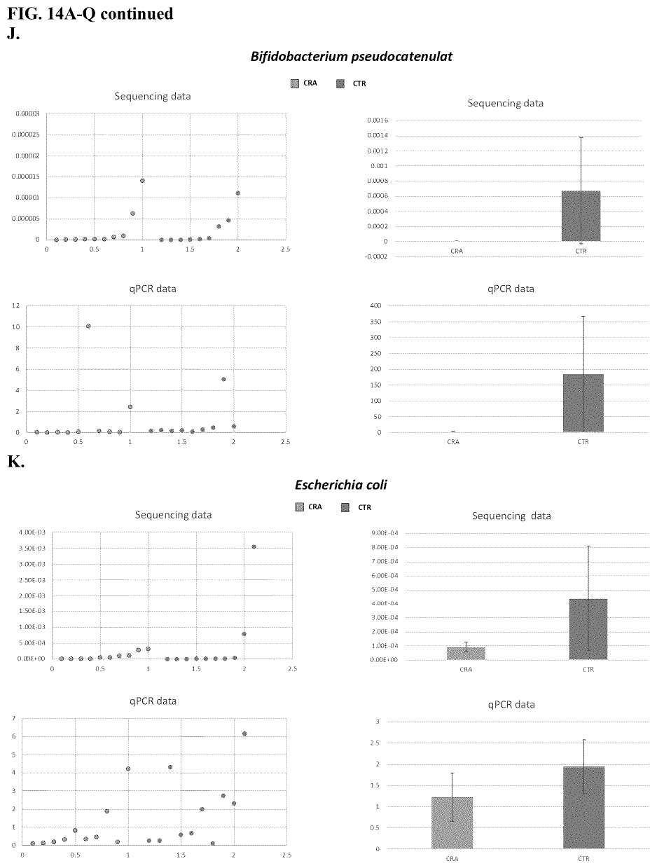

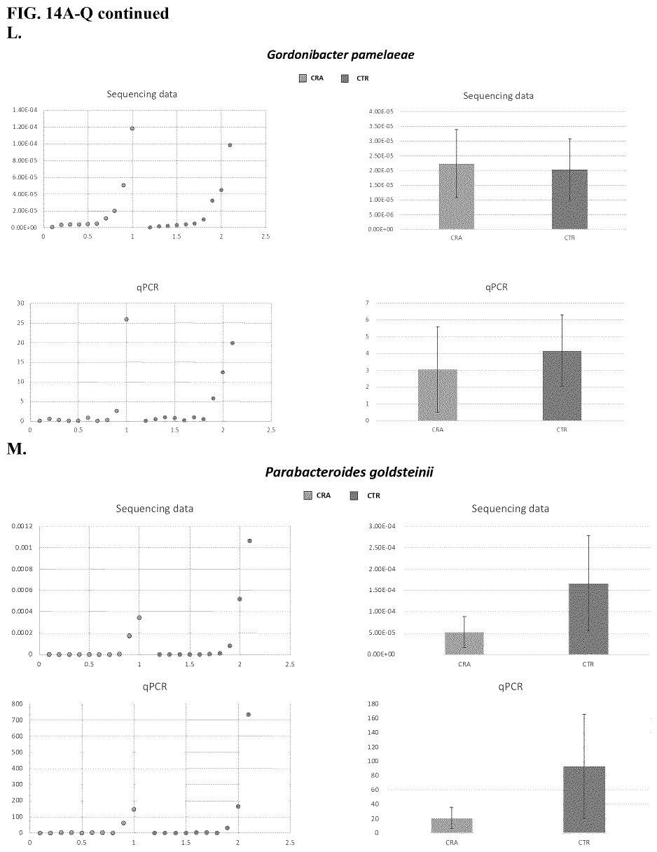

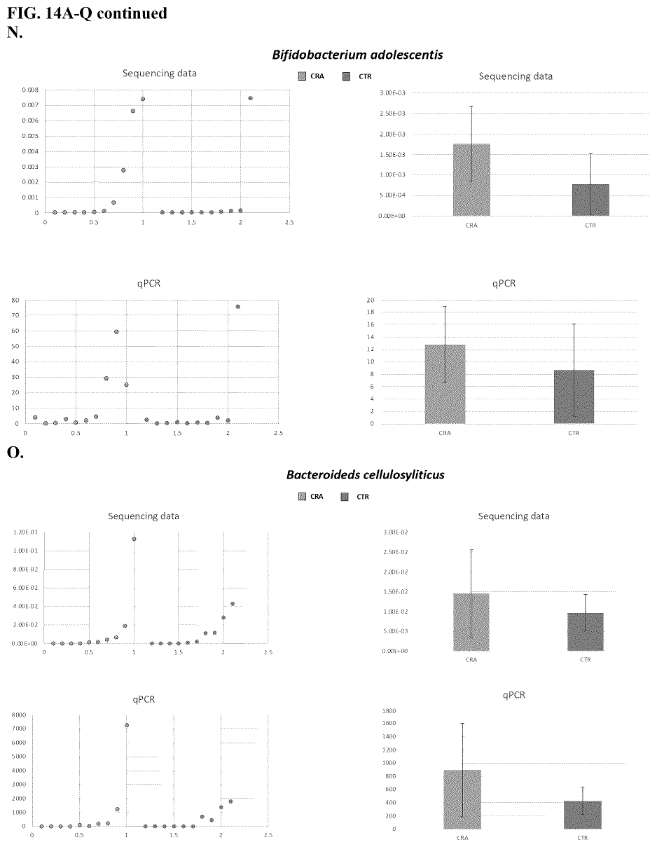

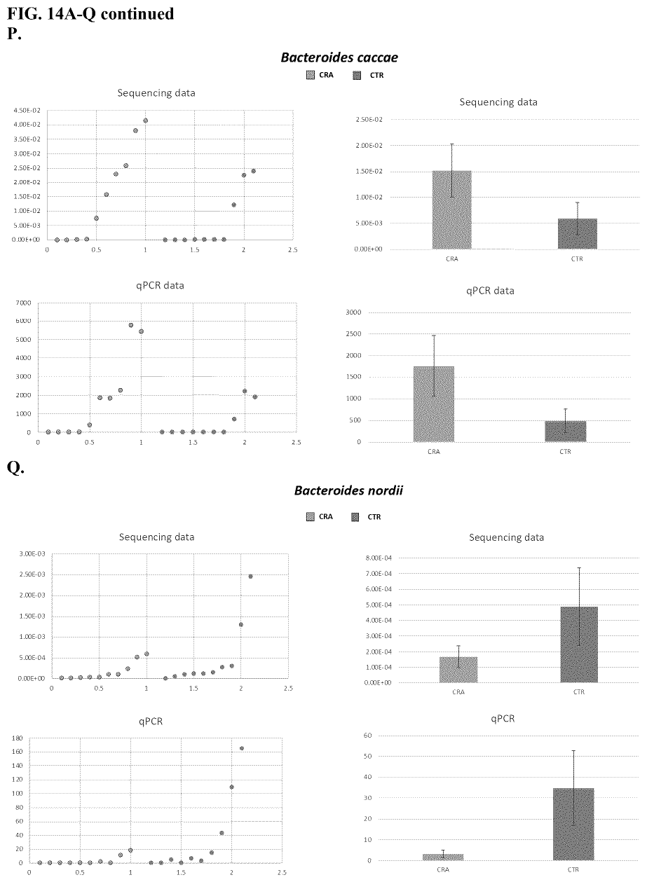

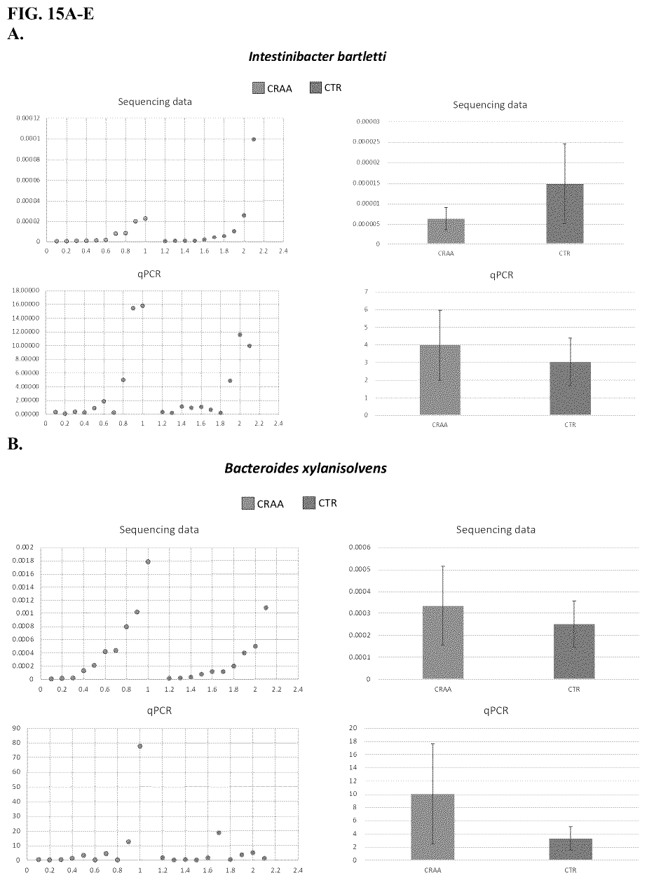

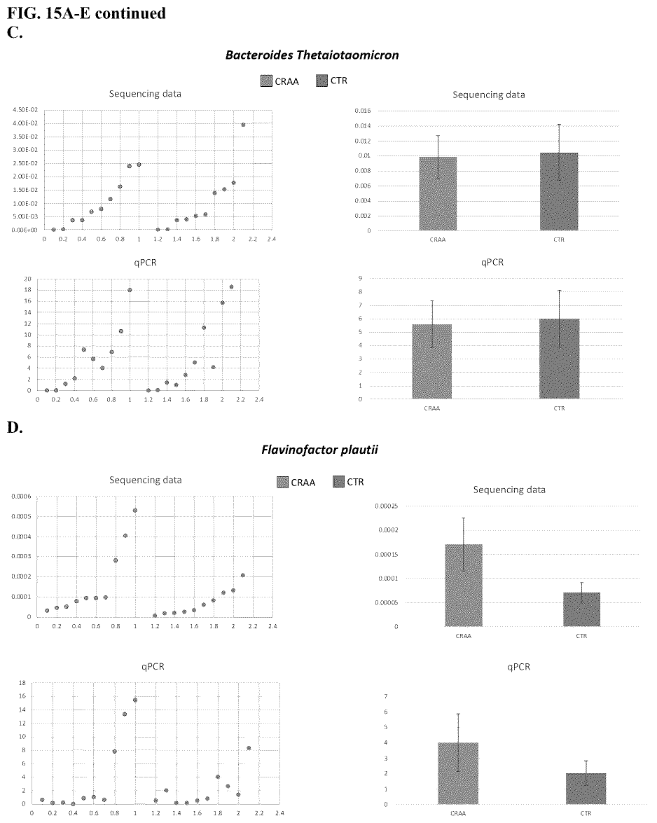

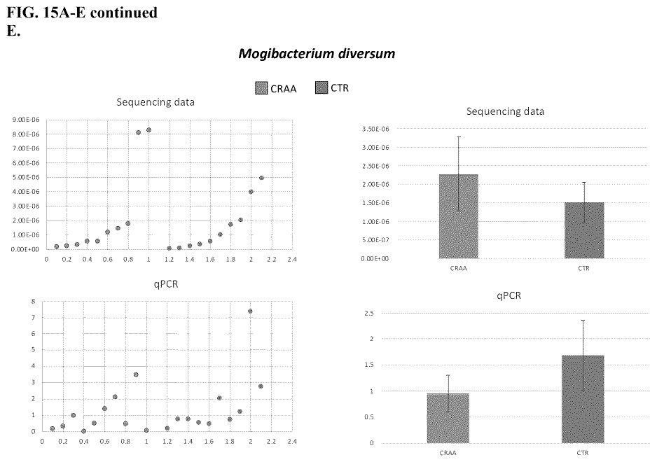

We evaluated the quantitative relative abundance values for each sample, comparing shotgun sequencing and qPCR data for each specific target taxa. The results are summarized in FIGS. 13A-N (CRC), FIGS. 14A-Q (CRA), and FIGS. 15A-E (CRAA). Scatter plots show the quantitative relative abundance values of each sample based on shotgun sequencing (left) and qPCR (right) for each specific target taxa. Each dot represents one sample. Sequencing samples are ordered from lowest to highest value, and qPCR samples are sorted according to the order of the sequencing samples. The y-axis of the sequencing graphs show the raw data value, while the y-axis of the qPCR graphs show the relative abundance values calculated by method 2 (−Delata Delta C (T)). The bar graphs show the average of sequencing and qPCR data for samples tested. The error bar represents the value of the standard error.

According to these data, 16 primer pairs for CRC, 18 primer pairs for CRA, and 6 primer pairs for CRAA reproduce the sequencing data. Certain primer pairs (not shown) demonstrated high specificity for the target but the abundance of taxa is very low. These include: Peptostreptococcus stomatis and Dialister pneumosintes for CRC; Caprococcus catus for CRA; and Actinomyces graevenitzii for CRAA.

TABLE 1
Selected Metagenomic projects representing different subject population cohorts used for modeling. Descriptive statistics of
gender, BMI, age, disease classification, raw reads, post-qc reads, and percentage of human reads for each project. For continuous
variables, mean and standard deviation are shown and for categorical variables number of samples within each category is shown.

| | | | | | | | | | (%) |
|---|---|---|---|---|---|---|---|---|---|
| | | | | | | | | | Reads |
| | | | | | | | | | from |
| | Num- | | | | | | | Post-QC | Human |
| Project | ber | Gender | Age | BMI | Country | Disease Type | Raw Reads | Reads | DNA |
| Feng | 156 | male: 88 | 66.9 ± | 27.4 ± 4.02 | Austria | CTR: 63; CRC: 46; | 52689474 ± 8343659 | 46088635 ± 7292627 | 4.63 ± 1.05 |
| | | female: 68 | 8.32 | | | CRA: 0; CRAA: 47 | | | |
| Gao, 2021 | 126 | Missing | Missing | Missing | China | CTR: 47; CRC: 39; | 46462323 ± 16612805 | 42959333 ± 15584240 | 1.45 ± 3.55 |
| | | | | | | CRA: 40; CRAA: 0 | | | |
| Guangxi, | 35 | male: 20 | 60 ± | Missing | China | CTR: 0; CRC: 0; | 78015590 ± 8536400 | 69451603 ± 8481558 | 1.97 ± 0.568 |
| 2018 | | female: 15 | 6.05 | | | CRA: 35; CRAA: 0 | | | |
| Gupta, | 30 | male: 18 | 59.8 ± | Missing | India | CTR: 0; CRC: 30; | 9229167 ± 4142109 | 8510190 ± 3816170 | 1.7 ± 0.651 |
| 2019 | | female: 11 | 7.81 | | | CRA: 0; CRAA: 0 | | | |
| | | missing: 1 | | | | | | | |
| Hale, 2018 | 7 | male: 4 | 65.4 ± | Missing | USA | CTR: 0; CRC: 7; | 152056692 ± 13931620 | 136751334 ± 12185083 | 2.14 ± 0.378 |
| | | female: 3 | 14.7 | | | CRA: 0; CRAA: 0 | | | |
| Hannigan | 81 | male: 46 | 58.6 ± | 28.1 ± 6.1 | Canada, | CTR: 28; CRC: 27; | 6593685 ± 3784609 | 4964982 ± 2801436 | 2.69 ± 0.718 |
| | | female: 35 | 10.8 | missing: 1 | USA | CRA: 26; CRAA: 0 | | | |
| Spanogian- | 10 | Missing | Missing | Missing | USA | CTR: 0; CRC: | 37213448 ± 6907944 | 34520932 ± 6316395 | 4.00 ± 9.49 |
| nopoulos, | | | | | | 10; CRA: | | | |
| 2022 | | | | | | 0; CRAA: 0 | | | |
| Thomas | 140 | male: 52 | 67.5 ± | 25.5 ± 3.93 | Italy | CTR: 52; CRC: 61; | 44984798 ± 24403021 | 41938748 ± 22955372 | 1.52 ± 0.501 |
| | | female: 28 | 8.73 | missing: 64 | | CRA: 27; CRAA: 0 | | | |
| | | missing: | missing: | | | | | | |
| | | 60 | 60 | | | | | | |
| Vogtmann | 104 | male: 74 | 61.5 ± | 25.1 ± 4.25 | USA | CTR: 52; CRC: 52; | 62406634 ± 15463669 | 55272649 ± 14006526 | 6.62 ± 2.51 |
| | | female: 30 | 12.3 | missing: 3 | | CRA: 0; CRAA: 0 | | | |
| Wirbel | 130 | male: 76 | 63.4 ± | 24.9 ± 4.2 | Germany | CTR: 60; CRC: 70; | 25277871 ± 9126431 | 23317638 ± 8560313 | 1.75 ± 0.791 |
| | | female: 54 | 12.1 | | | CRA: 0; CRAA: 0 | | | |
| Yachida | 611 | male: 353 | 61.8 ± | 22.9 ± 3.37 | Japan | CTR: 286; CRC: | 45765841 ± 12910710 | 41600333 ± 11694276 | 1.13 ± 0.576 |
| | | female: | 11 | missing: 10 | | 258; CRA: 67; | | | |
| | | 258 | | | | CRAA: 0 | | | |
| Yu | 128 | male: 81 | 64.2 ± | 23.8 ± 3.08 | China | CTR: 54; CRC: 74; | 56317665 ± 9956025 | 48374964 ± 9470724 | 4.35 ± 1.63 |
| | | female: 47 | 9.08 | missing: 1 | | CRA: 0; CRAA: 0 | | | |
| Zeller | 154 | male: 84 | 63.1 ± | 25.5 ± 4.04 | France, | CTR: 61; CRC: 91; | 58257017 ± 23112145 | 50340355 ± 20826272 | 6.21 ± 5.54 |
| | | female: 70 | 12 | missing: 4 | Germany | CRA: 1; CRAA: 1 | | | |

TABLE 2
FIRE Feature Selection. The table reports AUC values for the external (20%) data sets.

| FIRE | Annotation, target | | | | | | | | |
|---|---|---|---|---|---|---|---|---|---|
| feature set | KO, | | KO_taxa, | | taxa, | KO_taxa, | KO, | taxa, | KO_taxa, |
| size | CRC ¹ | taxa, CRC ¹ | CRC ² | KO, CRAA ² | CRAA ³ | CRAA ⁴ | CRA ⁴ | CRA ⁴ | CRA ³ |
| 800 | 0.7882 | 0.7843 | 0.8113 | 0.8708 | 0.8847 | 0.9653 | 0.8079 | 0.8101 | 0.7645 |
| 700 | 0.7749 | 0.7798 | 0.8183 | 0.8841 | 0.9278 | 0.9606 | 0.7949 | 0.8289 | 0.7589 |
| 600 | 0.781 | 0.7899 | 0.8189 | 0.8502 | 0.9261 | 0.9551 | 0.8106 | 0.8362 | 0.75 |
| 500 | 0.7649 | 0.7936 | 0.8147 | 0.8593 | 0.9366 | 0.9645 | 0.7949 | 0.8347 | 0.7648 |
| 400 | 0.7546 | 0.7984 | 0.8227 | 0.8394 | 0.941 | 0.9685 | 0.7968 | 0.8197 | 0.7573 |
| 300 | 0.7734 | 0.7815 | 0.7995 | 0.8539 | 0.9234 | 0.9582 | 0.7842 | 0.8245 | 0.8021 |
| 200 | 0.7713 | 0.7826 | 0.8094 | 0.9149 | 0.963 | 0.9661 | 0.7708 | 0.8386 | 0.7511 |
| 100 | 0.7334 | 0.7941 | 0.7981 | 0.93 | 0.9683 | 0.9574 | 0.7809 | 0.8392 | 0.7933 |
| 90 | 0.7326 | 0.7789 | 0.7972 | 0.933 | 0.9551 | 0.9511 | 0.7549 | 0.8309 | 0.7871 |
| 80 | 0.7438 | 0.7702 | 0.8217 | 0.8955 | 0.9076 | 0.9472 | 0.775 | 0.8184 | 0.7728 |
| 70 | 0.7441 | 0.7736 | 0.7844 | 0.901 | 0.8961 | 0.9519 | 0.7703 | 0.8561 | 0.7736 |
| 60 | 0.7499 | 0.7648 | 0.8057 | 0.8973 | 0.8961 | 0.9582 | 0.7852 | 0.8106 | 0.7863 |
| 50 | 0.7539 | 0.7591 | 0.7956 | 0.9275 | 0.8838 | 0.9567 | 0.7669 | 0.8269 | 0.7845 |
| 40 | 0.7673 | 0.7411 | 0.784 | 0.9408 | 0.875 | 0.9456 | 0.8132 | 0.8328 | 0.7661 |
| 30 | 0.7436 | 0.7278 | 0.7709 | 0.9396 | 0.8671 | 0.9456 | 0.7884 | 0.7647 | 0.7277 |

Bold underlined values are the maximal external test AUC achieved for a particular annotation, target, and FIRE features set size.
¹ eXtreme Gradient Boosted Trees Classifier
² Keras Slim Residual Neural Network Classifier using Training Schedule (1 Layer: 64 Units)
³ Elastic-Net Classifier (L2/Binomial Deviance)
⁴ Light Gradient Boosted Trees Classifier with Early Stopping.

TABLE 3
Feature List for Colorectal Adenoma (CRA). The Table presents the fold changes in relative abundance, prevalence shifts and
weight or Importance of the taxonomical features, changes, and shifts. The prevalence shift value between the two classes has a
positive value when there is a higher prevalence in CRA and a negative value when there is a higher prevalence in the control group.

| | Fold change | | Weight |
|---|---|---|---|
| | in relative | Prevalence | or |
| Taxonomic or Gene Feature | abundance | Shift | importance |
| k__Bacteria\|p__Bacteroidetes\|c__Bacteroidia\|o__Bacteroidales\|f__Rikenellaceae\|g__Alistipes\|s__Alistipes_shahii | 0.237223339 | 0.111415275 | 1 |
| k__Bacteria\|p__Firmicutes\|c__Negativicuteso_Selenomonadales\|f__Selenomonadaceae\|g__Megamonas | 0.02008992 | 0.018614837 | 2 |
| K06946>>uncharacterized protein | −0.002562457 | −0.02518478 | 3 |
| k__Bacteria\|p__Bacteroidetes\|c__Bacteroidia\|o__Bacteroidales\|f__Bacteroidaceae\|g__Bacteroides\|s__Bacteroides__salyersiae | 0.174505025 | 0.13258509 | 4 |
| K22302>>transcriptional repressor of cell division inhibition gene dicB | 0.10645232 | 0.103681905 | 5 |
| K02968>>small subunit ribosomal protein S20 | 0.071022448 | −0.022082307 | 6 |
| K18767>>beta-lactamase class A CTX-M [EC:3.5.2.6] | 0.084206174 | 0.127178575 | 7 |
| k__Bacteria\|p__Firmicutes\|c__Clostridia\|o__Clostridiales\|f__Ruminococcaceae\|g__Ruthenibacterium | 0.173680717 | 0.074162789 | 8 |
| K01247>>DNA-3-methyladenine glycosylase II [EC:3.2.2.21] | −0.301304588 | −0.133406333 | 9 |
| K07266>>capsular polysaccharide export protein | 0 | 0.032051282 | 10 |
| k__Bacteria\|p__Firmicutes\|c__Erysipelotrichia\|o__Erysipelotrichales\|f__Erysipelotrichaceae\|g__Erysipelatoclostridium | −0.031742525 | −0.076147459 | 11 |
| s__Clostridium spiroforme | | | |
| k__Bacteria\|p__Firmicutes\|c__Clostridia\|o__Clostridiales\|f__Eubacteriaceae\|g__Eubacterium\|s__Eubacterium ventriosum | −0.244136508 | −0.222967424 | 12 |
| K18887>>ATP-binding cassette, subfamily B, multidrug efflux pump | −0.261494128 | −0.174924719 | 13 |
| k__Bacteria\|p__Proteobacteria\|c__Betproteobacteria\|o__Burkholderiales\|f__Sutterellaceae\|g__Sutterella | 0.021043631 | 0.044917419 | 14 |
| K07800>>AgrD protein | −0.336159772 | −0.162195456 | 15 |
| k__Bacteria\|p__Firmicutes\|c__Negativicutes\|o__Veillonellales\|f__Veillonellaceae\|g__Veillonella\|s__Veillonella tobetsuensis | 0 | 0.022082307 | 16 |
| K02950>>small subunit ribosomal protein S12 | 0.043168833 | 0 | 17 |
| K03552>>holliday junction resolvase Hjr [EC:3.1.22.4] | −0.061268658 | −0.10710375 | 18 |
| K22684>>metacaspase-1 [EC:3.4.22.-]] | 0.05562604 | 0.05881011 | 19 |
| K01215>>glucan 1,6-alpha-glucosidase [EC:3.2.1.70] | −0.36544687 | −0.222876175 | 20 |
| k__Bacteria\|p__Firmicutes\|c__Bacilli\|o__Lactobacillales\|f__Streptococcaceae\|g__Lactococcus | −0.009173375 | −0.039488092 | 21 |
| K13053>>cell division inhibitor SulA | 0.19007252 | 0.157883931 | 22 |
| K17250>>GalNAc5-diNAcBac-PP-undecaprenol beta-1,3-glucosyltransferase [EC:2.4.1.293] | 0.01855502 | 0.070535633 | 23 |
| k__Bacteria\|p__Firmicutes\|c__Bacilli\|o__Bacillales\|f__Bacillales__unclassified g__Gemella\|s__Gemella sanguinis | −0.023427274 | 0.001824984 | 24 |
| K16087>>hemoglobin/transferrin/lactoferrin receptor protein | 0.160646835 | 0.142919062 | 25 |
| K03342>>para-aminobenzoate synthetase/4-amino-4-deoxychorismate lyase [EC:2.6.1.85 4.1.3.38] | −0.228532904 | −0.201729172 | 26 |
| K18830>>HTH-type transcriptional regulator/antitoxin PezA | −0.226911857 | −0.17741126 | 27 |
| K00411>>ubiquinol-cytochrome c__reductase iron-sulfur subunit [EC:7.1.1.8] | 0.007627254 | 0.068391277 | 28 |
| k__Bacteria\|p__Firmicutes\|c__Firmicute\|s__unclassified o__Firmicutes__unclassified\|f__Firmicutes__unclassified | −0.102802354 | −0.095629163 | 29 |
| g__Firmicutes__unclassified\|s__Firmicutes__bacterium CAG 110 | | | |
| K02919>>large subunit ribosomal protein L36 | −0.346257206 | −0.004630897 | 30 |
| K02455>>general secretion pathway protein F | 0.009669537 | 0.010151474 | 31 |
| K01886>>glutaminyl-tRNA synthetase [EC:6.1.1.18] | −0.027934885 | −0.056642942 | 32 |
| K03399>>cobalt-precorrin-7 (C5)-methyltransferase [EC:2.1.1.289] | 0.171074726 | 0.127224199 | 33 |
| K06904>>uncharacterized protein | −0.09585099 | −0.040583082 | 34 |
| K22373>>lactate racemase [EC:5.1.2.1] | −0.395605335 | −0.151496487 | 35 |
| k__Bacteria\|p__Firmicutes\|c__Bacilli\|o__Bacillales | −0.013697497 | 0.043503057 | 36 |
| K02913>>large subunit ribosomal protein L33 | 0.116174886 | −0.006410256 | 37 |
| K00330>>NADH-quinone oxidoreductase subunit A [EC:7.1.1.2] | 0.148415707 | −0.0228123 | 38 |
| K03738>>aldehyde:ferredoxin oxidoreductase [EC:1.2.7.5] | −0.199969387 | −0.192421754 | 39 |
| K13630>>multiple antibiotic__resistance protein MarB | 0.224184142 | 0.146842778 | 40 |
| k__Bacteria\|p__Firmicutes\|c__Clostridia\|o__Clostridiales\|f__Ruminococcaceae\|g__Ruminococcaceae unclassified | 0.005446961 | 0.017132038 | 41 |
| s__Ruminococcaceae bacterium D16 | | | |
| K00322>>NAD(P) transhydrogenase [EC:1.6.1.1] | 0.420138962 | 0.199356693 | 42 |
| K20483>>lantibiotic__biosynthesis__protein | −0.037916141 | −0.110662469 | 43 |
| K03325>>arsenite transporter | −0.407662275 | −0.114129939 | 44 |
| K01579>>aspartate 1-decarboxylase [EC:4.1.1.11] | 0.121928926 | −0.011041153 | 45 |
| K18698>>beta-lactamase class A TEM [EC:3.5.2.6] | 0.33237586 | 0.21518843 | 46 |
| k__Bacteria\|p__Firmicutes\|c__Clostridia\|o__Clostridiales\|f__Lachnospiraceae\|g__Roseburia\|s__Roseburia sp__CAG 309 | −0.00542487 | −0.061205402 | 47 |
| k__Bacteria\|p__Firmicutes\|c__Clostridia\|o__Clostridiales\|f__Eubacteriaceae | −0.419831145 | −0.048133954 | 48 |
| k__Bacteria\|p__Firmicutes\|c__Clostridia\|o__Clostridiales\|f__Eubacteriaceae\|g__Eubacterium | −0.420220659 | −0.048133954 | 49 |
| k__Bacteria\|p__Proteobacteria\|c__Betaproteobacteria\|o__Neisseriales\|f__Neisseriaceae\|g__Neisseria | 0.001524452 | 0.028492563 | 50 |
| k__Bacteria\|p__Firmicutes\|c_Clostridial\|o__Clostridiales\|f__Ruminococcaceae\|g__Ruminococcaceae unclassified\| | −0.071085337 | −0.055388265 | 51 |
| s__Clostridium leptum | | | |
| K07316>>adenine-specific__DNA-methyltransferase [EC:2.1.1.72] | −0.359546704 | −0.115202117 | 52 |
| K12144>>hydrogenase-4 component I [EC:1.-.-.-]] | 0.187408262 | 0.147207774 | 53 |
| K19954>>alcohol dehydrogenase [EC:1.1.1.-]] | −0.190158046 | −0.153663655 | 54 |
| K03427>>type I restriction enzyme M protein [EC:2.1.1.72] | −0.117661849 | −0.002851538 | 55 |
| K03116>>sec-independent protein translocase protein TatA | 0.043556448 | −0.009626791 | 56 |
| K14053>>outer membrane protein G | 0.32182116 | 0.14944338 | 57 |
| K16927>>energy-coupling factor transport system substrate-specific component | −0.546169548 | −0.238799161 | 58 |
| K06993>>ribonuclease H-related protein | −0.48293026 | −0.276621955 | 59 |
| k__Bacteria\|p__Bacteroidetes\|c__Bacteroidia\|o__Bacteroidales\|f__Bacteroidaceae\|g__Bacteroides\|s__Bacteroides nordii | 0.00420625 | −0.005680263 | 60 |
| K00561>>23S rRNA (adenine-N6)-dimethyltransferase [EC:2.1.1.184] | 0.34409869 | 0.041199015 | 61 |
| K19510>>fructoselysine-6-phosphate deglycase | −0.288849082 | −0.169426955 | 62 |
| K02671>>type IV pilus assembly protein PilV | −0.364342679 | −0.176704079 | 63 |
| K03207>>colanic acid biosynthesis protein WcaH [EC:3.6.1.-]] | 0.274276683 | 0.151177115 | 64 |
| K02744>>N-acetylgalactosamine PTS system EIIA component [EC:2.7.1.-]] | −0.448238222 | −0.217013414 | 65 |
| k__Bacteria\|p__Proteobacteria\|c_Betaproteobacteria\|o__Neisseriales\|f__Neisseriaceae\|g__Neisseria\|s__Neisseria flavescens | 0 | 0.028492563 | 66 |
| K02443>>glycerol uptake operon antiterminator | −0.074686342 | −0.042659002 | 67 |
| k__Bacteria\|p__Firmicutes\|c_Clostridia\|o_Clostridiales\|f_Oscillospiraceae\|g__Oscillibacter\|s__Oscillibacter sp__CAG 241 | −0.112960802 | −0.11483712 | 68 |
| K03826>>putative acetyltransferase [EC:2.3.1.-]] | −0.375749203 | −0.120061137 | 69 |
| K06042>>precorrin-8X/cobalt-precorrin-8 methylmutase [EC:5.4.99.61 5.4.99.60] | −0.214530886 | −0.066680354 | 70 |
| K03897>>lysine N6-hydroxylase [EC:1.14.13.59] | 0.165916217 | 0.14538279 | 71 |
| K10192>>oligogalacturonide transport system substrate-binding protein | −0.284961924 | −0.069896888 | 72 |
| K17331>>N,N'-diacetylchitobiose transport system permease protein | −0.388604478 | −0.158773611 | 73 |
| K00558>>DNA (cytosine-5)-methyltransferase 1 [EC:2.1.1.37] | −0.223561462 | −0.03490282 | 74 |
| K01960>>pyruvate carboxylase subunit B [EC:6.4.1.1] | 0.199042014 | 0.047472397 | 75 |
| K02027>>multiple sugar transport system substrate-binding protein | −0.377721054 | −0.01567205 | 76 |
| K18699>>beta-lactamase class A SHV [EC:3.5.2.6] | 0.142754944 | 0.121156127 | 77 |
| k__Bacteria\|p__Bacteroidetes\|c__Bacteroidia\|o__Bacteroidales\|f__Bacteroidaceae\|g__Bacteroide\|s__Bacteroides_plebeius | 0.127928513 | 0.077835569 | 78 |
| K04046>>hypothetical chaperone protein | 0.228273166 | 0.140500958 | 79 |
| k__Bacteria\|p__Bacteroidetes\|c__Bacteroidia\|o__Bacteroidales\|f__Odoribacteraceae\|g__Butyricimonas\|s__Butyricimonas_virosa | 0.089047268 | 0.060019162 | 80 |
| K07488>>transposase | 0.060822072 | 0.104731271 | 81 |
| K10022>>arginine/ornithine transport system substrate-binding protein | 0 | 0.036682179 | 82 |
| K19119>>CRISPR-associated protein Cas5d | −0.379714265 | −0.08052742 | 83 |
| K15577>>nitrate/nitrite transport system permease protein | 0.0836294 | 0.108677799 | 84 |
| K08363>>mercuric__ion transport protein | 0.150405655 | 0.12197737 | 85 |
| K07133>>uncharacterized protein | −0.124691544 | −0.004630897 | 86 |
| K11903>>type VI secretion system secreted protein Hop | 0.241557859 | 0.14369468 | 87 |
| K00177>>2-oxoglutarate ferredoxin oxidoreductase subunit gamma [EC:1.2.7.3] | −0.411385605 | −0.163929191 | 88 |
| k__Bacteria\|p__Firmicutes\|c_Negativicutes\|o__Veillonellales\|f__Veillonellaceae\|g__Veillonella\|s__Veillonella_atypica | 0.041927484 | 0.056004197 | 89 |
| k__Bacteria\|p__Firmicutes\|c__Bacilli\|o__Lactobacillus\|f__Streptococcaceae\|g__Streptococcus\|s__Streptococcus_thermophilus | −0.23240691 | −0.227324573 | 90 |
| K05341>>amylosucrase [EC:2.4.1.4] | −0.36359973 | −0.187243362 | 91 |
| K02196>>heme exporter protein D | 0.392853426 | 0.160872342 | 92 |
| K09116>>uncharacterized protein | −0.155914623 | −0.151177115 | 93 |
| K11962>>urea transport system ATP-binding protein | 0 | 0.053426408 | 94 |
| K09908>>uncharacterized protein | 0.380552873 | 0.181859659 | 95 |
| k__Bacteria\|p__Firmicutes\|c__Clostridia\|o__Clostridiales\|f__Ruminococcaceae\|g__Ruminococcus | −0.491152896 | −0.140843143 | 96 |
| K12296>>competence protein ComX | −0.327805639 | −0.230107674 | 97 |
| K09777>>uncharacterized protein | −0.372595366 | −0.078018067 | 98 |
| K06122>>glycerol dehydratase small subunit [EC:4.2.1.30] | 0.09405126 | 0.098343827 | 99 |
| K02523>>octaprenyl-diphosphate synthase [EC:2.5.1.90] | 0.425111621 | 0.083515832 | 100 |
| K09793>>uncharacterized protein | 0.35889625 | 0.113331508 | 101 |
| K11075>>putrescine transport system permease protein | 0.34053422 | 0.249178757 | 102 |
| K13771>>Rrf2 family transcriptional regulator, nitric oxide-sensitive transcriptional repressor | 0.378760793 | 0.153093348 | 103 |
| K05346>>deoxyribonucleoside regulator | −0.529980944 | −0.247741582 | 104 |
| K19506>>fructoselysine/glucoselysine PTS system EIIA component [EC:2.7.1.-]] | −0.432524803 | −0.094830733 | 105 |
| K06866>>autonomous glycyl radical cofactor | 0.347870539 | 0.143512182 | 106 |
| K07341>>death on curing protein | −0.427606332 | −0.21737841 | 107 |
| K06923>>uncharacterized protein | 0.360051459 | −0.056277945 | 108 |
| K01338>>ATP-dependent Lon protease [EC:3.4.21.53] | −0.100941991 | −0.009261794 | 109 |
| K07321>>CO dehydrogenase maturation factor | −0.536153114 | −0.128250753 | 110 |
| K07269>>uncharacterized protein | 0.36742192 | 0.195045168 | 111 |
| K06902>>MFS transporter, UMF1 family | −0.489959777 | −0.122547678 | 112 |
| K00172>>pyruvate ferredoxin oxidoreductase gamma subunit [EC:1.2.7.1] | −0.375684188 | −0.147184962 | 113 |
| K00702>>cellobiose phosphorylase [EC:2.4.1.20] | −0.506708496 | −0.098024455 | 114 |
| K11529>>glycerate 2-kinase [EC:2.7.1.165] | −0.429334371 | −0.147892143 | 115 |
| K06438>>similar to stage IV sporulation protein | −0.41795522 | −0.114494936 | 116 |
| K11741>>quaternary ammonium compound-resistance protein SugE | 0.471968494 | 0.155009581 | 117 |
| K15342>>CRISP-associated protein Cas1 | −0.217660434 | −0.022447304 | 118 |
| K18206>>beta-L-arabinobiosidase [EC:3.2.1.187] | −0.387974024 | −0.175517839 | 119 |
| K01744>>aspartate ammonia-lyase [EC:4.3.1.1] | 0.215677707 | −0.003216534 | 120 |
| K12678>>autotransporter family porin | 0.010271271 | 0.009398668 | 121 |
| K14088>>ech hydrogenase subunit C | −0.241284408 | −0.188794598 | 122 |
| K15533>>1,3-beta-galactosyl-N-acetylhexosamine phosphorylase [EC:2.4.1.211] | −0.366522374 | −0.152545853 | 123 |
| K07456>>DNA mismatch repair protein MutS2 | −0.140863935 | −0.010333972 | 124 |
| k__Bacteria\|p__Firmicutes\|c__Clostridia o__Clostridiales\|f__Eubacteriaceae\|g__Eubacterium\|s__Eubacterium hallii | −0.424140254 | −0.286933114 | 125 |
| K08191>>MFS transporter, ACS family, hexuronate transporter | 0.49071087 | 0.151496487 | 126 |
| K07493>>putative transposase | −0.452294835 | −0.275960398 | 127 |
| K06859>>glucose-6-phosphate isomerase, archaeal [EC:5.3.1.9] | −0.399935539 | −0.121521124 | 128 |
| K01182>>oligo-1,6-glucosidase [EC:3.2.1.10] | −0.371855397 | −0.057372935 | 129 |
| K14187>>chorismate mutase/prephenate dehydrogenase [EC:5.4.99.5 1.3.1.12] | 0.349272828 | 0.174080664 | 130 |
| K07814>>putative two-component system response regulator | −0.441570804 | −0.140044712 | 131 |
| K06891>>ATP-dependent C\|p protease adaptor protein ClpS | 0.370699337 | 0.096610092 | 132 |
| K21469>>serine-type D-Ala-D-Ala carboxypeptidase [EC:3.4.16.4] | −0.096801882 | −0.006661192 | 133 |
| k__Bacteria\|p__Firmicutes\|c__Erysipelotrichia\|o__Erysipelotrichales\|f__Erysipelotrichaceae\|g__Turicibacter | −0.010012811 | −0.00882836 | 134 |
| K02952>>small subunit ribosomal protein S13 | 0.063769399 | −0.006410256 | 135 |
| K13527>>proteasome-associated ATPase | −0.592535821 | −0.201432612 | 136 |
| K07397>>putative redox protein | 0.482103191 | 0.115521489 | 137 |
| K07040>>uncharacterized protein | −0.241065018 | −0.005703075 | 138 |
| K05343>>maltose alpha-D-glucosyltransferase/alpha-amylase [EC:5.4.99.16 3.2.1.1] | −0.452622843 | −0.156606442 | 139 |
| K13816>>DSF synthase | 0 | 0.036682179 | 140 |
| K20373>>HTH-type transcriptional regulator, SHP2-responsive activator | −0.281412618 | −0.20090793 | 141 |
| K02026>>multiple sugar transport system permease protein | −0.300877142 | −0.028492563 | 142 |
| k__Bacteria\|p__Firmicutes\|c__Clostridia\|o__Clostridiales\|f__Clostridiaceae\|g__Clostridium | −0.182720483 | −0.094283238 | 143 |
| k__Bacteria\|p__Proteobacteria\|c__Gammaproteobacteria\|o__Enterobacterales\|f__Enterobacteriaceae\|g__Enterobacter | 0.00029801 | 0.029222557 | 144 |
| K14170>>chorismate mutase/prephenate dehydratase [EC:5.4.99.5 4.2.1.51] | −0.285303372 | −0.050232685 | 145 |
| K00170>>pyruvate ferredoxin oxidoreductase beta subunit [EC:1.2.7.1] | −0.325999408 | −0.119080208 | 146 |
| K19285>>FMN reductase (NADPH) [EC:1.5.1.38] | −0.213621047 | −0.183479332 | 147 |
| K00331>>NADH-quinone oxidoreductase subunit B [EC:7.1.1.2] | 0.132105632 | −0.024226663 | 148 |
| K02412>>flagellum-specific ATP synthase [EC:7.4.2.8] | −0.215052441 | −0.049525504 | 149 |
| K03436>>DeoR family transcriptional regulator, fructose operon transcriptional repressor | −0.414892614 | −0.132174468 | 150 |
| K00937>>polyphosphate kinase [EC:2.7.4.1] | −0.215437116 | −0.030636919 | 151 |
| K07177>>Lon-like protease | −0.412054023 | −0.157655808 | 152 |
| K01802>>peptidylprolyl isomerase [EC:5.2.1.8] | −0.569349168 | −0.180741856 | 153 |
| K12632>>puromycin N-acetyltransferase [EC:2.3.-.-] | 0 | 0.038461538 | 154 |
| K02173>>putative kinase | −0.358466931 | −0.136577242 | 155 |
| K02687>>ribosomal protein L11 methyltransferase [EC:2.1.1.-]] | −0.077148496 | −0.004630897 | 156 |
| K02557>>chemotaxis protein MotB | −0.133132695 | −0.12396204 | 157 |
| K07106>>N-acetylmuramic acid 6-phosphate etherase [EC:4.2.1.126] | 0.083530249 | −0.06376038 | 158 |
| K06987>>uncharacterized protein | −0.473964515 | −0.065562551 | 159 |
| K18349>>two-component system, OmpR family, response regulator VanR | −0.38684769 | −0.009649603 | 160 |
| K07334>>toxin HigB-1 | 0.064221045 | −0.004379962 | 161 |
| K00690>>sucrose phosphorylase [EC:2.4.1.7] | −0.603508762 | −0.12396204 | 162 |
| K08169>>MFS transporter, DHA2 family, multidrug__resistance protein | 0.380634372 | 0.104434711 | 163 |
| K12141>>hydrogenase-4 component F [EC:1.-.-.-] | 0.383642257 | 0.162423579 | 164 |
| K06408>>stage V sporulation protein AF | −0.380858981 | −0.116981476 | 165 |
| K04085>>tRNA 2-thiouridine synthesizing__protein A [EC:2.8.1.-] | 0.372699276 | 0.154119901 | 166 |
| k__Bacteria\|p__Bacteroidetes\|c__Bacteroidia\|o__Bacteroidales\|f__Tannerellaceae\|g_Parabacteroides\|s__Parabacteroides_gordonii | 0.001517599 | 0.010333972 | 167 |
| K13280>>signal peptidase I [EC:3.4.21.89] | −0.533484168 | −0.213819692 | 168 |
| k__Bacteria\|p__Firmicutes\|c__Clostridia\|o__Clostridiales\|f__Lachnospiraceae\|g__Dorea | −0.4185234 | −0.146135596 | 169 |
| K03839>>flavodoxin I | 0.207100065 | −0.023565106 | 170 |
| K00483>>4-hydroxyphenylacetate 3-monooxygenase [EC:1.14.14.9] | 0.11091775 | 0.127566384 | 171 |
| K04018>>formate-dependent nitrite reductase complex subunit NrfG | 0.307332323 | 0.153389908 | 172 |
| K05570>>multicomponent Na+:H+ antiporter subunit F | −0.243356089 | −0.187060863 | 173 |
| K02077>>zinc/manganese transport system substrate-binding protein | −0.610212984 | −0.21281595 | 174 |
| K00769>>xanthine phosphoribosyltransferase [EC:2.4.2.22] | 0.342861462 | 0.160530158 | 175 |
| K01835>>phosphoglucomutase [EC:5.4.2.2] | −0.035877841 | −0.006410256 | 176 |
| K19294>>alginate O-acetyltransferase complex protein AlgI | −0.256998786 | −0.066634729 | 177 |
| K02391>>flagellar basal-body rod protein FlgF | 0.227553042 | 0.16719135 | 178 |
| k__Bacteria\|p__Bacteroidetes\|c__Bacteroidia\|o__Bacteroidales\|f__Prevotellaceae\|g__Prevotella\|s__Prevotella stercorea | 0.013916381 | 0.018569213 | 179 |
| K00613>>glycine amidinotransferase [EC:2.1.4.1] | −0.44024414 | −0.265124555 | 180 |
| K07588>>LAO/AO transport system kinase [EC:2.7.-.-] | −0.025261682 | −0.043845241 | 181 |
| K00549>>5-methyltetrahydropteroyltriglutamate--homocysteine methyltransferase [EC:2.1.1.14] | −0.326428125 | −0.043457432 | 182 |
| K05936>>precorrin-4/cobalt-precorrin-4 C11-methyltransferase [EC:2.1.1.133 2.1.1.271] | −0.231998436 | −0.075531527 | 183 |
| K00247>>fumarate reductase subunit D | 0.429381807 | 0.152477416 | 184 |
| K00131>>glyceraldehyde-3-phosphate dehydrogenase (NADP+) [EC:1.2.1.9] | −0.32621042 | −0.176179396 | 185 |
| K04784>>yersiniabactin nonribosomal peptide synthetase | 0.154137243 | 0.140432521 | 186 |
| K10117>>raffinose/stachyose/melibiose transport system substrate-binding protein | −0.412464103 | −0.011041153 | 187 |
| k__Bacteria\|p__Actinobacteria\|c__Coriobacteriia\|o__Eggerthellales\|f__Eggerthellaceae\|g__Gordonibacter | −0.081634917 | −0.104480336 | 188 |
| K05808>>putative sigma-54 modulation protein | −0.052034608 | −0.006775253 | 189 |
| K02278>>prepilin peptidase CpaA [EC:3.4.23.43] | −0.39127257 | −0.150789306 | 190 |
| K07029>>diacylglycerol kinase (ATP) [EC:2.7.1.107] | −0.559199366 | −0.230244548 | 191 |
| K13626>>flagellar assembly factor FliW | −0.364643301 | −0.072725614 | 192 |
| K13954>>alcohol dehydrogenase [EC:1.1.1.1] | −0.06804374 | −0.015945798 | 193 |
| K05833>>putative ABC transport system ATP-binding protein | −0.183663832 | −0.026713204 | 194 |
| K08168>>MFS transporter, DHA2 family, metal-tetracycline-proton antiporter | −0.386995629 | −0.087872981 | 195 |
| K04760>>transcription elongation factor GreB | 0.334518352 | 0.166643854 | 196 |
| K06960>>uncharacterized protein | −0.241451768 | −0.03312346 | 197 |
| K05838>>putative thioredoxin | 0.320171151 | 0.165229492 | 198 |
| K06921>>uncharacterized protein | −0.206635142 | −0.082283968 | 199 |
| K07080>>uncharacterized protein | −0.191576814 | −0.037047176 | 200 |
| K03700>>recombination protein U | −0.296724036 | −0.05632357 | 201 |
| K00372>>assimilatory nitrate reductase catalytic subunit [EC:1.7.99.-] | 0.21723546 | 0.112304955 | 202 |
| K11720>>lipopolysaccharide export system permease protein | 0.088606265 | −0.05771512 | 203 |
| K02081>>DeoR family transcriptional regulator, aga operon transcriptional repressor | 0.24391737 | 0.016995164 | 204 |
| K02385>>flagellar protein F1bD | −0.367887294 | −0.137558171 | 205 |
| K02911>>large subunit ribosomal protein L32 | 0.133827336 | −0.006410256 | 206 |
| K10006>>glutamate transport system permease protein | −0.55751013 | −0.167465097 | 207 |
| K22736>>vacuolar iron transporter family protein | −0.31383694 | −0.213180947 | 208 |
| K02647>>carbohydrate diacid regulator | −0.193446732 | −0.013892691 | 209 |
| K08369>>MFS transporter, putative metabolite:H+ symporter | −0.326810531 | −0.053129848 | 210 |
| K07404>>6-phosphogluconolactonase [EC:3.1.1.31] | −0.126035451 | −0.036704991 | 211 |
| K07768>>two-component system, OmpR family, sensor histidine kinase SenX3 [EC:2.7.13.3] | −0.449372603 | −0.183775892 | 212 |
| K20461>>lantibiotic transport system permease protein | −0.428892317 | −0.113331508 | 213 |
| K04564>>superoxide dismutase, Fe-Mn family [EC:1.15.1.1] | 0.147877756 | −0.046673967 | 214 |
| K07461>>putative endonuclease | 0.073890087 | −0.004995894 | 215 |
| K03559>>biopolymer transport protein ExbD | 0.114958608 | −0.060201661 | 216 |
| k__Bacteria\|p__Firmicutes\|c__Bacilli\|o__Lactobacillales\|f__Lactobacillaceae | −0.126423123 | −0.125513277 | 217 |
| K08217>>MFS transporter, DHA3 family, macrolide efflux protein | −0.239306544 | −0.035974998 | 218 |
| K05810>>polyphenol oxidase [EC:1.10.3.-]] | −0.086393128 | −0.01567205 | 219 |
| K04744>>LPS-assembly protein | 0.304936222 | 0.178346564 | 220 |
| K01697>>cystathionine beta-synthase [EC:4.2.1.22] | −0.557809936 | −0.198923259 | 221 |
| K17810>>D-aspartate ligase [EC:6.3.1.12] | −0.399094787 | −0.118669587 | 222 |
| K05568>>multicomponent Na+:H+ antiporter subunit D | −0.321007186 | −0.167738845 | 223 |
| K07700>>two-component system, CitB family, cit operon sensor histidine kinase CitA [EC:2.7.13.3] | 0.164177526 | 0.100967242 | 224 |
| K00303>>sarcosine oxidase, subunit beta [EC:1.5.3.1] | −0.146202928 | −0.130577607 | 225 |
| k__Bacteria\|p__Bacteroidetes\|c__Bacteroidia\|o__Bacteroidales\|f__Porphyromonadaceae\|g__Porphyromonas | 0.002110242 | 0.019595766 | 226 |
| K02004>>putative ABC transport system permease protein | −0.099922341 | −0.006410256 | 227 |
| k__Bacteria\|p__Actinobacteria\|c__Actinobacteria\|o__Bifidobacteriales\|f__Bifidobacteriaceae\|g__Bifidobacterium | −0.574583716 | −0.162514828 | 228 |
| K02007>>cobalt/nickel transport system permease protein | −0.493411951 | −0.20061137 | 229 |
| K10000>>arginine transport system ATP-binding protein [EC:7.4.2.1] | 0.311509504 | 0.149808377 | 230 |
| K05364>>penicillin-binding protein A | −0.438355155 | −0.164294187 | 231 |
| K00763>>nicotinate phosphoribosyltransferase [EC:6.3.4.21] | −0.207942307 | −0.030636919 | 232 |
| K05982>>deoxyribonuclease V [EC:3.1.21.7] | 0.345921194 | 0.172666302 | 233 |
| K05939>>acyl-[acyl-carrier-protein]-phospholipid O-acyltransferase/long-chain-fatty-acid--[acyl-carrier-protein] | 0.285191961 | 0.163381695 | 234 |
| ligase [EC:2.3.1.40 6.2.1.20] | | | |
| K12452>>CDP-4-dehydro-6-deoxyglucose reductase, E1 [EC:1.17.1.1] | −0.286608316 | −0.096929464 | 235 |
| K01739>>cystathionine gamma-synthase [EC:2.5.1.48] | −0.211185825 | −0.042408066 | 236 |
| K07480>>insertion element IS1 protein InsB | 0.434770178 | 0.124692034 | 237 |
| K10118>>raffinose/stachyose/melibiose transport system permease protein | −0.521282227 | −0.066976914 | 238 |
| K07699>>two-component system, response regulator, stage 0 sporulation protein A | −0.285226819 | −0.033488457 | 239 |
| K00338>>NADH-quinone oxidoreductase subunit I [EC:7.1.1.2] | 0.187810127 | −0.046331782 | 240 |
| K03670>>periplasmic glucans biosynthesis protein | 0.336116841 | 0.19397299 | 241 |
| K00394>>adenylylsulfate reductase, subunit A [EC:1.8.99.2] | −0.658918676 | −0.194543298 | 242 |
| K19300>>aminoglycoside 3'-phosphotransferase II [EC:2.7.1.95] | 0 | 0.062323205 | 243 |
| K13574>>uncharacterized oxidoreductase [EC:1.1.1.-]] | 0.243155256 | 0.159047358 | 244 |
| K21903>>ArsR family transcriptional regulator, lead/cadmium/zinc/bismuth-responsive transcriptional repressor | −0.290670855 | −0.053084223 | 245 |
| K06384>>stage II sporulation protein M | −0.426098159 | −0.183296834 | 246 |
| K03817>>ribosomal-protein-serine acetyltransferase [EC:2.3.1.-] | 0.470408491 | 0.139679715 | 247 |
| K06928>>nucleoside-triphosphatase [EC:3.6.1.15] | −0.283529915 | −0.126676704 | 248 |
| k__Bacteria\|p__Firmicutes\|c__Bacilli\|o__Lactobacillales\|f__Lactobacillaceae\|g__Lactobacillus | −0.123911343 | −0.123733917 | 249 |
| K06412>>stage V sporulation protein G | −0.501660059 | −0.094420111 | 250 |
| K16213>>cellobiose epimerase [EC:5.1.3.11] | −0.409134936 | −0.104092527 | 251 |
| K07533>>foldase protein PrsA [EC:5.2.1.8] | −0.460266505 | −0.121840496 | 252 |
| k__Bacteria\|p__Firmicutes\|c_Clostridia\|o_Clostridiales\|f__Lachnospiraceae\|g__Roseburia\|s__Roseburia sp__CAG 471 | −0.037487478 | −0.064376312 | 253 |
| K02063>>thiamine transport system permease protein | 0.293956536 | 0.147686833 | 254 |
| K15581>>oligopeptide transport system permease protein | −0.187849877 | −0.021375125 | 255 |
| K06919>>putative DNA primase/helicase | −0.209974546 | −0.013892691 | 256 |
| K19776>>GntR family transcriptional regulator, galactonate operon transcriptional repressor | 0.336560868 | 0.167647596 | 257 |
| K07498>>putative transposase | −0.37225502 | −0.250775618 | 258 |
| K08138>>MFS transporter, SP family, xylose:H+ symportor | 0.228111876 | 0.168081029 | 259 |
| K02809>>PTS system, sucrose-specific IIB component [EC:2.7.1.69] | −0.36599144 | −0.066269733 | 260 |
| K00024>>malate dehydrogenase [EC:1.1.1.37] | 0.120467898 | −0.008554613 | 261 |
| K06399>>stage IV sporulation protein B [EC:3.4.21.116] | −0.393543645 | −0.103362533 | 262 |
| K16957>>L-cystine transport system substrate-binding protein | −0.287343336 | −0.194087052 | 263 |
| K07237>>tRNA 2-thiouridine synthesizing protein B | 0.450255587 | 0.180604982 | 264 |
| K00847>>fructokinase [EC:2.7.1.4] | −0.233433469 | −0.013892691 | 265 |
| K17319>>putative aldouronate transport system permease protein | −0.26815162 | −0.026006022 | 266 |
| K03589>>cell division protein FtsQ | 0.036828608 | −0.01567205 | 267 |
| K07017>>uncharacterized protein | 0.133269183 | 0.083629893 | 268 |
| K00183>>prokaryotic molybdopterin-containing oxidoreductase family, molybdopterin binding subunit | −0.251757112 | −0.186353682 | 269 |
| k__Bacteria\|p__Actinobacteria\|c__Actinobacteria\|o__Bifidobacteriales | −0.573694048 | −0.162514828 | 270 |
| K15738>>ABC transport system ATP-binding/permease protein | −0.201363699 | −0.050232685 | 271 |
| K21011>>polysaccharide biosynthesis protein PelF | −0.396874346 | −0.147914956 | 272 |
| K20490>>lantibiotic transport system ATP-bindingprotein | −0.253694254 | −0.017109225 | 273 |
| K06048>>glutamate---cysteine ligase/carboxylate-amine ligase [EC:6.3.2.2 6.3.-.-] | 0.324763438 | 0.140957204 | 274 |
| K12339>>S-sulfo-L-cysteine synthase (O-acetyl-L-serine-dependent) [EC:2.5.1.144] | 0.338228779 | 0.073911853 | 275 |
| K06284>>AbrB family transcriptional regulator, transcriptional pleiotropic regulator of transition state genes | −0.386194791 | −0.070558445 | 276 |
| k__Bacteria\|p__Bacteroidetes\|c__Bacteroidia\|o__Bacteroidales\|f__Barnesiellaceae\|g__Barnesiella\| | −0.089253717 | −0.080162424 | 277 |
| s__Barnesiella intestinihominis | | | |
| K16785>>energy-coupling factor transport system permease protein | −0.259159784 | −0.002851538 | 278 |
| K00691>>maltose phosphorylase [EC:2.4.1.8] | 0.314178022 | 0.124965782 | 279 |
| K16924>>energy-coupling factor transport system substrate-specific component | −0.446624912 | −0.138949722 | 280 |
| K06447>>succinylglutamic semialdehyde dehydrogenase [EC:1.2.1.71] | 0.279526864 | 0.171137878 | 281 |
| K05567>>multicomponent Na+:H+ antiporter subunit C | −0.241800615 | −0.183502144 | 282 |
| K20487>>two-component system, OmpR family, lantibiotic biosynthesis sensor histidine kinase NisK/SpaK [EC:2.7.13.3] | −0.501716539 | −0.236266995 | 283 |
| K17320>>putative aldouronate transport system permease protein | −0.36991778 | −0.066976914 | 284 |
| K00853>>L-ribulokinase [EC:2.7.1.16] | 0.330530025 | 0.079227119 | 285 |
| K06167>>phosphoribosyl 1,2-cyclic phosphate phosphodiesterase [EC:3.1.4.55] | 0.286483703 | 0.080983666 | 286 |
| K06173>>tRNA pseudouridine38-40 synthase [EC:5.4.99.12] | −0.056063873 | −0.002851538 | 287 |
| K10189>>lactose/L-arabinose transport system permease protein | −0.377325564 | −0.185714937 | 288 |
| K02314>>replicative DNA helicase [EC:3.6.4.12] | −0.261252127 | −0.009261794 | 289 |
| K15372>>taurine---2-oxoglutarate transaminase [EC:2.6.1.55] | −0.418590676 | −0.131170727 | 290 |
| K03932>>polyhydroxybutyrate depolymerase | 0.302410751 | 0.114266813 | 291 |
| K06190>>intracellular septation protein | 0.341368694 | 0.161967333 | 292 |
| K07345>>major type 1 subunit fimbrin (pilin) | 0.224543875 | 0.118738024 | 293 |
| K06199>>fluoride exporter | −0.014681555 | −0.058764486 | 294 |
| K07014>>uncharacterized protein | 0.294853248 | 0.099872251 | 295 |
| K07776>>two-component system, OmpR family, response regulator RegX3 | −0.294062379 | −0.224723971 | 296 |
| K00817>>histidinol-phosphate aminotransferase [EC:2.6.1.9] | −0.079810642 | −0.012820513 | 297 |
| K00209>>enoyl-[acyl-carrier protein] reductase/trans-2-enoyl-CoA reductase (NAD+) [EC:1.3.1.9 1.3.1.44] | −0.518158 | −0.181038416 | 298 |
| K05896>>segregation and condensation protein A | −0.294623694 | −0.031709098 | 299 |
| k__Bacteria\|p__Bacteroidetes\|c__Bacteroidia\|o__Bacteroidales\|f__Prevotellaceae\|g__Prevotella\|s__Prevotella_copri | 0.378618287 | 0.173601606 | 300 |
| K06215>>pyridoxal 5'-phosphate synthase pdxS subunit [EC:4.3.3.6] | −0.376395699 | −0.056642942 | 301 |
| K07192>>flotillin | −0.121742975 | −0.041335888 | 302 |
| K06378>>stage II sporulation protein AA (anti-sigma F factor antagonist) | −0.292724637 | −0.098343827 | 303 |
| K01239>>purine nucleosidase [EC:3.2.2.1] | −0.38170334 | −0.21112784 | 304 |
| k__Bacteria\|p__Actinobacteria\|c__Actinobacteria\|o__Actinomycetales\|f__Actinomycetaceae\|g__Actinomyces\| | −0.013868518 | −0.001049366 | 305 |
| s__Actinomyces_odontolyticus | | | |
| K12660>>2-dehydro-3-deoxy-L-rhamnonate aldolase [EC:4.1.2.53] | 0.320845798 | 0.167624783 | 306 |
| K06390>>stage III sporulation protein AA | −0.298314986 | −0.058787298 | 307 |
| K01322>>prolyl oligopeptidase [EC:3.4.21.26] | 0.32312946 | 0.139907838 | 308 |
| K12149>>DNA-damage-inducible protein I | 0.344843042 | 0.168674149 | 309 |
| K11748>>glutathione-regulated potassium-efflux system ancillary protein KefG | 0.346400379 | 0.160119536 | 310 |
| k__Bacteria\|p__Actinobacteria\|c__Coriobacteriia\|o__Coriobacteriales\|f__Coriobacteriaceae | −0.204557532 | −0.059289169 | 311 |
| K07285>>outer membrane lipoprotein | 0.312727703 | 0.133223834 | 312 |
| K12251>>N-carbamoylputrescine amidase [EC:3.5.1.53] | −0.288056175 | −0.121566749 | 313 |
| K09706>>uncharacterized protein | −0.543719841 | −0.256980564 | 314 |
| K06972>>presequence protease [EC:3.4.24.-] | −0.38524225 | −0.077675883 | 315 |
| K12340>>outer membrane protein | 0.142522039 | −0.02112419 | 316 |
| K04751>>nitrogen regulatory protein P-II 1 | −0.257561504 | −0.025298841 | 317 |
| K10112>>multiple sugar transport system ATP-binding protein | −0.273182425 | −0.006775253 | 318 |
| K08990>>putative membrane protein | 0.378798271 | 0.173715667 | 319 |
| K03687>>molecular chaperone GrpE | −0.141229858 | −0.006410256 | 320 |
| K02124>>V/A-type H+/Na+-transporting ATPase subunit K | −0.272160085 | −0.041678073 | 321 |
| K11189>> PTS-HPR phosphocarrier protein | −0.178009877 | −0.018523588 | 322 |
| K11184>>catabolite repression HPr-like protein | −0.363525592 | −0.069851264 | 323 |
| k__Bacteria\|p__Bacteroidetes\|c__Bacteroidia\|o__Bacteroidales\|f__Bacteroidaceae\|g__Bacteroides\|s__Bacteroides_eggerthii | −0.021641842 | −0.019801077 | 324 |
| K07503>>endonuclease [EC:3.1.-.-] | −0.6001593 | −0.187859294 | 325 |
| K07640>>two-component system, OmpR family, sensor histidine kinase CpxA [EC:2.7.13.3] | 0.340556107 | 0.125764212 | 326 |
| K12661>>L-rhamnonate dehydratase [EC:4.2.1.90] | 0.287468905 | 0.163701068 | 327 |
| K11051>>multidrug/hemolysin transport system permease protein | −0.443165795 | −0.163313259 | 328 |
| K01689>>enolase [EC:4.2.1.11] | −0.058277662 | −0.006410256 | 329 |
| K01223>>6-phospho-beta-glucosidase [EC:3.2.1.86] | −0.278995287 | −0.0185464 | 330 |
| K03895>>aerobactin synthase [EC:6.3.2.39] | 0.142034585 | 0.12544484 | 331 |
| K07337>>penicillin-binding protein activator | 0.396492919 | 0.206428506 | 332 |
| K11216>>autoinducer-2 kinase [EC:2.7.1.189] | 0.293248292 | 0.203554156 | 333 |
| K03482>>GntR family transcriptional regulator, glv operon transcriptional regulator | 0.332577934 | 0.159777352 | 334 |
| K07722>>CopG family transcriptional regulator, nickel-responsive regulator | 0.17596365 | 0.015101743 | 335 |
| K18979>>epoxyqueuosine reductase [EC:1.17.99.6] | 0.398756056 | 0.084565198 | 336 |
| K04653>>hydrogenase expression/formation protein HypC | 0.028055233 | −0.006866502 | 337 |
| K03288>>MFS transporter, MHS family, citrate/tricarballylate:H+ symporter | 0.166535676 | 0.12544484 | 338 |
| K03607>>ProP effector | 0.389792828 | 0.191851446 | 339 |
| K10010>>L-cystine transport system ATP-binding protein [EC:7.4.2.1] | −0.236072087 | −0.08201022 | 340 |
| K10546>>putative multiple sugar transport system substrate-binding protein | −0.596103164 | −0.147504334 | 341 |
| K12972>>glyoxylate/hydroxypyruvate reductase [EC:1.1.1.79 1.1.1.81] | 0.309342803 | 0.170476321 | 342 |
| K01963>>acetyl-CoA carboxylase carboxyl transferase subunit beta [EC:6.4.1.2 2.1.3.15] | −0.206924728 | −0.01318551 | 343 |
| K02217>>ferritin [EC:1.16.3.2] | 0.009398733 | −0.024933844 | 344 |
| K10015>>histidine transport system permease protein | 0.325265995 | 0.158431426 | 345 |
| K01924>>UDP-N-acetylmuramate--alanine ligase [EC:6.3.2.8] | −0.083808972 | −0.006410256 | 346 |
| K04568>>elongation factor P--(R)-beta-lysine ligase [EC:6.3.1.-]] | 0.36251566 | 0.134957569 | 347 |
| K15830>>formate hydrogenlyase subunit 5 | 0.266122951 | 0.173327858 | 348 |
| K07813>>accessory gene regulator B | −0.405713915 | −0.072337805 | 349 |
| K07469>>aldehyde oxidoreductase [EC:1.2.99.7] | −0.592559266 | −0.130737294 | 350 |
| K06997>>PLP dependent protein | −0.120881831 | −0.011041153 | 351 |
| K07124>>uncharacterized protein | −0.224961822 | −0.009626791 | 352 |
| K07261>>penicillin-insensitive murein DD-endopeptidase [EC:3.4.24.-]] | 0.376916974 | 0.198261703 | 353 |
| K15921>>arabinoxylan arabinofuranohydrolase [EC:3.2.1.55] | −0.480010686 | −0.210694406 | 354 |
| K19302>>undecaprenyl-diphosphatase [EC:3.6.1.27] | −0.079961093 | −0.011041153 | 355 |
| K12266>>anaerobic nitric oxide reductase transcription regulator | 0.288206445 | 0.155853636 | 356 |
| K07171>>mRNA interferase MazF [EC:3.1.-.-]] | −0.256288555 | −0.004630897 | 357 |
| K01992>>ABC-2 type transport system permease protein | −0.109416504 | −0.006410256 | 358 |
| K01200>>pullulanase [EC:3.2.1.41] | −0.384268093 | −0.070535633 | 359 |
| K05571>>multicomponent Na+:H+ antiporter subunit G | −0.230572773 | −0.194543298 | 360 |
| k__Bacteria\|p__Actinobacteria\|c__Coriobacteriia\|o__Eggerthellales\|f__Eggerthellaceae\|g__Enterorhabdus | −0.00970843 | 0.006068072 | 361 |
| K06916>>cell division protein ZapE | 0.314043853 | 0.174422849 | 362 |
| K11535>>nucleoside transport protein | 0.345786994 | 0.184026827 | 363 |
| K00380>>sulfite reductase (NADPH) flavoprotein alpha-component [EC:1.8.1.2] | 0.324468184 | 0.187562734 | 364 |
| K00375>>GntR family transcriptional regulator/MocR family aminotransferase | 0.447014567 | −0.071972808 | 365 |
| K11734>>aromatic amino acid transport protein AroP | 0.314470186 | 0.191509262 | 366 |
| K07015>>uncharacterized protein | 0.395470257 | −0.069486267 | 367 |
| K06142>>outer membrane protein | 0.090678767 | −0.048453326 | 368 |
| K03723>>transcription-repair coupling factor (superfamily II helicase) [EC:3.6.4.-]] | −0.190085336 | −0.023861666 | 369 |
| K18785>>beta-1,4-mannooligosaccharide/beta-1,4-mannosyl-N-acetylglucosamine phosphorylase [EC:2.4.1.319 2.4.1.320] | −0.086370003 | −0.071607811 | 370 |
| K07663>>two-component system, OmpR family, catabolic regulation response regulator CreB | 0.301907936 | 0.154416461 | 371 |
| K10017>>histidine transport system ATP-binding protein [EC:7.4.2.1] | 0.331615497 | 0.170156949 | 372 |
| K02407>>flagellar hook-associated protein 2 | −0.326463695 | −0.069144082 | 373 |
| k__Bacteria\|p__Firmicutes\|c__Clostridia\|o__Clostridiales\|f__Lachnospiraceae\|g__Dorea\|s__Dorea_longicatena | −0.367104179 | −0.158682362 | 374 |
| k__Bacteria\|p__Firmicutes\|c__Clostridia\|o__Clostridiales\|f__Clostridiaceae | −0.149656478 | −0.069303769 | 375 |
| K02008>>cobalt/nickel transport system permease protein | −0.426792345 | −0.187836481 | 376 |
| K20444>>O-antigen biosynthesis__protein [EC:2.4.1.-]] | −0.338441451 | −0.078018067 | 377 |
| K11927>>ATP-dependent RNA helicase RhIE [EC:3.6.4.13] | 0.272755762 | 0.061456337 | 378 |
| K13012>>O-antigen biosynthesis protein WbqP | −0.34606866 | −0.117278036 | 379 |
| K11928>>sodium/proline symporter | −0.327417678 | −0.018523588 | 380 |
| K07219>>putative molybdopterin biosynthesis protein | −0.311677845 | −0.198649512 | 381 |
| K01709>>CDP-glucose 4,6-dehydratase [EC:4.2.1.45] | −0.053968866 | −0.048613012 | 382 |
| K13256>>protein PsiE | 0.45507109 | 0.167761657 | 383 |
| K05569>>multicomponent Na+:H+ antiporter subunit E | −0.183134955 | −0.152203668 | 384 |
| K02361>>isochorismate synthase [EC:5.4.4.2] | 0.045999784 | −0.046058034 | 385 |
| K13571>>proteasome accessory factor A [EC:6.3.1.19] | −0.588216987 | −0.184300575 | 386 |
| K01046>>triacylglycerol lipase [EC:3.1.1.3] | −0.380127575 | −0.173054111 | 387 |
| K00760>>hypoxanthine phosphoribosyltransferase [EC:2.4.2.8] | 0.119404081 | −0.001072178 | 388 |
| K19267>>NAD(P)H dehydrogenase (quinone) [EC:1.6.5.2] | 0.281053567 | 0.158043617 | 389 |
| K14682>>amino-acid N-acetyltransferase [EC:2.3.1.1] | 0.330984511 | 0.194680172 | 390 |
| K03634>>outer membrane lipoprotein carrier protein | 0.400783898 | 0.175586276 | 391 |
| K02810>>sucrose PTS system EIIBCA or EIIBC component [EC:2.7.1.211] | −0.36599144 | −0.066269733 | 392 |
| K19118>>CRISPR-associated protein Csd2 | −0.341189545 | −0.042088694 | 393 |
| K15583>>oligopeptide transport system ATP-binding protein | −0.068178236 | −0.02885756 | 394 |
| K03190>>urease accessory protein | −0.383230589 | −0.132927274 | 395 |
| k__Bacteria\|p__Actinobacteria\|c_Coriobacteriia\|o__Coriobacteriales\|f__Coriobacteriaceae\|g__Enorma\|s__[Collinsella]_massiliensis | −0.018328971 | −0.00139155 | 396 |
| K22306>>glucosyl-3-phosphoglycerate phosphatase [EC:3.1.3.85] | −0.372163932 | −0.143922803 | 397 |
| K02803>>N-acetylglucosamine PTS system EIIB component [EC:2.7.1.193] | −0.290268775 | −0.036362807 | 398 |
| K06310>>spore germination protein | −0.4782134 | −0.110092162 | 399 |
| K15772>>arabinogalactan oligomer/maltooligosaccharide transport system permease protein | −0.33786383 | −0.094762296 | 400 |
| K06073>>vitamin B12 transport system permease protein | 0.346628487 | 0.158362989 | 401 |
| K18640>>plasmid segregation protein ParM | −0.33843054 | −0.042408066 | 402 |
| K12140>>hydrogenase-4 component E [EC:1.-.-.-]] | 0.352269974 | 0.073524044 | 403 |
| K05776>>molybdate transport system ATP-binding protein | 0.305956329 | 0.179030933 | 404 |
| K06968>>23S rRNA (cytidine2498-2'-O)-methyltransferase [EC:2.1.1.186] | 0.335017585 | 0.124349849 | 405 |
| K03704>>cold shock protein | 0.000781592 | −0.006410256 | 406 |
| K07166>>ACT domain-containing protein | 0.192581845 | −0.014964869 | 407 |
| K04773>>protease IV [EC:3.4.21.-]] | 0.302489504 | 0.014850808 | 408 |
| K09772>>cell division inhibitor SepF | −0.260107481 | −0.004630897 | 409 |
| K02804>>N-acetylglucosamine PTS system EIICBA or EIICB component [EC:2.7.1.193] | −0.288529715 | −0.034583447 | 410 |
| K11530>>(4S)-4-hydroxy-5-phosphonooxypentane-2,3-dione isomerase [EC:5.3.1.32] | 0.30478847 | 0.19853545 | 411 |
| K06202>>CyaY protein | 0.454470843 | 0.158112054 | 412 |
| K19309>>bacitracin transport system ATP-binding protein | −0.20602966 | −0.031412538 | 413 |
| K08314>>fructose-6-phosphate aldolase 2 [EC:4.1.2.-]] | 0.386574789 | 0.2071585 | 414 |
| K16092>>vitamin B12 transporter | 0.333438645 | 0.059311981 | 415 |
| K09762>>uncharacterized protein | −0.439207668 | −0.027078201 | 416 |
| K16137>>TetR/AcrR family transcriptional regulator, transcriptional repressor for nem operon | 0.326109074 | 0.143055936 | 417 |
| K09998>>arginine transport system permease protein | 0.386343116 | 0.152431791 | 418 |
| K11904>>type VI secretion system secreted protein VgrG | 0.162154238 | 0.126197646 | 419 |
| K03466>>DNA segregation ATPase FtsK/SpoIIIE, S-DNA-T family | −0.11012489 | −0.011041153 | 420 |
| K16147>>starch synthase (maltosyl-transferring) [EC:2.4.99.16] | −0.377298504 | −0.151633361 | 421 |
| K03587>>cell division protein FtsI (penicillin-binding protein 3) [EC:3.4.16.4] | 0.253193959 | 0.014143626 | 422 |
| K03970>>phage shock protein B | 0.393358715 | 0.175449402 | 423 |
| K05522>>endonuclease VIII [EC:3.2.2.- 4.2.99.18] | 0.288989341 | 0.16402044 | 424 |
| K06374>>spore maturation protein B | −0.358990141 | −0.091910758 | 425 |
| K08154>>MFS transporter, DHA1 family, 2-module integral membrane pump EmrD | 0.258782908 | 0.167236974 | 426 |
| K12265>>nitric oxide reductase FIRd-NAD(+) reductase [EC:1.18.1.-]] | 0.33713014 | 0.172985674 | 427 |
| K21140>>[CysO sulfur-carrier protein]-S-L-cysteine hydrolase [EC:3.13.1.6] | −0.416053101 | −0.146204033 | 428 |
| K01989>>putative ABC transport system substrate-binding protein | −0.168218833 | −0.024933844 | 429 |
| K06382>>stage II sporulation protein E [EC:3.1.3.16] | −0.296650124 | −0.026006022 | 430 |
| K01515>>ADP-ribose pyrophosphatase [EC:3.6.1.13] | −0.200003516 | −0.005703075 | 431 |
| K10108>>maltose/maltodextrin transport system substrate-binding protein | 0.263146997 | 0.140843143 | 432 |
| K12996>>rhamnosyltransferase [EC:2.4.1.-]] | −0.151893642 | −0.106236883 | 433 |
| k__Bacteria\|p__Firmicutes\|c__Clostridia\|o__Clostridiales\|f__Ruminococcaceae | −0.266086579 | −0.007482435 | 434 |
| K03732>>ATP-dependent RNA helicase RhlB [EC:3.6.4.13] | 0.367404272 | 0.110137786 | 435 |
| K15461>>tRNA 5-methylaminomethyl-2-thiouridine biosynthesis bifunctional protein [EC:2.1.1.61 1.5.-.-] | 0.283071061 | 0.17939593 | 436 |
| K00927>>phosphoglycerate kinase [EC:2.7.2.3] | −0.087955302 | −0.006410256 | 437 |
| K06223>>DNA adenine methylase [EC:2.1.1.72] | −0.306230888 | −0.06029291 | 438 |
| K13923>>phosphate propanoyltransferase [EC:2.3.1.222] | 0.166330372 | 0.126882015 | 439 |
| K13628>>iron-sulfur cluster assembly protein | 0.398316196 | 0.153458345 | 440 |
| K11072>>spermidine/putrescine transport system ATP-binding protein [EC:7.6.2.11] | −0.091662264 | −0.004630897 | 441 |
| K19508>>fructoselysine/glucoselysine PTS system EIIC component | −0.355747856 | −0.113399945 | 442 |
| K13787>>geranylgeranyl diphosphate synthase, type I [EC:2.5.1.1 2.5.1.10 2.5.1.29] | −0.43637601 | −0.192992061 | 443 |
| K01091>>phosphoglycolate phosphatase [EC:3.1.3.18] | −0.085553609 | −0.019230769 | 444 |
| K16264>>cobalt-zinc-cadmium efflux system protein | 0.241135711 | 0.023770417 | 445 |
| K11938>>HMP-PP phosphatase [EC:3.6.1.-]] | 0.334528906 | 0.122114244 | 446 |
| K02003>>putative ABC transport system ATP-binding protein | −0.122328189 | −0.006410256 | 447 |
| K13789>>geranylgeranyl diphosphate synthase, type II [EC:2.5.1.1 2.5.1.10 2.5.1.29] | −0.045684142 | −0.020302947 | 448 |
| K12952>>cation-transporting P-type ATPase E [EC:7.2.2.-]] | −0.421819992 | −0.117255224 | 449 |
| K07662>>two-component system, OmpR family, response regulator CpxR | 0.368907161 | 0.163792317 | 450 |
| K06346>>spoIIIJ-associated protein | −0.321044499 | −0.040605895 | 451 |
| K03205>>type IV secretion system protein VirD4 [EC:7.4.2.8] | −0.269667418 | −0.025641026 | 452 |
| K07109>>uncharacterized protein | 0.386041879 | 0.091272014 | 453 |
| K03548>>putative permease | 0.296012647 | 0.149922438 | 454 |
| K11103>>aerobic C4-dicarboxylate transport protein | 0.353791147 | 0.182635277 | 455 |
| K13940>>dihydroneopterin aldolase/2-amino-4-hydroxy-6-hydroxymethyldihydropteridine diphosphokinase [EC:4.1.2.25 2.7.6.3] | −0.429381883 | −0.170339447 | 456 |
| K07121>>uncharacterized protein | 0.354484146 | 0.19942513 | 457 |
| K03702>>excinuclease ABC subunit B | −0.097309333 | −0.004630897 | 458 |
| K02040>>phosphate transport system substrate-binding protein | −0.171370593 | −0.012820513 | 459 |
| K04488>>nitrogen fixation protein NifU and related proteins | −0.14030148 | −0.012820513 | 460 |
| K05787>>DNA-binding protein HU-alpha | 0.373389082 | 0.121521124 | 461 |
| K01534>>Zn2+/Cd2+-exporting ATPase [EC:7.2.2.12 7.2.2.21] | −0.172087449 | −0.009284606 | 462 |
| K04485>>DNA repair protein RadA/Sms | −0.174901727 | −0.001072178 | 463 |
| K14534>>4-hydroxybutyryl-CoA dehydratase/vinylacetyl-CoA-Delta-isomerase [EC:4.2.1.120 5.3.3.3] | −0.046485429 | 0.006615567 | 464 |
| K09897>>uncharacterized protein | 0.369073846 | 0.152796788 | 465 |
| K03837>>serine transporter | 0.304275229 | 0.172620677 | 466 |
| K19745>>acrylyl-CoA reductase (NADPH) [EC:1.3.1.-]] | 0.316230961 | 0.159800164 | 467 |
| K01193>>beta-fructofuranosidase [EC:3.2.1.26] | −0.235429214 | −0.012113332 | 468 |
| K05798>>LysR family transcriptional regulator, transcriptional activator for leuABCD operon | 0.278076241 | 0.162971074 | 469 |
| K00002>>alcohol dehydrogenase (NADP+) [EC:1.1.1.2] | −0.48789249 | −0.168925084 | 470 |
| K14540>>ribosome biogenesis GTPase A | −0.229799073 | −0.02885756 | 471 |
| K06298>>germination protein M | −0.362901255 | −0.173350671 | 472 |
| K18139>>outer membrane protein, multidrug efflux system | 0.390311927 | 0.095583539 | 473 |
| K04023>>ethanolamine transporter | 0.022556377 | −0.016470481 | 474 |
| K17759>>NAD(P)H-hydrate epimerase [EC:5.1.99.6] | −0.079015937 | −0.007482435 | 475 |
| K14761>>ribosome-associated protein | −0.116251742 | −0.038119354 | 476 |
| K09692>>teichoic acid transport system permease protein | −0.491739168 | −0.191760197 | 477 |
| K01058>>phospholipase A1/A2 [EC:3.1.1.32 3.1.1.4] | 0.315007856 | 0.084177388 | 478 |
| K05351>>D-xylulose reductase [EC:1.1.1.9] | −0.452914594 | −0.186946802 | 479 |
| K04092>>chorismate mutase [EC:5.4.99.5] | −0.510256308 | −0.150173373 | 480 |
| K15268>>O-acetylserine/cysteine efflux transporter | 0.22516416 | 0.15827174 | 481 |
| K02834>>ribosome-binding factor A | −0.078187761 | −0.006410256 | 482 |
| K07027>>glycosyltransferase 2 family protein | −0.083786146 | −0.007482435 | 483 |
| K17758>>ADP-dependent NAD(P)H-hydrate dehydratase [EC:4.2.1.136] | −0.081636167 | −0.007482435 | 484 |
| K00020>>3-hydroxyisobutyrate dehydrogenase [EC:1.1.1.31] | −0.242450311 | −0.154963957 | 485 |
| K07448>>restriction system protein | −0.380454673 | −0.160370472 | 486 |
| K07742>>uncharacterized protein | −0.337664638 | −0.032781276 | 487 |
| K00556>>tRNA (guanosine-2'-O-)-methyltransferase [EC:2.1.1.34] | 0.432535225 | 0.10660188 | 488 |
| K22601>>oxamate carbamoyltransferase [EC:2.1.3.5] | 0.100004351 | 0.097636646 | 489 |
| K05368>>aquacobalamin reductase/NAD(P)H-flavin reductase [EC:1.16.1.3 1.5.1.41] | 0.353171287 | 0.174765033 | 490 |
| K05774>>ribose 1,5-bisphosphokinase [EC:2.7.4.23] | 0.330386976 | 0.213568756 | 491 |
| K20074>>PPM family protein phosphatase [EC:3.1.3.16] | −0.299758118 | −0.007482435 | 492 |
| K11066>>N-acetylmuramoyl-L-alanine amidase [EC:3.5.1.28] | 0.367930501 | 0.176202208 | 493 |
| K06409>>stage V sporulation protein B | −0.235321181 | −0.01140615 | 494 |
| K09747>>uncharacterized protein | −0.18904626 | −0.011041153 | 495 |
| K16898>>ATP-dependent helicase/nuclease subunit A [EC:3.1.-.- 3.6.4.12] | −0.271270436 | −0.033853454 | 496 |
| K03147>>phosphomethylpyrimidine synthase [EC:4.1.99.17] | 0.042463469 | −0.019230769 | 497 |
| K11531>>Isr operon transcriptional repressor | 0.277860259 | 0.155465827 | 498 |
| K06383>>stage II sporulation protein GA (sporulation sigma-E factor processing peptidase) [EC:3.4.23.-]] | −0.477883703 | −0.049571129 | 499 |
| K09712>>uncharacterized protein | 0.343480477 | 0.19109864 | 500 |
| K08972>>putative membrane protein | −0.487017522 | −0.2056757 | 501 |
| K09781>>uncharacterized protein | 0.339787314 | 0.1886121 | 502 |
| K14056>>HTH-type transcriptional regulator, repressor for puuD | 0.325177003 | 0.171913496 | 503 |
| K07173>>S-ribosylhomocysteine lyase [EC:4.4.1.21] | −0.093179702 | −0.006410256 | 504 |
| K03425>>sec-independent protein translocase protein TatE | 0.41823702 | 0.175517839 | 505 |
| K07175>>PhoH-like ATPase | 0.10369594 | 0.022219181 | 506 |
| K01609>>indole-3-glycerol phosphate synthase [EC:4.1.1.48] | −0.239532374 | −0.034195638 | 507 |
| K01630>>2-dehydro-3-deoxyglucarate aldolase [EC:4.1.2.20] | 0.30223674 | 0.137786294 | 508 |
| k__Bacteria\|p__Firmicutes\|c__Clostridia\|o__Clostridiales\|f__Lachnospiraceae\|g__Anaerostipes | −0.487041101 | −0.227347386 | 509 |
| K00427>>L-lactate permease | 0.339872661 | 0.170156949 | 510 |
| K01421>>putative membrane protein | −0.370819363 | −0.02885756 | 511 |
| K09023>>aminoacrylate hydrolase [EC:3.5.1.-]] | 0.304748957 | 0.188931472 | 512 |
| K06966>>pyrimidine/purine-5'-nucleotide nucleosidase [EC:3.2.2.10 3.2.2.-]] | −0.163964002 | −0.029929738 | 513 |
| K08289>>phosphoribosylglycinamide formyltransferase 2 [EC:2.1.2.2] | 0.054115689 | −0.047016151 | 514 |
| K07570>>general stress protein 13 | −0.379920355 | −0.202915412 | 515 |
| K11745>>glutathione-regulated potassium-efflux system ancillary protein KefC | 0.328807102 | 0.186513368 | 516 |
| K00813>>aspartate aminotransferase [EC:2.6.1.1] | 0.326407434 | 0.178095629 | 517 |
| K03636>>sulfur-carrier protein | 0.391698217 | 0.155625513 | 518 |
| K09118>>uncharacterized protein | −0.587638771 | −0.168263528 | 519 |
| K01060>>cephalosporin-C deacetylase [EC:3.1.1.41] | 0.040639491 | 0.065562551 | 520 |
| K07012>>CRISPR-associated endonuclease/helicase Cas3 [EC:3.1.-.- 3.6.4.-]] | −0.329936431 | −0.137284424 | 521 |
| K09121>>pyridinium-3,5-bisthiocarboxylic__acid mononucleotide nickel chelatase [EC:4.99.1.12] | −0.242206619 | −0.02032576 | 522 |
| K00137>>aminobutyraldehyde dehydrogenase [EC:1.2.1.19] | 0.200874665 | 0.199562004 | 523 |
| K21672>>2,4-diaminopentanoate dehydrogenase [EC:1.4.1.12 1.4.1.26] | −0.415708281 | −0.187220549 | 524 |
| K07442>>tRNA (adenine57-N1/adenine58-N1)-methyltransferase catalytic subunit [EC:2.1.1.219 2.1.1.220] | −0.388356397 | −0.201523862 | 525 |
| K06405>>stage V sporulation protein AC | −0.253287253 | −0.04452961 | 526 |
| K09013>>Fe-S cluster assembly ATP-binding protein | −0.218349378 | −0.014964869 | 527 |
| K09384>>uncharacterized protein | −0.283788727 | −0.128684187 | 528 |
| K09470>>gamma-glutamylputrescine synthase [EC:6.3.1.11] | 0.286623205 | 0.16084953 | 529 |
| K05501>>TetR/AcrR family transcriptional regulator | 0.367341779 | 0.121452687 | 530 |
| K06403>>stage V sporulation protein AA | −0.378876801 | −0.075919336 | 531 |
| K21575>>neopullulanase [EC:3.2.1.135] | −0.004918801 | −0.066999726 | 532 |
| K08348>>formate dehydrogenase-N, alpha subunit [EC:1.17.5.3] | 0.398064274 | 0.158066429 | 533 |
| K11085>>ATP-binding cassette, subfamily B, bacterial MsbA [EC:3.6.3.-]] | 0.234889141 | −0.003649968 | 534 |
| K09693>>teichoic acid transport system ATP-binding protein [EC:7.5.2.4] | −0.349513243 | −0.101583174 | 535 |
| K08978>>bacterial/archaeal transporter family protein | −0.313975808 | −0.063441007 | 536 |
| K09972>>general L-amino acid transport system ATP-binding protein [EC:7.4.2.1] | 0.087143533 | 0.046514281 | 537 |
| K19068>>UDP-2-acetamido-2,6-beta-L-arabino-hexul-4-ose reductase [EC:1.1.1.367] | −0.396769807 | −0.126585455 | 538 |
| K01775>>alanine racemase [EC:5.1.1.1] | −0.00991131 | −0.016744228 | 539 |
| K05566>>multicomponent Na+:H+ antiporter subunit B | −0.228925108 | −0.182042157 | 540 |
| K18471>>methylglyoxal reductase [EC:1.1.1.-]] | −0.282655372 | −0.070558445 | 541 |
| K07042>>probable rRNA maturation factor | −0.179512263 | −0.002851538 | 542 |
| K01571>>oxaloacetate decarboxylase (Na+ extruding) subunit alpha [EC:7.2.4.2] | −0.208968837 | −0.036339995 | 543 |
| K03525>>type III pantothenate kinase [EC:2.7.1.33] | −0.144459212 | −0.006410256 | 544 |
| K06162>>alpha-D-ribose 1-methylphosphonate 5-triphosphate diphosphatase [EC:3.6.1.63] | 0.079759062 | −0.007801807 | 545 |
| K07799>>membrane fusion protein, multidrug efflux system | 0.29520815 | 0.162993886 | 546 |
| K00705>>4-alpha-glucanotransferase [EC:2.4.1.25] | −0.096474518 | −0.012820513 | 547 |
| K16345>>xanthine permease XanP | 0.256405239 | 0.035838124 | 548 |
| K02746>>N-acetylgalactosamine PTS system EIIC component | −0.320558158 | −0.095218542 | 549 |
| K07806>>UDP-4-amino-4-deoxy-L-arabinose-oxoglutarate aminotransferase [EC:2.6.1.87] | 0.329665198 | 0.190072087 | 550 |
| K08311>>putative (di)nucleoside polyphosphate hydrolase [EC:3.6.1.-]] | 0.408711674 | 0.137170362 | 551 |
| K14055>>universal stress protein E | 0.301638627 | 0.155237704 | 552 |
| K08219>>MFS transporter, UMF2 family, putative MFS family transporter protein | 0.264424641 | 0.187882106 | 553 |
| K07751>>PepB aminopeptidase [EC:3.4.11.23] | 0.40715473 | 0.162423579 | 554 |
| K08137>>MFS transporter, SP family, galactose:H+ symporter | 0.320574374 | 0.158408614 | 555 |
| k__Bacteria\|p__Actinobacteria\|c__Coriobacteriia\|o__Coriobacteriales\|f__Coriobacteriaceae\|g__Enorma | −0.031963021 | −0.029496304 | 556 |
| K01716>>3-hydroxyacyl-[acyl-carrier protein] dehydratase/trans-2-decenoyl-[acyl-carrier protein] isomerase [EC:4.2.1.59 5.3.3.14] | 0.414300181 | 0.15886486 | 557 |
| K07502>>uncharacterized protein | −0.388408555 | −0.084131764 | 558 |
| K00077>>2-dehydropantoate 2-reductase [EC:1.1.1.169] | −0.149594153 | 0.000707181 | 559 |
| K03835>>tryptophan-specific transport protein | 0.40415763 | 0.157815494 | 560 |
| K07260>>zinc D-Ala-D-Ala carboxypeptidase [EC:3.4.17.14] | −0.460723328 | −0.056688566 | 561 |
| k__Bacteria\|p_Bacteroidetes\|c__Bacteroidia\|o__Bacteroidales\|f__Barnesiellaceae\|g__Barnesiella | −0.090969101 | −0.083721142 | 562 |
| K08227>>MFS transporter, LPLT family, lysophospholipid transporter | 0.326544218 | 0.180445296 | 563 |
| K02521>>LysR family transcriptional regulator, positive regulator for ilvC | 0.354794622 | 0.193630806 | 564 |
| K00899>>5-methylthioribose kinase [EC:2.7.1.100] | 0.243717518 | 0.140135961 | 565 |
| K02812>>sorbose PTS system EIIA component [EC:2.7.1.206] | −0.40129776 | −0.201569486 | 566 |
| K03557>>Fis family transcriptional regulator, factor for inversion stimulation protein | 0.433286124 | 0.159594854 | 567 |
| k__Bacteria\|p__Proteobacteria\|c_Gammaproteobacteria\|o__Aeromonadales\|f__Aeromonadaceae | 0 | 0.022082307 | 568 |
| K01304>>pyroglutamyl-peptidase [EC:3.4.19.3] | −0.409737959 | −0.094785108 | 569 |
| K09158>>uncharacterized protein | 0.472279192 | 0.162400766 | 570 |
| K07287>>outer membrane protein assembly factor BamC | 0.317230778 | 0.136326307 | 571 |
| K21695>>protein AaeX | 0.418714943 | 0.155602701 | 572 |
| K05589>>cell division protein FtsB | 0.400444653 | 0.138972534 | 573 |
| K06894>>alpha-2-macroglobulin | 0.28617129 | 0.049730815 | 574 |
| K05967>>uncharacterized protein | −0.4704494 | −0.221028379 | 575 |
| K03562>>biopolymer transport protein TolQ | 0.362624462 | 0.148211516 | 576 |
| K10008>>glutamate transport system ATP-binding protein [EC:7.4.2.1] | −0.602549878 | −0.213865316 | 577 |
| K15531>>oligosaccharide reducing-end xylanase [EC:3.2.1.156] | −0.38063248 | −0.098047267 | 578 |
| K03502>>DNA polymerase V | −0.184762453 | −0.012113332 | 579 |
| K09780>>uncharacterized protein | 0.325048576 | 0.142782188 | 580 |
| K22105>>TetR/AcrR family transcriptional regulator, fatty acid biosynthesis__regulator | 0.375049971 | 0.154872707 | 581 |
| K12132>>eukaryotic-like serine/threonine-protein kinase [EC:2.7.11.1] | −0.355276331 | −0.045236792 | 582 |
| K07340>>inner membrane protein | 0.381756396 | 0.20428415 | 583 |
| K02761>>cellobiose PTS system EIIC component | −0.02167071 | 0.011223652 | 584 |
| K06957>>tRNA(Met) cytidine acetyltransferase [EC:2.3.1.193] | 0.318726188 | 0.18941053 | 585 |
| K09779>>uncharacterized protein | −0.413157302 | −0.062414454 | 586 |
| K03554>>recombination associated protein RdgC | 0.428640635 | 0.18662743 | 587 |
| K02433>>aspartyl-tRNA(Asn)/glutamyl-tRNA(Gln) amidotransferase subunit A [EC:6.3.5.6 6.3.5.7] | −0.232566733 | −0.012113332 | 588 |
| K04749>>anti-sigma B factor antagonist | 0.095016395 | −0.007756182 | 589 |
| K18778>>cell division protein ZapD | 0.340977941 | 0.166940414 | 590 |
| K04654>>hydrogenase expression/formation protein HypD | −0.393103175 | −0.069486267 | 591 |
| K00215>>4-hydroxy-tetrahydrodipicolinate reductase [EC:1.17.1.8] | −0.154498738 | −0.006410256 | 592 |
| K11358>>aspartate aminotransferase [EC:2.6.1.1] | −0.394342299 | −0.037412173 | 593 |
| K03324>>phosphate:Na+ symporter | −0.122142591 | −0.004630897 | 594 |
| K03224>>ATP synthase in type III secretion protein N [EC:7.4.2.8] | 0.150955141 | 0.124942969 | 595 |
| K18332>>NADP-reducing hydrogenase subunit HndD [EC:1.12.1.3] | 0.035214333 | −0.045259604 | 596 |
| K09999>>arginine transport system permease protein | 0.36189947 | 0.10085318 | 597 |
| K03590>>cell division protein FtsA | 0.038202119 | −0.048818323 | 598 |
| K01880>>glycyl-tRNA synthetase [EC:6.1.1.14] | −0.358893923 | −0.011041153 | 599 |
| K02572>>ferredoxin-type protein NapF | 0.322644351 | 0.151359613 | 600 |
| K10544>>D-xylose transport system permease protein | 0.300087084 | 0.158043617 | 601 |
| K09858>>SEC-C motif domain protein | 0.37549491 | 0.161602336 | 602 |
| k__Bacteria\|p__Proteobacteria\|c_Gammaproteobacteria | 0.431430695 | 0.093096998 | 603 |
| K02662>>type IV pilus assembly protein PilM | −0.498808705 | −0.120426134 | 604 |
| K22719>>murein hydrolase activator | 0.389634384 | 0.177593759 | 605 |
| K07773>>two-component system, OmpR family, aerobic respiration control protein ArcA | 0.340709216 | 0.163815129 | 606 |
| K02650>>type IV pilus assembly protein PilA | −0.483434861 | −0.187220549 | 607 |
| K01704>>3-isopropylmalate/(R)-2-methylmalate dehydratase small subunit [EC:4.2.1.33 4.2.1.35] | −0.139946103 | −0.011041153 | 608 |
| k__Bacteria\|p__Firmicutes\|c__Clostridia\|o__Clostridiales\|f__Lachnospiraceae\|g__Anaerostipes\|s__Anaerostipes_hadrus | −0.481145158 | −0.220594945 | 609 |
| K10556>>AI-2 transport system permease protein | 0.232072861 | 0.171799434 | 610 |
| K06283>>putative DeoR family transcriptional regulator, stage III sporulation protein D | −0.306240712 | −0.057372935 | 611 |
| K10558>>AI-2 transport system ATP-binding protein | 0.27268224 | 0.183228397 | 612 |
| K01803>>triosephosphate isomerase (TIM) [EC:5.3.1.1] | 0.044516055 | −0.012820513 | 613 |
| K14261>>alanine-synthesizing transaminase [EC:2.6.1.-]] | 0.304774657 | 0.149511817 | 614 |
| K06207>>GTP-binding protein | −0.147418225 | −0.01567205 | 615 |
| K19225>>rhomboid protease GluP [EC:3.4.21.105] | −0.363228778 | −0.140751893 | 616 |
| K03272>>D-beta-D-heptose 7-phosphate kinase/D-beta-D-heptose 1-phosphate adenosyltransferase [EC:2.7.1.167 2.7.7.70] | 0.382865849 | 0.126859202 | 617 |
| K15723>>SecY interacting protein Syd | 0.309390989 | 0.181836846 | 618 |
| K08600>>sortase B [EC:3.4.22.71] | −0.289570239 | 0.0085318 | 619 |
| K20345>>membrane fusion protein, peptide pheromone/bacteriocin exporter | −0.359435655 | −0.143671868 | 620 |
| K03585>>membrane fusion protein, multidrug efflux system | 0.025946389 | −0.05771512 | 621 |
| K08659>>dipeptidase [EC:3.4.-.-]] | −0.252854457 | −0.009991788 | 622 |
| K06397>>stage III sporulation protein AH | −0.279398158 | −0.029929738 | 623 |
| K09749>>uncharacterized protein | −0.421085574 | −0.106966877 | 624 |
| K07019>>uncharacterized protein | 0.332206947 | 0.208572862 | 625 |
| K11617>>two-component system, NarL family, sensor histidine kinase LiaS [EC:2.7.13.3] | −0.361428203 | −0.229947988 | 626 |
| K01667>>tryptophanase [EC:4.1.99.1] | 0.262559655 | 0.000958117 | 627 |
| K03919>>DNA oxidative demethylase [EC:1.14.11.33] | 0.244937249 | 0.156173008 | 628 |
| K09765>>epoxyqueuosine reductase [EC:1.17.99.6] | −0.228864285 | −0.05771512 | 629 |
| K01990>>ABC-2 type transport system ATP-binding protein | −0.126088792 | −0.006410256 | 630 |
| K06211>>HTH-type transcriptional regulator, transcriptional repressor of NAD biosynthesis genes [EC:2.7.7.1 2.7.1.22] | 0.372058931 | 0.167716032 | 631 |
| k__Bacteria\|p__Bacteroidetes\|c__Bacteroidia\|o__Bacteroidales\|f__Tannerellaceae\|g__Parabacteroides\| | 0.036623161 | 0.054612647 | 632 |
| s__Parabacteroides_goldsteinii | | | |
| K09825>>Fur family transcriptional regulator, peroxide stress response regulator | −0.11642235 | −0.011041153 | 633 |
| K09899>>uncharacterized protein | 0.390881504 | 0.15033306 | 634 |
| K07088>>uncharacterized protein | −0.185332111 | −0.009261794 | 635 |
| K04655>>hydrogenase expression/formation protein HypE | −0.204312632 | −0.027831006 | 636 |
| K03586>>cell division protein FtsL | 0.478996304 | 0.146432156 | 637 |
| K00641>>homoserine O-acetyltransferase/O-succinyltransferase [EC:2.3.1.31 2.3.1.46] | 0.277079188 | 0.114996806 | 638 |
| K09787>>uncharacterized protein | −0.293239409 | −0.038119354 | 639 |
| K01462>>peptide deformylase [EC:3.5.1.88] | −0.09951429 | −0.006410256 | 640 |
| K03320>>ammonium transporter, Amt family | −0.21340741 | 0.004608085 | 641 |
| K06016>>beta-ureidopropionase/N-carbamoyl-L-amino-acid hydrolase [EC:3.5.1.6 3.5.1.87] | −0.334230834 | −0.094420111 | 642 |
| K09801>>uncharacterized protein | 0.28737093 | 0.175791587 | 643 |
| K13829>>shikimate kinase/3-dehydroquinate synthase [EC:2.7.1.71 4.2.3.4] | −0.41262046 | −0.190094899 | 644 |
| K03386>>peroxiredoxin (alkyl hydroperoxide reductase subunit C) [EC:1.11.1.15] | 0.264105889 | −0.02995255 | 645 |
| K09913>>purine/pyrimidine-nucleoside phosphorylase [EC:2.4.2.1 2.4.2.2] | 0.452576497 | 0.218610275 | 646 |
| K09982>>uncharacterized protein | 0.322165101 | 0.153367096 | 647 |
| K03429>>processive 1,2-diacylglycerol beta-glucosyltransferase [EC:2.4.1.315] | −0.271801409 | −0.040309335 | 648 |
| K12371>>dipeptide transport system ATP-binding protein | 0.378673228 | 0.157359248 | 649 |
| K02195>>heme exporter protein C | 0.129785581 | 0.007550871 | 650 |
| K02057>>simple sugar transport system permease protein | −0.158953767 | −0.006410256 | 651 |
| K01921>>D-alanine-D-alanine ligase [EC:6.3.2.4] | −0.056578398 | −0.004630897 | 652 |
| K07216>>hemerythrin | −0.366065662 | −0.088717036 | 653 |
| K09018>>pyrimidine oxygenase [EC:1.14.99.46] | 0.312231375 | 0.167647596 | 654 |
| K05350>>beta-glucosidase [EC:3.2.1.21] | −0.383660166 | −0.144698421 | 655 |
| K10012>>undecaprenyl-phosphate 4-deoxy-4-formamido-L-arabinose transferase [EC:2.4.2.53] | 0.308644087 | 0.187220549 | 656 |
| K16248>>probable glucitol transport protein GutA | −0.337635809 | −0.160872342 | 657 |
| K07784>>MFS transporter, OPA family, hexose phosphate transport protein UhpT | 0.33069086 | 0.161237339 | 658 |
| K11752>>diaminohydroxyphosphoribosylaminopyrimidine deaminase / | −0.173277655 | −0.022447304 | 659 |
| 5-amino-6-(5-phosphoribosylamino)uracil reductase [EC:3.5.4.26 1.1.1.193] | | | |
| K07235>>tRNA 2-thiouridine synthesizing protein D [EC:2.8.1.-]] | 0.409009883 | 0.149192445 | 660 |
| K07062>>toxin FitB [EC:3.1.-.-]] | −0.515549768 | −0.228556438 | 661 |
| K07105>>uncharacterized protein | −0.275494034 | −0.041335888 | 662 |
| K06407>>stage V sporulation protein AE | −0.394612675 | −0.045259604 | 663 |
| K16509>>regulatory protein spx | −0.341800747 | −0.193630806 | 664 |
| K04343>>streptomycin 6-kinase [EC:2.7.1.72] | 0.246627577 | 0.135824437 | 665 |
| K04769>>AbrB family transcriptional regulator, stage V sporulation protein T | −0.243146727 | −0.027785382 | 666 |
| K07586>>uncharacterized protein | −0.309487406 | −0.161533899 | 667 |
| K21567>>ferredoxin/flavodoxin---NADP+ reductase [EC:1.18.1.2 1.19.1.1] | −0.341283481 | −0.179008121 | 668 |
| K03410>>chemotaxis protein CheC | −0.311278495 | −0.06058947 | 669 |
| K01089>>imidazoleglycerol-phosphate dehydratase/histidinol-phosphatase [EC:4.2.1.19 3.1.3.15] | 0.147462414 | −0.046696779 | 670 |
| K01637>>isocitrate lyase [EC:4.1.3.1] | 0.345394315 | 0.176544393 | 671 |
| K00163>>pyruvate dehydrogenase El component [EC:1.2.4.1] | 0.349706783 | 0.156697691 | 672 |
| K12138>>hydrogenase-4 component C [EC:1.-.-.-]] | 0.107195629 | 0.060840405 | 673 |
| K08483>>phosphoenolpyruvate-protein phosphotransferase (PTS system enzyme I) [EC:2.7.3.9] | −0.159109604 | −0.01745141 | 674 |
| K01685>>altronate hydrolase [EC:4.2.1.7] | 0.1595153 | −0.011816772 | 675 |
| K00243>>uncharacterized protein | −0.237843754 | −0.031709098 | 676 |
| k__Bacteria\|p__Bacteroidetes\|c__Bacteroidia\|o__Bacteroidales\|f__Bacteroidaceae\|g__Bacteroides\|s__Bacteroides_stercoris | 0.183113548 | 0.089013596 | 677 |
| k__Bacteria\|p__Firmicutes\|c_Clostridia o__Clostridiales\|f__Ruminococcaceae\|g__Ruminococcus\|s__Ruminococcus_lactaris | −0.219534317 | −0.155032393 | 678 |
| K12554>>alanine adding enzyme [EC:2.3.2.-]] | −0.432475371 | −0.209325668 | 679 |
| K01209>>alpha-L-arabinofuranosidase [EC:3.2.1.55] | −0.127550927 | −0.022082307 | 680 |
| K15257>>tRNA (mo5U34)-methyltransferase [EC:2.1.1.-]] | 0.362122312 | 0.142805 | 681 |
| K02115>>F-type H+-transporting ATPase subunit gamma | −0.087146332 | −0.006410256 | 682 |
| k__Bacteria\|p__Actinobacteria\|c__Coriobacteriia\|o__Eggerthellales | −0.280167447 | −0.257984305 | 683 |
| K01736>>chorismate synthase [EC:4.2.3.5] | −0.047122667 | −0.006410256 | 684 |
| K12984>>(heptosyl)LPS beta-1,4-glucosyltransferase [EC:2.4.1.-]] | 0 | 0.059129483 | 685 |
| K06398>>stage IV sporulation protein A | −0.290514815 | −0.026371019 | 686 |
| K02907>>large subunit ribosomal protein L30 | 0.04865194 | −0.006410256 | 687 |
| K02552>>menaquinone-specific isochorismate synthase [EC:5.4.4.2] | 0.300904372 | 0.147048088 | 688 |
| K19336>>cyclic-di-GMP-binding biofilm dispersal mediator protein | 0.291055906 | 0.173624418 | 689 |
| K21064>>5-amino-6-(5-phospho-D-ribitylamino)uracil phosphatase [EC:3.1.3.104] | −0.404729139 | −0.258052742 | 690 |
| K01776>>glutamate racemase [EC:5.1.1.3] | −0.15482327 | −0.004630897 | 691 |
| K02992>>small subunit ribosomal protein S7 | 0.096484302 | −0.006410256 | 692 |
| K01784>>UDP-glucose 4-epimerase [EC:5.1.3.2] | −0.162398242 | −0.011041153 | 693 |
| K09975>>uncharacterized protein | 0.309056291 | 0.140181586 | 694 |
| K01790>>dTDP-4-dehydrorhamnose 3,5-epimerase [EC:5.1.3.13 | −0.00573146 | −0.059129483 | 695 |
| K07584>>uncharacterized protein | −0.299155851 | −0.063418195 | 696 |
| K07979>>GntR family transcriptional regulator | −0.260087124 | −0.016037047 | 697 |
| K05303>>O-methyltransferase [EC:2.1.1.-]] | −0.429615593 | −0.199744502 | 698 |
| K01676>>fumarate hydratase, class I [EC:4.2.1.2] | 0.279223921 | 0.008417739 | 699 |
| K08303>>putative protease [EC:3.4.-.-]] | −0.082871543 | −0.004630897 | 700 |
| k__Bacteria\|p__Proteobacteria\|c__Gammaproteobacteria\|o__Enterobacterales\|f__Enterobacteriaceae | 0.422081398 | 0.112601515 | 701 |
| K09906>>elongation factor P hydroxylase [EC:1.14.-.-]] | 0.379181215 | 0.158089242 | 702 |
| K05807>>outer membrane protein assembly factor BamD | 0.356688597 | 0.043343371 | 703 |
| K01179>>endoglucanase [EC:3.2.1.4] | −0.441416643 | −0.104160964 | 704 |
| K09473>>gamma-glutamyl-gamma-aminobutyrate hydrolase [EC:3.5.1.94] | 0.230415325 | 0.153663655 | 705 |
| K01677>>fumarate hydratase subunit alpha [EC:4.2.1.2] | −0.247179426 | −0.042773063 | 706 |
| K02342>>DNA polymerase III subunit epsilon [EC:2.7.7.7] | 0.065931279 | −0.041678073 | 707 |
| K18800>>2-polyprenylphenol 6-hydroxylase [EC:1.14.13.240] | 0.341120826 | 0.181129665 | 708 |
| K18955>>WhiB family transcriptional regulator, redox-sensing transcriptional regulator | −0.62287049 | −0.190323022 | 709 |
| K01214>>isoamylase [EC:3.2.1.68] | −0.35171226 | −0.099393193 | 710 |
| K06410>>dipicolinate synthase subunit A | −0.341724343 | −0.098731636 | 711 |
| K00975>>glucose-1-phosphate adenylyltransferase [EC:2.7.7.27] | −0.221271212 | −0.012820513 | 712 |
| k__Bacteria\|p__Firmicutes\|c__Bacilli\|o__Lactobacillies | −0.292450975 | −0.179943425 | 713 |
| K10003>>glutamate/aspartate transport system permease protein | 0.276374814 | 0.176863765 | 714 |
| K15832>>formate hydrogenlyase subunit 7 | 0.378979488 | 0.170521945 | 715 |
| K01227>>mannosyl-glycoprotein endo-beta-N-acetylglucosaminidase [EC:3.2.1.96] | −0.545932444 | −0.260539283 | 716 |
| K17318>>putative aldouronate transport system substrate-binding protein | −0.299117911 | −0.062711014 | 717 |
| K00620>>glutamate N-acetyltransferase/amino-acid N-acetyltransferase [EC:2.3.1.35 2.3.1.1] | −0.481084666 | −0.048453326 | 718 |
| K07400>>Fe/S biogenesis protein NfuA | 0.412306702 | 0.147139338 | 719 |
| K03486>>GntR family transcriptional regulator, trehalose operon transcriptional repressor | −0.50488646 | −0.244525048 | 720 |
| K06295>>spore germination protein KA | −0.440549839 | −0.164841683 | 721 |
| K01419>>ATP-dependent HslUV protease, peptidase subunit HslV [EC:3.4.25.2] | 0.405762918 | 0.104503148 | 722 |
| K01807>>ribose 5-phosphate isomerase A [EC:5.3.1.6] | −0.046333305 | −0.020667944 | 723 |
| K17074>>putative lysine transport system permease protein | −0.170802639 | −0.086686741 | 724 |
| K20460>>lantibiotic transport system permease protein | −0.309014681 | −0.016037047 | 725 |
| K20534>>polyisoprenyl-phosphate glycosyltransferase [EC:2.4.-.-]] | −0.353735406 | −0.047746145 | 726 |
| K09117>>uncharacterized protein | −0.318897998 | −0.059517292 | 727 |
| K07259>>serine-type D-Ala-D-Ala carboxypeptidase/endopeptidase (penicillin-binding protein 4) [EC:3.4.16.4 3.4.21.-]] | −0.089543732 | −0.031389725 | 728 |
| K05832>>putative ABC transport system permease protein | −0.227728123 | −0.007482435 | 729 |
| K02825>>pyrimidine operon attenuation protein/uracil phosphoribosyltransferase [EC:2.4.2.9] | −0.248790463 | −0.050255498 | 730 |
| K02055>>putative spermidine/putrescine transport system substrate-binding protein | −0.107369803 | −0.048841135 | 731 |
| K11070>>spermidine/putrescine transport system permease protein | −0.077082306 | −0.020302947 | 732 |
| K22927>>cyclic-di-AMP phosphodiesterase [EC:3.1.4.59] | −0.204588313 | −0.002144356 | 733 |
| K01521>>CDP-diacylglycerol pyrophosphatase [EC:3.6.1.26] | 0.358170386 | 0.14171001 | 734 |
| k__Bacteria\|p__Bacteroidetes\|c__Bacteroidia\|o__Bacteroidales\|f__Bacteroidaceae\|g__Bacteroides\|s__Bacteroides_intestinalis | 0.161197982 | 0.132242905 | 735 |
| k__Bacteria\|p__Firmicutes | −0.177062753 | 0 | 736 |
| K01525>>bis(5'-nucleosyl)-tetraphosphatase (symmetrical) [EC:3.6.1.41] | 0.419113323 | 0.211515649 | 737 |
| K06330>>spore coat protein H | −0.499428425 | −0.194657359 | 738 |
| K03683>>ribonuclease T [EC:3.1.13.-]] | 0.346458206 | 0.136440369 | 739 |
| K00882>>1-phosphofructokinase [EC:2.7.1.56] | −0.197878255 | −0.020667944 | 740 |
| K07783>>MFS transporter, OPA family, sugar phosphate sensor protein UhpC | 0.227943104 | −0.005406515 | 741 |
| K07139>>uncharacterized protein | −0.0793911 | −0.004630897 | 742 |
| K18795>>beta-lactamase class A CARB-1 [EC:3.5.2.6] | 0.052628966 | 0.092982936 | 743 |
| K00700>>1,4-alpha-glucan branching enzyme [EC:2.4.1.18] | −0.102938809 | −0.011041153 | 744 |
| K08997>>uncharacterized protein | 0.296386918 | 0.14766402 | 745 |
| K01956>>carbamoyl-phosphate synthase small subunit [EC:6.3.5.5] | −0.141197509 | −0.006410256 | 746 |
| K15548>>p-hydroxybenzoic acid efflux pump subunit AaeA | 0.340948015 | 0.139862214 | 747 |
| K01819>>galactose-6-phosphate isomerase [EC:5.3.1.26] | −0.421461559 | −0.137923168 | 748 |
| K02064>>thiamine transport system substrate-binding protein | 0.312559595 | 0.186125559 | 749 |
| K15722>>cell division activator | 0.316927166 | 0.156971439 | 750 |
| K14623>>DNA-damage-inducible protein D | −0.335936989 | −0.063098823 | 751 |
| K01938>>formate--tetrahydrofolate ligase [EC:6.3.4.3] | −0.095923285 | −0.006410256 | 752 |
| K02257>>heme o synthase [EC:2.5.1.141] | 0.362157122 | 0.156309882 | 753 |
| K03281>>chloride channel protein, CIC family | 0.161370916 | −0.032439091 | 754 |
| K00655>>1-acyl-sn-glycerol-3-phosphate acyltransferase [EC:2.3.1.51] | −0.104177748 | −0.011041153 | 755 |
| K03892>>ArsR family transcriptional regulator, arsenate/arsenite/antimonite-responsive transcriptional repressor | −0.325711511 | −0.06485537 | 756 |
| K19270>>sugar-phosphatase [EC:3.1.3.23] | 0.368555161 | 0.176932202 | 757 |
| K06911>>quercetin 2,3-dioxygenase [EC:1.13.11.24] | 0.29923493 | 0.006684004 | 758 |
| K02039>>phosphate transport system protein | −0.131627079 | −0.004630897 | 759 |
| K02386>>flagella basal body P-ring formation protein FlgA | 0.312058566 | 0.151975545 | 760 |
| K09456>>putative acyl-CoA dehydrogenase | 0.235285175 | 0.169723515 | 761 |
| K18581>>unsaturated chondroitin disaccharide hydrolase [EC:3.2.1.180] | −0.208397653 | −0.122456429 | 762 |
| K01512>>acylphosphatase [EC:3.6.1.7] | −0.350739433 | −0.043115248 | 763 |
| K19228>>cationic peptide transport system permease protein | 0.334240553 | 0.14914682 | 764 |
| K03657>>DNA helicase II/ATP-dependent DNA helicase PcrA [EC:3.6.4.12] | −0.140717835 | −0.012820513 | 765 |
| K06203>>CysZ protein | 0.421754366 | 0.190824893 | 766 |
| K13938>>dihydromonapterin reductase/dihydrofolate reductase [EC:1.5.1.50 1.5.1.3] | 0.373203182 | 0.170134136 | 767 |
| K07738>>transcriptional repressor NrdR | −0.207302832 | −0.012820513 | 768 |
| K01693>>imidazoleglycerol-phosphate dehydratase [EC:4.2.1.19] | −0.375801078 | −0.003581531 | 769 |
| K03640>>peptidoglycan-associated lipoprotein | 0.352472609 | 0.147823707 | 770 |
| K09892>>cell division protein ZapB | 0.447090837 | 0.135345378 | 771 |
| K00604>>methionyl-tRNA formyltransferase [EC:2.1.2.9] | −0.054324706 | −0.006410256 | 772 |
| K16148>>alpha-maltose-1-phosphate synthase [EC:2.4.1.342] | −0.441926208 | −0.142736564 | 773 |
| K03599>>stringent starvation protein A | 0.413519178 | 0.165229492 | 774 |
| K06145>>LacI family transcriptional regulator, | 0.343279888 | 0.164499498 | 775 |
| gluconate utilization system Gnt-I transcriptional repressor | | | |
| K06379>>stage II sporulation protein AB (anti-sigma F factor) [EC:2.7.11.1] | −0.365482234 | −0.054521398 | 776 |
| K04074>>cell division initiation protein | −0.548772669 | −0.169997263 | 777 |
| K02860>>16S rRNA processing protein RimM | −0.075882546 | −0.012820513 | 778 |
| K06442>>23S rRNA (cytidine 1920-2'-O)/16S rRNA (cytidine1409-2'-O)-methyltransferase [EC:2.1.1.226 2.1.1.227] | −0.308483748 | −0.102586915 | 779 |
| K02380>>FdhE protein | 0.363794171 | 0.093461995 | 780 |
| K02119>>V/A-type H+/Na+-transporting ATPase subunit C | −0.379160347 | −0.071630623 | 781 |
| K21600>>CsoR family transcriptional regulator, copper-sensing transcriptional repressor | −0.200390806 | −0.009261794 | 782 |
| K02028>>polar amino acid transport system ATP-binding protein [EC:7.4.2.1] | −0.203439698 | −0.028492563 | 783 |
| K02379>>FdhD protein | 0.328915633 | 0.080960854 | 784 |
| K02197>>cytochrome c-type biogenesis protein CcmE | 0.360679097 | 0.140706269 | 785 |
| K02283>>pilus assembly protein CpaF [EC:7.4.2.8] | −0.395320325 | −0.046673967 | 786 |
| K02836>>peptide chain release factor 2 | −0.183110964 | −0.009261794 | 787 |
| K03106>>signal recognition particle subunit SRP54 [EC:3.6.5.4] | −0.135445294 | −0.006410256 | 788 |
| K02122>>V/A-type H+/Na+-transporting ATPase subunit F | −0.214968162 | −0.044164614 | 789 |
| K07667>>two-component system, OmpR family, KDP operon response regulator KdpE | −0.211458747 | −0.012820513 | 790 |
| K00568>>2-polyprenyl-6-hydroxyphenyl methylase/3-demethylubiquinone-9 3-methyltransferase [EC:2.1.1.222 2.1.1.64] | 0.368306011 | 0.145268729 | 791 |
| K03573>>DNA mismatch repair protein MutH | 0.348709075 | 0.148508076 | 792 |
| K16786>>energy-coupling factor transport system ATP-binding protein [EC:3.6.3.-]] | −0.270774928 | −0.01567205 | 793 |
| K07729>>putative transcriptional regulator | −0.193965689 | −0.011041153 | 794 |
| K02435>>aspartyl-tRNA(Asn)/glutamyl-tRNA(Gln) amidotransferase subunit C [EC:6.3.5.6 6.3.5.7] | −0.313570243 | 0.003193722 | 795 |
| K02224>>cobyrinic acid a,c-diamide synthase [EC:6.3.5.9 6.3.5.11] | −0.111656937 | −0.039898713 | 796 |
| K02025>>multiple sugar transport system permease protein | −0.333465836 | −0.03490282 | 797 |
| K00286>>pyrroline-5-carboxylate reductase [EC:1.5.1.2] | −0.089733169 | −0.012820513 | 798 |
| k__Bacteria\|p__Firmicutes\|c__Bacilli | −0.288156515 | −0.181722785 | 799 |
| K19117>>CRISPR-associated protein Csd1 | −0.355764558 | −0.131170727 | 800 |

TABLE 4
Feature List for colorectal advanced adenoma (CRAA). The Table presents the fold changes in relative abundance, prevalence shifts and weight or Importance of the features, changes, and shifts.
The prevalence shift value between the two classes has a positive value when there is a higher prevalence in CRAA and a negative value when there is a higher prevalence in the control group.

| | Fold change in | Prevalence | Weight or |
|---|---|---|---|
| Taxonomic or Gene Feature | relative abundance | Shift | importance |
| k_{——}Bacteria\|p_{——}Firmicutes\|c_{——}Negativicutes\|o_{——}Acidaminococcales | −0.790026584 | −0.485448005 | 1 |
| k_{——}Bacteria\|p_{——}Actinobacteria\|c_{——}Coriobacteriia\|o_{——}Coriobacteriales | 0.084824206 | 0.13145847 | 2 |
| \|f_{——}Atopobiaceae | | | |
| K05995>>dipeptidase E [EC: 3.4.13.21] | 0.492336201 | 0.133747547 | 3 |
| K13687>>arabinofuranosyltransferase [EC: 2.4.2.—] | 0 | 0.043655984 | 4 |
| K20460>>lantibiotic transport system permease protein | 0.704993339 | 0.023381295 | 5 |
| K20491>>lantibiotic transport system permease protein | 0.026025985 | 0.021255723 | 6 |
| K20459>>lantibiotic transport system ATP-binding protein | 0.253282007 | 0.21206671 | 7 |
| k_{——}Bacteria\|p_{——}Firmicutes\|c_{——}Clostridia\|o_{——}Clostridiales\|f_{——}Oscillospiraceae | 0.247260394 | 0.103989536 | 8 |
| \|g_{——}Oscillibacter\|s_{——}Oscillibacter_sp_CAG_241 | | | |
| K03722>>ATP-dependent DNA helicase DinG [EC: 3.6.4.12] | 0.578857144 | 0.037769784 | 9 |
| k_{——}Bacteria\|p_{——}Firmicutes\|c_{——}Negativicutes\|o_{——}Acidaminococcales\|f_{——}Acidaminococcaceae | −0.659796271 | −0.443427077 | 10 |
| \|g_{——}Phascolarctobacterium | | | |
| K00651>>homoserine O-succinyltransferase/O-acetyltransferase [EC: 2.3.1.46 2.3.1.31] | −0.134150867 | 0.003597122 | 11 |
| K11068>>hemolysin III | −0.176588773 | 0.001798561 | 12 |
| k_{——}Bacteria\|p_{——}Actinobacteria\|c_{——}Actinobacteria\|o_{——}Actinomycetales\|f_{——}Actinomycetaceae | 0.152434595 | 0.206834532 | 13 |
| \|g_{——}Actinomyces\|s_{——}Actinomyces_sp_ICM47 | | | |
| K02913>>large subunit ribosomal protein L33 | −0.413853488 | 0 | 14 |
| K22501>>cytochrome bd-II ubiquinol oxidase subunit AppX [EC: 7.1.1.7] | 0.429783076 | 0.146827992 | 15 |
| K16907>>fluoroquinolone transport system ATP-binding protein [EC: 3.6.3.—] | −0.052658946 | −0.080935252 | 16 |
| K02914>>large subunit ribosomal protein L34 | −0.375090686 | 0.001798561 | 17 |
| K18475>>lysine-N-methylase [EC: 2.1.1.—] | 0.356630666 | 0.059352518 | 18 |
| K15553>>sulfonate transport system substrate-binding protein | −0.624018873 | −0.319980379 | 19 |
| K14524>>ribonuclease P/MRP protein subunit POP6 [EC: 3.1.26.5] | 0 | 0.045454545 | 20 |
| k_{——}Bacteria\|p_{——}Actinobacteria\|c_{——}Coriobacteriia\|o_{——}Eggerthellales\|f_{——}Eggerthellaceae | 0.14246061 | 0.205689993 | 21 |
| \|g_{——}Enterorhabdus | | | |
| K09922>>uncharacterized protein | −1.262255944 | −0.255232178 | 22 |
| K00099>>1-deoxy-D-xylulose-5-phosphate reductoisomerase [EC: 1.1.1.267] | −0.114573701 | 0.001798561 | 23 |
| K22298>>ArsR family transcriptional regulator, zinc-responsive transcriptional repressor | 0.076401079 | 0.153041203 | 24 |
| K16924>>energy-coupling factor transport system substrate-specific component | 0.455083669 | 0.046272073 | 25 |
| K12452>>CDP-4-dehydro-6-deoxyglucose reductase, E1 [EC: 1.17.1.1] | 0.225639367 | 0.044473512 | 26 |
| K19244>>alanine dehydrogenase [EC: 1.4.1.1] | 0.632292102 | 0.209450621 | 27 |
| K03458>>nucleobase: cation symporter-2, NCS2 family | −0.324844651 | −0.219751472 | 28 |
| K01271>>Xaa-Pro dipeptidase [EC: 3.4.13.9] | 0.372818401 | 0.09793983 | 29 |
| K15977>>putative oxidoreductase | −0.716961747 | −0.126716808 | 30 |
| K08298>>L-carnitine CoA-transferase [EC: 2.8.3.21] | 0.708311224 | 0.263080445 | 31 |
| K05341>>amylosucrase [EC: 2.4.1.4] | 0.844531949 | 0.311314585 | 32 |
| K15527>>cysteate synthase [EC: 2.5.1.76] | −0.380810029 | −0.301994768 | 33 |
| K04652>>hydrogenase nickel incorporation protein HypB | 0.451482384 | 0.095977763 | 34 |
| K01200>>pullulanase [EC: 3.2.1.41] | 0.047801462 | 0.007848267 | 35 |
| K07078>>uncharacterized protein | −0.499395262 | −0.000490517 | 36 |
| k_{——}Bacteria\|p_{——}Firmicutes\|c_{——}Clostridia\|o_{——}Clostridiales\|f_{——}Peptostreptococcaceae | 0.229334106 | 0.273544801 | 37 |
| \|g_{——}Intestinibacter | | | |
| k_{——}Bacteria\|p_{——}Actinobacteria\|c_{——}Actinobacteria\|o_{——}Bifidobacteriales\|f_{——}Bifidobacteriaceae | 0.252787089 | −0.003433617 | 38 |
| \|g_{——}Bifidobacterium\|s_{——}Bifidobacterium_longum | | | |
| k_{——}Bacteria\|p_{——}Firmicutes\|c_{——}Clostridia\|o_{——}Clostridiales\|f_{——}Clostridiales_Family_XIII_Incertae_Sedis | 0.035470972 | −0.004251145 | 39 |
| \|g_{——}Mogibacterium | | | |
| K19701>>aminopeptidase YwaD [EC: 3.4.11.6 3.4.11.10] | −0.417817826 | −0.344506213 | 40 |
| K02405>>RNA polymerase sigma factor for flagellar operon FliA | −0.509746643 | −0.183453237 | 41 |
| K19271>>chloramphenicol O-acetyltransferase type A [EC: 2.3.1.28] | −0.571736799 | −0.02207325 | 42 |
| k_{——}Bacteria\|p_{——}Bacteroidetes\|c_{——}Bacteroidia\|o_{——}Bacteroidales\|f_{——}Rikenellaceae\|g_{——}Alistipes | 0.354607435 | 0.232831916 | 43 |
| \|s_{——}Alistipes_inops | | | |
| k_{——}Bacteria\|p_{——}Firmicutes\|c_{——}Clostridia\|o_{——}Clostridiales\|f_{——}Lachnospiraceae\|g_{——}Lachnoclostridium | −0.145843288 | −0.199803793 | 44 |
| \|s_{——}Clostridium_bolteae | | | |
| K02911>>large subunit ribosomal protein L32 | −0.232030905 | 0 | 45 |
| K01002>>phosphoglycerol transferase [EC: 2.7.8.20] | 0.756509151 | 0.279594506 | 46 |
| k_{——}Bacteria | 0.019979004 | 0 | 47 |
| K17315>>glucose/mannose transport system substrate-binding protein | 0.024328092 | 0.099247874 | 48 |
| K07741>>anti-repressor protein | −0.247705039 | −0.159908437 | 49 |
| k_{——}Bacteria\|p_{——}Firmicutes\|c_{——}Bacilli\|o_{——}Lactobacillos\|f_{——}Lactobacilliae\|g_{——}Lactobacillus | 0.01162409 | 0.051994768 | 50 |
| \|s_{——}Lactobacillus_rhamnosus | | | |
| k_{——}Bacteria\|p_{——}Proteobacteria\|c_{——}Betaproteobacteria | −0.302139702 | −0.306572923 | 51 |
| K01719>>uroporphyrinogen-III synthase [EC: 4.2.1.75] | −0.905469493 | −0.110529758 | 52 |
| K01771>>1-phosphatidylinositol phosphodiesterase [EC: 4.6.1.13] | 0.472398943 | 0.297743623 | 53 |
| K07321>>CO dehydrogenase maturation factor | 0.837702893 | 0.06294964 | 54 |
| K22901>>tRNA (cytosine40_48-C5)-methyltransferase [EC: 2.1.1.—] | −0.343272727 | −0.29529104 | 55 |
| K07813>>accessory gene regulator B | 0.34462095 | 0.01030085 | 56 |
| K21993>>formate transporter | 0.001608398 | 0.065729235 | 57 |
| K17735>>carnitine 3-dehydrogenase [EC: 1.1.1.108] | 0.57259977 | 0.425931982 | 58 |
| k_{——}Bacteria\|p_{——}Firmicutes\|c_{——}Clostridia\|o_{——}Clostridiales\|f_{——}Oscillospiraceae | 0.003437361 | −0.065238718 | 59 |
| k_{——}Bacteria\|p_{——}Firmicutes\|c_{——}Clostridia\|o_{——}Clostridiales\|f_{——}Clostridiales_Family_XIII_Incertae_Sedis | 0.034871231 | −0.002452583 | 60 |
| \|g_{——}Mogibacterium\|s_{——}Mogibacterium_diversum | | | |
| K08093>>3-hexulose-6-phosphate synthase [EC: 4.1.2.43] | 0.753160411 | 0.489862655 | 61 |
| k_{——}Bacteria\|p_{——}Bacteroidetes\|c_{——}Bacteroidia\|o_{——}Bacteroidales\|f_{——}Rikenellaceae\|g_{——}Alistipes | −0.525297432 | −0.133093525 | 62 |
| \|s_{——}Alistipes_putredinis | | | |
| K00800>>3-phosphoshikimate 1-carboxyvinyltransferase [EC: 2.5.1.19] | −0.125328728 | 0.001798561 | 63 |
| k_{——}Bacteria\|p_{——}Firmicutes\|c_{——}Clostridia\|o_{——}Clostridiales\|f_{——}Lachnospiraceae\|g_{——}Roseburia | 0.157531388 | 0.069653368 | 64 |
| \|s_{——}Roseburia_faecis | | | |
| K18829>>antitoxin VapB | 0.515876971 | 0.310333551 | 65 |
| K03336>>3D-(3,5/4)-trihydroxycyclohexane-1,2-dione acylhydrolase (decyclizing) [EC: 3.7.1.22] | 0.550472 | 0.249836494 | 66 |
| K22116>>gamma-polyglutamate biosynthesis protein CapC | −0.509590938 | −0.369522564 | 67 |
| k_{——}Bacteria\|p_{——}Proteobacteria\|c_{——}Betaproteobacteria\|o_{——}Burkholderiales | −0.297648405 | −0.30117724 | 68 |
| k_{——}Bacteria\|p_{——}Proteobacteria\|c_{——}Betaproteobacteria\|o_{——}Burkholderiales\|f_{——}Sutterellaceae | −0.276699464 | −0.30412034 | 69 |
| k_{——}Bacteria\|p_{——}Firmicutes\|c_{——}Clostridia\|o_{——}Clostridiales\|f_{——}Lachnospiraceae | 0.309511559 | 0 | 70 |
| K18828>>tRNA(fMet)-specific endonuclease VapC [EC: 3.1.—.—] | 0.668648173 | 0.38734467 | 71 |
| k_{——}Bacteria\|p_{——}Actinobacteria\|c_{——}Coriobacteriia\|o_{——}Eggerthellales\|f_{——}Eggerthellaceae | 0.647675257 | 0.372465664 | 72 |
| \|g_{——}Asaccharobacter | | | |
| K03296>>hydrophobic/amphiphilic exporter-1 (mainly G- bacteria), HAE1 family | −0.994658203 | −0.133911053 | 73 |
| k_{——}Archaea | 0.359742155 | 0.175441465 | 74 |
| k_{——}Archaea\|p_{——}Euryarchaeota\|c_{——}Methanobacteria\|o_{——}Methanobacteriales\|f_{——}Methanobacteriaceae | 0.356836425 | 0.179038587 | 75 |
| \|g_{——}Methanobrevibacter\|s_{——}Methanobrevibacter_smithii | | | |
| k_{——}Archaea\|p_{——}Euryarchaeota\|c_{——}Methanobacteria\|o_{——}Methanobacteriales\|f_{——}Methanobacteriaceae | 0.355469296 | 0.177240026 | 76 |
| \|g_{——}Methanobrevibacter | | | |
| k_{——}Bacteria\|p_{——}Firmicutes\|c_{——}Negativicutes\|o_{——}Veillonellales | −0.034297725 | 0.0117724 | 77 |
| k_{——}Archaea\|p_{——}Euryarchaeota\|c_{——}Methanobacteria | 0.360644057 | 0.175441465 | 78 |
| k_{——}Bacteria\|p_{——}Firmicutes\|c_{——}Clostridia\|o_{——}Clostridiales\|f_{——}Lachnospiraceae\|g_{——}Dorea | 0.51323312 | 0.074558535 | 79 |
| K02121>>V/A-type H+/Na+-transporting ATPase subunit E | −0.079033477 | 0.021582734 | 80 |
| k_{——}Bacteria\|p_{——}Firmicutes\|c_{——}Firmicutes_unclassified\|o_{——}Firmicutes_unclassified\| | 0.170208761 | 0.185088293 | 81 |
| \|f_{——}Firmicutes_unclassified | | | |
| \|g_{——}Firmicutes_unclassified\|s_{——}Firmicutes_bacterium_CAG_94 | | | |
| k_{——}Bacteria\|p_{——}Actinobacteria\|c_{——}Coriobacteriia\|o_{——}Eggerthellales\|f_{——}Eggerthellaceae | #N/A | #N/A | 82 |
| \|g_{——}Denitrobacterium | | | |
| K03696>>ATP-dependent Clp protease ATP-binding subunit ClpC | 0.294585187 | 0.028776978 | 83 |
| k_{——}Bacteria\|p_{——}Firmicutes\|c_{——}Clostridia\|o_{——}Clostridiales\|f_{——}Lachnospiraceae\|g_{——}Blautia | 0.542710763 | 0.198168738 | 84 |
| \|s_{——}Blautia_wexlerae | | | |
| k_{——}Bacteria\|p_{——}Bacteroidetes\|c_{——}Bacteroidia\|o_{——}Bacteroidales\|f_{——}Bacteroidaceae\|g_{——}Bacteroides | −0.34979911 | −0.321288424 | 85 |
| \|s_{——}Bacteroides_xylanisolvens | \|s_{——} | | |
| K21527>>adenine modification enzyme [EC: 2.3.1.—] | 0 | 0.062786135 | 86 |
| K09748>>ribosome maturation factor RimP | −0.337271158 | 0.001798561 | 87 |
| K02217>>ferritin [EC: 1.16.3.2] | −0.404126838 | −0.017331589 | 88 |
| K03699>>putative hemolysin | −0.178373969 | 0.007194245 | 89 |
| K03767>>peptidyl-prolyl cis-trans isomerase A (cyclophilin A) [EC: 5.2.1.8] | 0.40848251 | 0.004905167 | 90 |
| k_{——}Bacteria\|p_{——}Firmicutes\|c_{——}Bacilli\|o_{——}Lactobacillales\|f_{——}Streptococcaceae\|g_{——}Streptococcus | 0.032980552 | −0.008011772 | 91 |
| \|s_{——}Streptococcus_mitis | | | |
| K03787>>5′-nucleotidase [EC: 3.1.3.5] | −0.649249325 | −0.065729235 | 92 |
| k_{——}Bacteria\|p_{——}Actinobacteria\|c_{——}Actinobacteria\|o_{——}Actinomycetales\|f_{——}Actinomycetaceae | 0 | 0.022727273 | 93 |
| \|g_{——}Actinomyces\|s_{——}Actinomyces_viscosus | | | |
| k_{——}Bacteria\|p_{——}Firmicutes\|c_{——}Negativicutes\|o_{——}Acidaminococcales\|f_{——}Acidaminococcaceae | −0.241946813 | −0.253106606 | 94 |
| \|g_{——}Phascolarctobacterium\|s_{——}Phascolarctobacterium_faecium | | | |
| K22477>>N-acetylglutamate synthase [EC: 2.3.1.1] | 0.60874974 | 0.189012426 | 95 |
| K16210>>oligogalacturonide transporter | 0.572945713 | 0.241824722 | 96 |
| K15974>>MarR family transcriptional regulator, negative regulator of the multidrug operon emrRAB | 0.529347432 | 0.182308698 | 97 |
| K03664>>SsrA-binding protein | −0.181146253 | 0.001798561 | 98 |
| K02907>>large subunit ribosomal protein L30 | −0.194135576 | 0 | 99 |
| K03602>>exodeoxyribonuclease VII small subunit [EC: 3.1.11.6] | −0.173806789 | 0.001798561 | 100 |
| K15876>>cytochrome c nitrite reductase small subunit | −0.923795041 | −0.323904513 | 101 |
| k_{——}Bacteria\|p_{——}Firmicutes\|c_{——}Clostridia\|o_{——}Clostridiales\|f_{——}Lachnospiraceae\|g_{——}Dorea | 0.374175556 | 0.171844343 | 102 |
| \|s_{——}Dorea_formicigenerans | | | |
| K03552>>holliday junction resolvase Hjr [EC: 3.1.22.4] | 0.374041303 | 0.150425114 | 103 |
| K03710>>GntR family transcriptional regulator | 0.417697726 | 0.014388489 | 104 |
| K03750>>molybdopterin molybdotransferase [EC: 2.10.1.1] | 0.522663478 | 0.195552649 | 105 |
| K03556>>LuxR family transcriptional regulator, maltose regulon positive regulatory protein | 0.685322934 | 0.304283846 | 106 |
| K03778>>D-lactate dehydrogenase [EC: 1.1.1.28] | −0.444648675 | 0.023381295 | 107 |
| K22015>>formate dehydrogenase (acceptor) [EC: 1.17.99.7] | 0.46072976 | 0.016023545 | 108 |
| K14623>>DNA-damage-inducible protein D | 0.16864198 | 0.055264879 | 109 |
| K03574>>8-oxo-dGTP diphosphatase [EC: 3.6.1.55] | 0.307181403 | 0.008992806 | 110 |
| k_{——}Bacteria\|p_{——}Firmicutes\|c_{——}Clostridia\|o_{——}Clostridiales\|f_{——}Peptostreptococcaceae | 0.22564127 | 0.264551995 | 111 |
| k_{——}Bacteria\|p_{——}Actinobacteria\|c_{——}Coriobacteriia\|o_{——}Eggerthellales | 0.753681764 | 0.156638326 | 112 |
| K03564>>thioredoxin-dependent peroxiredoxin [EC: 1.11.1.24] | −0.577677075 | −0.066873774 | 113 |
| k_{——}Bacteria\|p_{——}Actinobacteria\|c_{——}Coriobacteriia\|o_{——}Coriobacteriales\|f_{——}Coriobacteriaceae | 0.148604384 | 0.224166122 | 114 |
| \|g_{——}Enorma\|s_{——}[Collinsella]_massiliensis | | | |
| K03698>>3′-5′ exoribonuclease [EC: 3.1.—.—] | 0.431239077 | 0.032374101 | 115 |
| k_{——}Bacteria\|p_{——}Actinobacteria\|c_{——}Coriobacteriia\|o_{——}Coriobacteriales\|f_{——}Atopobiaceae | 0.001441787 | 0.022727273 | 116 |
| \|g_{——}Atopobium\|s_{——}Atopobium_rimae | | | |
| K03589>>cell division protein FtsQ | −0.209211491 | 0.005395683 | 117 |
| K03798>>cell division protease FtsH [EC: 3.4.24.—] | 0.188982108 | 0.007194245 | 118 |
| K03801>>lipoyl(octanoyl) transferase [EC: 2.3.1.181] | −0.772991105 | −0.253433617 | 119 |
| k_{——}Bacteria\|p_{——}Firmicutes\|c_{——}Clostridia\|o_{——}Clostridiales\|f_{——}Lachnospiraceae | 0.671735993 | 0.244931328 | 120 |
| \|g_{——}Coprococcus\|s_{——}Coprococcus_comes | | | |
| K02003>>putative ABC transport system ATP-binding protein | 0.161856583 | 0 | 121 |
| k_{——}Bacteria\|p_{——}Firmicutes\|c_{——}Clostridia\|o_{——}Clostridiales\|f_{——}Lachnospiraceae\|g_{——}Blautia | 0.493251551 | 0.028776978 | 122 |
| K03811>>nicotinamide mononucleotide transporter | −0.57961146 | −0.021419228 | 123 |
| k_{——}Bacteria\|p_{——}Actinobacteria\|c_{——}Actinobacteria\|o_{——}Actinomycetales\|f_{——}Actinomycetaceae | 0.028275015 | 0.032864617 | 124 |
| \|g_{——}Actinomyces\|s_{——}Actinomyces_graevenitzii | | | |
| K03762>>MFS transporter, MHS family, proline/betaine transporter | 0.550229474 | 0.177567037 | 125 |
| K20257>>2-aminobenzoylacetyl-CoA thioesterase [EC: 3.1.2.32] | 0.500527832 | 0.308371485 | 126 |
| K03608>>cell division topological specificity factor | 0.248719759 | 0.01618705 | 127 |
| K03771>>peptidyl-prolyl cis-trans isomerase SurA [EC: 5.2.1.8] | −0.92478458 | −0.117724003 | 128 |
| K03620>>Ni/Fe-hydrogenase 1 B-type cytochrome subunit | 0.507642457 | 0.2529431 | 129 |
| K03770>>peptidy1-prolyl cis-trans isomerase D [EC: 5.2.1.8] | −0.860194758 | −0.18410726 | 130 |
| K03737>>pyruvate-ferredoxin/flavodoxin oxidoreductase [EC: 1.2.7.1 1.2.7.—] | 0.317313937 | 0.008992806 | 131 |
| K07469>>aldehyde oxidoreductase [EC: 1.2.99.7] | 0.844284113 | 0.061151079 | 132 |
| K03700>>recombination protein U | 0.187044585 | 0.051013734 | 133 |
| K03741>>arsenate reductase (thioredoxin) [EC: 1.20.4.4] | 0.489536436 | 0.113963375 | 134 |
| K03736>>ethanolamine ammonia-lyase small subunit [EC: 4.3.1.7] | 0.481485744 | 0.174623937 | 135 |
| K22430>>caffeyl-CoA reductase-Etf complex subunit CarC [EC: 1.3.1.108] | 0.344588192 | 0.253270111 | 136 |
| K03650>>tRNA modification GTPase [EC: 3.6.—.—] | −0.161100429 | 0.001798561 | 137 |
| K03705>>heat-inducible transcriptional repressor | 0.268922667 | 0.007194245 | 138 |
| K03773>>FKBP-type peptidyl-prolyl cis-trans isomerase FklB [EC: 5.2.1.8] | −0.841249847 | −0.117724003 | 139 |
| K14088>>ech hydrogenase subunit C | 0.718966345 | 0.383584042 | 140 |
| K20974>>two-component system, sensor histidine kinase [EC: 2.7.13.3] | 0.609427924 | 0.253433617 | 141 |
| K03797>>carboxyl-terminal processing protease [EC: 3.4.21.102] | −0.161983673 | 0.003597122 | 142 |
| k_{——}Bacteria\|p_{——}Bacteroidetes\|c_{——}Bacteroidia\|o_{——}Bacteroidales\|f_{——}Tannerellaceae | −0.842489293 | −0.313113146 | 143 |
| K03588>>cell division protein FtsW | −0.213248061 | 0.003597122 | 144 |
| K21574>>glucan 1,4-alpha-glucosidase [EC: 3.2.1.3] | −1.114786276 | −0.233158927 | 145 |
| K03803>>sigma-E factor negative regulatory protein RseC | −0.658081876 | −0.298070634 | 146 |
| K03827>>putative acetyltransferase [EC: 2.3.1.—] | −0.676780635 | −0.103989536 | 147 |
| K03832>>periplasmic protein TonB | −0.88745277 | −0.153041203 | 148 |
| K03833>>selenocysteine-specific elongation factor | 0.542080681 | 0.166121648 | 149 |
| K02919>>large subunit ribosomal protein L36 | 0.397913361 | 0.001798561 | 150 |
| K03839>>flavodoxin I | −0.744688177 | −0.212720733 | 151 |
| K05340>>glucose uptake protein | −0.793293741 | −0.148790059 | 152 |
| k_{——}Bacteria\|p_{——}Firmicutes\|c_{——}Bacilli\|o_{——}Lactobacillales\|f_{——}Lactobacillaceae | 0 | 0.043655984 | 153 |
| \|g_{——}Lactobacillus\|s_{——}Lactobacillus_acidophilus | | | |
| K03637>>cyclic pyranopterin monophosphate synthase [EC: 4.6.1.17] | 0.407118949 | 0.021582734 | 154 |
| k_{——}Bacteria\|p_{——}Actinobacteria\|c_{——}Coriobacteriia\|o_{——}Coriobacteriales | 0.062770826 | 0.100392413 | 155 |
| \|f_{——}Atopobiaceae\|g_{——}Olsenella | | | |
| K03606>>putative colanic acid biosysnthesis UDP-glucose lipid carrier transferase | −0.893070475 | −0.18999346 | 156 |
| K05520>>protease I [EC: 3.5.1.124] | 0.295681306 | 0.127861347 | 157 |
| K05566>>multicomponent Na+: H+ antiporter subunit B | 0.617685687 | 0.413505559 | 158 |
| K05569>>multicomponent Na+: H+ antiporter subunit E | 0.522019694 | 0.379659908 | 159 |
| K03555>>DNA mismatch repair protein MutS | −0.251704467 | 0.005395683 | 160 |
| K16363>>UDP-3-O-[3-hydroxymyristoyl] N-acetylglucosamine deacetylase/ | −1.028293687 | −0.187704382 | 161 |
| 3-hydroxyacyl-[acyl-carrier-protein] dehydratase [EC: 3.5.1.108 4.2.1.59] | | | |
| k_{——}Bacteria\|p_{——}Actinobacteria\|c_{——}Actinobacteria\|o_{——}Actinomycetales | 0.214254237 | 0.2352845 | 162 |
| \|f_{——}Actinomycetaceae\|g_{——}Actinomyces | | | |
| K05567>>multicomponent Na+: H+ antiporter subunit C | 0.494850879 | 0.286788751 | 163 |
| K05568>>multicomponent Na+: H+ antiporter subunit D | 0.795202746 | 0.379823414 | 164 |
| K05570>>multicomponent Na+: H+ antiporter subunit F | 0.595734449 | 0.339437541 | 165 |
| K03657>>DNA helicase II / ATP-dependent DNA helicase PerA [EC: 3.6.4.12] | 0.237391205 | 0.001798561 | 166 |
| K03559>>biopolymer transport protein ExbD | −0.87762892 | −0.158436887 | 167 |
| K03561>>biopolymer transport protein ExbB | −0.935888725 | −0.130313931 | 168 |
| K03569>>rod shape-determining protein MreB and related proteins | 0.273636062 | 0.005395683 | 169 |
| K03572>>DNA mismatch repair protein MutL | −0.161608522 | 0.005395683 | 170 |
| K04654>>hydrogenase expression/formation protein HypD | 0.466644002 | 0.059352518 | 171 |
| K14441>>ribosomal protein S12 methylthiotransferase [EC: 2.8.4.4] | −0.144344863 | 0.005395683 | 172 |
| K03585>>membrane fusion protein, multidrug efflux system | −0.998695114 | −0.153041203 | 173 |
| K04567>>lysyl-tRNA synthetase, class II [EC: 6.1.1.6] | −0.171360804 | 0.003597122 | 174 |
| K03590>>cell division protein FtsA | −0.754496362 | −0.068672335 | 175 |
| K03856>>3-deoxy-7-phosphoheptulonate synthase [EC: 2.5.1.54] | 0.336754618 | 0.050359712 | 176 |
| K03594>>bacterioferritin [EC: 1.16.3.1] | −0.663135593 | −0.014879006 | 177 |
| K03880>>NADH-ubiquinone oxidoreductase chain 3 [EC: 7.1.1.2] | 0.049180139 | 0.112982341 | 178 |
| K03601>>exodeoxyribonuclease VII large subunit [EC: 3.1.11.6] | −0.277885696 | 0.014388489 | 179 |
| K03881>>NADH-ubiquinone oxidoreductase chain 4 [EC: 7.1.1.2] | 0.031145291 | 0.101046436 | 180 |
| K05303>>O-methyltransferase [EC: 2.1.1.—] | 0.92204306 | 0.335349902 | 181 |
| K03694>>ATP-dependent Clp protease ATP-binding subunit ClpA | −0.932576608 | −0.171517332 | 182 |
| k_{——}Bacteria\|p_{——}Firmicutes\|c_{——}Erysipelotrichia | 0.387318499 | 0.146010464 | 183 |
| K15984>>16S rRNA (guanine1516-N2)-methyltransferase [EC: 2.1.1.242] | 0.498196606 | 0.233158927 | 184 |
| K15533>>1,3-beta-galactosyl-N-acetylhexosamine phosphorylase [EC: 2.4.1.211] | 0.424498924 | 0.059025507 | 185 |
| K03615>>Na+-translocating ferredoxin: NAD+ oxidoreductase subunit C [EC: 7.2.1.2] | −0.556016693 | 0.010791367 | 186 |
| K03624>>transcription elongation factor GreA | 0.129983624 | 0.001798561 | 187 |
| K04797>>prefoldin alpha subunit | 0.439521847 | 0.204218443 | 188 |
| K19286>>FMN reductase [NAD(P)H] [EC: 1.5.1.39] | −0.747430341 | −0.25 | 189 |
| K11645>>fructose-bisphosphate aldolase, class I [EC: 4.1.2.13] | −0.474931359 | 0.012589928 | 190 |
| K03628>>transcription termination factor Rho | 0.229433422 | 0.008992806 | 191 |
| k_{——}Bacteria\|p_{——}Actinobacteria\|c_{——}Actinobacteria\|o_{——}Propionibacteriales | 0.055830269 | 0.101046436 | 192 |
| \|f_{——}Propionibacteriaceae\|g_{——}Propionibacterium | | | |
| K05515>>penicillin-binding protein 2 [EC: 3.4.16.4] | −0.365763585 | 0.010791367 | 193 |
| K03644>>lipoyl synthase [EC: 2.8.1.8] | −0.833259204 | −0.154839765 | 194 |
| K03884>>NADH-ubiquinone oxidoreductase chain 6 [EC: 7.1.1.2] | 0.054992341 | 0.114780903 | 195 |
| k_{——}Bacteria\|p_{——}Actinobacteria\|c_{——}Actinobacteria\|o_{——}Propionibacteriales | 0.056265789 | 0.101046436 | 196 |
| K03654>>ATP-dependent DNA helicase RecQ [EC: 3.6.4.12] | −0.827166564 | −0.102190974 | 197 |
| k_{——}Bacteria\|p_{——}Actinobacteria\|c_{——}Actinobacteria\|o_{——}Actinomycetales | 0.213701331 | 0.231687377 | 198 |
| K03679>>exosome complex component RRP4 | 0.446516008 | 0.194081099 | 199 |
| K03687>>molecular chaperone GrpE | 0.202805932 | 0 | 200 |
| K03688>>ubiquinone biosynthesis protein | 0.426782158 | 0.041366906 | 201 |
| k_{——}Bacteria\|p_{——}Actinobacteria\|c_{——}Actinobacteria\|o_{——}Propionibacteriales | 0.050592639 | 0.101046436 | 202 |
| \|f_{——}Propionibacteriaceae\|g_{——}Propionibacterium\|s_{——}Propionibacterium_freudenreichii | | | |
| K04019>>ethanolamine utilization protein EutA | 0.450339111 | 0.198659254 | 203 |
| K03882>>NADH-ubiquinone oxidoreductase chain 4L [EC: 7.1.1.2] | 0.015923246 | 0.095650752 | 204 |
| K05579>>NAD(P)H-quinone oxidoreductase subunit H [EC: 7.1.1.2] | 0.023260342 | 0.05853499 | 205 |
| k_{——}Bacteria\|p_{——}Firmicutes\|c_{——}Clostridia\|o_{——}Clostridiales\|f_{——}Eubacteriaceae | 0.779052609 | 0.242478744 | 206 |
| \|g_{——}Eubacterium\|s_{——}Eubacterium_hallii | | | |
| K04799>>flap endonuclease-1 [EC: 3.—.—.—] | 0.475149762 | 0.226945716 | 207 |
| K04801>>replication factor C small subunit | 0.481685752 | 0.204218443 | 208 |
| K18122>>4-hydroxybutyrate CoA-transferase [EC: 2.8.3.—] | −0.443005214 | −0.306245912 | 209 |
| K04802>>proliferating cell nuclear antigen | 0.455658629 | 0.244277305 | 210 |
| K04084>>thioredoxin:protein disulfide reductase [EC: 1.8.4.16] | −0.654182429 | −0.112328319 | 211 |
| K03549>>KUP system potassium uptake protein | 0.507143452 | 0.026487901 | 212 |
| K04079>>molecular chaperone HtpG | −0.48578308 | 0.019784173 | 213 |
| K05305>>fucokinase [EC: 2.7.1.52] | 0.789187886 | 0.370176586 | 214 |
| K04516>>chorismate mutase [EC: 5.4.99.5] | −0.553192789 | −0.013080445 | 215 |
| K05306>>phosphonoacetaldehyde hydrolase [EC: 3.11.1.1] | −0.869708446 | −0.286788751 | 216 |
| K04487>>cysteine desulfurase [EC: 2.8.1.7] | 0.227390171 | 0.008992806 | 217 |
| K03977>>GTPase | −0.199965948 | 0.003597122 | 218 |
| K05346>>deoxyribonucleoside regulator | 0.839469172 | 0.217298888 | 219 |
| K01153>>type I restriction enzyme, R subunit [EC: 3.1.21.3] | 0.089732058 | 0.007194245 | 220 |
| k_{——}Bacteria\|p_{——}Actinobacteria\|c_{——}Coriobacteriia\|o_{——}Eggerthellales | 0.429579483 | 0.332897319 | 221 |
| \|f_{——}Eggerthellaceae\|g_{——}Adlercreutzia | | | |
| K03885>>NADH dehydrogenase [EC: 1.6.99.3] | −0.685580553 | −0.220405494 | 222 |
| k_{——}Bacteria\|p_{——}Firmicutes\|c_{——}Clostridia\|o_{——}Clostridiales\|f_{——}Lachnospiraceae | 0.565502261 | 0.114290386 | 223 |
| \|g_{——}Dorea\|s_{——}Dorea_longicatena | | | |
| K03931>>putative isomerase | −0.881962324 | −0.174460432 | 224 |
| K03976>>Cys-tRNA(Pro)/Cys-tRNA(Cys) deacylase [EC: 3.1.1.—] | 0.270232026 | −0.010137345 | 225 |
| K05571>>multicomponent Na+: H+ antiporter subunit G | 0.511329984 | 0.32030739 | 226 |
| K05573>>NAD(P)H-quinone oxidoreductase subunit 2 [EC: 7.1.1.2] | 0.131893615 | 0.200130804 | 227 |
| K03980>>putative peptidoglycan lipid II flippase | 0.50883522 | 0.167756704 | 228 |
| K05574>>NAD(P)H-quinone oxidoreductase subunit 3 [EC: 7.1.1.2] | 0.01787019 | 0.102844997 | 229 |
| K00096>>glycerol-1-phosphate dehydrogenase [NAD(P)+] [EC: 1.1.1.261] | 0.40615523 | 0.080935252 | 230 |
| K01995>>branched-chain amino acid transport system ATP-binding protein | 0.225683812 | −0.015533028 | 231 |
| K04761>>LysR family transcriptional regulator, hydrogen peroxide-inducible genes activator | −0.672764763 | −0.139797253 | 232 |
| K19225>>rhomboid protease GluP [EC: 3.4.21.105] | 0.433164667 | 0.092380641 | 233 |
| K04029>>ethanolamine utilization protein EutP | 0.54740792 | 0.021582734 | 234 |
| K14653>>2-amino-5-formylamino-6-ribosylaminopyrimidin-4(3H)-one 5′-monophosphate deformylase | 0.385168327 | 0.19587966 | 235 |
| [EC: 3.5.1.102] | | | |
| K20626>>lactoyl-CoA dehydratase subunit alpha [EC: 4.2.1.54] | 0.370722316 | 0.176749509 | 236 |
| K03058>>DNA-directed RNA polymerase subunit N [EC: 2.7.7.6] | 0.500115798 | 0.283191629 | 237 |
| K03060>>DNA-directed RNA polymerase subunit omega [EC: 2.7.7.6] | 0.260938274 | 0.007194245 | 238 |
| K03111>>single-strand DNA-binding protein | 0.301281974 | 0.001798561 | 239 |
| K15986>>manganese-dependent inorganic pyrophosphatase [EC: 3.6.1.1] | 0.231920923 | 0.003597122 | 240 |
| K03073>>preprotein translocase subunit SecE | −0.137305537 | 0.001798561 | 241 |
| K04798>>prefoldin beta subunit | 0.378393059 | 0.225801177 | 242 |
| K04771>>serine protease Do [EC: 3.4.21.107] | 0.26096984 | 0.012589928 | 243 |
| K04488>>nitrogen fixation protein NifU and related proteins | 0.1797096 | 0.001798561 | 244 |
| K04072>>acetaldehyde dehydrogenase / alcohol dehydrogenase [EC: 1.2.1.10 1.1.1.1] | 0.377124609 | 0.025833878 | 245 |
| K03892>>ArsR family transcriptional regulator, arsenate/arsenite/ | 0.41018815 | 0.052158273 | 246 |
| antimonite-responsive transcriptional repressor | | | |
| k_{——}Bacteria\|p_{——}Actinobacteria\|c_{——}Coriobacteriia\|o_{——}Coriobacteriales | 0.058450245 | 0.102190974 | 247 |
| \|f_{——}Atopobiaceae\|g_{——}Olsenella\|s_{——}Olsenella_scatoligenes | | | |
| K03978>>GTP-binding protein | −0.390798752 | 0.008992806 | 248 |
| K03150>>2-iminoacetate synthase [EC: 4.1.99.19] | −0.170467913 | 0.008992806 | 249 |
| K03550>>holliday junction DNA helicase RuvA [EC: 3.6.4.12] | −0.173083712 | 0.005395683 | 250 |
| K03154>>sulfur carrier protein | −0.520966608 | −0.006540222 | 251 |
| K14274>>xylono-1,5-lactonase [EC: 3.1.1.110] | −0.884542643 | −0.429365598 | 252 |
| K04031>>ethanolamine utilization protein EutS | 0.528417553 | 0.053956835 | 253 |
| K04041>>fructose-1,6-bisphosphatase III [EC: 3.1.3.11] | −0.20547687 | 0.010791367 | 254 |
| K04074>>cell division initiation protein | 0.671549293 | 0.164323087 | 255 |
| K04769>>AbrB family transcriptional regulator, stage V sporulation protein T | 0.297519911 | 0.012589928 | 256 |
| K03177>>tRNA pseudouridine55 synthase [EC: 5.4.99.25] | −0.230910305 | 0.008992806 | 257 |
| K03152>>protein deglycase [EC: 3.5.1.124] | −0.247328366 | 0.010791367 | 258 |
| K04078>>chaperonin GroES | −0.101537416 | 0 | 259 |
| k_{——}Bacteria\|p_{——}Firmicutes\|c_{——}Bacilli\|o_{——}Lactobacillies\|f_{——}Leuconostocaceae | 0 | −0.001798561 | 260 |
| \|g_{——}Leuconostoc\|s_{——}Leuconostoc_carnosum | | | |
| K02992>>small subunit ribosomal protein S7 | −0.22717284 | 0 | 261 |
| K02888>>large subunit ribosomal protein L21 | −0.167599415 | 0.001798561 | 262 |
| K04564>>superoxide dismutase, Fe-Mn family [EC: 1.15.1.1] | −0.713453968 | −0.083060824 | 263 |
| K04653>>hydrogenase expression/formation protein HypC | 0.584478753 | 0.082897319 | 264 |
| K04655>>hydrogenase expression/formation protein HypE | 0.420903141 | 0.037279267 | 265 |
| K02897>>large subunit ribosomal protein L25 | −0.196420505 | 0.003597122 | 266 |
| K04759>>ferrous iron transport protein B | 0.205447047 | 0.005395683 | 267 |
| K03136>>transcription initiation factor TFIIE subunit alpha | 0.433075313 | 0.216808371 | 268 |
| K04762>>ribosome-associated heat shock protein Hsp15 | −0.810456054 | −0.145846959 | 269 |
| K02915>>large subunit ribosomal protein L34e | 0.428106338 | 0.19587966 | 270 |
| K02916>>large subunit ribosomal protein L35 | −0.135167811 | 0.001798561 | 271 |
| K03238>>translation initiation factor 2 subunit 2 | 0.437782376 | 0.232995422 | 272 |
| K03547>>DNA repair protein SbcD/Mre11 | 0.197210768 | 0.008992806 | 273 |
| K03167>>DNA topoisomerase VI subunit B [EC: 5.6.2.2] | 0.448988315 | 0.200621321 | 274 |
| K02921>>large subunit ribosomal protein L37Ae | 0.424889071 | 0.197024199 | 275 |
| K03106>>signal recognition particle subunit SRP54 [EC: 3.6.5.4] | 0.202260096 | 0 | 276 |
| K03043>>DNA-directed RNA polymerase subunit beta [EC: 2.7.7.6] | −0.150250946 | 0 | 277 |
| K05592>>ATP-dependent RNA helicase DeaD [EC: 3.6.4.13] | −0.543159868 | 0.021582734 | 278 |
| K03044>>DNA-directed RNA polymerase subunit B′ [EC: 2.7.7.6] | 0.481358926 | 0.226945716 | 279 |
| K03047>>DNA-directed RNA polymerase subunit D [EC: 2.7.7.6] | 0.496764251 | 0.219751472 | 280 |
| K02935>>large subunit ribosomal protein L7/L12 | −0.119254586 | 0 | 281 |
| K03070>>preprotein translocase subunit SecA [EC: 7.4.2.8] | −0.12793826 | 0.001798561 | 282 |
| K02991>>small subunit ribosomal protein S6e | 0.460516797 | 0.206017005 | 283 |
| K03077>>L-ribulose-5-phosphate 4-epimerase [EC: 5.1.3.4] | 0.142909457 | 0.005395683 | 284 |
| K03086>>RNA polymerase primary sigma factor | 0.212141513 | 0.003597122 | 285 |
| K03089>>RNA polymerase sigma-32 factor | −0.297370351 | −0.292674951 | 286 |
| K03090>>RNA polymerase sigma-B factor | 0.642176895 | 0.107913669 | 287 |
| K03205>>type IV secretion system protein VirD4 [EC: 7.4.2.8] | 0.340361148 | 0 | 288 |
| K03122>>transcription initiation factor TFIIA large subunit | 0 | 0.043655984 | 289 |
| K19883>>bifunctional aminoglycoside 6′-N-acetyltransferase / aminoglycoside 2″-phosphotransferase | −0.490184527 | −0.291203401 | 290 |
| [EC: 2.3.1.82 2.7.1.190] | | | |
| K02970>>small subunit ribosomal protein S21 | −0.193922391 | 0 | 291 |
| K02974>>small subunit ribosomal protein S24e | 0.448971049 | 0.241334205 | 292 |
| K05716>>cyclic 2,3-diphosphoglycerate synthetase [EC: 4.6.1.—] | 0.443483099 | 0.209614127 | 293 |
| K02902>>large subunit ribosomal protein L28 | −0.098836762 | 0.001798561 | 294 |
| K03124>>transcription initiation factor TFIIB | 0.49919706 | 0.212557227 | 295 |
| K02912>>large subunit ribosomal protein L32e | 0.4772532 | 0.243132767 | 296 |
| K02988>>small subunit ribosomal protein S5 | −0.135107061 | 0 | 297 |
| K03151>>tRNA uracil 4-sulfurtransferase [EC: 2.8.1.4] | 0.314146935 | 0.007194245 | 298 |
| K18138>>multidrug efflux pump | −0.537053267 | −0.348757358 | 299 |
| K03469>>ribonuclease HI [EC: 3.1.26.4] | −0.229431749 | 0.017985612 | 300 |
| K21471>>peptidoglycan DL-endopeptidase CwlO [EC: 3.4.—.—] | 0.31660507 | 0.01618705 | 301 |
| K03183>>demethylmenaquinone methyltransferase / 2-methoxy-6-polyprenyl-1,4-benzoquinol methylase | −0.714766686 | −0.090255069 | 302 |
| [EC: 2.1.1.163 2.1.1.201] | | | |
| K03186>>flavin prenyltransferase [EC: 2.5.1.129] | 0.652290257 | 0.145683453 | 303 |
| K03474>>pyridoxine 5-phosphate synthase [EC: 2.6.99.2] | −0.607190296 | −0.069326357 | 304 |
| K08974>>putative membrane protein | −0.361240513 | −0.047743623 | 305 |
| k_{——}Bacteria\|p_{——}Bacteroidetes\|c_{——}Bacteroidia\|o_{——}Bacteroidales\|f_{——}Prevotellaceae | −0.53007898 | −0.097449313 | 306 |
| K02995>>small subunit ribosomal protein S8e | 0.376365337 | 0.207815566 | 307 |
| K03216>>tRNA (cytidine/uridine-2′-O-)-methyltransferase [EC: 2.1.1.207] | 0.437798857 | 0.012589928 | 308 |
| K03488>>beta-glucoside operon transcriptional antiterminator | 0.362492363 | −0.004741661 | 309 |
| K02956>>small subunit ribosomal protein S15 | −0.133235224 | 0 | 310 |
| K22373>>lactate racemase [EC: 5.1.2.1] | 0.701281404 | 0.117724003 | 311 |
| K03540>>ribonuclease P protein subunit RPR2 [EC: 3.1.26.5] | 0.41631088 | 0.220405494 | 312 |
| K02929>>large subunit ribosomal protein L44e | 0.412180476 | 0.216808371 | 313 |
| K03495>>tRNA uridine 5-carboxymethylaminomethyl modification enzyme | −0.160046685 | 0.003597122 | 314 |
| K02967>>small subunit ribosomal protein S2 | −0.145397968 | 0 | 315 |
| K02968>>small subunit ribosomal protein S20 | −0.269009351 | 0.003597122 | 316 |
| k_{——}Bacteria\|p_{——}Actinobacteria\|c_{——}Coriobacteriia\|o_{——}Coriobacteriales\|f_{——}Coriobacteriaceae\|g_{——}Enorma | 0.187229384 | 0.294637018 | 317 |
| K02889>>large subunit ribosomal protein L21e | 0.402851354 | 0.199476782 | 318 |
| K02896>>large subunit ribosomal protein L24e | 0.435126352 | 0.216808371 | 319 |
| k_{——}Bacteria\|p_{——}Firmicutes\|c_{——}Firmicutes_unclassified\|o_{——}Firmicutes_unclassified | 0.004661582 | 0.006376717 | 320 |
| \|f_{——}Firmicutes_unclassified\|g_{——}Firmicutes_unclassified\|s_{——}Firmicutes_bacterium_CAG_170 | | | |
| K03496>>chromosome partitioning protein | 0.316818106 | 0 | 321 |
| K13016>>UDP-N-acetyl-2-amino-2-deoxyglucuronate dehydrogenase [EC: 1.1.1.335] | −0.458540894 | −0.325376063 | 322 |
| K02922>>large subunit ribosomal protein L37e | 0.524793564 | 0.272400262 | 323 |
| K02924>>large subunit ribosomal protein L39e | 0.525137376 | 0.258011772 | 324 |
| K03465>>thymidylate synthase (FAD) [EC: 2.1.1.148] | 0.286042088 | 0.005395683 | 325 |
| K03531>>cell division protein FtsZ | 0.308993492 | 0.008992806 | 326 |
| K03533>>TorA specific chaperone | 0.434111139 | 0.172825376 | 327 |
| K02931>>large subunit ribosomal protein L5 | −0.111965819 | 0 | 328 |
| K12511>>tight adherence protein C | 0.611521444 | 0.172988882 | 329 |
| K05780>>alpha-D-ribose 1-methylphosphonate 5-triphosphate synthase subunit PhnL [EC: 2.7.8.37] | 0.542402406 | 0.232668411 | 330 |
| K03498>>trk system potassium uptake protein | 0.111538998 | 0.001798561 | 331 |
| K02936>>large subunit ribosomal protein L7Ae | 0.501742128 | 0.249672989 | 332 |
| K11235>>mating pheromone A-factor | 0 | 0.045454545 | 333 |
| K13019>>UDP-GlcNAc3NAcA epimerase [EC: 5.1.3.23] | −0.322397192 | −0.30706344 | 334 |
| K02947>>small subunit ribosomal protein S10e | 0 | 0.043655984 | 335 |
| K03217>>YidC/Oxa1 family membrane protein insertase | 0.017539554 | 0 | 336 |
| K02961>>small subunit ribosomal protein S17 | −0.119617687 | 0 | 337 |
| K13640>>MerR family transcriptional regulator, heat shock protein HspR | 0.590957549 | 0.180837148 | 338 |
| K02963>>small subunit ribosomal protein S18 | −0.109486316 | 0 | 339 |
| K18349>>two-component system, OmpR family, response regulator VanR | 0.522460956 | 0.04676259 | 340 |
| K03263>>translation initiation factor 5A | 0.46638818 | 0.230542838 | 341 |
| K03455>>monovalent cation: H+ antiporter-2, CPA2 family | −0.821758643 | −0.140451275 | 342 |
| K02984>>small subunit ribosomal protein S3Ae | 0.496894447 | 0.228744277 | 343 |
| K03521>>electron transfer flavoprotein beta subunit | 0.529213114 | 0.023381295 | 344 |
| K03232>>elongation factor 1-beta | 0.492033561 | 0.207815566 | 345 |
| K03269>>UDP-2,3-diacylglucosamine hydrolase [EC: 3.6.1.54] | −0.790648894 | −0.123119686 | 346 |
| K03234>>elongation factor 2 | 0.470861015 | 0.207161543 | 347 |
| K16936>>thiosulfate dehydrogenase (quinone) small subunit [EC: 1.8.5.2] | −0.548425762 | −0.257030739 | 348 |
| K03236>>translation initiation factor 1A | 0.465997629 | 0.223348594 | 349 |
| K03270>>3-deoxy-D-manno-octulosonate 8-phosphate phosphatase (KDO 8-P phosphatase) | −0.76028162 | −0.227763244 | 350 |
| [EC: 3.1.3.45] | | | |
| k_{——}Bacteria\|p_{——}Actinobacteria\|c_{——}Coriobacteriia\|o_{——}Eggerthellales\|f_{——}Eggerthellaceae | 0.336983621 | 0.243296272 | 351 |
| \|g_{——}Gordonibacter | | | |
| K03274>>ADP-L-glycero-D-manno-heptose 6-epimerase [EC: 5.1.3.20] | −0.415297014 | −0.318508829 | 352 |
| K03430>>2-aminoethylphosphonate-pyruvate transaminase [EC: 2.6.1.37] | −0.989428903 | −0.199640288 | 353 |
| K03442>>small conductance mechanosensitive channel | −0.214668318 | 0.010791367 | 354 |
| K03281>>chloride channel protein, CIC family | −0.690642021 | −0.187704382 | 355 |
| K03529>>chromosome segregation protein | 0.232159246 | 0.012589928 | 356 |
| K06013>>STE24 endopeptidase [EC: 3.4.24.84] | −0.407678201 | −0.330117724 | 357 |
| K16937>>thiosulfate dehydrogenase (quinone) large subunit [EC: 1.8.5.2] | −0.525921095 | −0.237900589 | 358 |
| K06023>>HPr kinase/phosphorylase [EC: 2.7.11.—2.7.4.—] | 0.466754331 | 0.008992806 | 359 |
| K03534>>L-rhamnose mutarotase [EC: 5.1.3.32] | −0.59514939 | 0.007848267 | 360 |
| K03325>>arsenite transporter | 0.611124829 | 0.104643558 | 361 |
| K03475>>ascorbate PTS system EIIC component | 0.537426494 | 0.101373447 | 362 |
| K03476>>L-ascorbate 6-phosphate lactonase [EC: 3.1.1.—] | 0.295591628 | 0.057553957 | 363 |
| K03484>>LacI family transcriptional regulator, sucrose operon repressor | 0.514647979 | 0.032374101 | 364 |
| K03486>>GntR family transcriptional regulator, trehalose operon transcriptional repressor | 0.662500048 | 0.178384565 | 365 |
| K03500>>16S rRNA (cytosine967-C5)-methyltransferase [EC: 2.1.1.176] | 0.35780794 | 0.032374101 | 366 |
| K03389>>heterodisulfide reductase subunit B2 [EC: 1.8.7.3 1.8.98.4 1.8.98.5 1.8.98.6] | −0.156270537 | −0.137671681 | 367 |
| K03407>>two-component system, chemotaxis family, sensor kinase CheA [EC: 2.7.13.3] | −0.391667163 | −0.050523218 | 368 |
| K03517>>quinolinate synthase [EC: 2.5.1.72] | 0.287768871 | 0.017985612 | 369 |
| K08600>>sortase B [EC: 3.4.22.71] | 0.500312822 | 0.023381295 | 370 |
| K05770>>translocator protein | −1.028057197 | −0.232995422 | 371 |
| K03518>>aerobic carbon-monoxide dehydrogenase small subunit [EC: 1.2.5.3] | 0.356027605 | 0.035971223 | 372 |
| K03522>>electron transfer flavoprotein alpha subunit | 0.29282851 | 0.012589928 | 373 |
| K18887>>ATP-binding cassette, subfamily B, multidrug efflux pump | 0.547182272 | 0.339928058 | 374 |
| K03525>>type III pantothenate kinase [EC: 2.7.1.33] | 0.155342247 | 0 | 375 |
| K03527>>4-hydroxy-3-methylbut-2-en-1-yl diphosphate reductase [EC: 1.17.7.4] | −0.215390878 | 0.005395683 | 376 |
| K05801>>DnaJ like chaperone protein | −0.936291119 | −0.215173316 | 377 |
| K03338>>5-dehydro-2-deoxygluconokinase [EC: 2.7.1.92] | 0.423622732 | 0.152550687 | 378 |
| K03429>>processive 1,2-diacylglycerol beta-glucosyltransferase [EC: 2.4.1.315] | 0.321515352 | 0.055264879 | 379 |
| k_{——}Bacteria\|p_{——}Firmicutes\|c_{——}Clostridia\|o_{——}Clostridiales\|f_{——}Ruminococcaceae | −0.358493897 | −0.325212557 | 380 |
| \|g_{——}Flavonifractor\|s_{——}Flavonifractor_plautii | | | |
| K03538>>ribonuclease P protein subunit POP4 [EC: 3.1.26.5] | 0.420312045 | 0.19587966 | 381 |
| K08325>>NADP-dependent alcohol dehydrogenase [EC: 1.1.—.—] | −0.854533321 | −0.133257031 | 382 |
| K03327>>multidrug resistance protein, MATE family | −0.780059057 | −0.24264225 | 383 |
| K03539>>ribonuclease P/MRP protein subunit RPP1 [EC: 3.1.26.5] | 0 | 0.081916285 | 384 |
| K03433>>proteasome beta subunit [EC: 3.4.25.1] | 0.495121822 | 0.223348594 | 385 |
| K03431>>phosphoglucosamine mutase [EC: 5.4.2.10] | 0.322848246 | 0 | 386 |
| K08368>>MFS transporter, putative metabolite transport protein | 0.489360439 | 0.258502289 | 387 |
| K03385>>nitrite reductase (cytochrome c-552) [EC: 1.7.2.2] | −0.539626513 | −0.090255069 | 388 |
| K03243>>translation initiation factor 5B | 0.498763433 | 0.223348594 | 389 |
| K20830>>beta-porphyranase [EC: 3.2.1.178] | −0.650917253 | −0.392086331 | 390 |
| k_{——}Bacteria\|p_{——}Actinobacteria\|c_{——}Coriobacteriia\|o_{——}Coriobacteriales | 0.068454077 | 0.066873774 | 391 |
| \|f_{——}Coriobacteriaceae\|g_{——}Enorma\|s_{——}Enorma_massiliensis | | | |
| K03264>>translation initiation factor 6 | 0.488259538 | 0.200621321 | 392 |
| K03265>>peptide chain release factor subunit 1 | 0.441683238 | 0.204218443 | 393 |
| k_{——}Bacteria\|p_{——}Firmicutes\|c_{——}Negativicutes | −0.53773866 | −0.205526488 | 394 |
| K08998>>uncharacterized protein | −0.309017955 | 0.003597122 | 395 |
| K08981>>putative membrane protein | 0.43408601 | 0.254578156 | 396 |
| K03282>>large conductance mechanosensitive channel | −0.225172148 | 0.007194245 | 397 |
| K09013>>Fe-S cluster assembly ATP-binding protein | 0.289262463 | 0.010791367 | 398 |
| K03303>>lactate permease | −0.304060686 | 0.01030085 | 399 |
| k_{——}Bacteria\|p_{——}Firmicutes\|c_{——}Clostridia\|o_{——}Clostridiales\|f_{——}Lachnospiraceae | 0.486571191 | 0.292511445 | 400 |
| \|g_{——}Coprococcus\|s_{——}Coprococcus_catus | | | |
| K08302>>tagatose 1,6-diphosphate aldolase GatY/KbaY [EC: 4.1.2.40] | 0.668159806 | 0.121811642 | 401 |
| K03312>>glutamate: Na+ symporter, ESS family | −0.825507298 | −0.107586658 | 402 |
| K17733>>peptidoglycan LD-endopeptidase CwlK [EC: 3.4.—.—] | −0.504610106 | −0.284499673 | 403 |
| k_{——}Bacteria\|p_{——}Bacteroidetes\|c_{——}Bacteroidia\|o_{——}Bacteroidales\|f_{——}Tannerellaceae | −0.721178013 | −0.346958797 | 404 |
| \|g_{——}Parabacteroides\|s_{——}Parabacteroides_distasonis | | | |
| K09121>>pyridinium-3,5-bisthiocarboxylic acid mononucleotide nickel chelatase [EC: 4.99.1.12] | 0.468182517 | 0.011445389 | 405 |
| K09022>>2-iminobutanoate/2-iminopropanoate deaminase [EC: 3.5.99.10] | 0.292024787 | 0.008992806 | 406 |
| K08357>>tetrathionate reductase subunit A | 0.311575405 | 0.227599738 | 407 |
| K09117>>uncharacterized protein | 0.438617696 | 0.031229562 | 408 |
| K03330>>glutamyl-tRNA(Gln) amidotransferase subunit E [EC: 6.3.5.7] | 0.434656957 | 0.202419882 | 409 |
| K03332>>fructan beta-fructosidase [EC: 3.2.1.80] | −0.975003081 | −0.106932636 | 410 |
| K03526>>(E)-4-hydroxy-3-methylbut-2-enyl-diphosphate synthase [EC: 1.17.7.1 1.17.7.3] | −0.065595562 | 0 | 411 |
| K08223>>MFS transporter, FSR family, fosmidomycin resistance protein | −0.729228538 | −0.24558535 | 412 |
| K03340>>diaminopimelate dehydrogenase [EC: 1.4.1.16] | −0.537415863 | −0.044800523 | 413 |
| K08961>>chondroitin-sulfate-ABC endolyase/exolyase [EC: 4.2.2.20 4.2.2.21] | −0.887941751 | −0.295781557 | 414 |
| k_{——}Bacteria\|p_{——}Firmicutes\|c_{——}Clostridia\|o_{——}Clostridiales\|f_{——}Ruminococcaceae | −0.359433144 | −0.332406802 | 415 |
| \|g_{——}Flavonifractor | | | |
| K03410>>chemotaxis protein CheC | −0.576483866 | −0.11706998 | 416 |
| K03411>>chemotaxis protein CheD [EC: 3.5.1.44] | −0.694582693 | −0.246075867 | 417 |
| K05820>>MFS transporter, PPP family, 3-phenylpropionic acid transporter | 0.632650281 | 0.234793983 | 418 |
| K12137>>hydrogenase-4 component B [EC: 1.—.—.—] | 0.48616163 | 0.24852845 | 419 |
| K07719>>two-component system, response regulator YcbB | −0.484982969 | −0.326193591 | 420 |
| K07720>>two-component system, response regulator YesN | 0.285941777 | 0.023381295 | 421 |
| K08744>>cardiolipin synthase (CMP-forming) [EC: 2.7.8.41] | 0.584165629 | 0.10088293 | 422 |
| K08289>>phosphoribosylglycinamide formyltransferase 2 [EC: 2.1.2.2] | −0.274148589 | −0.013734467 | 423 |
| K19172>>DNA sulfur modification protein DndE | 0.055007443 | 0.073904513 | 424 |
| K08352>>thiosulfate reductase / polysulfide reductase chain A [EC: 1.8.5.5] | 0.282347304 | 0.230052322 | 425 |
| K08222>>MFS transporter, YQGE family, putative transporter | −0.748096886 | −0.355788097 | 426 |
| K08217>>MFS transporter, DHA3 family, macrolide efflux protein | 0.411732629 | 0.012589928 | 427 |
| K07533>>foldase protein PrsA [EC: 5.2.1.8] | 0.470400059 | 0.070143885 | 428 |
| K01079>>phosphoserine phosphatase [EC: 3.1.3.3] | −0.324152467 | −0.011935906 | 429 |
| k_{——}Bacteria\|p_{——}Actinobacteria\|c_{——}Coriobacteriia\|o_{——}Coriobacteriales | 0.585679876 | 0.064748201 | 430 |
| \|f_{——}Coriobacteriaceae\|g_{——}Collinsella\|s_{——}Collinsella_aerofaciens | | | |
| K08369>>MFS transporter, putative metabolite: H+ symporter | 0.577139878 | 0.055264879 | 431 |
| K08372>>putative serine protease PepD [EC: 3.4.21.—] | 0.240012327 | 0.228253761 | 432 |
| K07742>>uncharacterized protein | 0.298222785 | 0.028776978 | 433 |
| K07812>>trimethylamine-N-oxide reductase (cytochrome c) [EC: 1.7.2.3] | 0.52940207 | 0.336821452 | 434 |
| K18640>>plasmid segregation protein ParM | 0.340116266 | 0.043165468 | 435 |
| K07814>>putative two-component system response regulator | 0.480531818 | 0.10971223 | 436 |
| K08641>>zinc D-Ala-D-Ala dipeptidase [EC: 3.4.13.22] | −0.810135566 | −0.307717462 | 437 |
| K07776>>two-component system, OmpR family, response regulator RegX3 | 0.589808561 | 0.320634402 | 438 |
| K11755>>phosphoribosyl-AMP cyclohydrolase / phosphoribosyl-ATP pyrophosphohydrolase | −0.387850504 | 0.01618705 | 439 |
| [EC: 3.5.4.19 3.6.1.31] | | | |
| K08218>>MFS transporter, PAT family, beta-lactamase induction signal transducer AmpG | −0.930726616 | −0.148790059 | 440 |
| K08681>>5′-phosphate synthase pdxT subunit [EC: 4.3.3.6] | 0.310465036 | 0.028776978 | 441 |
| K08963>>methylthioribose-1-phosphate isomerase [EC: 5.3.1.23] | −0.775361227 | −0.219751472 | 442 |
| K07862>>serine/threonine transporter | 0.564391407 | 0.012589928 | 443 |
| K08972>>putative membrane protein | 0.577921597 | 0.170536298 | 444 |
| K07991>>archaeal preflagellin peptidase FlaK [EC: 3.4.23.52] | 0.37879535 | 0.224002616 | 445 |
| K08999>>uncharacterized protein | 0.679767895 | 0.150261609 | 446 |
| k_{——}Bacteria\|p_{——}Actinobacteria\|c_{——}Coriobacteriia\|o_{——}Coriobacteriales | 0.205270114 | 0.332733813 | 447 |
| \|f_{——}Coriobacteriaceae\|g_{——}Collinsella\|s_{——}Collinsella_stercoris | | | |
| K08094>>6-phospho-3-hexuloisomerase [EC: 5.3.1.27] | 0.642799483 | 0.362818836 | 448 |
| K09015>>Fe-S cluster assembly protein SufD | 0.149811266 | −0.011118378 | 449 |
| K12267>>peptide methionine sulfoxide reductase msrA/msrB [EC: 1.8.4.11 1.8.4.12] | −0.497582128 | 0.017985612 | 450 |
| K09167>>uncharacterized protein | 0.405405395 | 0.342380641 | 451 |
| K08159>>MFS transporter, DHA1 family, L-arabinose/ | 0.605605806 | 0.086003924 | 452 |
| isopropyl-beta-D-thiogalactopyranoside export protein | | | |
| K09251>>putrescine aminotransferase [EC: 2.6.1.82] | 0.761999454 | 0.252616089 | 453 |
| K07735>>putative transcriptional regulator | −0.833378683 | −0.138652714 | 454 |
| K12510>>tight adherence protein B | 0.403479741 | 0.034172662 | 455 |
| K09126>>uncharacterized protein | 0.452654347 | 0.21795291 | 456 |
| K09157>>uncharacterized protein | 0.325084581 | 0 | 457 |
| k_{——}Bacteria\|p_{——}Actinobacteria\|c_{——}Actinobacteria\|o_{——}Actinomycetales\|f_{——}Actinomycetaceae | 0.018487495 | −0.008992806 | 458 |
| \|g_{——}Actinomyces\|s_{——}Actinomyces_sp_HMSC035G02 | | | |
| K05810>>polyphenol oxidase [EC: 1.10.3.—] | 0.230819389 | 0.007194245 | 459 |
| K10112>>multiple sugar transport system ATP-binding protein | 0.569373724 | 0.01618705 | 460 |
| K07777>>two-component system, NarL family, sensor histidine kinase DegS [EC: 2.7.13.3] | 0.170798945 | 0.142249836 | 461 |
| K10117>>raffinose/stachyose/melibiose transport system substrate-binding protein | 0.34076148 | 0.001798561 | 462 |
| K07755>>arsenite methyltransferase [EC: 2.1.1.137] | 0.415762533 | 0.295127534 | 463 |
| K19954>>alcohol dehydrogenase [EC: 1.1.1.—] | 0.561275325 | 0.360039241 | 464 |
| K07718>>two-component system, sensor histidine kinase YesM [EC: 2.7.13.3] | 0.296929253 | 0.010791367 | 465 |
| K07816>>putative GTP pyrophosphokinase [EC: 2.7.6.5] | 0.442984383 | 0.023381295 | 466 |
| K07722>>CopG family transcriptional regulator, nickel-responsive regulator | 0.696905434 | 0.222040549 | 467 |
| K10245>>fatty acid elongase 2 [EC: 2.3.1.199] | 0 | 0.045454545 | 468 |
| K07727>>putative transcriptional regulator | −0.253796185 | 0.012589928 | 469 |
| K07728>>putative transcriptional regulator | 0.457624685 | 0.213211249 | 470 |
| K07729>>putative transcriptional regulator | 0.237042681 | 0.003597122 | 471 |
| K09474>>acid phosphatase (class A) [EC: 3.1.3.2] | −1.061015213 | −0.234793983 | 472 |
| K10542>>methyl-galactoside transport system ATP-binding protein [EC: 7.5.2.11] | 0.322346951 | 0.010791367 | 473 |
| K07736>>CarD family transcriptional regulator | 0.28703147 | 0.017985612 | 474 |
| K07738>>transcriptional repressor NrdR | 0.214525512 | 0.003597122 | 475 |
| K10689>>peroxin-4 [EC: 2.3.2.23] | 0 | 0.043655984 | 476 |
| K20370>>phosphoenolpyruvate carboxykinase (diphosphate) [EC: 4.1.1.38] | 0.032192593 | 0.106442119 | 477 |
| K10974>>cytosine permease | 0.801923277 | 0.169228254 | 478 |
| k_{——}Bacteria\|p_{——}Actinobacteria\|c_{——}Coriobacteriia\|o_{——}Coriobacteriales | 0.563397381 | 0.053302812 | 479 |
| \|f_{——}Coriobacteriaceae\|g_{——}Collinsella | | | |
| K10979>>DNA end-binding protein Ku | 0.421388021 | 0.224166122 | 480 |
| K11002>>phosphatidylinositol N-acetylglucosaminyltransferase ERI1 subunit | 0 | 0.045454545 | 481 |
| k_{——}Bacteria\|p_{——}Bacteroidetes\|c_{——}Bacteroidia\|o_{——}Bacteroidales | −0.842152017 | −0.313113146 | 482 |
| \|f_{——}Tannerellaceae\|g_{——}Parabacteroides | | | |
| K09680>>type II pantothenate kinase [EC: 2.7.1.33] | −0.741477202 | −0.062622629 | 483 |
| K14170>>chorismate mutase / prephenate dehydratase [EC: 5.4.99.5 4.2.1.51] | 0.337283231 | 0.026978417 | 484 |
| K06142>>outer membrane protein | −0.774164324 | −0.088456508 | 485 |
| K11130>>H/ACA ribonucleoprotein complex subunit 3 | 0.486067529 | 0.260464356 | 486 |
| K10805>>acyl-CoA thioesterase II [EC: 3.1.2.—] | 0.55050466 | 0.104643558 | 487 |
| K11145>>ribonuclease III family protein [EC: 3.1.26.—] | 0.309482515 | 0.025179856 | 488 |
| K08096>>GTP cyclohydrolase IIa [EC: 3.5.4.29] | 0.447397194 | 0.225147155 | 489 |
| K11175>>phosphoribosylglycinamide formyltransferase 1 [EC: 2.1.2.2] | 0.200086484 | 0.003597122 | 490 |
| K14591>>protein AroM | 0.468507991 | 0.240189666 | 491 |
| K09931>>uncharacterized protein | 0.352302679 | 0.326847613 | 492 |
| K08153>>MFS transporter, DHA1 family, multidrug resistance protein | −0.489530908 | −0.344179202 | 493 |
| K14742>>tRNA threonylcarbamoyladenosine biosynthesis protein TsaB | −0.541929328 | −0.276324395 | 494 |
| K07717>>two-component system, sensor histidine kinase YcbA [EC: 2.7.13.3] | −0.411859145 | −0.28057554 | 495 |
| K09482>>glutamyl-tRNA(Gln) amidotransferase subunit D [EC: 6.3.5.7] | 0.492237413 | 0.214355788 | 496 |
| K09974>>uncharacterized protein | 0.646276982 | 0.180510137 | 497 |
| K22476>>N-acetylglutamate synthase [EC: 2.3.1.1] | 0 | 0.043655984 | 498 |
| K11991>>tRNA(adenine34) deaminase [EC: 3.5.4.33] | −0.23286185 | 0.003597122 | 499 |
| K09014>>Fe-S cluster assembly protein SufB | −0.33701408 | −0.015533028 | 500 |
| K09684>>PucR family transcriptional regulator, purine catabolismregulatory protein | 0.684283546 | 0.244277305 | 501 |
| K07696>>two-component system, NarL family, response regulator NreC | 0.346758191 | 0.294637018 | 502 |
| K20488>>two-component system, OmpR family, lantibiotic biosynthesis response regulator NisR/SpaR | −0.02480256 | −0.105788097 | 503 |
| K10041>>aspartate/glutamate/glutamine transport system ATP-binding protein [EC: 7.4.2.1] | 0.073574449 | 0.144048398 | 504 |
| K09693>>teichoic acid transport system ATP-binding protein [EC: 7.5.2.4] | 0.423753778 | 0.097776324 | 505 |
| K02864>>large subunit ribosomal protein L10 | 0.352018352 | 0 | 506 |
| K10118>>raffinose/stachyose/melibiose transport system permease protein | 0.517720447 | 0.035971223 | 507 |
| K09706>>uncharacterized protein | 0.20102642 | 0.122138653 | 508 |
| K10119>>raffinose/stachyose/melibiose transport system permease protein | 0.375101241 | 0.021582734 | 509 |
| K10206>>LL-diaminopimelate aminotransferase [EC: 2.6.1.83] | −0.164073691 | 0.003597122 | 510 |
| K10439>>ribose transport system substrate-binding protein | 0.192299238 | 0.005395683 | 511 |
| K09767>>cyclic-di-GMP-binding protein | 0.439299414 | 0.084041857 | 512 |
| K13626>>flagellar assembly factor FliW | −0.552732608 | −0.130150425 | 513 |
| K10546>>putative multiple sugar transport system substrate-binding protein | 0.832179237 | 0.041530412 | 514 |
| K10559>>rhamnose transport system substrate-binding protein | 0.323085815 | 0.228580772 | 515 |
| K09789>>pimeloyl-[acyl-carrier protein] methyl ester esterase [EC: 3.1.1.85] | −0.688039976 | −0.301013734 | 516 |
| K10794>>D-proline reductase (dithiol) PrdB [EC: 1.21.4.1] | 0.049471207 | 0.118378025 | 517 |
| K12132>>eukaryotic-like serine/threonine-protein kinase [EC: 2.7.11.1] | 0.321330884 | 0.01618705 | 518 |
| K09790>>uncharacterized protein | 0.024740176 | 0.000654022 | 519 |
| K09797>>uncharacterized protein | −1.072737844 | −0.186396337 | 520 |
| k_{——}Eukaryota\|p_{——}Ascomycota | 0.002550711 | −0.001798561 | 521 |
| K10026>>7-carboxy-7-deazaguanine synthase [EC: 4.3.99.3] | 0.205139493 | 0.008992806 | 522 |
| K09807>>uncharacterized protein | 0.143139 | 0.010791367 | 523 |
| K12978>>lipid A 4′-phosphatase [EC: 3.1.3.—] | 0.553159428 | 0.192609549 | 524 |
| K11720>>lipopolysaccharide export system permease protein | −0.773034024 | −0.12851537 | 525 |
| K09811>>cell division transport system permease protein | 0.246780302 | 0.010791367 | 526 |
| K11050>>multidrug/hemolysin transport system ATP-binding protein | 0.38069617 | 0.012099411 | 527 |
| K09730>>uncharacterized protein | 0.435786406 | 0.218606933 | 528 |
| K09810>>lipoprotein-releasing system ATP-binding protein [EC: 3.6.3.—] | −0.880012391 | −0.149444081 | 529 |
| K11069>>spermidine/putrescine transport system substrate-binding protein | −0.136285439 | 0.007194245 | 530 |
| K09733>>(5-formylfuran-3-yl)methyl phosphate synthase [EC: 4.2.3.153] | 0.465982347 | 0.211412688 | 531 |
| K11105>>cell volume regulation protein A | −0.487186115 | 0.015042511 | 532 |
| K09815>>zinc transport system substrate-binding protein | −0.243263635 | 0.014388489 | 533 |
| K09939>>uncharacterized protein | −1.049852083 | −0.263570961 | 534 |
| k_{——}Bacteria\|p_{——}Bacteroidetes\|c_{——}Bacteroidia\|o_{——}Bacteroidales\|f_{——}Bacteroidaceae | −0.5215767 | −0.329463702 | 535 |
| \|g_{——}Bacteroides\|s_{——}Bacteroides_thetaiotaomicron | | | |
| K00794>>6,7-dimethyl-8-ribityllumazine synthase [EC: 2.5.1.78] | −0.173350395 | 0.001798561 | 536 |
| K09861>>uncharacterized protein | −0.16837503 | 0.010791367 | 537 |
| K09768>>uncharacterized protein | 0.387707005 | 0.032374101 | 538 |
| K13252>>putrescine carbamoyltransferase [EC: 2.1.3.6] | 0.687759571 | 0.217135383 | 539 |
| K11176>>IMP cyclohydrolase [EC: 3.5.4.10] | 0.501542287 | 0.2323414 | 540 |
| K09903>>uridylate kinase [EC: 2.7.4.22] | −0.151976609 | 0 | 541 |
| K11184>>catabolite repression HPr-like protein | 0.35470916 | 0.017495095 | 542 |
| K10040>>aspartate/glutamate/glutamine transport system permease protein | 0.666625906 | 0.054774362 | 543 |
| K09690>>lipopolysaccharide transport system permease protein | −0.852938876 | −0.178057554 | 544 |
| K06200>>carbon starvation protein | 0.340404477 | 0.014388489 | 545 |
| K14415>>RNA-splicing ligase RtcB (3′-phosphate/5′-hydroxy nucleic acid ligase) [EC: 6.5.1.8] | −0.659923932 | −0.181000654 | 546 |
| K07653>>two-component system, OmpR family, sensor histidine kinase MprB [EC: 2.7.13.3] | 0.03152486 | 0.101046436 | 547 |
| K06896>>maltose 6′-phosphate phosphatase [EC: 3.1.3.90] | 0.611845518 | 0.228744277 | 548 |
| K06207>>GTP-binding protein | 0.512924873 | 0.003597122 | 549 |
| k_{——}Eukaryota\|p_{——}Ascomycota\|c_{——}Saccharomyceses\|o_{——}Saccharomycesles | 0.002550711 | −0.001798561 | 550 |
| \|f_{——}Saccharomycesceae | | | |
| K09888>>cell division protein ZapA | −0.419478341 | 0.014388489 | 551 |
| k_{——}Bacteria\|p_{——}Firmicutes\|c_{——}Clostridia\|o_{——}Clostridiales\|f_{——}Clostridiales_Family_XIII_Incertae_Sedis | 0.063811422 | 0.053139307 | 552 |
| K09808>>lipoprotein-releasing system permease protein | −0.931858875 | −0.154839765 | 553 |
| K06334>>spore coat protein JC | 0.3297695 | 0.035971223 | 554 |
| K09747>>uncharacterized protein | 0.302247318 | 0.001798561 | 555 |
| K09762>>uncharacterized protein | 0.363392582 | 0.017985612 | 556 |
| K06346>>spoIIIJ-associated protein | 0.260540848 | 0.010791367 | 557 |
| K06379>>stage II sporulation protein AB (anti-sigma F factor) [EC: 2.7.11.1] | 0.337732511 | 0.018639634 | 558 |
| K09773>>[pyruvate, water dikinase]-phosphate phosphotransferase / [pyruvate, water dikinase] kinase | 0.523061066 | 0.04823414 | 559 |
| [EC: 2.7.4.28 2.7.11.33] | | | |
| k_{——}Eukaryota | 0.006411993 | 0.008338784 | 560 |
| K09803>>uncharacterized protein | −0.330013185 | −0.112982341 | 561 |
| K06410>>dipicolinate synthase subunit A | −0.404403848 | −0.057717462 | 562 |
| K09816>>zinc transport system permease protein | −0.570393716 | 0.017985612 | 563 |
| K15342>>CRISP-associated protein Cas1 | 0.286318105 | 0.014388489 | 564 |
| K11924>>DtxR family transcriptional regulator, manganese transport regulator | 0.534150958 | 0.221877044 | 565 |
| K06872>>uncharacterized protein | −0.167581208 | 0.010791367 | 566 |
| K06875>>programmed cell death protein 5 | 0.526028488 | 0.253270111 | 567 |
| K07667>>two-component system, OmpR family, KDP operon response regulator KdpE | 0.413408332 | 0 | 568 |
| K09936>>bacterial/archaeal transporter family-2 protein | 0.423850011 | 0.025179856 | 569 |
| K10441>>ribose transport system ATP-binding protein [EC: 7.5.2.7] | 0.342489076 | 0.012589928 | 570 |
| K06942>>ribosome-binding ATPase | −0.231215585 | 0.003597122 | 571 |
| K06398>>stage IV sporulation protein A | 0.341726739 | 0.026978417 | 572 |
| K19509>>fructoselysine/glucoselysine PTS system EIID component | 0.620121082 | 0.100555919 | 573 |
| K06952>>uncharacterized protein | 0.62046703 | 0.114617397 | 574 |
| K06925>>tRNA threonylcarbamoyladenosine biosynthesis protein TsaE | 0.394777912 | −0.006540222 | 575 |
| K16899>>ATP-dependent helicase/nuclease subunit B [EC: 3.1.—.— 3.6.4.12] | 0.392701121 | 0.055755396 | 576 |
| K06861>>lipopolysaccharide export system ATP-binding protein [EC: 3.6.3.—] | −0.767674355 | −0.309843035 | 577 |
| K06978>>uncharacterized protein | −0.914111202 | −0.123610203 | 578 |
| K06206>>sugar fermentation stimulation protein A | 0.308790619 | 0.01618705 | 579 |
| K06934>>uncharacterized protein | 0.5187761 | 0.071451929 | 580 |
| K06191>>glutaredoxin-like protein NrdH | 0.036732856 | −0.029431001 | 581 |
| K05813>>sn-glycerol 3-phosphate transport system substrate-binding protein | 0.591296896 | 0.10676913 | 582 |
| K12240>>pyochelin synthetase | 0.572707376 | 0.226618705 | 583 |
| K06180>>23S rRNA pseudouridine1911/1915/1917 synthase [EC: 5.4.99.23] | 0.10831246 | 0 | 584 |
| K05833>>putative ABC transport system ATP-binding protein | 0.295984941 | 0.005395683 | 585 |
| K05822>>tetrahydrodipicolinate N-acetyltransferase [EC: 2.3.1.89] | 0.712834166 | 0.26618705 | 586 |
| K05832>>putative ABC transport system permease protein | 0.297665234 | 0.007194245 | 587 |
| K06859>>glucose-6-phosphate isomerase, archaeal [EC: 5.3.1.9] | 0.554981017 | 0.124100719 | 588 |
| K16786>>energy-coupling factor transport system ATP-binding protein [EC: 3.6.3.—] | 0.290986776 | 0.003597122 | 589 |
| K05878>>phosphoenolpyruvate---glycerone phosphotransferase subunit DhaK [EC: 2.7.1.121] | 0.672959231 | 0.139143231 | 590 |
| K05837>>rod shape determining protein RodA | −0.447327101 | 0.010791367 | 591 |
| K21132>>alpha-mannan endo-1,2-alpha-mannanase / glycoprotein endo-alpha-1,2-mannosidase | −0.506622858 | −0.30412034 | 592 |
| [EC: 3.2.1.198 3.2.1.130] | | | |
| K05879>>phosphoenolpyruvate---glycerone phosphotransferase subunit DhaL [EC: 2.7.1.121] | 0.472644036 | 0.128351864 | 593 |
| K11180>>dissimilatory sulfite reductase alpha subunit [EC: 1.8.99.5] | 0.426709786 | 0.327501635 | 594 |
| K05952>>uncharacterized protein | −0.307616645 | −0.302812296 | 595 |
| K06901>>putative MFS transporter, AGZA family, xanthine/uracil permease | 0.323425606 | 0.003597122 | 596 |
| K05919>>superoxide reductase [EC: 1.15.1.2] | 0.579173871 | 0.15353172 | 597 |
| K05985>>ribonuclease M5 [EC: 3.1.26.8] | −0.293762482 | 0.010954872 | 598 |
| K21498>>antitoxin HigA-1 | 0.406553226 | 0.046435579 | 599 |
| K05967>>uncharacterized protein | 0.730900082 | 0.219097449 | 600 |
| K06298>>germination protein M | 0.818141303 | 0.311314585 | 601 |
| K05970>>sialate O-acetylesterase [EC: 3.1.1.53] | −0.4227436 | −0.037606279 | 602 |
| K06944>>uncharacterized protein | 0.352089393 | 0.246729889 | 603 |
| K06015>>N-acyl-D-amino-acid deacylase [EC: 3.5.1.81] | −0.374747436 | −0.326847613 | 604 |
| K06001>>tryptophan synthase beta chain [EC: 4.2.1.20] | −0.532385072 | −0.048397646 | 605 |
| K06949>>ribosome biogenesis GTPase / thiamine phosphate phosphatase [EC: 3.6.1.—3.1.3.100] | −0.190398323 | 0.003597122 | 606 |
| K13940>>dihydroneopterin aldolase / | 0.632014939 | 0.158273381 | 607 |
| 2-amino-4-hydroxy-6-hydroxymethyldihydropteridine diphosphokinase [EC: 4.1.2.25 2.7.6.3] | | | |
| K06041>>arabinose-5-phosphate isomerase [EC: 5.3.1.13] | −0.900868141 | −0.110529758 | 608 |
| K21464>>penicillin-binding protein 2D [EC: 2.4.1.129 3.4.16.4] | 0.208472977 | 0.193590582 | 609 |
| K06958>>RNase adapter protein RapZ | 0.255589246 | 0.001798561 | 610 |
| K11749>>regulator of sigma E protease [EC: 3.4.24.—] | −0.23533814 | 0.007194245 | 611 |
| K06042>>precorrin-8X/cobalt-precorrin-8 methylmutase [EC: 5.4.99.61 5.4.99.60] | 0.269216573 | 0.088783519 | 612 |
| K06960>>uncharacterized protein | 0.354585013 | 0.003597122 | 613 |
| K06962>>uncharacterized protein | 0.672747349 | 0.114290386 | 614 |
| K06076>>long-chain fatty acid transport protein | −0.954011661 | −0.159581426 | 615 |
| K02773>>galactitol PTS system EIIA component [EC: 2.7.1.200] | 0.761287339 | 0.190647482 | 616 |
| k_{——}Bacteria\|p_{——}Actinobacteria\|c_{——}Coriobacteriia\|o_{——}Coriobacteriales | 0.00597906 | 0.010137345 | 617 |
| \|f_{——}Atopobiaceae\|g_{——}Atopobium | | | |
| K06113>>arabinan endo-1,5-alpha-L-arabinosidase [EC: 3.2.1.99] | −0.746167878 | −0.30412034 | 618 |
| K06133>>4′-phosphopantetheinyl transferase [EC: 2.7.8.—] | 0.443421859 | 0.058207979 | 619 |
| K06970>>23S rRNA (adenine1618-N6)-methyltransferase [EC: 2.1.1.181] | −0.632414121 | −0.201929366 | 620 |
| K06987>>uncharacterized protein | 0.375254379 | 0.022236756 | 621 |
| K06310>>spore germination protein | 0.529742639 | 0.04381949 | 622 |
| K06143>>inner membrane protein | −0.886046181 | −0.271909745 | 623 |
| K06147>>ATP-binding cassette, subfamily B, bacterial | 0.24712341 | 0 | 624 |
| K06990>>MEMO1 family protein | 0.49798334 | 0.231033355 | 625 |
| K02108>>F-type H+-transporting ATPase subunit a | 0.345280141 | 0.001798561 | 626 |
| K21575>>neopullulanase [EC: 3.2.1.135] | −0.781695426 | −0.200784827 | 627 |
| K06079>>copper homeostasis protein (lipoprotein) | −0.937646501 | −0.202583388 | 628 |
| K07306>>anaerobic dimethyl sulfoxide reductase subunit A [EC: 1.8.5.3] | 0.551406906 | 0.227109222 | 629 |
| K07588>>LAO/AO transport system kinase [EC: 2.7.—.—] | −0.943321575 | −0.218770438 | 630 |
| K07309>>Tat-targeted selenate reductase subunit YnfE [EC: 1.97.1.9] | 0.507636502 | 0.258502289 | 631 |
| K07447>>putative holliday junction resolvase [EC: 3.1.—.—] | −0.036833673 | 0.003597122 | 632 |
| K13652>>AraC family transcriptional regulator | −0.683560309 | −0.347449313 | 633 |
| K14086>>ech hydrogenase subunit A | 0.626800877 | 0.114453891 | 634 |
| K13378>>NADH-quinone oxidoreductase subunit C/D [EC: 7.1.1.2] | −0.901023038 | −0.121321125 | 635 |
| K07335>>basic membrane protein A and related proteins | 0.475769377 | 0.014388489 | 636 |
| K13479>>xanthine dehydrogenase FAD-binding subunit [EC: 1.17.1.4] | 0.577357034 | 0.184434271 | 637 |
| K07402>>xanthine dehydrogenase accessory factor | 0.31096214 | 0.014388489 | 638 |
| K06168>>tRNA-2-methylthio-N6-dimethylallyladenosine synthase [EC: 2.8.4.3] | −0.199375202 | 0.003597122 | 639 |
| K11358>>aspartate aminotransferase [EC: 2.6.1.1] | 0.514636544 | 0.006049706 | 640 |
| K11189>>PTS-HPR_{——}phosphocarrier protein | −0.025970773 | 0.007194245 | 641 |
| K02843>>heptosyltransferase II [EC: 2.4.—.—] | −0.743394416 | −0.348266841 | 642 |
| K07473>>DNA-damage-inducible protein J | 0.219372963 | 0.001798561 | 643 |
| K07478>>putative ATPase | 0.24593475 | 0 | 644 |
| K06997>>PLP dependent protein | 0.208813148 | 0.005395683 | 645 |
| K07493>>putative transposase | 0.479055354 | 0.287606279 | 646 |
| K07496>>putative transposase | 0.526253647 | 0.003597122 | 647 |
| K07322>>regulator of cell morphogenesis and NO signaling | −0.841015156 | −0.138652714 | 648 |
| K07443>>methylated-DNA-protein-cysteine methyltransferase related protein | −0.49291353 | −0.075866579 | 649 |
| K07568>>S-adenosylmethionine:RNA ribosyltransferase-isomerase [EC: 2.4.99.17] | −0.268826839 | 0.003597122 | 650 |
| k_{——}Bacteria\|p_{——}Actinobacteria\|c_{——}Coriobacteriia\|o_{——}Coriobacteriales\|f_{——}Coriobacteriaceae | 0.540524558 | 0.031720078 | 651 |
| K07580>>Zn-ribbon RNA-binding protein | 0.577779874 | 0.25147155 | 652 |
| K07219>>putative molybdopterin biosynthesis protein | 0.623334538 | 0.213865271 | 653 |
| K14652>>3,4-dihydroxy 2-butanone 4-phosphate synthase / GTP cyclohydrolase II | −0.21278996 | 0.003597122 | 654 |
| TEC:4.1.99.12 3.5.4.25] | | | |
| k_{——}Bacteria\|p_{——}Actinobacteria\|c_{——}Coriobacteriia | 0.706785055 | 0.037279267 | 655 |
| K07307>>anaerobic dimethyl sulfoxide reductase subunit B | 0.581970799 | 0.249672989 | 656 |
| k_{——}Bacteria\|p_{——}Actinobacteria\|c_{——}Actinobacteria\|o_{——}Corynebacteriales | 0.009464522 | 0.032864617 | 657 |
| K06993>>ribonuclease H-related protein | 0.648478451 | 0.21206671 | 658 |
| K20492>>lantibiotic transport system permease protein | 0.385946597 | −0.013734467 | 659 |
| K07263>>zinc protease [EC: 3.4.24.—] | −0.946079024 | −0.190647482 | 660 |
| K18707>>threonylcarbamoyladenosine tRNA methylthiotransferase MtaB [EC: 2.8.4.5] | −0.522188153 | −0.02207325 | 661 |
| K07001>>NTE family protein | −0.531865172 | −0.086657946 | 662 |
| K07332>>archaeal flagellar protein FlaI | 0 | 0.081916285 | 663 |
| K13049>>carboxypeptidase PM20D1 [EC: 3.4.17.—] | −0.607096791 | −0.379496403 | 664 |
| K07012>>CRISPR-associated endonuclease/helicase Cas3 [EC: 3.1.—.—3.6.4.—] | 0.634780721 | 0.157292348 | 665 |
| K07560>>D-aminoacyl-tRNA deacylase [EC: 3.1.1.96] | −0.132839681 | 0.003597122 | 666 |
| K07033>>uncharacterized protein | 0.684743239 | 0.33763898 | 667 |
| K07446>>tRNA (guanine10-N2)-dimethyltransferase [EC: 2.1.1.213] | 0.419336713 | 0.213211249 | 668 |
| K07464>>CRISPR-associated exonuclease Cas4 [EC: 3.1.12.1] | −0.204420365 | 0.013897973 | 669 |
| k_{——}Bacteria\|p_{——}Firmicutes\|c_{——}Clostridia\|o_{——}Clostridiales\|f_{——}Lachnospiraceae | 0.463532772 | 0.11706998 | 670 |
| \|g_{——}Fusicatenibacter | | | |
| K07040>>uncharacterized protein | 0.430281364 | 0.005395683 | 671 |
| K07584>>uncharacterized protein | 0.291454862 | 0.035971223 | 672 |
| K02396>>flagellar hook-associated protein 1 FlgK | −0.472114934 | −0.152387181 | 673 |
| K07585>>tRNA methyltransferase | 0 | 0.080117724 | 674 |
| k_{——}Bacteria\|p_{——}Actinobacteria\|c_{——}Coriobacteriia\|o_{——}Coriobacteriales\|f_{——}Coriobacteriaceae | 0.066932597 | 0.07880968 | 675 |
| \|g_{——}Collinsella\|s_{——}Collinsella_intestinalis | | | |
| K07005>>uncharacterized protein | −0.148878564 | 0.007848267 | 676 |
| K07037>>cyclic-di-AMP phosphodiesterase PgpH [EC: 3.1.4.—] | −1.123278421 | −0.2588293 | 677 |
| K07301>>cation: H+ antiporter | 0.176587601 | 0.019784173 | 678 |
| K07502>>uncharacterized protein | 0.543280028 | 0.120503597 | 679 |
| K07029>>diacylglycerol kinase (ATP) [EC: 2.7.1.107] | 0.85373378 | 0.198005232 | 680 |
| K07079>>uncharacterized protein | −0.347852147 | 0.019784173 | 681 |
| K07048>>phosphotriesterase-related protein | 0.630401088 | 0.198168738 | 682 |
| K07507>>putative Mg2+ transporter-C (MgtC) family protein | −0.438984972 | 0.025179856 | 683 |
| K07557>>archaeosine synthase alpha-subunit [EC: 2.6.1.97 2.6.1.—] | 0.495279534 | 0.21795291 | 684 |
| K07088>>uncharacterized protein | 0.173780895 | 0.003597122 | 685 |
| K07075>>uncharacterized protein | −0.544316488 | 0.018639634 | 686 |
| K07042>>probable rRNA maturation factor | 0.2091966 | 0.003597122 | 687 |
| K07095>>uncharacterized protein | 0.256560043 | 0.003597122 | 688 |
| K07574>>RNA-binding protein | 0.255203781 | −0.015533028 | 689 |
| K13043>>N-succinyl-L-ornithine transcarbamylase [EC: 2.1.3.11] | −0.894246 | −0.238554611 | 690 |
| K07058>>membrane protein | 0.341122755 | 0.010791367 | 691 |
| K16053>>miniconductance mechanosensitive channel | −0.806572121 | −0.13145847 | 692 |
| k_{——}Bacteria\|p_{——}Firmicutes\|c_{——}Clostridia\|o_{——}Clostridiales\|f_{——}Clostridiales_Family_XIII_Incertae_Sedis | 0.02404343 | 0.053793329 | 693 |
| \|g_{——}Clostridiales_Family_XIII_Incertae_Sedis_unclassified | | | |
| K07141>>molybdenum cofactor cytidylyltransferase [EC: 2.7.7.76] | 0.501477584 | 0.01324395 | 694 |
| K07082>>UPF0755 protein | 0.273903932 | −0.020928712 | 695 |
| K07085>>putative transport protein | −0.922451571 | −0.118378025 | 696 |
| K07171>>mRNA interferase MazF [EC: 3.1.—.—] | 0.37630996 | 0.003597122 | 697 |
| K16785>>energy-coupling factor transport system permease protein | 0.281838624 | 0.003597122 | 698 |
| K07112>>uncharacterized protein | 0.489359486 | 0.121811642 | 699 |
| K07177>>Lon-like protease | 0.212209601 | 0.043328973 | 700 |
| K21573>>TonB-dependent starch-binding outer membrane protein SusC | −0.654546226 | −0.277141923 | 701 |
| K07192>>flotillin | −0.500856654 | −0.014879006 | 702 |
| K07103>>uncharacterized protein | 0.425216497 | 0.222204055 | 703 |
| K02589>>nitrogen regulatory protein PII 1 | 0.616238718 | 0.249836494 | 704 |
| K07106>>N-acetylmuramic acid 6-phosphate etherase [EC: 4.2.1.126] | −0.553098493 | −0.02207325 | 705 |
| K02825>>pyrimidine operon attenuation protein / uracil phosphoribosyltransferase [EC: 2.4.2.9] | 0.388012852 | 0.040222368 | 706 |
| K00999>>CDP-diacylglycerol--inositol 3-phosphatidyltransferase [EC: 2.7.8.11] | 0 | 0.020928712 | 707 |
| K21688>>resuscitation-promoting factor RpfB | 0.099708732 | 0.182962721 | 708 |
| K07164>>uncharacterized protein | −0.896413686 | −0.168574232 | 709 |
| K01006>>pyruvate, orthophosphate dikinase [EC: 2.7.9.1] | 0.189749547 | 0.008992806 | 710 |
| K01008>>selenide, water dikinase [EC: 2.7.9.3] | 0.340915929 | 0.008992806 | 711 |
| K07277>>outer membrane protein insertion porin family | −0.741338821 | −0.157128842 | 712 |
| K21571>>starch-binding outer membrane protein SusE/F | −0.829343693 | −0.259973839 | 713 |
| K01012>>biotin synthase [EC: 2.8.1.6] | 0.01262001 | 0.003597122 | 714 |
| K16787>>energy-coupling factor transport system ATP-binding protein [EC: 3.6.3.—] | 0.284908638 | 0.001798561 | 715 |
| K01023>>arylsulfate sulfotransferase [EC: 2.8.2.22] | 0.257052089 | 0.084368869 | 716 |
| K01495>>GTP cyclohydrolase IA [EC: 3.5.4.16] | −0.224688538 | 0 | 717 |
| K07148>>uncharacterized protein | −1.014498142 | −0.157782865 | 718 |
| K01029>>3-oxoacid CoA-transferase subunit B [EC: 2.8.3.5] | 0 | −0.031229562 | 719 |
| K01003>>oxaloacetate decarboxylase [EC: 4.1.1.112] | 0 | −0.005395683 | 720 |
| K01004>>phosphatidylcholine synthase [EC: 2.7.8.24] | 0 | 0.01913015 | 721 |
| K07221>>phosphate-selective porin OprO and OprP | −1.073271552 | −0.252125572 | 722 |
| K01039>>glutaconate CoA-transferase, subunit A [EC: 2.8.3.12] | −0.1841727 | −0.187704382 | 723 |
| K01028>>3-oxoacid CoA-transferase subunit A [EC: 2.8.3.5] | 0 | −0.01030085 | 724 |
| K01040>>glutaconate CoA-transferase, subunit B [EC: 2.8.3.12] | −0.420691139 | −0.267495095 | 725 |
| K01046>>triacylglycerol lipase [EC: 3.1.1.3] | −0.10427047 | −0.001308044 | 726 |
| K01011>>thiosulfate/3-mercaptopyruvate sulfurtransferase [EC: 2.8.1.1 2.8.1.2] | 0.026769976 | 0.023054284 | 727 |
| K01048>>lysophospholipase [EC: 3.1.1.5] | 0.323516612 | 0.096958797 | 728 |
| K00980>>glycerol-3-phosphate cytidylyltransferase [EC: 2.7.7.39] | 0.642302445 | 0.283682145 | 729 |
| K02796>>mannose PTS system EIID component | 0.566482104 | 0.02763244 | 730 |
| K01000>>phospho-N-acetylmuramoyl-pentapeptide-transferase [EC: 2.7.8.13] | −0.065702881 | 0 | 731 |
| K00997>>holo-[acyl-carrier protein] synthase [EC: 2.7.8.7] | 0.392069221 | −0.004741661 | 732 |
| k_{——}Bacteria\|p_{——}Actinobacteria\|c_{——}Coriobacteriia\|o_{——}Coriobacteriales | 0.547910648 | 0.028122956 | 733 |
| K01001>>UDP-N-acetylglucosamine--dolichyl-phosphate N-acetylglucosaminephosphotransferase | 0 | 0.053793329 | 734 |
| [EC: 2.7.8.15] | | | |
| K01034>>acetate CoA/acetoacetate CoA-transferase alpha subunit [EC: 2.8.3.8 2.8.3.9] | 0.184762287 | 0.043328973 | 735 |
| k_{——}Bacteria\|p_{——}Firmicutes\|c_{——}Clostridia\|o_{——}Clostridiales\|f_{——}Lachnospiraceae | 0.783788714 | 0.337148463 | 736 |
| \|g_{——}Blautia\|s_{——}Ruminococcus_torques | | | |
| K00819>>ornithine--oxo-acid transaminase [EC: 2.6.1.13] | −1.000828287 | −0.232995422 | 737 |
| K18012>>L-erythro-3,5-diaminohexanoate dehydrogenase [EC: 1.4.1.11] | −0.510470487 | −0.283845651 | 738 |
| K01007>>pyruvate, water dikinase [EC: 2.7.9.2] | −0.224173451 | −0.116088947 | 739 |
| K00836>>diaminobutyrate-2-oxoglutarate transaminase [EC: 2.6.1.76] | 0.577109564 | 0.364617397 | 740 |
| K00850>>6-phosphofructokinase 1 [EC: 2.7.1.11] | −0.386105197 | 0.005395683 | 741 |
| K00847>>fructokinase [EC: 2.7.1.4] | 0.287106894 | 0.005395683 | 742 |
| k_{——}Bacteria\|p_{——}Actinobacteria\|c_{——}Actinobacteria\|o_{——}Corynebacteriales\|f_{——}Corynebacteriaceae | 0.006963493 | 0.034663179 | 743 |
| K00975>>glucose-1-phosphate adenylyltransferase [EC: 2.7.7.27] | 0.269771235 | 0 | 744 |
| K00854>>xylulokinase [EC: 2.7.1.17] | −0.175789012 | 0.005395683 | 745 |
| K01026>>propionate CoA-transferase [EC: 2.8.3.1] | 0.491931583 | 0.009156311 | 746 |
| K00857>>thymidine kinase [EC: 2.7.1.21] | −0.562083509 | −0.011935906 | 747 |
| K00979>>3-deoxy-manno-octulosonate cytidylyltransferase (CMP-KDO synthetase) [EC: 2.7.7.38] | −0.718154721 | −0.063930674 | 748 |
| K13051>>L-asparaginase / beta-aspartyl-peptidase [EC: 3.5.1.1 3.4.19.5] | −0.942624069 | −0.127207325 | 749 |
| K00874>>2-dehydro-3-deoxygluconokinase [EC: 2.7.1.45] | −0.31538401 | 0.008992806 | 750 |
| K01032>>3-oxoadipate CoA-transferase, beta subunit [EC: 2.8.3.6] | 0 | −0.001798561 | 751 |
| K00926>>carbamate kinase [EC: 2.7.2.2] | 0.365242964 | 0.017985612 | 752 |
| K00876>>uridine kinase [EC: 2.7.1.48] | −0.32235642 | 0.005395683 | 753 |
| K17830>>digeranylgeranylglycerophospholipid reductase [EC: 1.3.1.101 1.3.7.11] | 0.476296543 | 0.226945716 | 754 |
| K01035>>acetate CoA/acetoacetate CoA-transferase beta subunit [EC: 2.8.3.8 2.8.3.9] | −0.02462675 | 0.008011772 | 755 |
| K00845>>glucokinase [EC: 2.7.1.2] | 0.168868108 | 0.003597122 | 756 |
| K01042>>L-seryl-tRNA(Ser) seleniumtransferase [EC: 2.9.1.1] | −0.073424934 | −0.0029431 | 757 |
| K00945>>CMP/dCMP kinase [EC: 2.7.4.25] | −0.135294003 | 0 | 758 |
| K00848>>rhamnulokinase [EC: 2.7.1.5] | −0.487830186 | −0.084859385 | 759 |
| K17104>>phosphoglycerol geranylgeranyltransferase [EC: 2.5.1.41] | 0.461918105 | 0.209614127 | 760 |
| K00860>>adenylylsulfate kinase [EC: 2.7.1.25] | −0.799055129 | −0.081262263 | 761 |
| K01051>>pectinesterase [EC: 3.1.1.11] | −0.889785919 | −0.170372793 | 762 |
| K01053>>gluconolactonase [EC: 3.1.1.17] | 0 | −0.003597122 | 763 |
| K00957>>sulfate adenylyltransferase subunit 2 [EC: 2.7.7.4] | −0.249050097 | 0.003597122 | 764 |
| K19508>>fructoselysine/glucoselysine PTS system EIIC component | 0.716987421 | 0.175768476 | 765 |
| K00912>>tetraacyldisaccharide 4′-kinase [EC: 2.7.1.130] | −0.900619948 | −0.170372793 | 766 |
| K00973>>glucose-1-phosphate thymidylyltransferase [EC: 2.7.7.24] | 0.235083553 | 0.003597122 | 767 |
| K01058>>phospholipase A1/A2 [EC: 3.1.1.32 3.1.1.4] | −0.258755704 | −0.190647482 | 768 |
| K00950>>2-amino-4-hydroxy-6-hydroxymethyldihydropteridine diphosphokinase [EC: 2.7.6.3] | −0.53473081 | −0.104480052 | 769 |
| K01120>>3′,5′-cyclic-nucleotide phosphodiesterase [EC: 3.1.4.17] | 0 | −0.007194245 | 770 |
| K00954>>pantetheine-phosphate adenylyltransferase [EC: 2.7.7.3] | −0.210208649 | 0.001798561 | 771 |
| K00864>>glycerol kinase [EC: 2.7.1.30] | 0.184076324 | 0.003597122 | 772 |
| K00971>>mannose-1-phosphate guanylyltransferase [EC: 2.7.7.13] | −0.484317619 | −0.027468934 | 773 |
| K00929>>butyrate kinase [EC: 2.7.2.7] | −0.985175042 | −0.185905821 | 774 |
| K00930>>acetylglutamate kinase [EC: 2.7.2.8] | −0.113110164 | 0 | 775 |
| K01163>>uncharacterized protein | −0.200049941 | 0.021582734 | 776 |
| K01173>>endonuclease G, mitochondrial | −0.683440312 | −0.265042511 | 777 |
| K00946>>thiamine-monophosphate kinase [EC: 2.7.4.16] | −0.859168134 | −0.124918247 | 778 |
| K00948>>ribose-phosphate pyrophosphokinase [EC: 2.7.6.1] | 0.179467439 | 0 | 779 |
| K01056>>peptidyl-tRNA hydrolase, PTH1 family [EC: 3.1.1.29] | −0.067507354 | 0 | 780 |
| K01179>>endoglucanase [EC: 3.2.1.4] | 0.063500099 | 0.088456508 | 781 |
| K01181>>endo-1,4-beta-xylanase [EC: 3.2.1.8] | −0.061658742 | −0.102027469 | 782 |
| K01126>>glycerophosphoryl diester phosphodiesterase [EC: 3.1.4.46] | −0.424820191 | 0.014388489 | 783 |
| K00965>>UDPglucose--hexose-1-phosphate uridylyltransferase [EC: 2.7.7.12] | 0.427154897 | 0.012589928 | 784 |
| K00969>>nicotinate-nucleotide adenylyltransferase [EC: 2.7.7.18] | −0.166088099 | 0.010791367 | 785 |
| K23004>>L-galactono-1,5-lactonase [EC: 3.1.1.—] | −0.914084943 | −0.359058208 | 786 |
| K01130>>arylsulfatase [EC: 3.1.6.1] | −0.20914784 | −0.10824068 | 787 |
| K01185>>lysozyme [EC: 3.2.1.17] | −0.419489388 | −0.118868542 | 788 |
| K01054>>acylglycerol lipase [EC: 3.1.1.23] | #N/A | #N/A | 789 |
| K01138>>uncharacterized sulfatase [EC: 3.1.6.—] | −0.02148227 | −0.045618051 | 790 |
| K01186>>sialidase-1 [EC: 3.2.1.18] | −0.746797031 | −0.123119686 | 791 |
| K01187>>alpha-glucosidase [EC: 3.2.1.20] | −0.317618 | −0.023871812 | 792 |
| K01139>>GTP diphosphokinase / guanosine-3′,5′-bis(diphosphate) 3′-diphosphatase | −0.103584067 | −0.13145847 | 793 |
| [EC: 2.7.6.5 3.1.7.2] | | | |
| K01188>>beta-glucosidase [EC: 3.2.1.21] | 0 | −0.028776978 | 794 |
| K01190>>beta-galactosidase [EC: 3.2.1.23] | −0.229541898 | 0.003597122 | 795 |
| K01191>>alpha-mannosidase [EC: 3.2.1.24] | −0.176228495 | −0.073413996 | 796 |
| K01057>>6-phosphogluconolactonase [EC: 3.1.1.31] | −0.377611345 | −0.103335513 | 797 |
| K13573>>proteasome accessory factor C | 0.722677469 | 0.172171354 | 798 |
| K01192>>beta-mannosidase [EC: 3.2.1.25] | 0.068446135 | 0.01324395 | 799 |
| K01119>>2′,3′-cyclic-nucleotide 2′-phosphodiesterase / 3′-nucleotidase | −0.25686947 | −0.011281884 | 800 |
| EC:3.1.4.16 3.1.3.6] | | | |

TABLE 5
Feature List for Colorectal Cancer (CRC). The Table presents the fold changes in relative abundance, prevalence shifts and weight
or Importance of the features, changes, and shifts. The prevalence shift value between the two classes has a positive value
when there is a higher prevalence in CRC and a negative value when there is a higher prevalence in the control group.

| | Fold change | | Weight |
|---|---|---|---|
| | in relative | Prevalence | or im- |
| Taxonomic or Gene Feature | abundance | Shift | portance |
| K02919 >> large subunit ribosomal protein L36 | 0.021461679 | 0.001801802 | 1 |
| k_Bacteria\|p_Firmicutes\|c_Negativicutes\|o_Veillonellales\|f_Veillonellaceae | 0.044636943 | 0.134888232 | 2 |
| \|g_Dialister\|s_Dialister_{—}pneumosintes | | | |
| k_Bacteria\|p_Bacteroidetes\|c_Bacteroidia\|o_Bacteroidales\|f_Prevotellaceae | 0 | 0.041755948 | 3 |
| \|g_Prevotella\|s_Prevotella sp CAG 520 | | | |
| k_Bacteria\|p_Fusobacteria\|c_Fusobacteriia\|o_Fusobacteriales\|f_Fusobacteriaceae | 0.025759797 | 0.097708802 | 4 |
| \|g_Fusobacterium\|s_Fusobacterium_{—}nucleatum | | | |
| k_Bacteria\|p_Firmicutes\|c_Bacilli\|o_Bacillales\|f_Bacillales_unclassified | 0.041214552 | 0.118745499 | 5 |
| \|g_Gemella\|s_Gemella_{—}morbillorum | | | |
| k_Bacteria\|p_Firmicutes\|c_Bacilli\|o_Lactobacillies\|f_Streptococcaceae | 0 | 0.021815617 | 6 |
| \|g Streptococcus\|s Streptococcus pasteurianus | | | |
| K07132 >> ATP-dependent target DNA activator [EC: 3.6.1.3] | 0.057106172 | 0.0605764 | 7 |
| K06385 >> stage II sporulation protein P | −0.097928958 | −0.006207839 | 8 |
| k_Bacteria\|p_Firmicutes\|c_Tissierellia\|o_Tissierellales\|f_Peptoniphilaceae\|g_Parvimonas | 0.104812366 | 0.184947754 | 9 |
| k_Bacteria\|p_Actinobacterial\|c_Actinobacteria\|o_Bifidobacteriales\|f_Bifidobacteriaceae | −0.007735145 | −0.025957115 | 10 |
| \|g_Bifidobacterium\|s_Bifidobacterium_{—}catenulatum | | | |
| k_Bacteria\|p_Firmicutes\|c_Bacilli\|o_Lactobacillies\|f_Streptococcaceae\|g_Streptococcus | −0.116143117 | −0.123898123 | 11 |
| s Streptococcus salivarius | | | |
| k_Bacteria\|p_Firmicutes\|c_Clostridia\|o_Clostridiales\|f_Peptostreptococcaceae | 0.060957765 | 0.143556281 | 12 |
| \|g_Peptostreptococcus | | | |
| k_Bacteria\|p_Firmicutes\|c_Clostridia\|o_Clostridiales\|f_Peptostreptococcaceae | 0 | 0.050570962 | 13 |
| \|g_Peptostreptococcus\|s_Peptostreptococcus_{—}anaerobius | | | |
| k_Bacteria\|p_Firmicutes\|c_Clostridia\|o_Clostridiales\|f_Clostridiaceae | 0.083208728 | 0.05764884 | 14 |
| \|g Clostridium\|s Clostridium sp CAG 58 | | | |
| K15587 >> nickel transport system ATP-binding protein [EC: 7.2.2.11] | 0.063200048 | 0.0216275 | 15 |
| k_Bacteria\|p_Firmicutes\|c_{——}Clostridia\|o_Clostridiales\|f_Peptostreptococcaceae | 0.040174043 | 0.117455139 | 16 |
| \|g Peptostreptococcus\|s Peptostreptococcus_{—}stomatis | | | |
| K01628 >> L-fuculose-phosphate aldolase [EC: 4.1.2.17] | 0.037521974 | 0.002216246 | 17 |
| K07736 >> CarD family transcriptional regulator | −0.00557051 | −0.009543965 | 18 |
| K09702 >> uncharacterized protein | −0.060705715 | −0.003627118 | 19 |
| K15533 >> 1,3-beta-galactosyl-N-acetylhexosamine phosphorylase [EC: 2.4.1.211] | −0.284327589 | −0.133033523 | 20 |
| k_Bacteria\|p_Actinobacteria\|c_Coriobacteriia\|o_Coriobacteriales\|f_Atopobiaceae | 0.004313797 | 0.007133724 | 21 |
| \|g_Atopobium | | | |
| K00375 >> GntR family transcriptional regulator/MocR family aminotransferase | −0.239571137 | −0.050520994 | 22 |
| k_Bacteria\|p_Firmicutes\|c_Negativicutes\|o_Veillonellales\|f_Veillonellaceae | 0.003188606 | 0.008350602 | 23 |
| \|g_Veillonella\|s_Veillonella_sp_T11011_6 | | | |
| K07012 >> CRISPR-associated endonuclease/helicase Cas3 [EC: 3.1.—.— 3.6.4.—] | −0.098047682 | −0.050224123 | 24 |
| K15871 >> bile acid CoA-transferase [EC: 2.8.3.25] | 0.049129908 | 0.058871596 | 25 |
| K20459 >> lantibiotic transport system ATP-binding protein | 0.041249221 | 0.045918022 | 26 |
| k_Bacteria\|p_Proteobacteria\|c_Proteobacteria_unclassified | −0.06864258 | −0.053766001 | 27 |
| K06881 >> bifunctional oligoribonuclease and PAP phosphatase NrnA [EC: 3.1.3.7 3.1.13.3] | 0.069085493 | −0.003018679 | 28 |
| k_Bacteria\|p_Firmicutes\|c_Negativicutes\|o_Veillonellales\|f_Veillonellaceae | 0.013328974 | 0.028461414 | 29 |
| \|g_Allisonella | | | |
| k_Bacteria\|p_Bacteroidetes\|c_Bacteroidia\|o_Bacteroidales\|f_Porphyromonadaceae | 0.027047006 | 0.102432285 | 30 |
| \|g_Porphyromonas\|s_Porphyromonas_{—}asaccharolytica | | | |
| k_Bacteria\|p_Bacteroidetes\|c_Bacteroidia\|o_Bacteroidales\|f_Odoribacteraceae | 0.120117214 | 0.109924606 | 31 |
| \|g Butyricimonas\|s Butyricimonas virosa | | | |
| K07214 >> iron(III)-enterobactin esterase [EC: 3.1.1.108] | 0.018215577 | −0.003424305 | 32 |
| K07484 >> transposase | −0.057598806 | 0.018164984 | 33 |
| K11529 >> glycerate 2-kinase [EC: 2.7.1.165] | −0.257390576 | −0.066978234 | 34 |
| k_Bacteria\|p_Bacteroidetes\|c_Bacteroidia\|o_Bacteroidales\|f_Odoribacteraceae | 0.118889518 | 0.089543377 | 35 |
| \|g_Butyricimonas | | | |
| K19158 >> toxin YoeB [EC: 3.1.—.—] | 0.195588249 | 0.048845583 | 36 |
| K05919 >> superoxide reductase [EC: 1.15.1.2] | −0.182769891 | −0.084508326 | 37 |
| k_Bacteria\|p_Firmicutes\|c_Negativicutes\|o_Veillonellales\|f_Veillonellaceae | 0 | −0.000511441 | 38 |
| \|g_Veillonella\|s_Veillonella_{—}tobetsuensis | | | |
| K07160 >> 5-oxoprolinase (ATP-hydrolysing) subunit A [EC: 3.5.2.9] | −0.032589141 | −0.035401143 | 39 |
| K01905 >> acetate---CoA ligase (ADP-forming) subunit alpha [EC: 6.2.1.13] | 0.064796993 | 0.032979146 | 40 |
| K01156 >> type III restriction enzyme [EC: 3.1.21.5] | 0.145611668 | 0.003800538 | 41 |
| k_Bacteria\|p_Firmicutes\|c_Clostridia\|o_Clostridiales\|f_Eubacteriaceae\|g_Eubacterium | −0.126519819 | −0.096186235 | 42 |
| \|s Eubacterium ventriosum | | | |
| K18475 >> lysine-N-methylase [EC: 2.1.1.—] | −0.22035152 | −0.050958952 | 43 |
| K10797 >> 2-enoate reductase [EC: 1.3.1.31] | −0.079430456 | −0.082991638 | 44 |
| k_Bacteria\|p_Bacteroidetes\|c_Bacteroidial\|o_Bacteroidales\|f_Bacteroidaceae | 0.01069683 | 0.039857149 | 45 |
| \|g Bacteroides\|s Bacteroides nordii | | | |
| K17398 >> DNA (cytosine-5)-methyltransferase 3A [EC: 2.1.1.37] | −0.131898276 | −0.108842938 | 46 |
| K13573 >> proteasome accessory factor C | 0.203040372 | 0.108457887 | 47 |
| K03713 >> MerR family transcriptional regulator, glutamine synthetase repressor | 0.095244769 | 0.113119645 | 48 |
| k_Bacteria\|p_Firmicutes\|c_Tissierellia\|o_Tissierellales\|f_Peptoniphilaceae | 0.090358313 | 0.170265861 | 49 |
| \|g_Parvimonas\|s_Parvimonas_{—}micra | | | |
| K02472 >> UDP-N-acetyl-D-mannosaminuronic acid dehydrogenase [EC: 1.1.1.336] | 0.180110644 | 0.005652308 | 50 |
| K20276 >> large repetitive protein | 0.087997153 | 0.082562497 | 51 |
| K01955 >> carbamoyl-phosphate synthase large subunit [EC: 6.3.5.5] | −0.079530275 | −0.001631321 | 52 |
| K00216 >> 2,3-dihydro-2,3-dihydroxybenzoate dehydrogenase [EC: 1.3.1.28] | 0.040282448 | 0.02059286 | 53 |
| k_Bacteria\|p_Bacteroidetes\|c_Bacteroidial\|o_Bacteroidales\|f_Prevotellaceae | 0.077566127 | 0.07311553 | 54 |
| \|g_Prevotella\|s_Prevotella_{—}stercorea | | | |
| K20626 >> lactoyl-CoA dehydratase subunit alpha [EC: 4.2.1.54] | 0.130762619 | 0.026791882 | 55 |
| K19509 >> fructoselysine/glucoselysine PTS system EIID component | 0.149453456 | 0.052281645 | 56 |
| K07491 >> putative transposase | −0.059982747 | 0.005916847 | 57 |
| k_Bacteria\|p_Firmicutes\|c_Tissierellia\|o_Tissierellales | 0.115132747 | 0.19293388 | 58 |
| K19954 >> alcohol dehydrogenase [EC: 1.1.1.—] | 0.017965938 | 0.014358567 | 59 |
| K08602 >> oligoendopeptidase F [EC: 3.4.24.—] | −0.233317976 | −0.07630469 | 60 |
| k_Bacterial\|p_Firmicutes\|c_Tissierellia | 0.115119028 | 0.194565201 | 61 |
| K06012 >> spore protease [EC: 3.4.24.78] | −0.17139055 | −0.041658951 | 62 |
| K10439 >> ribose transport system substrate-binding protein | 0.002830102 | −0.002921682 | 63 |
| K06438 >> similar to_stage IV sporulation protein | −0.320176919 | −0.130232353 | 64 |
| K17234 >> arabinosaccharide transport system substrate-binding protein | −0.309056995 | −0.138168511 | 65 |
| K18011 >> beta-lysine 5,6-aminomutase beta subunit [EC: 5.4.3.3] | 0.321307174 | 0.137965698 | 66 |
| K00702 >> cellobiose phosphorylase [EC: 2.4.1.20] | −0.362166658 | −0.085337213 | 67 |
| K01205 >> alpha-N-acetylglucosaminidase [EC: 3.2.1.50] | −0.006276101 | −0.010393428 | 68 |
| K01623 >> fructose-bisphosphate aldolase, class I [EC: 4.1.2.13] | 0.18947731 | 0.194321238 | 69 |
| K00005 >> glycerol dehydrogenase [EC: 1.1.1.6] | 0.031523378 | −0.001290361 | 70 |
| K07706 >> two-component system, LytTR family, sensor histidine kinase AgrC [EC: 2.7.13.3] | −0.112918325 | −0.082821157 | 71 |
| K09895 >> uncharacterized protein | 0.107800153 | 0.009970166 | 72 |
| K02527 >> 3-deoxy-D-manno-octulosonic-acid transferase | 0.191852981 | 0.032117925 | 73 |
| EC: 2.4.99.12 2.4.99.13 2.4.99.14 2.4.99.15 | | | |
| K06042 >> precorrin-8X/cobalt-precorrin-8 methylmutase [EC: 5.4.99.61 5.4.99.60] | −0.081405865 | −0.035448172 | 74 |
| K07341 >> death on curing protein | −0.243577929 | −0.084384874 | 75 |
| K06393 >> stage III sporulation protein AD | −0.153047657 | −0.026465617 | 76 |
| K02909 >> large subunit ribosomal protein L31 | −0.013329779 | 0 | 77 |
| k_Bacterial\|p_Firmicutes\|c_Clostridial\|o_Clostridiales\|f_Christensenellaceae | 0.004311344 | 0.020792734 | 78 |
| K10914 >> CRP/FNR family transcriptional regulator, cyclic_AMP receptor protein | 0.270313871 | 0.090971885 | 79 |
| K19081 >> two-component system, OmpR family, sensor histidine kinase BraS/BceS [EC: 2.7.13.3] | 0.08060587 | 0.140999074 | 80 |
| K21578 >> betaine reductase complex component B subunit alpha [EC: 1.21.4.4] | 0 | 0.054709522 | 81 |
| K00558 >> DNA (cytosine-5)-methyltransferase 1 [EC: 2.1.1.37] | −0.009993856 | −0.003092162 | 82 |
| K01191 >> alpha-mannosidase [EC: 3.2.1.24] | −0.203680554 | −0.041171024 | 83 |
| K02931 >> large subunit ribosomal protein L5 | 0.017551826 | 0 | 84 |
| k_Bacteria\|p_Bacteroidetes\|c_Bacteroidia\|o_Bacteroidales\|f_Prevotellaceae | 0.42113697 | 0.191634702 | 85 |
| \|g Prevotella | | | |
| K02456 >> general secretion pathway protein G | 0.266435956 | 0.115083109 | 86 |
| K02669 >> twitching motility protein PilT | −0.035507736 | 0.00504093 | 87 |
| K21417 >> acetoin: 2,6-dichlorophenolindophenol oxidoreductase subunit beta [EC: 1.1.1.—] | 0.317618796 | 0.093626089 | 88 |
| K06024 >> segregation and condensation protein B | −0.106979244 | −0.015290331 | 89 |
| K05815 >> sn-glycerol 3-phosphate transport system permease protein | −0.01109367 | −0.046282498 | 90 |
| k_Bacteria\|p_Firmicutes\|c_Negativicutes\|o_Veillonellales\|f_Veillonellaceae | 0.059827228 | 0.069946945 | 91 |
| \|g Veillonella\|s Veillonella parvula | | | |
| K03502 >> DNA polymerase V | −0.064432544 | 0.000681922 | 92 |
| K05520 >> protease I [EC: 3.5.1.124] | 0.225392438 | 0.057125641 | 93 |
| K01595 >> phosphoenolpyruvate carboxylase [EC: 4.1.1.31] | −0.119099698 | −0.066075864 | 94 |
| K10671 >> glycine reductase complex component B subunit alpha and beta [EC: 1.21.4.2] | 0.206430201 | 0.135102803 | 95 |
| k_Bacteria\|p_Firmicutes\|c_Erysipelotrichia\|o_Erysipelotrichales\|f_Erysipelotrichaceae | 0.022454762 | 0.096247961 | 96 |
| \|g_Solobacterium | | | |
| K01246 >> DNA-3-methyladenine glycosylase I [EC: 3.2.2.20] | −0.001016361 | 0.001146334 | 97 |
| K19165 >> antitoxin Phd | −0.259395986 | −0.093734844 | 98 |
| K20885 >> beta-1,2-mannobiose phosphorylase/1,2-beta-oligomannan phosphorylase | 0.04309563 | 0.012592037 | 99 |
| [EC: 2.4.1.339 2.4.1.340] | | | |
| K07002 >> uncharacterized protein | 0 | 0.052566759 | 100 |
| K04488 >> nitrogen fixation protein NifU and related proteins | −0.0849492 | −0.001460841 | 101 |
| K09749 >> uncharacterized protein | −0.205015172 | −0.030163279 | 102 |
| K05350 >> beta-glucosidase [EC: 3.2.1.21] | −0.277386637 | −0.098070338 | 103 |
| K03708 >> transcriptional regulator of_stress_and heat shock_response | 0.272028619 | 0.140331849 | 104 |
| K16650 >> galactofuranosylgalactofuranosylrhamnosyl-N-acetylglucosaminyl-diphospho- | 0 | −0.031386035 | 105 |
| decaprenol beta-1,5/1,6-galactofuranosyltransferase [EC: 2.4.1.288] | | | |
| K02103 >> GntR family transcriptional regulator, arabinose operon transcriptional repressor | −0.238400901 | −0.035692136 | 106 |
| k_Bacteria\|p_Actinobacteria\|c_Coriobacteriia\|o_Coriobacteriales\|f_Coriobacteriaceae | 0.166969334 | 0.098008612 | 107 |
| \|g Collinsella\|s Collinsella aerofaciens | | | |
| K03306 >> inorganic phosphate transporter, PiT family | 0.124108846 | 0.034190144 | 108 |
| K09949 >> uncharacterized protein | 0.322532374 | 0.199503255 | 109 |
| K01647 >> citrate synthase [EC: 2.3.3.1] | −0.052949622 | 0.001801802 | 110 |
| K01991 >> polysaccharide biosynthesis/export protein | 0.255736668 | 0.035039607 | 111 |
| K00172 >> pyruvate ferredoxin oxidoreductase gamma subunit [EC: 1.2.7.1] | −0.268484571 | −0.101747424 | 112 |
| K02809 >> PTS system, sucrose-specific_IIB component [EC: 2.7.1.69] | 0.007412887 | 0.000658407 | 113 |
| k Bacteria\|p Firmicutes\|c Clostridia\|o Clostridiales\|f Eubacteriaceae | −0.03800836 | −0.075466984 | 114 |
| \|g_Eubacterium\|s_Eubacterium_{—}ramulus | | | |
| K01308 >> g-D-glutamyl-meso-diaminopimelate peptidase [EC: 3.4.19.11] | −0.230696364 | −0.12567935 | 115 |
| K09770 >> uncharacterized protein | −0.143098471 | −0.10254104 | 116 |
| K07814 >> putative two-component system response regulator | −0.248070504 | −0.08302397 | 117 |
| K01644 >> citrate lyase subunit beta/citryl-CoA lyase [EC: 4.1.3.34] | 0.255289105 | 0.047558162 | 118 |
| K12452 >> CDP-4-dehydro-6-deoxyglucose reductase, E1 [EC: 1.17.1.1] | −0.150055599 | −0.086092618 | 119 |
| K10189 >> lactose/L-arabinose transport system permease protein | −0.264445856 | −0.136684156 | 120 |
| K03790 >> [ribosomal protein S5]-alanine N-acetyltransferase [EC: 2.3.1.267] | −0.164482658 | −0.0055994 | 121 |
| K21472 >> peptidoglycan LD-endopeptidase LytH [EC: 3.4.—.—] | −0.300558301 | −0.180626956 | 122 |
| K12506 >> 2-C-methyl-D-erythritol 4-phosphate cytidylyltransferase/ | 0.099443365 | 0.036388754 | 123 |
| 2-C-methyl-D-erythritol 2,4-cyclodiphosphate synthase [EC: 2.7.7.60 4.6.1.12] | | | |
| K13890 >> glutathione transport system permease protein | 0.142325646 | 0.05058272 | 124 |
| K19171 >> DNA sulfur modification protein DndD | −0.015963752 | −0.03524536 | 125 |
| K21449 >> trimeric autotransporter adhesin | 0.234793861 | 0.074003204 | 126 |
| K06016 >> beta-ureidopropionase/N-carbamoyl-L-amino-acid hydrolase [EC: 3.5.1.6 3.5.1.87] | −0.168498704 | −0.063060124 | 127 |
| K07043 >> uncharacterized protein | −0.10938411 | −0.0009494 | 128 |
| K00027 >> malate dehydrogenase (oxaloacetate-decarboxylating) [EC: 1.1.1.38] | 0.144269511 | 0.017727026 | 129 |
| K01560 >> 2-haloacid dehalogenase [EC: 3.8.1.2] | −0.302917814 | −0.074820334 | 130 |
| K10188 >> lactose/L-arabinose transport system substrate-binding protein | −0.262742194 | −0.132004762 | 131 |
| K18581 >> unsaturated chondroitin disaccharide hydrolase [EC: 3.2.1.180] | −0.306933154 | −0.157083021 | 132 |
| k_Bacteria\|p_Firmicutes\|c_Clostridia\|o_Clostridiales\|f_Oscillospiraceae | 0.097344006 | 0.017882809 | 133 |
| K00821 >> acetylornithine/N-succinyldiaminopimelate aminotransferase [EC: 2.6.1.11 2.6.1.17] | −0.057489708 | 0 | 134 |
| K17236 >> arabinosaccharide transport system permease protein | −0.333003034 | −0.159302206 | 135 |
| K09125 >> uncharacterized protein | 0.321628259 | 0.113201946 | 136 |
| K09790 >> uncharacterized protein | 0.091656814 | −0.006110842 | 137 |
| K08963 >> methylthioribose-1-phosphate isomerase [EC: 5.3.1.23] | 0.294777194 | 0.071587085 | 138 |
| K12524 >> bifunctional aspartokinase/homoserine dehydrogenase 1 [EC: 2.7.2.4 1.1.1.3] | 0.065126263 | 0.025545611 | 139 |
| K00652 >> 8-amino-7-oxononanoate synthase [EC: 2.3.1.47] | 0.131735299 | 0.017994503 | 140 |
| k_Bacteria\|p_Firmicutes\|c_Clostridial\|o_Clostridiales\|f_Lachnospiraceae | 0.026445098 | 0.020416501 | 141 |
| \|g_Coprococcus\|s_Coprococcus_{—}catus | | | |
| K15584 >> nickel transport system substrate-binding protein | 0.056165374 | 0.015757683 | 142 |
| K13652 >> AraC family transcriptional regulator | 0.227370836 | 0.035700954 | 143 |
| k_Bacteria\|p_Firmicutes\|c_Clostridia\|o_Clostridiales\|f_Lachnospiraceae | 0.124777597 | 0.152147906 | 144 |
| \|g_Lachnoclostridium\|s_Clostridium_{—}symbiosum | | | |
| K09692 >> teichoic acid transport system permease protein | −0.28911054 | −0.112117338 | 145 |
| K00901 >> diacylglycerol kinase (ATP) [EC: 2.7.1.107] | 0.236541035 | 0.021016122 | 146 |
| K01194 >> alpha,alpha-trehalase [EC: 3.2.1.28] | 0.119952087 | 0.055670679 | 147 |
| K02040 >> phosphate transport system substrate-binding protein | −0.072785066 | −0.003262643 | 148 |
| K19161 >> antitoxin YafN | 0.166193203 | 0.08603971 | 149 |
| K07726 >> putative transcriptional regulator | −0.007956527 | −0.004382523 | 150 |
| K03768 >> peptidyl-prolyl cis-trans_isomerase B (cyclophilin B) [EC: 5.2.1.8] | −0.064659662 | −0.001631321 | 151 |
| K05343 >> maltose alpha-D-glucosyltransferase/alpha-amylase [EC: 5.4.99.16 3.2.1.1] | −0.219363997 | −0.075202446 | 152 |
| K07273 >> lysozyme | 0.22033625 | 0.038108255 | 153 |
| K00239 >> succinate dehydrogenase/fumarate reductase, flavoprotein subunit | 0.17407584 | 0.011907176 | 154 |
| [EC: 1.3.5.1 1.3.5.4] | | | |
| K21011 >> polysaccharide biosynthesis_protein PelF | −0.158565951 | −0.045209647 | 155 |
| k_Bacteria\|p_Firmicutes\|c_Clostridial\|o_Clostridiales\|f_Ruminococcaceae | 0.008194436 | 0.00973796 | 156 |
| \|g Ruminococcaceae unclassified\|s Ruminococcaceae bacterium D16 | | | |
| K18829 >> antitoxin VapB | 0.004904283 | 1.76359E−05 | 157 |
| K16248 >> probable glucitol transport protein GutA | −0.275184574 | −0.130473377 | 158 |
| K05306 >> phosphonoacetaldehyde hydrolase [EC: 3.11.1.1] | 0.245699387 | 0.040007054 | 159 |
| K10672 >> glycine reductase complex component B subunit gamma [EC: 1.21.4.2] | 0.206399694 | 0.127043193 | 160 |
| K09973 >> uncharacterized protein | 0.255609532 | 0.054277442 | 161 |
| K00243 >> uncharacterized protein | −0.119891793 | −0.006281322 | 162 |
| K20480 >> HTH-type transcriptional regulator, quorum sensing regulator NprR | −0.258781242 | −0.178822215 | 163 |
| K09766 >> uncharacterized protein | −0.319664484 | −0.140772747 | 164 |
| K06404 >> stage V sporulation protein AB | −0.326459187 | −0.063839043 | 165 |
| K03832 >> periplasmic protein TonB | 0.118828806 | 0.011566215 | 166 |
| K12340 >> outer membrane protein | 0.287435171 | 0.044610026 | 167 |
| K03706 >> transcriptional pleiotropic repressor | −0.184464456 | −0.059723998 | 168 |
| K02123 >> V/A-type H+/Na+-transporting ATPase subunit I | 0.056563329 | −0.00111988 | 169 |
| K12510 >> tight adherence protein B | −0.137932958 | −0.035912585 | 170 |
| k_Bacteria\|p_Actinobacteria\|c_Actinobacteria\|o_Actinomycetales | 0 | 0.022327058 | 171 |
| \|f_Actinomycetaceae\|g_Actinomyces\|s_Actinomyces_{—}turicensis | | | |
| K09009 >> uncharacterized protein | −0.086142537 | −0.046417706 | 172 |
| K09803 >> uncharacterized protein | −0.220689438 | −0.089052511 | 173 |
| K09700 >> uncharacterized protein | 0 | 0.05083844 | 174 |
| K07722 >> CopG family transcriptional regulator, nickel-responsive regulator | 0.242612742 | 0.059709301 | 175 |
| K12944 >> nucleoside triphosphatase [EC: 3.6.1.—] | 0.20074722 | 0.122243287 | 176 |
| K01661 >> naphthoate synthase [EC: 4.1.3.36] | 0.105353968 | 0.003753509 | 177 |
| K11710 >> manganese/zinc/iron transport system ATP- binding protein [EC: 7.2.2.5] | 0.017890247 | 0.092132916 | 178 |
| K11707 >> manganese/zinc/iron transport system substrate-binding protein | 0 | 0.078741384 | 179 |
| K00010 >> myo-inositol 2-dehydrogenase/D-chiro-inositol 1-dehydrogenase | −0.154013072 | −0.068562527 | 180 |
| [EC: 1.1.1.18 1.1.1.369] | | | |
| K10793 >> D-proline reductase (dithiol) PrdA [EC: 1.21.4.1] | 0.027706112 | 0.100727481 | 181 |
| K07811 >> trimethylamine-N-oxide reductase (cytochrome c) [EC: 1.7.2.3] | 0.087953754 | 0.077930132 | 182 |
| K02081 >> DeoR family transcriptional regulator, aga operon transcriptional repressor | 0.147895877 | 0.021454081 | 183 |
| K00756 >> pyrimidine-nucleoside phosphorylase [EC: 2.4.2.2] | −0.243399652 | −0.099942683 | 184 |
| K10824 >> nickel transport system ATP-binding protein [EC: 7.2.2.11] | 0.105278362 | 0.028296812 | 185 |
| k_Bacteria\|p_Bacteroidetes\|c_Bacteroidia\|o_Bacteroidales | 0 | 0.032626427 | 186 |
| \|f_Prevotellaceael\|g_Alloprevotella | | | |
| K10536 >> agmatine deiminase [EC: 3.5.3.12] | −0.238781193 | −0.097752892 | 187 |
| K07792 >> anaerobic C4-dicarboxylate transporter DcuB | 0.266375147 | 0.073462369 | 188 |
| k_Bacteria\|p_Firmicutes\|c_Clostridia\|o_Clostridiales | −0.180232016 | −0.013291595 | 189 |
| \|f_Lachnospiraceae\|g_Roseburia | | | |
| K10985 >> galactosamine PTS system EIIC component | 0.151629785 | 0.115938451 | 190 |
| K04024 >> ethanolamine utilization protein EutJ | 0.206859517 | 0.076289993 | 191 |
| K11051 >> multidrug/hemolysin transport system permease protein | −0.281690988 | −0.112067369 | 192 |
| K12516 >> putative surface-exposed virulence protein | 0 | 0.067833576 | 193 |
| K11085 >> ATP-binding cassette, subfamily B, bacterial MsbA [EC: 3.6.3.—] | 0.247996578 | 0.039495613 | 194 |
| K03091 >> RNA polymerase sporulation-specific sigma factor | −0.124597897 | −0.002751201 | 195 |
| k_Bacteria\|p_Fusobacteria\|c_Fusobacteriia\|o_Fusobacteriales\|f_Fusobacteriaceae | 0.217345618 | 0.189668298 | 196 |
| K02996 >> small subunit ribosomal protein S9 | 0.013607961 | 0 | 197 |
| K00426 >> cytochrome bd ubiquinol oxidase subunit II [EC: 7.1.1.7] | 0.12226608 | 0.013708978 | 198 |
| K04655 >> hydrogenase expression/formation protein HypE | 0.083227334 | 0.012054142 | 199 |
| K03077 >> L-ribulose-5-phosphate 4-epimerase [EC: 5.1.3.4] | −0.104263066 | 0.000681922 | 200 |
| k Bacteria\|p Bacteroidetes\|c Bacteroidia\|o Bacteroidales\|f Prevotellaceae | 0.40852486 | 0.178387196 | 201 |
| k_Bacteria\|p_Firmicutes\|c_Clostridia\|o_Clostridiales\|f_Eubacteriaceae | −0.238887444 | −0.119759564 | 202 |
| \|g_Eubacterium\|s_Eubacterium_{—}eligens | | | |
| K02863 >> large subunit ribosomal protein L1 | 0.026474626 | 0 | 203 |
| K07120 >> uncharacterized protein | 0.169567329 | 0.11033905 | 204 |
| K02045 >> sulfate/thiosulfate transport system ATP-binding protein [EC: 7.3.2.3] | −0.170038793 | −0.035742104 | 205 |
| K04784 >> yersiniabactin nonribosomal peptide synthetase | 0.085538481 | 0.071116794 | 206 |
| K07124 >> uncharacterized protein | −0.08263522 | −0.011686728 | 207 |
| k_Bacteria\|p_Fusobacteria | 0.21739474 | 0.187866496 | 208 |
| K03765 >> transcriptional activator of cad operon | 0.133691677 | 0.101744485 | 209 |
| K01924 >> UDP-N-acetylmuramate--alanine ligase [EC: 6.3.2.8] | −0.040371101 | 0.001801802 | 210 |
| k_Bacteria\|p_Firmicutes\|c_Clostridial\|o_Clostridiales\|f_Lachnospiraceae | −0.013250977 | −0.045095013 | 211 |
| \|g_Roseburial\|s_Roseburia_sp_CAG_303 | | | |
| K05595 >> multiple antibiotic resistance protein | 0.269970665 | 0.019164352 | 212 |
| k_Bacteria\|p_Fusobacteria\|c_Fusobacteriia\|o_Fusobacteriales | 0.215817798 | 0.188207457 | 213 |
| \|f_Fusobacteriaceae\|g_Fusobacterium | | | |
| K01190 >> beta-galactosidase [EC: 3.2.1.23] | 0.025898891 | −0.001290361 | 214 |
| K06384 >> stage II sporulation protein M | −0.302442768 | −0.129353497 | 215 |
| K03040 >> DNA-directed RNA polymerase subunit alpha [EC: 2.7.7.6] | −0.00710495 | 0 | 216 |
| K07404 >> 6-phosphogluconolactonase [EC: 3.1.1.31] | −0.137999361 | −0.012733125 | 217 |
| K02027 >> multiple sugar transport system substrate-binding protein | −0.130580964 | −0.006013844 | 218 |
| K09024 >> flavin reductase [EC: 1.5.1.—] | 0.209807034 | 0.113695751 | 219 |
| K13018 >> UDP-2-acetamido-3-amino-2,3-dideoxy-glucuronate N-acetyltransferase | 0.06000983 | 0.109225049 | 220 |
| [EC: 2.3.1.201] | | | |
| K00334 >> NADH-quinone oxidoreductase subunit E [EC: 7.1.1.2] | 0.262251204 | 0.032826301 | 221 |
| K02068 >> putative ABC transport system ATP-binding protein | 0.215750428 | 0.059318372 | 222 |
| K00975 >> glucose-1-phosphate adenylyltransferase [EC: 2.7.7.27] | −0.089495482 | −0.004893964 | 223 |
| K06966 >> pyrimidine/purine-5′-nucleotide nucleosidase [EC: 3.2.2.10 3.2.2.—] | 0.053158084 | 0.015266817 | 224 |
| K10117 >> raffinose/stachyose/melibiose transport system substrate-binding protein | −0.143276662 | −0.016142733 | 225 |
| K08159 >> MFS transporter, DHA1 family, L-arabinose/ | 0.136401925 | 0.061678644 | 226 |
| isopropyl-beta-D-thiogalactopyranoside export protein | | | |
| K06970 >> 23S rRNA (adenine1618-N6)-methyltransferase [EC: 2.1.1.181] | 0.1512853 | 0.021404112 | 227 |
| K02217 >> ferritin [EC: 1.16.3.2] | 0.035656604 | 0.000511441 | 228 |
| K07148 >> uncharacterized protein | 0.083155083 | 0.016022221 | 229 |
| K16092 >> vitamin B12 transporter | 0.230088275 | 0.059438884 | 230 |
| K07006 >> uncharacterized protein | −0.095251342 | −0.058319004 | 231 |
| K08224 >> MFS transporter, YNFM family, putative membrane transport protein | 0.182129316 | 0.065432153 | 232 |
| K07015 >> uncharacterized protein | −0.207304453 | −0.043313787 | 233 |
| K07259 >> serine-type D-Ala-D-Ala carboxypeptidase/endopeptidase (penicillin-binding protein 4) | 0.142660174 | 0.033943242 | 234 |
| [EC: 3.4.16.4 3.4.21.—] | | | |
| K07258 >> serine-type D-Ala-D-Ala carboxypeptidase (penicillin-binding protein 5/6) [EC: 3.4.16.4] | −0.098913593 | 0 | 235 |
| K05826 >> alpha-aminoadipate/glutamate carrier protein LysW | 0 | 0.048695678 | 236 |
| K08153 >> MFS transporter, DHA1 family, multidru\|g_resistance protein | −0.061649625 | −0.042843496 | 237 |
| K21140 >> [CysO sulfur-carrier protein]-S-L-cysteine hydrolase [EC: 3.13.1.6] | −0.442869152 | −0.160813015 | 238 |
| K07030 >> uncharacterized protein | −0.133467083 | −0.014340931 | 239 |
| K01571 >> oxaloacetate decarboxylase (Na+ extruding) subunit alpha [EC: 7.2.4.2] | −0.132837445 | −0.010493364 | 240 |
| K07042 >> probable rRNA maturation factor | −0.088356349 | −0.001290361 | 241 |
| K04769 >> AbrB family transcriptional regulator, stage V sporulation protein T | −0.11838085 | −0.025078259 | 242 |
| K14761 >> ribosome-associated protein | −0.076823476 | −0.007742163 | 243 |
| K07405 >> alpha-amylase [EC: 3.2.1.1] | 0.251310871 | 0.050259395 | 244 |
| K00040 >> fructuronate reductase [EC: 1.1.1.57] | −0.133746036 | −0.023884896 | 245 |
| K10974 >> cytosine permease | 0.07684468 | 0.010743207 | 246 |
| K07480 >> insertion element IS1 protein InsB | 0.294668808 | 0.11620005 | 247 |
| K08309 >> soluble lytic_murein transglycosylase [EC: 4.2.2.—] | 0.244156855 | 0.113860353 | 248 |
| k_Bacteria\|p_Firmicutes\|c_Clostridia\|o_Clostridiales\|f_Clostridiales_Family_XII_Incertae_Sedis | 0.015144225 | 0.043460753 | 249 |
| \|g Mogibacterium\|s Mogibacterium diversum | | | |
| K07502 >> uncharacterized protein | −0.265876694 | −0.078811928 | 250 |
| K08217 >> MFS transporter, DHA3 family, macrolide efflux protein | −0.111798051 | −0.029972223 | 251 |
| K01173 >> endonuclease G, mitochondrial | 0.209884658 | 0.061752127 | 252 |
| K00351 >> Na+-transporting NADH: ubiquinone oxidoreductase subunit F [EC: 7.2.1.1] | 0.13121916 | 0.032067957 | 253 |
| K16153 >> glycogen phosphorylase/synthase [EC: 2.4.1.1 2.4.1.11] | 0.181588781 | 0.023064827 | 254 |
| K11751 >> 5′-nucleotidase/UDP-sugar diphosphatase [EC: 3.1.3.5 3.6.1.45] | 0.188860837 | 0.016730597 | 255 |
| K08369 >> MFS transporter, putative metabolite: H+ symporter | −0.069186781 | −0.001360904 | 256 |
| K09773 >> [pyruvate, water dikinase]-phosphate phosphotransferase/ | 0.210908466 | 0.044536543 | 257 |
| [pyruvate, water dikinase] kinase [EC: 2.7.4.28 2.7.11.33] | | | |
| K08384 >> stage V sporulation protein D (sporulation-specific penicillin-binding protein) | −0.178075981 | −0.023811413 | 258 |
| K01787 >> N-acylglucosamine 2-epimerase [EC: 5.1.3.8] | 0.134319873 | −0.008909072 | 259 |
| K09121 >> pyridinium-3,5-bisthiocarboxylic acid mononucleotide nickel chelatase [EC: 4.99.1.12] | 0.10002349 | 0.001948768 | 260 |
| K06987 >> uncharacterized protein | −0.213365538 | −0.039613186 | 261 |
| K02687 >> ribosomal protein L11 methyltransferase [EC: 2.1.1.—] | −0.047052678 | 0.00017048 | 262 |
| K17320 >> putative aldouronate transport system permease protein | −0.265154112 | −0.034281263 | 263 |
| K03972 >> phage shock protein E | 0.237685955 | 0.132860103 | 264 |
| K08679 >> UDP-glucuronate 4-epimerase [EC: 5.1.3.6] | 0.246423494 | 0.071983893 | 265 |
| K02014 >> iron complex outermembrane recepter protein | 0.201190686 | 0.008206575 | 266 |
| K08296 >> phosphohistidine phosphatase [EC: 3.1.3.—] | 0.10330985 | 0.047387681 | 267 |
| K09002 >> CRISPR-associated protein Csm3 | −0.266963821 | −0.136046324 | 268 |
| K06403 >> stage V sporulation protein AA | −0.247989532 | −0.057581235 | 269 |
| K09772 >> cell division inhibitor SepF | −0.082492074 | −0.001290361 | 270 |
| K03300 >> citrate-Mg2+: H+ or citrate-Ca2+: H+ symporter, CitMHS family | −0.121109585 | −0.073721029 | 271 |
| K07091 >> lipopolysaccharide export system permease protein | 0.257275765 | 0.064382817 | 272 |
| K07552 >> MFS transporter, DHA1 family, multidrug resistance protein | 0.232054499 | 0.066769543 | 273 |
| K01963 >> acetyl-CoA carboxylase carboxyl transferase subunit beta [EC: 6.4.1.2 2.1.3.15] | −0.131301007 | 0.001022883 | 274 |
| K07230 >> periplasmic iron binding protein | 0.113236568 | 0.152856282 | 275 |
| K03814 >> monofunctional glycosyltransferase [EC: 2.4.1.129] | 0.226473852 | 0.021039637 | 276 |
| K07646 >> two-component system, OmpR family, sensor histidine kinase KdpD [EC: 2.7.13.3] | −0.077333077 | −0.002921682 | 277 |
| K07652 >> two-component system, OmpR family, sensor histidine kinase VicK [EC: 2.7.13.3] | 0.133956388 | 0.054794762 | 278 |
| K05813 >> sn-glycerol 3-phosphate transport system substrate-binding protein | −0.108774903 | −0.040950575 | 279 |
| K07216 >> hemerythrin | −0.200983615 | −0.04545655 | 280 |
| K00161 >> pyruvate dehydrogenase E1 component alpha subunit [EC: 1.2.4.1] | 0.190185741 | 0.1630322 | 281 |
| K01425 >> glutaminase [EC: 3.5.1.2] | 0.080691546 | −0.001290361 | 282 |
| K07720 >> two-component system, response regulator YesN | −0.162976467 | −0.032382464 | 283 |
| K11534 >> DeoR family transcriptional regulator, deoxyribose operon repressor | 0.211665104 | 0.087077289 | 284 |
| K02427 >> 23S rRNA (uridine2552-2′-O)-methyltransferase [EC: 2.1.1.166] | 0.253443139 | 0.083303205 | 285 |
| K21757 >> LysR family transcriptional regulator, | 0.029427943 | 0.088017871 | 286 |
| benzoate and cis,cis-muconate-responsive activator of ben and cat genes | | | |
| k_Bacteria\|p_Firmicutes\|c_Clostridia\|o_Clostridiales\|f_Ruminococcaceae | −0.003715517 | −0.009182429 | 287 |
| \|g Anaeromassilibacillus | | | |
| K02278 >> prepilin peptidase CpaA [EC: 3.4.23.43] | −0.246178886 | −0.089863763 | 288 |
| K07777 >> two-component system, NarL family, sensor histidine kinase DegS [EC: 2.7.13.3] | −0.331557245 | −0.145249328 | 289 |
| K03608 >> cell division topological specificity factor | −0.133808777 | 7.34829E−05 | 290 |
| K07783 >> MFS transporter, OPA family, sugar phosphate sensor protein UhpC | 0.169400634 | 0.01719207 | 291 |
| K01960 >> pyruvate carboxylase subunit B [EC: 6.4.1.1] | 0.205071101 | 0.060123745 | 292 |
| K06374 >> spore maturation protein B | −0.176402748 | −0.031456579 | 293 |
| k_Bacteria\|p_Firmicutes\|c_Clostridia\|o_Clostridiales | 0.021197248 | 0.054709522 | 294 |
| \|f_Clostridiales_Family_XIII_Incertae_Sedis\|g_Mogibacterium | | | |
| K07130 >> arylformamidase [EC: 3.5.1.9] | −0.103574897 | −0.059224314 | 295 |
| K00833 >> adenosylmethionine---8-amino-7-oxononanoate aminotransferase [EC: 2.6.1.62] | 0.098263454 | −0.001557838 | 296 |
| K23004 >> L-galactono-1,5-lactonase [EC: 3.1.1.—] | −0.024323356 | −0.012389224 | 297 |
| K06382 >> stage II sporulation protein E [EC: 3.1.3.16] | −0.156417725 | −0.023276458 | 298 |
| K03801 >> lipoyl(octanoyl) transferase [EC: 2.3.1.181] | 0.204233844 | 0.030777597 | 299 |
| K03437 >> RNA methyltransferase, TrmH family | 0.002907838 | −0.0009494 | 300 |
| K09021 >> aminoacrylate peracid reductase | 0.241349233 | 0.10887527 | 301 |
| K00965 >> UDPglucose--hexose-1-phosphate uridylyltransferase [EC: 2.7.7.12] | −0.12446063 | −0.008424085 | 302 |
| K10550 >> D-allose transport system permease protein | 0.176889345 | 0.107952324 | 303 |
| k_Bacteria\|p_Bacteroidetes\|c_Bacteroidia\|o_Bacteroidales\|f_Prevotellaceae | 0 | 0.022838499 | 304 |
| \|g_Alloprevotella\|s_Alloprevotella_{—}tannerae | | | |
| k_Bacteria\|p_Bacteroidetes\|c_Bacteroidia\|o_Bacteroidales\|f_Prevotellaceae | 0 | 0.070146819 | 305 |
| \|g_Prevotella\|s_Prevotella_{—}intermedia | | | |
| K02395 >> flagellar protein FlgJ | 0.23341559 | 0.079258704 | 306 |
| K11720 >> lipopolysaccharide export system permease protein | 0.132600614 | 0.004699969 | 307 |
| k Bacteria\|p Firmicutes\|c Bacilli\|o Bacillales | 0.075696653 | 0.146184031 | 308 |
| K01646 >> citrate lyase subunit gamma (acyl carrier protein) | 0.190914549 | 0.014758315 | 309 |
| K10708 >> fructoselysine 6-phosphate deglycase [EC: 3.5.—.—] | −0.099045016 | −0.082603648 | 310 |
| k Bacteria\|p Firmicutes\|c Bacilli\|o Bacillales\|f Bacillales unclassified | 0.077043072 | 0.147985832 | 311 |
| K09911 >> uncharacterized protein | 0.237928746 | 0.111041547 | 312 |
| K01241 >> AMP nucleosidase [EC: 3.2.2.4] | 0.349884407 | 0.106920624 | 313 |
| K13571 >> proteasome accessory factor A [EC: 6.3.1.19] | −0.139110102 | −0.036247667 | 314 |
| K11184 >> catabolite repression HPr-like protein | −0.238345697 | −0.04786679 | 315 |
| k_Bacteria\|p_Firmicutes\|c_Clostridia\|o_Clostridiales\|f_Clostridiaceae\|g_Hungatella | 0.052047642 | 0.069438443 | 316 |
| k_Bacteria\|p_Firmicutes\|c_Clostridia\|o_Clostridiales\|f_Clostridiaceae | 0.052047642 | 0.069438443 | 317 |
| \|g_Hungatella\|s_Hungatella_{—}hathewayi | | | |
| K16264 >> cobalt-zinc-cadmium efflux system protein | 0.162420189 | 0.018799877 | 318 |
| K17865 >> 3-hydroxybutyryl-CoA dehydratase [EC: 4.2.1.55] | 0.225763317 | 0.085228458 | 319 |
| K11926 >> sigma factor-binding protein Crl | 0.225809161 | 0.128354129 | 320 |
| K07014 >> uncharacterized protein | 0.239365961 | 0.069350264 | 321 |
| K02426 >> cysteine desulfuration protein SufE | 0.192591128 | 0.034043179 | 322 |
| K09181 >> acetyltransferase | 0.202805748 | 0.060244257 | 323 |
| K03415 >> two-component system, chemotaxis family, chemotaxis_protein Che V | −0.189591505 | −0.023761445 | 324 |
| k_Bacteria\|p_Actinobacteria\|c_Actinobacteria\|o_Actinomycetales\|f_Actinomycetaceae | 0 | 0.009787928 | 325 |
| \|g Actinomyces\|s Actinomyces cardiffensis | | | |
| k_Bacteria\|p_Firmicutes\|c_Clostridia\|o_Clostridiales\|f_Lachnospiraceae | −0.087127051 | −0.009787928 | 326 |
| K00076 >> 7-alpha-hydroxysteroid dehydrogenase [EC: 1.1.1.159] | 0.219194029 | 0.149878753 | 327 |
| K03568 >> TldD protein | 0.232705856 | 0.051305792 | 328 |
| K10201 >> N-acetylglucosamine transport system permease protein | 0.122524527 | 0.085043281 | 329 |
| K22044 >> moderate conductance mechanosensitive channel | 0.136381401 | 0.03455462 | 330 |
| K11527 >> two-component system, sensor histidine kinase and response regulator [EC: 2.7.13.3] | 0.252035108 | 0.072786326 | 331 |
| K17235 >> arabinosaccharide transport system permease protein | −0.322540894 | −0.14325647 | 332 |
| K22431 >> caffeyl-CoA reductase-Etf complex subunit CarD [EC: 1.3.1.108] | 0.213107914 | 0.081501404 | 333 |
| K07019 >> uncharacterized protein | 0.159066452 | 0.13468542 | 334 |
| K22927 >> cyclic-di-AMP phosphodiesterase [EC: 3.1.4.59] | −0.10914455 | −0.003700601 | 335 |
| K08093 >> 3-hexulose-6-phosphate synthase [EC: 4.1.2.43 | 0.006538776 | 0.00267184 | 336 |
| K18640 >> plasmid segregation protein ParM | −0.077996996 | −0.031456579 | 337 |
| K05896 >> segregation and condensation protein A | −0.121971792 | −0.006451803 | 338 |
| k Bacterial\|p Actinobacterial\|c Actinobacteria | −0.067126685 | 0.040835942 | 339 |
| K10254 >> oleate hydratase [EC: 4.2.1.53] | −0.187903065 | −0.031262584 | 340 |
| k_Bacteria\|p_Bacteroidetes\|c_Bacteroidia\|o_Bacteroidales | 0.067627432 | 0.134547272 | 341 |
| \|f Porphyromonadaceae\|g Porphyromonas | | | |
| K00823 >> 4-aminobutyrate aminotransferase [EC: 2.6.1.19] | 0.126138826 | 0.108028747 | 342 |
| K03823 >> phosphinothricin acetyltransferase [EC: 2.3.1.183] | −0.043253959 | −0.002921682 | 343 |
| K07284 >> sortase A [EC: 3.4.22.70] | −0.16916122 | 0.002995165 | 344 |
| K13628 >> iron-sulfur cluster assembly protein | 0.246725194 | 0.103590377 | 345 |
| K06143 >> inner membrane protein | 0.161753222 | 0.022426995 | 346 |
| K17318 >> putative aldouronate transport system substrate-binding protein | −0.289752201 | −0.047940273 | 347 |
| K19167 >> protein AbiQ | −0.285460968 | −0.142471672 | 348 |
| K00156 >> pyruvate dehydrogenase (quinone) [EC: 1.2.5.1] | 0.200127648 | 0.085445968 | 349 |
| k_Bacterial\|p_Bacteroidetes\|c_Bacteroidia\|o_Bacteroidales\|f_Bacteroidaceae | 0.101723034 | 0.03873139 | 350 |
| \|g Bacteroides\|s Bacteroides plebeius | | | |
| K02112 >> F-type H+/Na+-transporting ATPase subunit beta [EC: 7.1.2.2 7.2.2.1] | −0.048401594 | 0 | 351 |
| k_Bacterial\|p_Bacteroidetes\|c_Bacteroidia\|o_Bacteroidales\|f_Odoribacteraceae | 0.162986282 | 0.065285187 | 352 |
| K02502 >> ATP phosphoribosyltransferase regulatory subunit | −0.154876843 | −0.009567479 | 353 |
| K02529 >> LacI family transcriptional regulator | −0.039435749 | 0 | 354 |
| K18119 >> succinate-semialdehyde dehydrogenase [EC: 1.2.1.76] | 0.068774639 | 0.112999133 | 355 |
| K07662 >> two-component system, OmpR family, response regulator CpxR | 0.241330032 | 0.11133254 | 356 |
| K22477 >> N-acetylglutamate synthase [EC: 2.3.1.1] | −0.125355345 | −0.034957306 | 357 |
| K03772 >> FKBP-type peptidyl-prolyl cis-trans_isomerase FkpA [EC: 5.2.1.8] | 0.268211041 | 0.052572638 | 358 |
| K06158 >> ATP-binding cassette, subfamily F, member 3 | 0.021352934 | −0.000778919 | 359 |
| K03436 >> DeoR family transcriptional regulator, fructose operon transcriptional repressor | −0.328789219 | −0.116211807 | 360 |
| k_Bacterial\|p_Proteobacteria\|c_Betaproteobacteria\|o_Neisseriales\|f_Neisseriaceae | 0 | 0.016313214 | 361 |
| \|g_Eikenella\|s_Eikenella_{—}corrodens | | | |
| K21929 >> uracil-DNA glycosylase [EC: 3.2.2.27] | 0.160593266 | 0.018238467 | 362 |
| K20460 >> lantibiotic transport system permease protein | −0.103182829 | −0.030580662 | 363 |
| K11709 >> manganese/zinc/iron transport system permease protein | 0.027738018 | 0.096685919 | 364 |
| K16511 >> adapter protein MecA 1/2 | −0.185121679 | −0.021157209 | 365 |
| K20490 >> lantibiotic_transport system ATP-binding protein | −0.122773443 | 0.000755405 | 366 |
| K00936 >> two-component system, sensor histidine kinase PdtaS [EC: 2.7.13.3] | 0.191894791 | 0.09004894 | 367 |
| k_Bacteria\|p_Fusobacteria\|c_Fusobacteriia\|o_Fusobacteriales\|f_Fusobacteriaceae | 0 | 0.021207178 | 368 |
| \|g Fusobacterium\|s Fusobacterium naviforme | | | |
| K19354 >> heptose III glucuronosyltransferase [EC: 2.4.1.—] | 0.181886779 | 0.103131843 | 369 |
| K00428 >> cytochrome c peroxidase [EC: 1.11.1.5] | 0.178689535 | 0.106124069 | 370 |
| K02058 >> simple sugar transport system substrate-binding protein | −0.136524311 | −0.023446938 | 371 |
| K05936 >> precorrin-4/cobalt-precorrin-4 C11-methyltransferase [EC: 2.1.1.133 2.1.1.271] | −0.166244352 | −0.051373396 | 372 |
| K08299 >> crotonobetainyl-CoA hydratase [EC: 4.2.1.149] | 0.037895033 | 0.014717164 | 373 |
| k_Bacteria\|p_Proteobacteria\|c_Betaproteobacteria\|o_Neisseriales\|f_Neisseriaceae | 0 | 0.017944535 | 374 |
| \|g Eikenella | | | |
| K06407 >> stage V sporulation protein AE | −0.221630771 | −0.028364417 | 375 |
| K01945 >> phosphoribosylamine---glycine ligase [EC: 6.3.4.13] | −0.007599771 | 0 | 376 |
| k_Bacterial\|p_Proteobacteria\|c_Gammaproteobacteria\|o_Enterobacterales | 0 | 0.041732434 | 377 |
| \|f_Morganellaceae | | | |
| K07175 >> PhoH-like ATPase | 0.252415002 | 0.056373176 | 378 |
| K15876 >> cytochrome c nitrite reductase small subunit | 0.177138234 | 0.014781829 | 379 |
| k_Bacteria\|p_Proteobacteria\|c_Gammaproteobacteria\|o_Enterobacterales | 0 | 0.032455947 | 380 |
| f_Morganellaceae\|g_Morganella | | | |
| k_Bacteria\|p_Firmicutes\|c_Bacilli\|o_Lactobacillales\|f_Streptococcaceae | −0.077112787 | −0.045380127 | 381 |
| \|g_Streptococcus | | | |
| k_Bacteria\|p_Proteobacteria\|c_Gammaproteobacteria\|o_Enterobacterales | 0.210260162 | 0.112719898 | 382 |
| \|f Enterobacteriaceae\|g Escherichia\|s Escherichia coli | | | |
| K01627 >> 2-dehydro-3-deoxyphosphooctonate aldolase (KDO 8-P synthase) [EC: 2.5.1.55] | 0.211343353 | 0.039789545 | 383 |
| K03340 >> diaminopimelate dehydrogenase [EC: 1.4.1.16] | 0.100168194 | 0.002898167 | 384 |
| k_Bacteria\|p_Firmicutes\|c_Clostridia\|o_Clostridiales\|f_Lachnospiraceae\|g_Sellimonas | 0.002948352 | 0.004306101 | 385 |
| K02065 >> phospholipid/cholesterol/gamma-HCH transport system ATP-binding protein | 0.17750756 | −0.001313875 | 386 |
| K02913 >> large subunit ribosomal protein L33 | −0.005577191 | 0 | 387 |
| K07718 >> two-component system, sensor histidine kinase YesM [EC: 2.7.13.3] | −0.216124349 | −0.039419191 | 388 |
| K03179 >> 4-hydroxybenzoate polyprenyltransferase [EC: 2.5.1.39] | 0.232661856 | 0.099231368 | 389 |
| k_Bacteria\|p_Firmicutes\|c_Clostridia\|o_Clostridiales\|f_Lachnospiraceae | 0.040969412 | 0.070023368 | 390 |
| \|g_Eisenbergiella\|s_Eisenbergiella_{—}tayi | | | |
| k_Bacteria\|p_Firmicutes\|c_Clostridia\|o_Clostridiales\|f_Lachnospiraceae | 0.171639172 | 0.133415634 | 391 |
| \|g_Lachnoclostridium | | | |
| k_Bacteria\|p_Proteobacteria\|c_Gammaproteobacteria\|o_Enterobacterales | 0.256005884 | 0.099228429 | 392 |
| K08312 >> ADP-ribose diphosphatase [EC: 3.6.1.—] | 0.217412711 | 0.110774069 | 393 |
| K03700 >> recombination protein U | −0.243755302 | −0.0615258 | 394 |
| K02946 >> small subunit ribosomal protein S10 | 0.034191422 | 0 | 395 |
| K06975 >> uncharacterized protein | 0.300508364 | 0.05381303 | 396 |
| k_Bacteria\|p_Firmicutes\|c_Clostridia\|o_Clostridiales\|f_Lachnospiraceae | −0.292647548 | −0.154769778 | 397 |
| \|g_Roseburia\|s_Roseburia_{—}intestinalis | | | |
| K06960 >> uncharacterized protein | −0.140962897 | −0.01270961 | 398 |
| K21071 >> ATP-dependent phosphofructokinase/diphosphate-dependent phosphofructokinase | −0.059027991 | −0.001290361 | 399 |
| [EC: 2.7.1.11 2.7.1.90] | | | |
| K00209 >> enoyl-[acyl-carrier protein] reductase/trans-2-enoyl-CoA reductase (NAD+) | −0.320220208 | −0.136125685 | 400 |
| [EC: 1.3.1.9 1.3.1.44] | | | |
| k_Bacteria\|p_Proteobacteria\|c_Gammaproteobacteria | 0.231686738 | 0.057493056 | 401 |
| K11189 >> PTS-HPR phosphocarrier protein | −0.103209034 | 0.002313243 | 402 |
| K15771 >> arabinogalactan oligomer/maltooligosaccharide transport system permease protein | −0.246038779 | −0.062542804 | 403 |
| k_Bacteria\|p_Firmicutes\|c_Clostridia\|o_Clostridiales\|f_Peptostreptococcaceae | 0.095007581 | 0.101697456 | 404 |
| K09789 >> pimeloyl-[acyl-carrier protein] methyl ester esterase [EC: 3.1.1.85] | 0.260798701 | 0.029416692 | 405 |
| K00348 >> Na+-transporting NADH: ubiquinone oxidoreductase subunit C [EC: 7.2.1.1] | 0.262672122 | 0.050573902 | 406 |
| K17992 >> NADP-reducin\|g_hydrogenase subunit HndB [EC: 1.12.1.3] | 0.107532955 | 0.002436694 | 407 |
| K05606 >> methylmalonyl-CoA/ethylmalonyl-CoA epimerase [EC: 5.1.99.1] | 0.118394212 | 0.007407081 | 408 |
| k_Bacteria\|p_Firmicutes\|c_Clostridia\|o_Clostridiales\|f_Ruminococcaceae | −0.215537798 | −0.053710154 | 409 |
| \|g_Faecalibacterium | | | |
| K01676 >> fumarate hydratase, class I [EC: 4.2.1.2] | 0.267409917 | 0.052596152 | 410 |
| k_Bacteria\|p_Firmicutes\|c_Erysipelotrichia\|o_Erysipelotrichales | 0 | 0.006525285 | 411 |
| \|f Erysipelotrichaceae\|g Bulleidia | | | |
| K02834 >> ribosome-binding factor A | −0.032964385 | 0 | 412 |
| K02523 >> octaprenyl-diphosphate synthase [EC: 2.5.1.90] | 0.237295884 | 0.047143718 | 413 |
| K12992 >> rhamnosyltransferase [EC: 2.4.1.—] | −0.341655668 | −0.107396793 | 414 |
| K21613 >> N-acetylcysteine deacetylase [EC: 3.5.1.—] | −0.314296753 | −0.168646297 | 415 |
| K21579 >> betaine reductase complex component B subunit beta [EC: 1.21.4.4] | 0 | 0.072824537 | 416 |
| K03705 >> heat-inducible transcriptional repressor | −0.147813432 | −0.01382949 | 417 |
| K06395 >> stage III sporulation protein AF | −0.278528656 | −0.096536014 | 418 |
| K16203 >> D-amino_peptidase [EC: 3.4.11.—] | 0.205863031 | 0.112719898 | 419 |
| K16898 >> ATP-dependent helicase/nuclease subunit A [EC: 3.1.—.— 3.6.4.12] | −0.12739061 | −0.021571653 | 420 |
| K04751 >> nitrogen regulatory protein P-II 1 | −0.219670147 | −0.043460753 | 421 |
| K09800 >> translocation and assembly module TamB | 0.21571907 | 0.121755361 | 422 |
| K17319 >> putative aldouronate transport system permease protein | −0.194543002 | −0.015290331 | 423 |
| K21746 >> AraC family transcriptional regulator, | 0.215194018 | 0.121708331 | 424 |
| reactive chlorine species (RCS)-specific activator of rcl operon | | | |
| K05802 >> potassium-dependent mechanosensitive channel | 0.175674931 | 0.11766677 | 425 |
| K06379 >> stage II sporulation protein AB (anti-sigma F factor) [EC: 2.7.11.1] | −0.232445736 | −0.040124627 | 426 |
| K17816 >> 8-oxo-dGTP diphosphatase/2-hydroxy-dATP diphosphatase [EC: 3.6.1.55 3.6.1.56] | 0.021294081 | 0.097538321 | 427 |
| K01689 >> enolase [EC: 4.2.1.11] | −0.010632954 | 0 | 428 |
| K00259 >> alanine dehydrogenase [EC: 1.4.1.1] | 0.171110856 | 0.009208883 | 429 |
| K18012 >> L-erythro-3,5-diaminohexanoate dehydrogenase [EC: 1.4.1.11] | 0.267572606 | 0.094017019 | 430 |
| K12573 >> ribonuclease R [EC: 3.1.13.1] | −0.052858569 | 0.002142763 | 431 |
| K04028 >> ethanolamine utilization protein EutN | 0.160873502 | −0.013388593 | 432 |
| K02781 >> glucitol/sorbitol PTS system EIIA component [EC: 2.7.1.198] | −0.199119687 | −0.056411387 | 433 |
| K02558 >> UDP-N-acetylmuramate L-alanyl-gamma-D-glutamyl-meso-diaminopimelate ligase | 0.215103489 | 0.07190747 | 434 |
| [EC: 6.3.2.45] | | | |
| k_Bacterial\|p_Proteobacteria | 0.19664631 | 0.04037153 | 435 |
| K02803 >> N-acetylglucosamine PTS system EIIB component [EC: 2.7.1.193] | −0.161802615 | −0.031456579 | 436 |
| K08659 >> dipeptidase [EC: 3.4.—.—] | −0.13373114 | −0.021742134 | 437 |
| K08301 >> ribonuclease G [EC: 3.1.26.—] | 0.140603299 | −0.00335964 | 438 |
| K02965 >> small subunit ribosomal protein S19 | −0.02236616 | 0 | 439 |
| K03789 >> [ribosomal protein S18]-alanine N-acetyltransferase [EC: 2.3.1.266] | −0.127912717 | 0.001022883 | 440 |
| K01195 >> beta-glucuronidase [EC: 3.2.1.31] | −0.157903831 | 0.001169848 | 441 |
| K07277 >> outer membrane protein insertion porin family | 0.15358115 | 0.028026395 | 442 |
| K07149 >> uncharacterized protein | −0.17728277 | −0.057848713 | 443 |
| K00950 >> 2-amino-4-hydroxy-6-hydroxymethyldihydropteridine diphosphokinase [EC: 2.7.6.3] | 0.220241053 | 0.012224623 | 444 |
| K14065 >> universal stress protein D | 0.246314004 | 0.107608424 | 445 |
| K08194 >> MFS transporter, ACS family, D-galactonate transporter | 0.185257544 | 0.12326617 | 446 |
| K07090 >> uncharacterized protein | 0.018599418 | −0.0009494 | 447 |
| K14977 >> (S)-ureidoglycine aminohydrolase [EC: 3.5.3.26] | 0.174250931 | 0.106027071 | 448 |
| K15125 >> filamentous_hemagglutinin | 0.077662781 | 0.079470335 | 449 |
| K14534 >> 4-hydroxybutyryl-CoA dehydratase/vinylacetyl-CoA-Delta-isomerase | 0.180922228 | 0.01960525 | 450 |
| [EC: 4.2.1.120 5.3.3.3] | | | |
| K20342 >> HTH-type transcriptional regulator, regulator for ComX | 0.008365534 | 0.002036947 | 451 |
| K13566 >> omega-amidase [EC: 3.5.1.3] | 0.169093102 | 0.019869788 | 452 |
| K00858 >> NAD+ kinase [EC: 2.7.1.23] | −0.065163879 | 0.00017048 | 453 |
| K03642 >> rare lipoprotein A | 0.227265086 | 0.039813059 | 454 |
| K00425 >> cytochrome bd ubiquinol oxidase subunit I [EC: 7.1.1.7] | 0.209417235 | 0.024108284 | 455 |
| K16212 >> 4-O-beta-D-mannosyl-D-glucose phosphorylase [EC: 2.4.1.281] | 0.151202992 | 0.008209515 | 456 |
| K03305 >> proton-dependent oligopeptide transporter, POT family | 0.194581915 | −2.35145E−05 | 457 |
| K03271 >> D-sedoheptulose 7-phosphate isomerase [EC: 5.3.1.28] | 0.200048786 | 0.02167159 | 458 |
| K12661 >> L-rhamnonate dehydratase [EC: 4.2.1.90] | 0.211295257 | 0.101621034 | 459 |
| K18234 >> virginiamycin A acetyltransferase [EC: 2.3.1.—] | −0.062986038 | −0.031353703 | 460 |
| K02518 >> translation initiation factor IF-1 | −0.006510191 | 0.001801802 | 461 |
| K03113 >> translation initiation factor 1 | 0.283450633 | 0.053378011 | 462 |
| K09697 >> sodium transport system ATP-binding protein [EC: 7.2.2.4] | −0.283499504 | −0.146466205 | 463 |
| K09903 >> uridylate kinase [EC: 2.7.4.22] | 0.016950829 | 0 | 464 |
| K00137 >> aminobutyraldehyde dehydrogenase [EC: 1.2.1.19] | 0.125446702 | 0.111826345 | 465 |
| K16937 >> thiosulfate dehydrogenase (quinone) large subunit [EC: 1.8.5.2] | 0.231715251 | 0.051211734 | 466 |
| K03543 >> membrane fusion protein, multidrug efflux system | 0.165151561 | 0.033093779 | 467 |
| K01677 >> fumarate hydratase subunit alpha [EC: 4.2.1.2] | −0.132854226 | −0.044166189 | 468 |
| K20116 >> PTS system, glucose-specific IIA component | −0.020930091 | −0.026218715 | 469 |
| K20117 >> PTS system, glucose-specific IIB component | 0.000114291 | −0.01986391 | 470 |
| K01751 >> diaminopropionate ammonia-lyase [EC: 4.3.1.15] | 0.136212351 | 0.009593933 | 471 |
| K07003 >> uncharacterized protein | −0.064476842 | 0.005625854 | 472 |
| K20265 >> glutamate: GABA antiporter | 0.143264687 | −0.000972914 | 473 |
| k_Bacterial\|p_Fusobacteria\|c_Fusobacteriia\|o_Fusobacteriales\|f_Fusobacteriaceae | 0 | 0.027732463 | 474 |
| \|g_Fusobacterium\|s_Fusobacterium_sp_oral_taxon_370 | | | |
| K16328 >> pseudouridine kinase [EC: 2.7.1.83] | −0.189010725 | −0.015434358 | 475 |
| K20344 >> ATP-binding cassette, subfamily C, bacteriocin exporter | −0.20242373 | −0.066249284 | 476 |
| K05364 >> penicillin-binding protein A | −0.262817712 | −0.106761901 | 477 |
| K20444 >> O-antigen biosynthesis_protein [EC: 2.4.1.—] | −0.220026908 | −0.048110753 | 478 |
| K18324 >> multidrug efflux pump | 0.171092806 | 0.116082477 | 479 |
| K01035 >> acetate CoA/acetoacetate CoA-transferase beta subunit [EC: 2.8.3.8 2.8.3.9] | 0.320364032 | 0.021477595 | 480 |
| K18928 >> L-lactate dehydrogenase complex protein LldE | 0.134114142 | 0.005284893 | 481 |
| K09696 >> sodium transport system permease protein | −0.278104801 | −0.117128874 | 482 |
| K02118 >> V/A-type H+/Na+-transporting ATPase subunit B | −0.00741193 | 0.00017048 | 483 |
| K07742 >> uncharacterized protein | −0.13790016 | −0.020110812 | 484 |
| K01962 >> acetyl-CoA carboxylase carboxyl transferase subunit alpha [EC: 6.4.1.2 2.1.3.15] | −0.120794379 | −0.005502403 | 485 |
| K21416 >> acetoin: 2,6-dichlorophenolindophenol oxidoreductase subunit alpha [EC: 1.1.1.—] | 0.051360671 | 0.033572888 | 486 |
| K18013 >> 3-keto-5-aminohexanoate cleavage enzyme [EC: 2.3.1.247] | 0.306949862 | 0.115350587 | 487 |
| K01095 >> phosphatidylglycerophosphatase A [EC: 3.1.3.27] | 0.244513799 | 0.051208794 | 488 |
| k_Bacteria\|p_Firmicutes\|c_Clostridia\|o_Clostridiales\|f_Ruminococcaceae | 0.181819482 | 0.097961583 | 489 |
| \|g_Ruthenibacterium | | | |
| K06726 >> D-ribose pyranase [EC: 5.4.99.62] | 0.301238635 | 0.076143027 | 490 |
| K06381 >> stage II sporulation protein D | −0.142096702 | −0.025078259 | 491 |
| K03281 >> chloride channel protein, CIC family | 0.173950794 | 0.025298708 | 492 |
| K01092 >> myo-inositol-1(or 4)-monophosphatase [EC: 3.1.3.25] | 0.12882577 | 0.008741531 | 493 |
| K20074 >> PPM family protein phosphatase [EC: 3.1.3.16] | −0.112922058 | −0.004041562 | 494 |
| K19715 >> 8-amino-3,8-dideoxy-alpha-D-manno-octulosonate transaminase | 0 | 0.041391473 | 495 |
| [EC: 2.6.1.109] | | | |
| K18446 >> triphosphatase [EC: 3.6.1.25] | 0.180355907 | 0.129717972 | 496 |
| K01297 >> muramoyltetrapeptide carboxypeptidase [EC: 3.4.17.13] | 0.162523635 | 0.027076995 | 497 |
| K19064 >> lysine 6-dehydrogenase [EC: 1.4.1.18] | 0 | 0.068442015 | 498 |
| K08641 >> zinc D-Ala-D-Ala dipeptidase [EC: 3.4.13.22] | 0.203135204 | 0.03494261 | 499 |
| K15772 >> arabinogalactan oligomer/maltooligosaccharide transport system permease protein | −0.177155035 | −0.034889702 | 500 |
| K09740 >> uncharacterized protein | 0.209300724 | 0.063997766 | 501 |
| k_Bacteria\|p_Proteobacteria\|c_Gammaproteobacteria\|o_{——}Enterobacterales | 0.259599798 | 0.108748879 | 502 |
| \|f_Enterobacteriaceae | | | |
| K18691 >> membrane-bound lytic murein transglycosylase F [EC: 4.2.2.—] | 0.242217567 | 0.042637744 | 503 |
| K00338 >> NADH-quinone oxidoreductase subunit I [EC: 7.1.1.2] | 0.14673906 | −0.000461473 | 504 |
| K18850 >> 50S ribosomal protein L16 3-hydroxylase [EC: 1.14.11.47] | 0.124349998 | 0.109657129 | 505 |
| K18120 >> 4-hydroxybutyrate dehydrogenase [EC: 1.1.1.61] | 0.209752517 | 0.100066135 | 506 |
| K12257 >> SecD/SecF fusion protein | −0.040844896 | −0.008862043 | 507 |
| K02804 >> N-acetylglucosamine PTS system EIICBA or EIICB component [EC: 2.7.1.193] | −0.161718941 | −0.031456579 | 508 |
| K01547 >> potassium-transporting ATPase ATP-binding subunit [EC: 7.2.2.6] | 0.094541358 | −0.004138559 | 509 |
| K05782 >> benzoate membrane transport protein | 0.204640077 | 0.100962627 | 510 |
| K07038 >> inner membrane protein | 0.233618957 | 0.085619388 | 511 |
| K19591 >> MerR family transcriptional regulator, copper efflux regulator | 0.238877704 | 0.100742178 | 512 |
| K03570 >> rod shape-determining protein MreC | 0.06310658 | 0.001801802 | 513 |
| K19138 >> CRISPR-associated protein Csm2 | −0.271676606 | −0.113328336 | 514 |
| K01424 >> L-asparaginase [EC: 3.5.1.1] | 0.084685835 | 0.000852402 | 515 |
| K15986 >> manganese-dependent inorganic pyrophosphatase [EC: 3.6.1.1] | −0.194584418 | −0.030654145 | 516 |
| K00281 >> glycine dehydrogenase [EC: 1.4.4.2] | 0.264799979 | 0.008888497 | 517 |
| K19268 >> methylaspartate mutase epsilon subunit [EC: 5.4.99.1] | 0.388533261 | 0.187472628 | 518 |
| K11928 >> sodium/proline symporter | −0.141107365 | −0.010055406 | 519 |
| K06518 >> holin-like protein | 0.220236998 | 0.015828226 | 520 |
| K06412 >> stage V sporulation protein G | −0.191000428 | −0.022788531 | 521 |
| K04027 >> ethanolamine utilization protein EutM | 0.246864503 | 0.064797261 | 522 |
| K19333 >> IclR family transcriptional regulator, KDG regulon repressor | 0 | 0.044995077 | 523 |
| K02026 >> multiple sugar transport system permease protein | −0.11708798 | −0.006013844 | 524 |
| K03707 >> thiaminase (transcriptional activator TenA) [EC: 3.5.99.2] | −0.104942142 | −0.068874094 | 525 |
| K00384 >> thioredoxin reductase (NADPH) [EC: 1.8.1.9] | −0.061457118 | −0.003262643 | 526 |
| K19353 >> heptose-I-phosphate ethanolaminephosphotransferase [EC: 2.7.8.—] | 0.106231242 | 0.110197963 | 527 |
| K06406 >> stage V sporulation protein AD | −0.178774834 | −0.019963846 | 528 |
| K06934 >> uncharacterized protein | −0.296002629 | −0.11667328 | 529 |
| K03307 >> solute: Na+ symporter, SSS family | 0.148282408 | 0.004699969 | 530 |
| K06398 >> stage IV sporulation protein A | −0.117681529 | −0.01813853 | 531 |
| K01624 >> fructose-bisphosphate aldolase, class_II [EC: 4.1.2.13] | −0.080960128 | −0.001460841 | 532 |
| K00705 >> 4-alpha-glucanotransferase [EC: 2.4.1.25] | −0.047728001 | 0 | 533 |
| K04083 >> molecular chaperone Hsp33 | −0.106206011 | 0.000340961 | 534 |
| K01847 >> methylmalonyl-CoA mutase [EC: 5.4.99.2] | 0.264865409 | 0.051111797 | 535 |
| K01880 >> glycyl-tRNA synthetase [EC: 6.1.1.14] | −0.169780501 | −0.009617448 | 536 |
| K01897 >> long-chain acyl-CoA synthetase [EC: 6.2.1.3] | 0.048355064 | 0.00017048 | 537 |
| K05833 >> putative ABC transport system ATP-binding protein | −0.097456793 | −0.007815646 | 538 |
| K10538 >> L-arabinose transport system permease protein | 0.178177371 | 0.1083168 | 539 |
| K01911 >> O-succinylbenzoic acid---CoA ligase [EC: 6.2.1.26] | 0.283013371 | 0.082571315 | 540 |
| K01512 >> acylphosphatase [EC: 3.6.1.7] | −0.150657365 | −0.012903605 | 541 |
| K01802 >> peptidylprolyl isomerase [EC: 5.2.1.8] | −0.083576611 | 0.010452214 | 542 |
| K04032 >> ethanolamine utilization cobalamin adenosyltransferase [EC: 2.5.1.17] | 0.238399906 | 0.07996708 | 543 |
| K02454 >> general secretion pathway protein E [EC: 7.4.2.8] | 0.235169767 | 0.124383111 | 544 |
| K01000 >> phospho-N-acetylmuramoyl-pentapeptide-transferase [EC: 2.7.8.13] | 0.008541204 | 0 | 545 |
| K06923 >> uncharacterized protein | −0.145197559 | −0.011516247 | 546 |
| K07590 >> large subunit ribosomal protein L7A | 0.202852902 | 0.113260732 | 547 |
| K03624 >> transcription elongation factor GreA | −0.055322062 | 0 | 548 |
| K01940 >> argininosuccinate synthase [EC: 6.3.4.5] | −0.080903863 | 0.001801802 | 549 |
| K21573 >> TonB-dependent starch-binding outer membrane protein SusC | 0.254461812 | 0.028223329 | 550 |
| K01667 >> tryptophanase [EC: 4.1.99.1] | 0.312341317 | 0.051840748 | 551 |
| K00571 >> site-specific DNA-methyltransferase (adenine-specific) [EC: 2.1.1.72] | −0.079786653 | −0.025101774 | 552 |
| K03406 >> methyl-accepting chemotaxis_protein | −0.269876812 | −0.03610658 | 553 |
| K01990 >> ABC-2 type transport system ATP-binding protein | −0.053996817 | 0 | 554 |
| K21012 >> polysaccharide biosynthesis_protein PelG | −0.288891008 | −0.127939685 | 555 |
| K01992 >> ABC-2 type transport system permease protein | −0.048233599 | 0 | 556 |
| k_Bacteria\|p_Proteobacteria\|c_Gammaproteobacteria\|o_Enterobacterales | 0.206806357 | 0.112549417 | 557 |
| \|f Enterobacteriaceae\|g Escherichia | | | |
| K00868 >> pyridoxine kinase [EC: 2.7.1.35] | −0.096256735 | 0.000340961 | 558 |
| K01993 >> HlyD family secretion protein | 0.120922499 | 0.014220419 | 559 |
| K01778 >> diaminopimelate epimerase [EC: 5.1.1.7] | −0.022173583 | 0.00017048 | 560 |
| K00912 >> tetraacyldisaccharide 4′-kinase [EC: 2.7.1.130] | 0.117855793 | 0.006672251 | 561 |
| K07274 >> MipA family protein | 0.260476264 | 0.115106624 | 562 |
| K01744 >> aspartate ammonia-lyase [EC: 4.3.1.1] | 0.150465156 | 0.006428288 | 563 |
| k_Bacteria\|p_Proteobacteria\|c_Epsilonproteobacteria\|o_Campylobacterales | 0 | 0.003262643 | 564 |
| \|f_Campylobacteraceae\|g_Campylobacter\|s_Campylobacter_{—}ureolyticus | | | |
| K04835 >> methylaspartate ammonia-lyase [EC: 4.3.1.2] | 0.300018317 | 0.102811457 | 565 |
| K01738 >> cysteine synthase [EC: 2.5.1.47] | −0.067066912 | 0.00017048 | 566 |
| K00432 >> glutathione peroxidase [EC: 1.11.1.9] | 0.214894919 | 0.037452787 | 567 |
| K01457 >> allophanate hydrolase [EC: 3.5.1.54] | −0.338715926 | −0.162267978 | 568 |
| K18142 >> multidrug efflux pump | 0.134246838 | 0.130035419 | 569 |
| K01546 >> potassium-transporting ATPase potassium-binding subunit | 0.094733534 | 0.006939729 | 570 |
| K21063 >> 5-amino-6-(5-phospho-D-ribitylamino)uracil phosphatase [EC: 3.1.3.104] | 0.311418089 | 0.0691063 | 571 |
| K06891 >> ATP-dependent Clp_protease adaptor protein ClpS | 0.314807083 | 0.096938701 | 572 |
| K01582 >> lysine decarboxylase [EC: 4.1.1.18] | 0.24556645 | 0.129009597 | 573 |
| K09759 >> nondiscriminating aspartyl-tRNA synthetase [EC: 6.1.1.23] | −0.183421538 | −0.02188616 | 574 |
| K00003 >> homoserine dehydrogenase [EC: 1.1.1.3] | −0.153355072 | −0.009714445 | 575 |
| K15977 >> putative oxidoreductase | 0.090072136 | 0.011225255 | 576 |
| K09762 >> uncharacterized protein | −0.187980486 | −0.011004806 | 577 |
| K03839 >> flavodoxin I | 0.205925947 | 0.029049278 | 578 |
| K01639 >> N-acetylneuraminate lyase [EC: 4.1.3.3] | 0.206231421 | 0.016948106 | 579 |
| K09794 >> uncharacterized protein | 0.114629876 | 0.079196978 | 580 |
| K09158 >> uncharacterized protein | 0.26552667 | 0.087665153 | 581 |
| K00981 >> phosphatidate cytidylyltransferase [EC: 2.7.7.41] | 0.079749809 | 0.002313243 | 582 |
| k_Bacteria\|p_Bacteroidetes\|c_Bacteroidia\|o_Bacteroidales\|f_Bacteroidaceae | 0.231383405 | 0.146469145 | 583 |
| \|g_Bacteroides_s_Bacteroides_{—}fragilis | | | |
| K01657 >> anthranilate synthase component I [EC: 4.1.3.27] | −0.062519578 | 0.000681922 | 584 |
| K06958 >> RNase adapter protein RapZ | −0.088334119 | 0.001801802 | 585 |
| K01251 >> adenosylhomocysteinase [EC: 3.3.1.1] | 0.100279448 | 0.000414444 | 586 |
| k_Bacteria\|p_Firmicutes\|c_Clostridia\|o_Clostridiales\|f_Eubacteriaceae | −0.243071649 | −0.041000544 | 587 |
| K08223 >> MFS transporter, FSR family, fosmidomycin resistance protein | 0.174432172 | 0.037449848 | 588 |
| K07447 >> putative holliday junction resolvase [EC: 3.1.—.—] | −0.052568343 | 0.003603604 | 589 |
| K01704 >> 3-isopropylmalate/(R)-2-methylmalate dehydratase small subunit | −0.100489701 | −0.001460841 | 590 |
| [EC: 4.2.1.33 4.2.1.35] | | | |
| K01706 >> glucarate dehydratase [EC: 4.2.1.40] | 0.212928861 | 0.078726688 | 591 |
| K09013 >> Fe—S cluster assembly ATP-binding protein | −0.099941541 | 0.001022883 | 592 |
| K02745 >> N-acetylgalactosamine PTS system EIIB component [EC: 2.7.1.—] | 0.174584799 | 0.047390621 | 593 |
| K09825 >> Fur family transcriptional regulator, peroxide stress_response regulator | −0.088116087 | −0.01270961 | 594 |
| K02003 >> putative ABC transport system ATP-binding protein | −0.054616227 | 0 | 595 |
| K03667 >> ATP-dependent HslUV protease ATP-binding subunit HslU | 0.249652045 | 0.084546537 | 596 |
| K05827 >> [lysine-biosynthesis-protein LysW]---L-2-aminoadipate ligase [EC: 6.3.2.43] | 0 | 0.05066796 | 597 |
| K06911 >> quercetin 2,3-dioxygenase [EC: 1.13.11.24] | 0.351465955 | 0.055979307 | 598 |
| K02245 >> competence protein ComGC | 0.034011645 | 8.81795E−05 | 599 |
| K02535 >> UDP-3-O-[3-hydroxymyristoyl] N-acetylglucosamine deacetylase [EC: 3.5.1.108] | 0.274863987 | 0.088123686 | 600 |
| K01515 >> ADP-ribose pyrophosphatase [EC: 3.6.1.13] | −0.093481342 | −0.007645166 | 601 |
| K02004 >> putative ABC transport system permease protein | −0.037161673 | 0 | 602 |
| K06310 >> spore germination protein | −0.345114097 | −0.09741487 | 603 |
| K03702 >> excinuclease ABC subunit B | −0.032835132 | 0.001801802 | 604 |
| K02025 >> multiple sugar transport system permease protein | −0.138149863 | 0.000511441 | 605 |
| k_Bacteria\|p_Firmicutes\|c_Clostridia\|o_Clostridiales\|f_Eubacteriaceae\|g_Eubacterium | −0.245986484 | −0.045894508 | 606 |
| K03587 >> cell division protein FtsI (penicillin-binding protein 3) [EC: 3.4.16.4] | 0.190269953 | 0.039301618 | 607 |
| K07029 >> diacylglycerol kinase (ATP) [EC: 2.7.1.107] | −0.29505136 | −0.136951634 | 608 |
| K04047 >> starvation-inducible DNA-binding protein | 0.245206155 | 0.059144952 | 609 |
| K02377 >> GDP-L-fucose synthase [EC: 1.1.1.271] | 0.274296193 | 0.039592611 | 610 |
| K03386 >> peroxiredoxin (alkyl hydroperoxide reductase subunit C) [EC: 1.11.1.15] | 0.214203871 | 0.013952942 | 611 |
| K00805 >> heptaprenyl diphosphate synthase [EC: 2.5.1.30] | −0.14257106 | −0.013488529 | 612 |
| K01273 >> membrane dipeptidase [EC: 3.4.13.19] | −0.211010975 | −0.053028232 | 613 |
| k_Bacteria\|p_Firmicutes\|c_Clostridia\|o_Clostridiales\|f_Lachnospiraceae\|g_Eisenbergiella | 0.067939479 | 0.100627544 | 614 |
| K00147 >> glutamate-5-semialdehyde dehydrogenase [EC: 1.2.1.41] | −0.047968392 | 0.00017048 | 615 |
| K03148 >> sulfur carrier protein ThiS adenylyltransferase [EC: 2.7.7.73] | 0.24151813 | 0.109701218 | 616 |
| K02386 >> flagella basal body P-ring formation protein FlgA | 0.226201774 | 0.10456623 | 617 |
| K02434 >> aspartyl-tRNA(Asn)/glutamyl-tRNA(Gln) amidotransferase subunit B [EC: 6.3.5.6 6.3.5.7] | −0.124939495 | −0.010566847 | 618 |
| K12952 >> cation-transporting P-type ATPase E [EC: 7.2.2.—] | −0.123534807 | −0.028849404 | 619 |
| k_Bacteria\|p_Actinobacteria\|c_Coriobacteriia\|o_Coriobacteriales\|f_Atopobiaceae | 0.021441684 | 0.037981864 | 620 |
| K02443 >> glycerol uptake operon antiterminator | −0.2166552 | −0.094119895 | 621 |
| K11065 >> thiol peroxidase, atypical 2-Cys_peroxiredoxin [EC: 1.11.1.15] | 0.249298386 | 0.039545581 | 622 |
| K02446 >> fructose-1,6-bisphosphatase II [EC: 3.1.3.11] | 0.369358639 | 0.138453625 | 623 |
| K13990 >> glutamate formiminotransferase/formiminotetrahydrofolate cyclodeaminase | 0.077654394 | 0.016836412 | 624 |
| [EC: 2.1.2.5 4.3.1.4] | | | |
| K00113 >> glycerol-3-phosphate dehydrogenase subunit C | 0.186016109 | 0.110994518 | 625 |
| K02343 >> DNA polymerase III subunit gamma/tau [EC: 2.7.7.7] | −0.097438115 | −0.004382523 | 626 |
| K02473 >> UDP-N-acetylglucosamine 4-epimerase [EC: 5.1.3.7] | 0.050701286 | 0.098925679 | 627 |
| K02230 >> cobaltochelatase CobN [EC: 6.6.1.2] | 0.309758549 | 0.031900416 | 628 |
| K03975 >> membrane-associated protein | −0.032823139 | 0.005819849 | 629 |
| K02169 >> malonyl-CoA O-methyltransferase [EC: 2.1.1.197] | 0.308506836 | 0.053739547 | 630 |
| K02116 >> ATP synthase protein I | 0.22872691 | 0.108266831 | 631 |
| K22132 >> tRNA threonylcarbamoyladenosine dehydratase | −0.067660356 | −0.006184325 | 632 |
| K03116 >> sec-independent protein translocase protein TatA | 0.026943496 | 0.004796967 | 633 |
| K02890 >> large subunit ribosomal protein L22 | 0.005318222 | 0 | 634 |
| K02029 >> polar amino acid transport system permease protein | −0.083340534 | 0.000340961 | 635 |
| K07816 >> putative GTP pyrophosphokinase [EC: 2.7.6.5] | −0.207552943 | −0.033672825 | 636 |
| K02057 >> simple sugar transport system permease protein | −0.086962089 | −0.003262643 | 637 |
| K11183 >> multiphosphoryl transfer protein [EC: 2.7.1.202] | 0.155162775 | 0.10792587 | 638 |
| K03438 >> 16S rRNA (cytosine1402-N4)-methyltransferase [EC: 2.1.1.199] | −0.041942317 | 0 | 639 |
| K02047 >> sulfate/thiosulfate transport system permease protein | −0.155784448 | −0.030336699 | 640 |
| k_Bacterial\|p_Bacteroidetes\|c_Bacteroidia\|o_Bacteroidales\|f_Porphyromonadaceae | 0 | 0.034257749 | 641 |
| \|g_Porphyromonas\|s_Porphyromonas_{—}uenonis | | | |
| K00926 >> carbamate kinase [EC: 2.7.2.2] | 0.11787737 | 0.000243963 | 642 |
| K18349 >> two-component system, OmpR family, response regulator VanR | −0.210938536 | −0.036180063 | 643 |
| K01941 >> urea carboxylase [EC: 6.3.4.6] | −0.292266078 | −0.151360169 | 644 |
| K03629 >> DNA replication and repair protein RecF | −0.03400587 | 0.003603604 | 645 |
| K02066 >> phospholipid/cholesterol/gamma-HCH transport system permease protein | 0.26997836 | 0.0143909 | 646 |
| K06390 >> stage III sporulation protein AA | −0.163608539 | −0.037373426 | 647 |
| K00275 >> pyridoxamine 5′-phosphate oxidase [EC: 1.4.3.5] | 0.327035593 | 0.049868466 | 648 |
| K02077 >> zinc/manganese transport system substrate-binding protein | −0.147101048 | −0.043575386 | 649 |
| K02337 >> DNA polymerase III subunit alpha [EC: 2.7.7.7] | −0.011466598 | 0 | 650 |
| K11927 >> ATP-dependent RNA helicase RhlE [EC: 3.6.4.13] | 0.296200495 | 0.068715371 | 651 |
| K02106 >> short-chain fatty acids_transporter | 0.18452885 | 0.011910116 | 652 |
| K02113 >> F-type H+-transporting ATPase subunit delta | −0.03397425 | 0.001801802 | 653 |
| K00033 >> 6-phosphogluconate dehydrogenase [EC: 1.1.1.44 1.1.1.343] | 0.197431981 | 0.043346119 | 654 |
| K00655 >> 1-acyl-sn-glycerol-3-phosphate acyltransferase [EC: 2.3.1.51] | −0.074287999 | 0 | 655 |
| K05979 >> 2-phosphosulfolactate phosphatase [EC: 3.1.3.71] | 0.128124682 | 0.110368443 | 656 |
| K06861 >> lipopolysaccharide export system ATP-binding protein [EC: 3.6.3.—] | 0.342297721 | 0.093332158 | 657 |
| K02843 >> heptosyltransferase II [EC: 2.4.—.—] | 0.289375123 | 0.057319636 | 658 |
| K03387 >> NADH-dependent peroxiredoxin subunit F [EC: 1.8.1.—] | 0.183427807 | 0.002680658 | 659 |
| K03311 >> branched-chain amino_acid:cation transporter, LIVCS family | 0.26674278 | 0.070543627 | 660 |
| K08987 >> putative membrane protein | 0.032194277 | 0.029163911 | 661 |
| K01933 >> phosphoribosylformylglycinamidine cyclo-ligase [EC: 6.3.3.1] | −0.090847157 | −0.003262643 | 662 |
| k_Bacteria\|p_Firmicutes\|c_Tissierellia\|o_Tissierellales\|f_Peptoniphilaceae | 0 | 0.016142733 | 663 |
| \|g Anaerococcus | | | |
| K09967 >> uncharacterized protein | −0.393188504 | −0.160686625 | 664 |
| K00395 >> adenylylsulfate reductase, subunit B [EC: 1.8.99.2] | −0.266350047 | −0.120153432 | 665 |
| k_Bacteria\|p_Bacteroidetes\|c_Bacteroidia\|o_Bacteroidales\|f_Prevotellaceae | 0 | 0.030824626 | 666 |
| \|g Prevotella\|s Prevotella nigrescens | | | |
| K07813 >> accessory gene regulator B | −0.209473936 | −0.03532766 | 667 |
| K02992 >> small subunit ribosomal protein S7 | 0.050807485 | −0.001631321 | 668 |
| K03605 >> hydrogenase maturation protease [EC: 3.4.23.—] | 0.285790437 | 0.112669929 | 669 |
| K03666 >> host factor-I protein | 0.204143507 | 0.032385403 | 670 |
| K05942 >> citrate (Re)-synthase [EC: 2.3.3.3] | −0.196634749 | −0.030701174 | 671 |
| K03565 >> regulatory protein | −0.054772323 | 0.001801802 | 672 |
| K00557 >> tRNA (uracil-5-)-methyltransferase [EC: 2.1.1.35] | 0.253740842 | 0.11150302 | 673 |
| K12339 >> S-sulfo-L-cysteine synthase (O-acetyl-L-serine-dependent) [EC: 2.5.1.144] | 0.257662611 | 0.04071543 | 674 |
| K00627 >> pyruvate dehydrogenase E2 component (dihydrolipoamide acetyltransferase) | 0.240153238 | 0.028514322 | 675 |
| [EC: 2.3.1.12] | | | |
| K05311 >> central glycolytic genes regulator | 0.229634706 | 0.102617462 | 676 |
| K01844 >> beta-lysine 5,6-aminomutase alpha subunit [EC: 5.4.3.3] | 0.334089291 | 0.222903164 | 677 |
| K01428 >> urease subunit alpha [EC: 3.5.1.5] | −0.124347984 | −0.092929471 | 678 |
| K07035 >> uncharacterized protein | −0.246827032 | −0.071119733 | 679 |
| K05831 >> [amino group carrier protein]-lysine/ornithine hydrolase [EC: 3.5.1.130 3.5.1.132] | 0 | 0.052810723 | 680 |
| K03796 >> Bax protein | 0.219267536 | 0.104128272 | 681 |
| K06142 >> outer membrane protein | 0.109651079 | 0.00599033 | 682 |
| K02404 >> flagellar biosynthesis_protein FlhF | −0.241125798 | −0.143812001 | 683 |
| K15024 >> putative phosphotransacetylase [EC: 2.3.1.8] | −0.030961177 | −0.055655982 | 684 |
| K03385 >> nitrite reductase (cytochrome c-552) [EC: 1.7.2.2] | 0.132527891 | 0.005649369 | 685 |
| K05807 >> outer membrane protein assembly factor BamD | 0.269115712 | 0.051670267 | 686 |
| K00700 >> 1,4-alpha-glucan branching enzyme [EC: 2.4.1.18] | −0.062433256 | 0.000340961 | 687 |
| K00024 >> malate dehydrogenase [EC: 1.1.1.37] | 0.087509433 | 0.000852402 | 688 |
| k_Bacteria\|p_Firmicutes\|c_Firmicutes_unclassified\|o_Firmicutes_unclassified | 0.041693552 | 0.060014991 | 689 |
| \|f_Firmicutes_unclassified\|g_Firmicutes_unclassified\|s_Firmicutes_bacterium_CAG_94 | | | |
| K00786 >> beta-1,6-galactosyltransferase [EC: 2.4.1.—] | −0.270297132 | −0.120194583 | 690 |
| K11050 >> multidrug/hemolysin transport system ATP-binding protein | −0.219741191 | −0.036788501 | 691 |
| K09797 >> uncharacterized protein | 0.065559006 | 0.009376424 | 692 |
| K03310 >> alanine or glycine: cation symporter, AGCS family | −0.068988876 | 0 | 693 |
| K09798 >> uncharacterized protein | 0.24523538 | 0.106732507 | 694 |
| k_Bacteria\|p_Firmicutes\|c_Clostridia\|o_Clostridiales | 0.029670003 | 0.082879944 | 695 |
| \|f_Clostridiales_Family_XIII_Incertae_Sedis | | | |
| K00302 >> sarcosine oxidase, subunit alpha [EC: 1.5.3.1] | 0 | 0.059603486 | 696 |
| K08307 >> membrane-bound lytic murein transglycosylase D [EC: 4.2.2.—] | 0.277147437 | 0.062992519 | 697 |
| K03735 >> ethanolamine ammonia-lyase large subunit [EC: 4.3.1.7] | 0.24020115 | 0.027541408 | 698 |
| K00163 >> pyruvate dehydrogenase E1 component [EC: 1.2.4.1] | 0.213376783 | 0.108604853 | 699 |
| K03388 >> heterodisulfide reductase subunit A2 [EC: 1.8.7.3 1.8.98.4 1.8.98.5 1.8.98.6] | 0.059647471 | −0.036982496 | 700 |
| K01058 >> phospholipase A1/A2 [EC: 3.1.1.32 3.1.1.4] | 0.277075517 | 0.092165248 | 701 |
| K01734 >> methylglyoxal synthase [EC: 4.2.3.3] | −0.029703545 | −0.001290361 | 702 |
| K07533 >> foldase protein PrsA [EC: 5.2.1.8] | −0.230654714 | −0.058945079 | 703 |
| K01649 >> 2-isopropylmalate synthase [EC: 2.3.3.13] | −0.062991624 | −0.00111988 | 704 |
| K07397 >> putative redox protein | 0.291523031 | 0.048654527 | 705 |
| K01163 >> uncharacterized protein | 0.109703668 | −0.002848199 | 706 |
| K06206 >> sugar fermentation stimulation protein A | −0.027911431 | −0.004820481 | 707 |
| K00266 >> glutamate synthase (NADPH) small chain [EC: 1.4.1.13] | −0.12888046 | 0.001972282 | 708 |
| K17810 >> D-aspartate ligase [EC: 6.3.1.12] | 0.0115255 | −0.013218112 | 709 |
| K02926 >> large subunit ribosomal protein L4 | −0.051777402 | 0 | 710 |
| K15549 >> membrane fusion protein, multidru\|g_efflux system | 0.164710064 | 0.126358332 | 711 |
| K07657 >> two-component system, OmpR family, phosphate regulon response regulator PhoB | 0.245556124 | 0.094775363 | 712 |
| K16936 >> thiosulfate dehydrogenase (quinone) small subunit [EC: 1.8.5.2] | 0.232026695 | 0.053184016 | 713 |
| K07738 >> transcriptional repressor NrdR | −0.073448255 | −0.002921682 | 714 |
| K02626 >> arginine decarboxylase [EC: 4.1.1.19] | 0.250453922 | 0.108904663 | 715 |
| K01462 >> peptide deformylase [EC: 3.5.1.88] | −0.058615372 | 0 | 716 |
| K01227 >> mannosyl-glycoprotein endo-beta-N-acetylglucosaminidase [EC: 3.2.1.96] | −0.26868367 | −0.131910704 | 717 |
| K09747 >> uncharacterized protein | −0.078531963 | −0.004723484 | 718 |
| K11933 >> NADH oxidoreductase Her [EC: 1.—.—.—] | 0.131300536 | 0.089763826 | 719 |
| K01258 >> tripeptide aminopeptidase [EC: 3.4.11.4] | 0.067257546 | −0.000778919 | 720 |
| K06411 >> dipicolinate synthase subunit B | −0.33902647 | −0.130840792 | 721 |
| K11474 >> GntR family transcriptional regulator, glc operon transcriptional activator | 0.168059419 | 0.105542084 | 722 |
| K01089 >> imidazoleglycerol-phosphate dehydratase/histidinol-phosphatase | 0.188364921 | 0.024252311 | 723 |
| [EC: 4.2.1.19 3.1.3.15] | | | |
| K01271 >> Xaa-Pro dipeptidase [EC: 3.4.13.9] | 0.29977843 | 0.124283174 | 724 |
| K03644 >> lipoyl synthase [EC: 2.8.1.8] | 0.118025847 | 0.011225255 | 725 |
| K03431 >> phosphoglucosamine mutase [EC: 5.4.2.10] | −0.078317826 | −0.004893964 | 726 |
| K01356 >> repressor LexA [EC: 3.4.21.88] | −0.098833227 | −0.004723484 | 727 |
| K01270 >> dipeptidase D [EC: 3.4.13.—] | 0.113750315 | 0.002045765 | 728 |
| K06378 >> stage II sporulation protein AA (anti-sigma F factor antagonist) | −0.173411043 | −0.035083697 | 729 |
| K00974 >> tRNA nucleotidyltransferase (CCA-adding enzyme) [EC: 2.7.7.72 3.1.3.— 3.1.4.—] | −0.063482833 | −0.011686728 | 730 |
| K02361 >> isochorismate synthase [EC: 5.4.4.2] | 0.325954366 | 0.074144291 | 731 |
| K06405 >> stage V sporulation protein AC | −0.131347573 | −0.006281322 | 732 |
| K06189 >> magnesium and cobalt transporter | 0.23406358 | 0.090636803 | 733 |
| K00940 >> nucleoside-diphosphate kinase [EC: 2.7.4.6] | 0.094242755 | 0.002824684 | 734 |
| K11145 >> ribonuclease III family protein [EC: 3.1.26.—] | −0.131925873 | −0.014096968 | 735 |
| k_Bacteria\|p_Bacteroidetes\|c_Bacteroidia\|o_Bacteroidales\|f_Porphyromonadaceae | 0 | 0.026101142 | 736 |
| \|g_Porphyromonas\|s_Porphyromonas_{—}endodontalis | | | |
| K07099 >> uncharacterized protein | 0.271180945 | 0.108431433 | 737 |
| K00853 >> L-ribulokinase [EC: 2.7.1.16] | 0.230788297 | 0.068594859 | 738 |
| K00761 >> uracil phosphoribosyltransferase [EC: 2.4.2.9] | −0.061499058 | 0 | 739 |
| K04744 >> LPS-assembly protein | 0.265260196 | 0.157570948 | 740 |
| K01712 >> urocanate hydratase [EC: 4.2.1.49] | 0.249092482 | 0.027150478 | 741 |
| K02536 >> UDP-3-O-[3-hydroxymyristoyl] glucosamine N-acyltransferase [EC: 2.3.1.191] | 0.114702602 | 0.00616081 | 742 |
| K01337 >> lysyl endopeptidase [EC: 3.4.21.50] | 0.040490322 | 0.103722646 | 743 |
| K06880 >> erythromycin esterase [EC: 3.1.1.—] | −0.308928361 | −0.188489632 | 744 |
| K03686 >> molecular chaperone DnaJ | −0.053233351 | 0 | 745 |
| K00609 >> aspartate carbamoyltransferase catalytic subunit [EC: 2.1.3.2] | 0.044498631 | 0 | 746 |
| K07054 >> uncharacterized protein | 0.282171106 | 0.143521009 | 747 |
| K05832 >> putative ABC transport system permease protein | −0.122431011 | −0.01253913 | 748 |
| K01580 >> glutamate decarboxylase [EC: 4.1.1.15] | 0.098028095 | 0.010522758 | 749 |
| K00325 >> H+-translocating NAD(P) transhydrogenase subunit beta [EC: 1.6.1.2 7.1.1.1] | 0.306666731 | 0.097914554 | 750 |
| K01007 >> pyruvate, water dikinase [EC: 2.7.9.2] | 0.242511921 | 0.116103052 | 751 |
| K05501 >> TetR/AcrR family transcriptional regulator | 0.26013108 | 0.086812751 | 752 |
| K15581 >> oligopeptide transport system permease protein | 0.000209917 | −0.00206928 | 753 |
| K01008 >> selenide, water dikinase [EC: 2.7.9.3] | −0.101454983 | −0.010737328 | 754 |
| K00690 >> sucrose phosphorylase [EC: 2.4.1.7] | −0.232819273 | −0.041511985 | 755 |
| K06145 >> LacI family transcriptional regulator, | 0.206892121 | 0.111917464 | 756 |
| gluconate utilization system Gnt-I transcriptional repressor | | | |
| K08303 >> putative protease [EC: 3.4.—.—] | −0.053909573 | 0.001801802 | 757 |
| K06183 >> 16S rRNA pseudouridine516 synthase [EC: 5.4.99.19] | −0.105748356 | −0.011345767 | 758 |
| K10986 >> galactosamine PTS system EIID component | 0.058037564 | 0.061034934 | 759 |
| K00760 >> hypoxanthine phosphoribosyltransferase [EC: 2.4.2.8] | 0.089997048 | 0.001972282 | 760 |
| K10016 >> histidine transport system permease protein | 0.241119953 | 0.112284879 | 761 |
| K11645 >> fructose-bisphosphate aldolase, class I [EC: 4.1.2.13] | 0.038837634 | −0.010225887 | 762 |
| K05601 >> hydroxylamine reductase [EC: 1.7.99.1] | −0.002720524 | 0.007377688 | 763 |
| K04487 >> cysteine desulfurase [EC: 2.8.1.7] | −0.111921078 | −0.0009494 | 764 |
| K06177 >> tRNA pseudouridine32 synthase/23S rRNA pseudouridine746 synthase | 0.211897993 | 0.041029937 | 765 |
| [EC: 5.4.99.28 5.4.99.29] | | | |
| K07001 >> NTE family protein | 0.10882883 | 0.002727687 | 766 |
| K03518 >> aerobic_carbon-monoxide dehydrogenase small subunit [EC: 1.2.5.3] | −0.210239328 | −0.047161354 | 767 |
| K03484 >> LacI family transcriptional regulator, sucrose operon repressor | −0.189851483 | −0.017797569 | 768 |
| K00639 >> glycine C-acetyltransferase [EC: 2.3.1.29] | 0.460511492 | 0.172664345 | 769 |
| K02008 >> cobalt/nickel transport system permease protein | −0.243998594 | −0.096656526 | 770 |
| K03815 >> xanthosine phosphorylase [EC: 2.4.2.—] | 0.1602779 | 0.076348779 | 771 |
| k_Bacteria\|p_Actinobacteria\|c_Coriobacteriia\|o_Eggerthellales | 0 | 0.013050571 | 772 |
| \|f_Eggerthellaceae\|g_Slackia\|s_Slackia_{—}exigua | | | |
| K14742 >> RNA threonylcarbamoyladenosine biosynthesis_protein TsaB | 0.306718943 | 0.075217142 | 773 |
| K03685 >> ribonuclease III [EC: 3.1.26.3] | −0.041644448 | 0 | 774 |
| k_Bacteria\|p_Bacteroidetes\|c_Bacteroidia\|o_Bacteroidales\|f_Porphyromonadaceae | 0.084969644 | 0.132331026 | 775 |
| K18138 >> multidrug efflux pump | 0.270629907 | 0.078747263 | 776 |
| k_Bacteria\|p_Firmicutes\|c_Tissierellia\|o_Tissierellales\|f_Peptoniphilaceae | 0 | 0.014511412 | 777 |
| \|g_Anaerococcus\|s_Anaerococcus_{—}vaginalis | | | |
| K03781 >> catalase [EC: 1.11.1.6] | 0.130191526 | 0.006672251 | 778 |
| K00340 >> NADH-quinone oxidoreductase subunit K [EC: 7.1.1.2] | 0.286984293 | 0.057566539 | 779 |
| K18140 >> TetR/AcrR family transcriptional regulator, acrEF/envCD operon repressor | 0.126466676 | 0.079173464 | 780 |
| K07112 >> uncharacterized protein | 0.234519119 | 0.047190747 | 781 |
| K03885 >> NADH dehydrogenase [EC: 1.6.99.3] | 0.216761329 | 0.046020899 | 782 |
| K03741 >> arsenate reductase (thioredoxin) [EC: 1.20.4.4] | −0.21240563 | −0.03761445 | 783 |
| K06333 >> spore coat protein JB | −0.223973007 | −0.055414958 | 784 |
| K14261 >> alanine-synthesizing transaminase [EC: 2.6.1.—] | 0.220815374 | 0.130349926 | 785 |
| K03813 >> molybdenum transport protein [EC: 2.4.2.—] | 0.141030336 | 0.079928869 | 786 |
| K03803 >> sigma-E factor negative regulatory protein RseC | 0.327766539 | 0.058098555 | 787 |
| K04773 >> protease IV [EC: 3.4.21.—] | 0.249515271 | 0.035527534 | 788 |
| K04030 >> ethanolamine utilization protein EutQ | 0.225782689 | 0.045147921 | 789 |
| K02566 >> NagD protein | 0.181396666 | 0.027785371 | 790 |
| K06894 >> alpha-2-macroglobulin | 0.287061603 | 0.04821363 | 791 |
| K03775 >> FKBP-type peptidyl-prolyl cis-trans isomerase SlyD [EC: 5.2.1.8] | 0.268800409 | 0.032970328 | 792 |
| K06409 >> stage V sporulation protein B | −0.079345948 | −0.011516247 | 793 |
| K17734 >> serine protease AprX [EC: 3.4.21.—] | −0.339303151 | −0.124950399 | 794 |
| K00763 >> nicotinate phosphoribosyltransferase [EC: 6.3.4.21] | −0.114647573 | −0.001387358 | 795 |
| K03599 >> stringent starvation protein A | 0.240872708 | 0.110480138 | 796 |
| K06396 >> stage III sporulation protein AG | −0.207841612 | −0.035742104 | 797 |
| K00784 >> ribonuclease Z [EC: 3.1.26.11] | −0.052731972 | 0.00017048 | 798 |
| K06383 >> stage II sporulation protein GA (sporulation sigma-E factor processing peptidase) | −0.196428497 | −0.04075658 | 799 |
| [EC: 3.4.23.—] | | | |
| K13771 >> Rrf2 family transcriptional regulator, nitric oxide-sensitive transcriptional repressor | 0.236220582 | 0.094825331 | 800 |

TABLE 6
Representative qPCR Primer Examples.

| | | | | Amplicon_ |
|---|---|---|---|---|
| Target | Species | PrimerF_Seq | PrimerR_Seq | Length |
| CRC | Streptococcus_salivarius | GCTTTCACGCTCAGTCCAGA | ATTCGAGCCCACTCGTTACC | 101 |
| | | (SEQ ID NO: 3) | (SEQ ID NO: 4) | |
| CRC | Fusobacterium_nucleatum | CAACTTGGTGAGAACGAGGTATC | TGCTGGTGGTAGAGGTATGG | 134 |
| | | (SEQ ID NO: 5) | (SEQ ID NO: 6) | |
| CRC | Parvimonas micra | TGGACCTGCTGCACTTTTAGT | GCTTCTGCAAAACACATCGC | 75 |
| | | (SEQ ID NO: 7) | (SEQ ID NO: 8) | |
| CRC. | Roseburia intestinalis | AGGGATTGTCTGTGTTGCCA | GATCAGTCCTTCCGCACGAT | 79 |
| CRA, | | (SEQ ID NO: 9) | (SEQ ID NO: 10) | |
| CRAA | | | | |
| CRC | Eubacterium ventriosum | GCATGCCCTCCAATGGTAGT | CCTAAGCCTCCCGGTCCTAT | 182 |
| | | (SEQ ID NO: 11) | (SEQ ID NO: 12) | |
| CRC | Clostridium symbiosum | GTGTTTTGTATGCGGGGCAG | GTAGGTGTGCCACAGGACAA | 118 |
| | | (SEQ ID NO: 13) | (SEQ ID NO: 14) | |
| CRC | Cloacibacillus evryensis | CGACATCGCCTGTTATTCGC | CGGTCATATCGGGCAGTACG | 137 |
| | | (SEQ ID NO: 15) | (SEQ ID NO: 16) | |
| CRC, | Dialister_pneumosintes | TTAGTGGTTCCTCGCGTAGC | CCGACACCCCATGTTGAAGT | 118 |
| CRA | | (SEQ ID NO: 17) | (SEQ ID NO: 18) | |
| CRC, | Gemella_morbillorum | ACGCAAGTGATGGAAAAGATGA | TCTCCACCAGAAGCCACTTT | 94 |
| CRA | | (SEQ ID NO: 19) | (SEQ ID NO: 20) | |
| CRC | Peptostreptococcus_stomatis | CCCCTGGCAAGACCAAACTA | TCACTAGACCTGTGGTCGGA | 136 |
| | | SEQ ID NO: 21) | (SEQ ID NO: 22) | |
| CRC | Bacteroides_stercoris | CTCTTTCAGGGTGGAGAGGC | CGGCAACTATCTTCCATGCC | 100 |
| | | (SEQ ID NO: 23) | (SEQ ID NO: 24) | |
| CRC | Butyricimonas_virosa | GGGAACAATGGAAGGGTGTG | ACGAACGCATCATCTCCTCT | 114 |
| | | (SEQ ID NO: 25) | (SEQ ID NO: 26) | |
| CRC | Collinsella_stercoris | TGGATATGAGCCGAGCGATT | TAGCTCGAAGGCGTGTCGTA | 198 |
| | | SEQ ID NO: 27) | (SEQ ID NO: 28) | |
| CRC | Faecalibacterium prausnitzii | GCGTCATGGTCGATTTTGAC | GGCATCCAGCAGCTTCCACT | 119 |
| | | (SEQ ID NO: 29) | (SEQ ID NO: 30) | |
| CRC | Intestinimonas | ACCAGATCATAACCGACGCC | CCTGGAGCGCACGATAGAAT | 162 |
| | butyriciproducens | (SEQ ID NO: 31) | (SEQ ID NO: 32) | |
| CRC | Veillonella parvula | CAGCCATTGCGGTATCATGG | AGAAACCAGCGAAGAAGTGGT | 117 |
| | | (SEQ ID NO: 33) | (SEQ ID NO: 34) | |
| CRA | Bifidobacterium_adolescentis | CAATCCTTGGGTATCAGCGCCA | TTATGCCGTGGCTTCCTTGG | 95 |
| | | (SEQ ID NO: 35) | (SEQ ID NO: 36) | |
| CRA | Bifidobacterium animalis | TACCAGGACCCGAATGCCTA | CATACTTGTTGCGCGGAAGG | 91 |
| | | (SEQ ID NO: 37) | (SEQ ID NO: 38) | |
| CRA | Bifidobacterium_ | CTGATGTCACTAGGGTGGCG | CGGTCAAGAGAAGCCAATGC | 137 |
| | pseudocatenulatum | SEQ ID NO: 39) | (SEQ ID NO: 40) | |
| CRA | Clostridium_spiroforme | TGTGGTAGCTGTGGTTATGCT | ACATAAGGAATCATCTGCTTCACC | 76 |
| | | (SEQ ID NO: 41) | SEQ ID NO: 42) | |
| CRA | Coprococcus_catus | GGGCAAGATTCACGGGTTTG | TATAAGCACTGCGGCCTTCC | 116 |
| | | (SEQ ID NO: 43) | (SEQ ID NO: 44) | |
| CRA | Dorea_formicigenerans | TACACTTCTTGGCGGAGAACC | AGGCTCTTCCACCTGGTATC | 152 |
| | | (SEQ ID NO: 45) | (SEQ ID NO: 46) | |
| CRA | Dorea_longicatena | CTGTTTTACGAAGGCGGCTG | CTGTACCTCTCCTGCTGCTG | 108 |
| | | (SEQ ID NO: 47) | (SEQ ID NO: 48) | |
| CRA | Escherichia_coli | CTCAAATCGTTAAGCGCCCG | TCCAGATGCGCTTTTACCGT | 119 |
| | | (SEQ ID NO: 49) | (SEQ ID NO: 50) | |
| CRA | Ruminococcus bicirculans | CATTTACGGCGGACATGGTG | CCGCTATGCTGTCCTTCAGA | 153 |
| | | (SEQ ID NO: 51) | (SEQ ID NO: 52) | |
| CRA | Streptococcus thermophilus | ATTTGGACGGACACCTCAGC | CGGAATCCCTGTCGCAATCT | 131 |
| | | (SEQ ID NO: 53) | (SEQ ID NO: 54) | |
| CRA | Alistipes shahii | TATATGTCTGTGGGCGCTGC | ATGATCCTGCCTTTGGGTGG | 108 |
| | | (SEQ ID NO: 55) | (SEQ ID NO: 56) | |
| CRA | Bacteroides cellulosilyticus | GCAGAGTGGTGTTGTCTTGC | TCTCGGCTACGGCTTTTTCT | 157 |
| | | SEQ ID NO: 57) | (SEQ ID NO: 58) | |
| CRA | Bacteroides nordii | TATCCCTGACTCCCGACTGG | TGCAATGGCAGAGGTCTCAA | 168 |
| | | (SEQ ID NO: 59) | (SEQ ID NO: 60) | |
| CRA | Bacteroides_salyersiae | AATTCAGCAGAAAGGCGGGA | GATTGCGATTGATGGCACCG | 126 |
| | | (SEQ ID NO: 61) | (SEQ ID NO: 62) | |
| CRA | Parabacteroides_goldsteinii | AAACAGGTGGGGAGATCGTG | TTCATAACCCACCCGAAGCG | 100 |
| | | (SEQ ID NO: 63) | (SEQ ID NO: 64) | |
| CRA | Gordonibacter pamelaeae | GCATGTACCGTTGAAGAGGG | CGGCCTGTTCCAACAAGAAT | 112 |
| | | (SEQ ID NO: 65) | SEQ ID NO: 66) | |
| CRA | Bacteroides_caccae | GTGGAAGCTACTGACGTGACA | TTGGCGGAGAGTATCTGTGG | 96 |
| | | (SEQ ID NO: 67) | (SEQ ID NO: 68) | |
| CRA | Gemella_sanguinis | ACGTTTTCGTTATGTGCGAGA | AACCAATTTTCCAAACCATTCTGTT | 108 |
| | | (SEQ ID NO: 69) | (SEQ ID NO: 70) | |
| CRAA | Actinomyces graevenitzii | GCTTTACATCGGTTCGGTCTC | TGCTGGCTATGGGTGCAAAT | 105 |
| | | (SEQ ID NO: 71) | (SEQ ID NO: 72) | |
| CRAA | Bacteroides thetaiotaomicron | ACGGTGCGTCTTACGGATAC | ACGGGTTTCGCTCTTATCGG | 90 |
| | | (SEQ ID NO: 73) | (SEQ ID NO: 74) | |
| CRAA | Bacteroides xylanisolvens | TTGTGGGGACAAGAAACGGT | ATGATCGGAACAAGCCTCCC | 75 |
| | | (SEQ ID NO: 75) | (SEQ ID NO: 76) | |
| CRAA | Flavonifractor plautii | TGCCACTTCTGTTTGCGGTA | AAAGACCAAAAGGGCCAGCA | 104 |
| | | (SEQ ID NO: 77) | (SEQ ID NO: 78) | |
| CRAA | Intestinibacter bartlettii | AAAACAGCATATGCAGGCCA | CCTCAACAGAACCAGTTTGTGC | 103 |
| | | (SEQ ID NO: 79) | (SEQ ID NO: 80) | |
| CRAA | Mogibacterium diversum | ACAACACAGGGCAGTTTGGA | GGTACTGAGTCTTACGGCCA | 138 |
| | | (SEQ ID NO: 81) | (SEQ ID NO: 82) | |

1. A method for evaluating a subject for the presence of a colorectal neoplasm, comprising:
quantifying genetic elements from a biological sample from the subject, wherein the abundance or prevalence of the genetic elements is associated with colorectal cancer (CRC), colorectal adenoma (CRA), or colorectal advanced adenoma (CRAA) to thereby prepare an abundance profile of the genetic elements, and wherein the genetic elements comprise elements associated with microbial taxonomic classification and/or genetic elements associated with one or more microbial gene functions;
evaluating the abundance profile for a signature indicating the presence of CRC, CRA, and/or CRAA in the subject, and
determining whether the subject is likely to have CRC, CRA, or CRAA.

2. The method of claim 1, wherein the subject is at low risk for CRC, CRA, CRAA, or colorectal polyps.

3. The method of claim 1 or 2, wherein the subject has no previous incidence of CRC, CRA, or CRAA.

4. The method of claim 2 or 3, wherein the method is performed as an alternative to colonoscopy.

5. The method of claim 1, wherein the subject is at high or medium risk for CRC, CRA, CRAA, or colorectal polyps.

6. The method of claim 5, wherein the subject has prior incidence of CRC, CRA, or CRAA, and/or family history of CRC.

7. The method of claim 5 or 6, wherein the method is performed at least once annually or at least every other year.

8. The method of any one of claims 1 to 7, wherein the subject is at least 45, or at least 50 years of age, or at least 55 years of age, or at least 60 years of age.

9. The method of any one of claims 1 to 7, wherein the subject is less than 45 years of age, or less than 50 years of age.

10. The method of any one of claims 1 to 9, wherein the biological sample is a fecal, blood, serum, intestinal mucosa, mucosal swab, colonoscopy aspirant, lavage, or biopsy tissue sample or other biological sample.

11. The method of any one of claims 1 to 10, wherein the genetic elements are quantified by a procedure comprising nucleic acid sequencing, PCR, qPCR, or microarray.

12. The method of claim 11, wherein the genetic elements are quantified by nucleic acid sequencing, and which involves sequencing at least about 20,000,000 reads.

13. The method of claim 12, wherein the nucleic acid sequencing involves sequencing at least about 40,000,000 reads.

14. The method of any one of claims 11 to 13, wherein the nucleic acid sequencing comprises one or more of shotgun metagenomic sequencing, rDNA sequencing, targeted amplicon nucleic acid sequencing, or hybridization capture probe sequencing.

15. The method of claim 14, wherein the nucleic acid sequencing comprises 16S rDNA, 18S rDNA, or ITS amplicon sequencing; and comprises targeted amplicon nucleic acid sequencing or hybridization capture probe sequencing.

16. The method of claim 14 or 15, wherein one or more genetic elements are quantified by capturing from a sequencing library, and optionally amplified by PCR, followed by sequencing.

17. The method of any one of claims 1 to 16, wherein the genetic elements are indicative of or correlated with colorectal adenoma (CRA).

18. The method of claim 17, wherein the genetic elements comprise at least five taxonomic or gene function features listed in Table 3.

19. The method of claim 18, wherein the genetic elements comprise at least about 10, at least about 25, at least about 50, or at least about 100 taxonomic or gene function features listed in Table 3.

20. The method of claim 19, wherein the genetic elements comprise at least one, at least two, at least five, at least about 10, at least about 20, or at least about 50 taxonomic features listed in Table 3; and at least one, at least two, at least five, at least about 10, at least about 20, or at least about 50 gene function features listed in Table 3.

21. The method of any one of claims 18 to 20, wherein the genetic elements have differential abundance or differential prevalence in samples from CRA subjects, as compared to control subjects.

22. The method of any one of claims 1 to 16, wherein the genetic elements are associated with colorectal advanced adenoma (CRAA).

23. The method of claim 22, wherein the genetic elements comprise at least five taxonomic or gene function features listed in Table 4.

24. The method of claim 23, wherein the genetic elements comprise at least about 10, at least about 25, at least about 50, or at least about 100 taxonomic or gene function features listed in Table 4.

25. The method of claim 24, wherein the genetic elements comprise at least one, at least two, at least five, at least about 10, at least about 20, or at least about 50 taxonomic features listed in Table 4; and at least one, at least two, at least five, at least about 10, at least about 20, or at least about 50 gene function features listed in Table 4.

26. The method of any one of claims 23 to 25, wherein the genetic elements have differential abundance or differential prevalence in samples from CRAA subjects, as compared to control subjects.

27. The method of any one of claims 1 to 16, wherein the genetic elements are associated with colorectal cancer (CRC).

28. The method of claim 27, wherein the genetic elements comprise at least five taxonomic or gene function features listed in Table 5.

29. The method of claim 28, wherein the genetic elements comprise at least about 10, at least about 25, at least about 50, or at least about 100 taxonomic or gene function features listed in Table 5.

30. The method of claim 28 or 29, wherein the genetic elements comprise at least one, at least two, at least five, at least about 10, at least about 20, or at least about 50 taxonomic features listed in Table 5; and at least one, at least two, at least five, at least about 10, at least about 20, or at least about 50 gene function features listed in Table 5.

31. The method of any one of claims 27 to 30, wherein the genetic elements have differential abundance or differential prevalence in samples from CRC subjects, as compared to control subjects.

32. The method of any one of claims 1 to 31, wherein the abundance profile is evaluated for signatures indicating the presence or absence of each of CRC, CRA, and CRAA.

33. The method of any one of claims 1 to 32, wherein:
(a) the signature indicating the presence or absence of CRA is trained with samples from a CRA cohort and samples from a control cohort by machine learning;
(b) the signature indicating the presence or absence of CRC is trained with samples from a CRC cohort and samples from a control cohort by machine learning; and
(c) the signature indicating the presence or absence of CRAA is trained with samples from a CRAA cohort and samples from a control cohort by machine learning.

34. The method of claim 33, wherein the signature(s) are trained using a plurality of machine learning algorithms.

35. The method of claim 33 or 34, wherein at least one machine learning algorithm is supervised machine learning.

36. The method of claim 35, wherein the machine learning algorithms further comprise one or more of unsupervised and semi-supervised machine learning.

37. The method of any one of claims 33 to 36, wherein the machine learning comprises one or more of parametric/non-parametric distance measures, logistic regression, support vector machines, decision trees, random forests, neural networks, probit regression, Fisher's linear discriminant, Naive Bayes classifier, perceptron, quadratic classifiers, kernel estimation, k-nearest neighbor, learning vector quantization, and principal components analysis.

38. The method of any one of claims 33 to 36, wherein the machine learning comprises comparative machine learning modeling, optimization, testing, evaluation, and ranking of models, including with one or more of deep learning, gradient boosted, neural networks, ensemble, or blender modeling algorithms, including one or more of Gradient Boosted Trees Classifier, eXtreme Gradient Boosted Trees Classifiers, Light Gradient Boosted Trees Classifiers, Light Gradient Boosting on Elastic Net Predictions, Keras Slim Residual Neural Network Classifiers, Generalized Additive Models, Elastic Net Classifiers, Random Forest Classifiers, Deep Forest Classifiers, Average Blender Classifiers, TensorFlow Multilayer Perceptron Classifiers, TensorFlow Neural Network Classifiers, and Rule-Fit Classifiers.

39. The method of any one of claims 33 to 38, wherein the signature(s) comprise features selected from training cohorts by ensemble ranking of feature importance and by statistical significance of individual features.

40. The method of claim 39, wherein the signature(s) comprise features selected from training cohorts by Feature Importance Rank Ensembling (FIRE) and by statistical inference of associations between microbial communities and phenotypes (SIAMCAT).

41. The method of any one of claims 33 to 40, wherein the signature(s) have a sensitivity for classifying samples for the presence or absence of CRA, CRAA, or CRC of at least about 0.70, or at least about 0.75, or at least about 0.80, or at least about 0.90, or at least about 0.95.

42. The method of any one of claims 33 to 41, wherein the signature(s) have a specificity for classifying samples for the presence or absence of CRA, CRAA, or CRC of at least about 0.70, or at least about 0.75, or at least about 0.80, or at least about 0.90, or at least about 0.95.

43. The method of any one of claims 1 to 42, wherein:
(a) if the subject is not identified as likely to have CRA, CRAA, or CRC, no further procedure is conducted, and
(b) if the subject is identified as likely having one or more of CRA, CRAA, or CRC, a further procedure or treatment is initiated.

44. The method of claim 43, wherein the further procedure comprises imaging of the colon.

45. The method of claim 44, wherein the procedure is a colonoscopy, which optionally involves removal of one or more polyps and/or biopsy of growths suspected of comprising CRC.

46. The method of claim 44 or 45, wherein if the subject is confirmed to have CRC, the subject is treated for CRC by one or more of surgery, chemotherapy, radiation therapy, and immunotherapy.

47. A method for preparing a genetic signature of genetic elements indicative of the presence of a colorectal neoplasm, the method comprising:
providing a training cohort of biological samples from subjects confirmed to have CRA, CRAA, or CRC, or RNA or DNA isolated therefrom;
conducting genomic nucleic acid sequencing of DNA isolated from the samples;
training a gene signature that classifies samples for the presence or absence of CRA, CRAA, or CRC, wherein the gene signature comprises microbial taxonomic classification features and microbial gene function features.

48. The method of claim 47, wherein the samples are selected from fecal, blood, serum, plasma, urine, saliva, biopsy tissues, mucosa tissue sample or swab, and intestinal lavage or aspirant.

49. The method of claim 48, wherein samples are fecal samples or mucosa tissue samples.

50. The method of any one of claims 47 to 49, wherein the nucleic acid sequencing involves sequencing at least about 20,000,000 reads per sample.

51. The method of claim 50, wherein the nucleic acid sequencing involves sequencing at least about 40,000,000 reads.

52. The method of any one of claims 47 to 51, wherein the nucleic acid sequencing comprises one or more of shotgun metagenomic sequencing, rDNA sequencing, and targeted nucleic acid sequencing.

53. The method of claim 52, wherein the nucleic acid sequencing comprises 16S rDNA sequencing and/or 18S rDNA sequencing and/or ITS sequencing; and one or more of shotgun sequencing and targeted nucleic acid sequencing.

54. The method of any one of claims 47 to 53, wherein genetic elements within the samples are amplified, optionally by PCR.

55. The method of any one of claims 47 to 54, wherein genetic elements are assigned to a reference genome for taxonomic classification, and/or genetic elements are assigned to a gene function.

56. The method of any one of claims 47 to 55, wherein microbial taxonomic classification features and microbial gene function features are selected that have a differential abundance or differential prevalence in samples from CRA subjects, as compared to control subjects.

57. The method of claim 56, wherein the features comprise at least five taxonomic and/or gene function features, and which are optionally listed in Table 3.

58. The method of claim 57, wherein the features comprise at least about 10, at least about 25, at least about 50, or at least about 100 taxonomic or gene function features, which are optionally listed in Table 3.

59. The method of claim 57 or 58, wherein the features comprise at least one, at least two, at least five, at least about 10, at least about 20, or at least about 50 taxonomic features that are optionally listed in Table 3; and at least one, at least two, at least five, at least about 10, at least about 20, or at least about 50 gene function features that are optionally listed in Table 3.

60. The method of any one of claims 47 to 55, wherein microbial taxonomic classification features and microbial gene function features are selected that have a differential abundance or differential prevalence in samples from CRAA subjects, as compared to control subjects.

61. The method of claim 60, wherein the features comprise at least five taxonomic and/or gene function features, and which are optionally listed in Table 4.

62. The method of claim 61, wherein the features comprise at least about 10, at least about 25, at least about 50, or at least about 100 taxonomic or gene function features, and which are optionally listed in Table 4.

63. The method of claim 61 or 62, wherein the features comprise at least one, at least two, at least five, at least about 10, at least about 20, or at least about 50 taxonomic features that are optionally listed in Table 4; and at least one, at least two, at least five, at least about 10, at least about 20, or at least about 50 gene function features that are optionally listed in Table 4.

64. The method of any one of claims 47 to 55, wherein microbial taxonomic classification features and microbial gene function features are selected that have a differential abundance or differential prevalence in samples from CRC subjects, as compared to control subjects.

65. The method of claim 64, wherein the features comprise at least five taxonomic and/or gene function features, and which are optionally listed in Table 5.

66. The method of claim 65, wherein the features comprise at least about 10, at least about 25, at least about 50, or at least about 100 taxonomic or gene function features, and which are optionally listed in Table 5.

67. The method of claim 65 or 66, wherein the features comprise at least one, at least two, at least five, at least about 10, at least about 20, or at least about 50 taxonomic features that are optionally listed in Table 5; and at least one, at least two, at least five, at least about 10, at least about 20, or at least about 50 gene function features that are optionally listed in Table 5.

68. The method of any one of claims 47 to 67, wherein at least three gene signatures are trained that:
(1) classify samples for the presence or absence of CRA,
(2) classify samples for the presence or absence of CRAA, and
(3) classify samples for the presence or absence of CRC.

69. The method of claim 68, wherein:
(a) the signature classifying samples for the presence or absence of CRA is trained with samples from a CRA cohort and samples from a control cohort by machine learning;
(b) the signature classifying samples for the presence or absence of CRC is trained with samples from a CRC cohort and samples from a control cohort by machine learning; and
(c) the signature classifying samples for the presence or absence of CRAA is trained with samples from a CRAA cohort and samples from a control cohort by machine learning.

70. The method of claim 69, wherein the signature(s) are trained using a plurality of machine learning algorithms.

71. The method of claim 69 or 70, wherein at least one machine learning algorithm is supervised machine learning.

72. The method of claim 71, wherein the machine learning algorithms further comprise one or more of unsupervised or semi-supervised machine learning.

73. The method of any one of claims 69 to 72, wherein the machine learning comprises one or more of parametric/non-parametric distance measures, logistic regression, support vector machines, decision trees, random forests, neural networks, probit regression, Fisher's linear discriminant, Naive Bayes classifier, perceptron, quadratic classifiers, kernel estimation, k-nearest neighbor, learning vector quantization, and principal components analysis.

74. The method of any one of claims 71 to 73, wherein the machine learning comprises comparative machine learning modeling, optimization, testing, evaluation, and ranking of models, including with one or more of deep learning, gradient boosted, neural networks, ensemble, or blender modeling algorithms, including one or more of Gradient Boosted Trees Classifier, eXtreme Gradient Boosted Trees Classifiers, Light Gradient Boosted Trees Classifiers, Light Gradient Boosting on Elastic Net Predictions, Keras Slim Residual Neural Network Classifiers, Generalized Additive Models, Elastic Net Classifiers, Random Forest Classifiers, Deep Forest Classifiers, Average Blender Classifiers, TensorFlow Multilayer Perceptron Classifiers, TensorFlow Neural Network Classifiers, and Rule-Fit Classifiers.

75. The method of any one of claims 69 to 74, wherein the signature(s) comprise features selected from training cohorts by ensemble ranking of feature importance and by statistical significance of individual features.

76. The method of claim 75, wherein the signature(s) comprise features selected from training cohorts by Feature Importance Rank Ensembling (FIRE) and by statistical inference of associations between microbial communities and phenotypes (SIAMCAT).

77. The method of any one of claims 47 to 76, wherein the signature(s) have a sensitivity for classifying samples for the presence or absence of CRA, CRAA, or CRC of at least about 0.70, or at least about 0.75, or at least about 0.80, or at least about 0.90, or at least about 0.95.

78. The method of any one of claims 47 to 77, wherein the signature(s) have a specificity for classifying samples for the presence or absence of CRA, CRAA, or CRC of at least about 0.70, or at least about 0.75, or at least about 0.80, or at least about 0.90, or at least about 0.95.

79. A method for preparing a genetic signature of genetic elements indicative of a colon disorder, the method comprising:
providing a training cohort of biological samples from subjects confirmed to have a colon disorder and control subjects, or DNA isolated therefrom;
conducting genomic nucleic acid sequencing of DNA isolated from the samples;
training a gene signature that classifies samples for (1) the presence of the colon disorder, and (2) for the absence of the colon disorder, wherein the gene signature comprises features selected from training cohorts by ensemble ranking of feature importance and by statistical significance of individual features.

80. The method of claim 79, wherein the biological sample is selected from fecal, blood, serum, plasma, urine, saliva, biopsy tissues, mucosa tissue sample or swab, and intestinal lavage or aspirant.

81. The method of claim 80, wherein samples are fecal samples or mucosa tissue samples.

82. The method of any one of claims 79 to 81, wherein the signature(s) comprise features selected from training cohorts by Feature Importance Rank Ensembling (FIRE) and by statistical inference of associations between microbial communities and phenotypes (SIAMCAT).

83. The method of any one of claims 79 to 82, wherein the colon disorder is selected from Crohn's disease, ulcerative colitis, irritable bowel syndrome (IBS), diverticulitis, colorectal adenoma (CRA), colorectal advanced adenoma (CRAA), and colorectal cancer (CRC).

84. The method of claims 79 to 83, wherein the features comprise microbial taxonomic classification features and microbial gene function features.

85. The method of any one of claims 79 to 84, wherein the nucleic acid sequencing involves sequencing at least about 20,000,000 reads per sample.

86. The method of claim 85, wherein the nucleic acid sequencing involves sequencing at least about 40,000,000 reads, or at least about 50,000,000 reads, or at least about 60,000,000 reads, or at least about 75,000,000 reads, or at least about 100,000,000 reads, or at least about 150,000,000 reads per sample.

87. The method of any one of claims 79 to 86, wherein the nucleic acid sequencing comprises one or more of shotgun metagenomic sequencing, 16S rDNA sequencing, and targeted nucleic acid sequencing.

88. The method of claim 87, wherein the nucleic acid sequencing comprises 16S rDNA sequencing and one or more of shotgun sequencing and targeted nucleic acid sequencing.

89. The method of any one of claims 79 to 88, wherein genetic elements within the samples are amplified, optionally by PCR.

90. The method of any one of claims 79 to 89, wherein genetic elements are assigned to a reference genome for taxonomic classification, and/or genetic elements are assigned to a gene function.

91. The method of any one of claims 79 to 90, wherein microbial taxonomic classification features and microbial gene function features are selected that have a differential abundance or differential prevalence in samples from colon disorder subjects, as compared to control subjects.

92. The method of claim 91, wherein the features comprise at least five taxonomic and/or gene function features.

93. The method of claim 92, wherein the features comprise at least about 10, at least about 25, at least about 50, or at least about 100 taxonomic or gene function features.

94. The method of claim 92 or 93, wherein the features comprise at least one, at least two, at least five, at least about 10, at least about 20, or at least about 50 taxonomic features; and at least one, at least two, at least five, at least about 10, at least about 20, or at least about 50 gene function features.

95. The method of any one of claims 79 to 94, wherein the signature(s) are trained using a plurality of machine learning algorithms.

96. The method of claim 95, wherein at least one machine learning algorithm is supervised machine learning.

97. The method of claim 96, wherein the machine learning algorithms further comprise one or more of unsupervised or semi-supervised machine learning.

98. The method of any one of claims 95 to 97, wherein the machine learning comprises one or more of parametric/non-parametric distance measures, logistic regression, support vector machines, decision trees, random forests, neural networks, probit regression, Fisher's linear discriminant, Naive Bayes classifier, perceptron, quadratic classifiers, kernel estimation, k-nearest neighbor, learning vector quantization, and principal components analysis.

99. The method of any one of claims 96 to 98, wherein the machine learning comprises comparative machine learning modeling, optimization, testing, evaluation, and ranking of models, including with one or more of deep learning, gradient boosted, neural networks, ensemble, or blender modeling algorithms, including one or more of Gradient Boosted Trees Classifier, eXtreme Gradient Boosted Trees Classifiers, Light Gradient Boosted Trees Classifiers, Light Gradient Boosting on Elastic Net Predictions, Keras Slim Residual Neural Network Classifiers, Generalized Additive Models, Elastic Net Classifiers, Random Forest Classifiers, Deep Forest Classifiers, Average Blender Classifiers, TensorFlow Multilayer Perceptron Classifiers, TensorFlow Neural Network Classifiers, and Rule-Fit Classifiers.

100. The method of any one of claims 79 to 99, wherein the signature(s) have a sensitivity for classifying samples for the presence or absence of the colon disorder of at least about 0.70, or at least about 0.75, or at least about 0.80, or at least about 0.90, or at least about 0.95.

101. The method of any one of claims 79 to 100, wherein the signature(s) have a specificity for classifying samples for the presence or absence of the colon disorder of at least about 0.70, or at least about 0.75, or at least about 0.80, or at least about 0.90, or at least about 0.95.


SEQUENCE LISTING (82 sequences)

SEQ ID NO: 1 - DNA, 22 bp, synthetic construct
  source 1..22: mol_type = other DNA
gcaggcctaacacatgcaagtc

SEQ ID NO: 2 - DNA, 20 bp, synthetic construct
  source 1..20: mol_type = other DNA
ctgctgcctcccgtaggagt

SEQ ID NO: 3 - DNA, 20 bp, Streptococcus salivarius
  source 1..20: mol_type = genomic DNA
gctttcacgctcagtccaga

SEQ ID NO: 4 - DNA, 20 bp, Streptococcus salivarius
  source 1..20: mol_type = genomic DNA
attcgagcccactcgttacc

SEQ ID NO: 5 - DNA, 23 bp, Fusobacterium nucleatum
  source 1..23: mol_type = genomic DNA
caacttggtgagaacgaggtatc

SEQ ID NO: 6 - DNA, 20 bp, Fusobacterium nucleatum
  source 1..20: mol_type = genomic DNA
tgctggtggtagaggtatgg

SEQ ID NO: 7 - DNA, 21 bp, Parvimonas micra
  source 1..21: mol_type = genomic DNA
tggacctgctgcacttttagt

SEQ ID NO: 8 - DNA, 20 bp, Parvimonas micra
  source 1..20: mol_type = genomic DNA
gcttctgcaaaacacatcgc

SEQ ID NO: 9 - DNA, 20 bp, Roseburia intestinalis
  source 1..20: mol_type = genomic DNA
agggattgtctgtgttgcca

SEQ ID NO: 10 - DNA, 20 bp, Roseburia intestinalis
  source 1..20: mol_type = genomic DNA
gatcagtccttccgcacgat

SEQ ID NO: 11 - DNA, 20 bp, Eubacterium ventriosum
  source 1..20: mol_type = genomic DNA
gcatgccctccaatggtagt

SEQ ID NO: 12 - DNA, 20 bp, Eubacterium ventriosum
  source 1..20: mol_type = genomic DNA
cctaagcctcccggtcctat

SEQ ID NO: 13 - DNA, 20 bp, Clostridium symbiosum
  source 1..20: mol_type = genomic DNA
gtgttttgtatgcggggcag

SEQ ID NO: 14 - DNA, 20 bp, Clostridium symbiosum
  source 1..20: mol_type = genomic DNA
gtaggtgtgccacaggacaa

SEQ ID NO: 15 - DNA, 20 bp, Cloacibacillus evryensis
  source 1..20: mol_type = genomic DNA
cgacatcgcctgttattcgc

SEQ ID NO: 16 - DNA, 20 bp, Cloacibacillus evryensis
  source 1..20: mol_type = genomic DNA
cggtcatatcgggcagtacg

SEQ ID NO: 17 - DNA, 20 bp, Dialister pneumosintes
  source 1..20: mol_type = genomic DNA
ttagtggttcctcgcgtagc

SEQ ID NO: 18 - DNA, 20 bp, Dialister pneumosintes
  source 1..20: mol_type = genomic DNA
ccgacaccccatgttgaagt

SEQ ID NO: 19 - DNA, 22 bp, Gemella morbillorum
  source 1..22: mol_type = genomic DNA
acgcaagtgatggaaaagatga

SEQ ID NO: 20 - DNA, 20 bp, Gemella morbillorum
  source 1..20: mol_type = genomic DNA
tctccaccagaagccacttt

SEQ ID NO: 21 - DNA, 20 bp, Peptostreptococcus stomatis
  source 1..20: mol_type = genomic DNA
cccctggcaagaccaaacta

SEQ ID NO: 22 - DNA, 20 bp, Peptostreptococcus stomatis
  source 1..20: mol_type = genomic DNA
tcactagacctgtggtcgga

SEQ ID NO: 23 - DNA, 20 bp, Bacteroides stercoris
  source 1..20: mol_type = genomic DNA
ctctttcagggtggagaggc

SEQ ID NO: 24 - DNA, 20 bp, Bacteroides stercoris
  source 1..20: mol_type = genomic DNA
cggcaactatcttccatgcc

SEQ ID NO: 25 - DNA, 20 bp, Butyricimonas virosa
  source 1..20: mol_type = genomic DNA
gggaacaatggaagggtgtg

SEQ ID NO: 26 - DNA, 20 bp, Butyricimonas virosa
  source 1..20: mol_type = genomic DNA
acgaacgcatcatctcctct

SEQ ID NO: 27 - DNA, 20 bp, Collinsella stercoris
  source 1..20: mol_type = genomic DNA
tggatatgagccgagcgatt

SEQ ID NO: 28 - DNA, 20 bp, Collinsella stercoris
  source 1..20: mol_type = genomic DNA
tagctcgaaggcgtgtcgta

SEQ ID NO: 29 - DNA, 20 bp, Faecalibacterium prausnitzii
  source 1..20: mol_type = genomic DNA
gcgtcatggtcgattttgac

SEQ ID NO: 30 - DNA, 20 bp, Faecalibacterium prausnitzii
  source 1..20: mol_type = genomic DNA
ggcatccagcagcttccact

SEQ ID NO: 31 - DNA, 20 bp, synthetic construct
  source 1..20: mol_type = other DNA
accagatcataaccgacgcc

SEQ ID NO: 32 - DNA, 20 bp, synthetic construct
  source 1..20: mol_type = other DNA
cctggagcgcacgatagaat

SEQ ID NO: 33 - DNA, 20 bp, Veillonella parvula
  source 1..20: mol_type = genomic DNA
cagccattgcggtatcatgg

SEQ ID NO: 34 - DNA, 21 bp, Veillonella parvula
  source 1..21: mol_type = genomic DNA
agaaaccagcgaagaagtggt

SEQ ID NO: 35 - DNA, 22 bp, Bifidobacterium adolescentis
  source 1..22: mol_type = genomic DNA
caatccttgggtatcagcgcca

SEQ ID NO: 36 - DNA, 20 bp, Bifidobacterium adolescentis
  source 1..20: mol_type = genomic DNA
ttatgccgtggcttccttgg

SEQ ID NO: 37 - DNA, 20 bp, Bifidobacterium animalis
  source 1..20: mol_type = genomic DNA
taccaggacccgaatgccta

SEQ ID NO: 38 - DNA, 20 bp, Bifidobacterium animalis
  source 1..20: mol_type = genomic DNA
catacttgttgcgcggaagg

SEQ ID NO: 39 - DNA, 20 bp, Bifidobacterium pseudocatenulatum
  source 1..20: mol_type = genomic DNA
ctgatgtcactagggtggcg

SEQ ID NO: 40 - DNA, 20 bp, Bifidobacterium pseudocatenulatum
  source 1..20: mol_type = genomic DNA
cggtcaagagaagccaatgc

SEQ ID NO: 41 - DNA, 21 bp, Clostridium spiroforme
  source 1..21: mol_type = genomic DNA
tgtggtagctgtggttatgct

SEQ ID NO: 42 - DNA, 24 bp, Clostridium spiroforme
  source 1..24: mol_type = genomic DNA
acataaggaatcatctgcttcacc

SEQ ID NO: 43 - DNA, 20 bp, Coprococcus catus
  source 1..20: mol_type = genomic DNA
gggcaagattcacgggtttg

SEQ ID NO: 44 - DNA, 20 bp, Coprococcus catus
  source 1..20: mol_type = genomic DNA
tataagcactgcggccttcc

SEQ ID NO: 45 - DNA, 21 bp, Dorea formicigenerans
  source 1..21: mol_type = genomic DNA
tacacttcttggcggagaacc

SEQ ID NO: 46 - DNA, 20 bp, Dorea formicigenerans
  source 1..20: mol_type = genomic DNA
aggctcttccacctggtatc

SEQ ID NO: 47 - DNA, 20 bp, Dorea longicatena
  source 1..20: mol_type = genomic DNA
ctgttttacgaaggcggctg

SEQ ID NO: 48 - DNA, 20 bp, Dorea longicatena
  source 1..20: mol_type = genomic DNA
ctgtacctctcctgctgctg

SEQ ID NO: 49 - DNA, 20 bp, Escherichia coli
  source 1..20: mol_type = genomic DNA
ctcaaatcgttaagcgcccg

SEQ ID NO: 50 - DNA, 20 bp, Escherichia coli
  source 1..20: mol_type = genomic DNA
tccagatgcgcttttaccgt

SEQ ID NO: 51 - DNA, 20 bp, synthetic construct
  source 1..20: mol_type = other DNA
catttacggcggacatggtg

SEQ ID NO: 52 - DNA, 20 bp, synthetic construct
  source 1..20: mol_type = other DNA
ccgctatgctgtccttcaga

SEQ ID NO: 53 - DNA, 20 bp, Streptococcus thermophilus
  source 1..20: mol_type = genomic DNA
atttggacggacacctcagc

SEQ ID NO: 54 - DNA, 20 bp, Streptococcus thermophilus
  source 1..20: mol_type = genomic DNA
cggaatccctgtcgcaatct

SEQ ID NO: 55 - DNA, 20 bp, Alistipes shahii
  source 1..20: mol_type = genomic DNA
tatatgtctgtgggcgctgc

SEQ ID NO: 56 - DNA, 20 bp, Alistipes shahii
  source 1..20: mol_type = genomic DNA
atgatcctgcctttgggtgg

SEQ ID NO: 57 - DNA, 20 bp, Bacteroides cellulosilyticus
  source 1..20: mol_type = genomic DNA
gcagagtggtgttgtcttgc

SEQ ID NO: 58 - DNA, 20 bp, Bacteroides cellulosilyticus
  source 1..20: mol_type = genomic DNA
tctcggctacggctttttct

SEQ ID NO: 59 - DNA, 20 bp, Bacteroides nordii
  source 1..20: mol_type = genomic DNA
tatccctgactcccgactgg

SEQ ID NO: 60 - DNA, 20 bp, Bacteroides nordii
  source 1..20: mol_type = genomic DNA
tgcaatggcagaggtctcaa

SEQ ID NO: 61 - DNA, 20 bp, Bacteroides salyersiae
  source 1..20: mol_type = genomic DNA
aattcagcagaaaggcggga

SEQ ID NO: 62 - DNA, 20 bp, Bacteroides salyersiae
  source 1..20: mol_type = genomic DNA
gattgcgattgatggcaccg

SEQ ID NO: 63 - DNA, 20 bp, Parabacteroides goldsteinii
  source 1..20: mol_type = genomic DNA
aaacaggtggggagatcgtg

SEQ ID NO: 64 - DNA, 20 bp, Parabacteroides goldsteinii
  source 1..20: mol_type = genomic DNA
ttcataacccacccgaagcg

SEQ ID NO: 65 - DNA, 20 bp, Gordonibacter pamelaeae
  source 1..20: mol_type = genomic DNA
gcatgtaccgttgaagaggg

SEQ ID NO: 66 - DNA, 20 bp, Gordonibacter pamelaeae
  source 1..20: mol_type = genomic DNA
cggcctgttccaacaagaat

SEQ ID NO: 67 - DNA, 21 bp, Bacteroides caccae
  source 1..21: mol_type = genomic DNA
gtggaagctactgacgtgaca

SEQ ID NO: 68 - DNA, 20 bp, Bacteroides caccae
  source 1..20: mol_type = genomic DNA
ttggcggagagtatctgtgg

SEQ ID NO: 69 - DNA, 21 bp, Gemella sanguinis
  source 1..21: mol_type = genomic DNA
acgttttcgttatgtgcgaga

SEQ ID NO: 70 - DNA, 25 bp, Gemella sanguinis
  source 1..25: mol_type = genomic DNA
aaccaattttccaaaccattctgtt

SEQ ID NO: 71 - DNA, 21 bp, Actinomyces graevenitzii
  source 1..21: mol_type = genomic DNA
gctttacatcggttcggtctc

SEQ ID NO: 72 - DNA, 20 bp, Actinomyces graevenitzii
  source 1..20: mol_type = genomic DNA
tgctggctatgggtgcaaat

SEQ ID NO: 73 - DNA, 20 bp, Bacteroides thetaiotaomicron
  source 1..20: mol_type = genomic DNA
acggtgcgtcttacggatac

SEQ ID NO: 74 - DNA, 20 bp, Bacteroides thetaiotaomicron
  source 1..20: mol_type = genomic DNA
acgggtttcgctcttatcgg

SEQ ID NO: 75 - DNA, 20 bp, Bacteroides xylanisolvens
  source 1..20: mol_type = genomic DNA
ttgtggggacaagaaacggt

SEQ ID NO: 76 - DNA, 20 bp, Bacteroides xylanisolvens
  source 1..20: mol_type = genomic DNA
atgatcggaacaagcctccc

SEQ ID NO: 77 - DNA, 20 bp, Flavonifractor plautii
  source 1..20: mol_type = genomic DNA
tgccacttctgtttgcggta

SEQ ID NO: 78 - DNA, 20 bp, Flavonifractor plautii
  source 1..20: mol_type = genomic DNA
aaagaccaaaagggccagca

SEQ ID NO: 79 - DNA, 20 bp, synthetic construct
  source 1..20: mol_type = other DNA
aaaacagcatatgcaggcca

SEQ ID NO: 80 - DNA, 22 bp, synthetic construct
  source 1..22: mol_type = other DNA
cctcaacagaaccagtttgtgc

SEQ ID NO: 81 - DNA, 20 bp, Mogibacterium diversum
  source 1..20: mol_type = genomic DNA
acaacacagggcagtttgga

SEQ ID NO: 82 - DNA, 20 bp, Mogibacterium diversum
  source 1..20: mol_type = genomic DNA
ggtactgagtcttacggcca